SEQUENCE LISTING (187 sequences)

SEQ ID NO: 1 - DNA, 3006 bp, H. sapiens
agtcggcggcggctgctgctgcctgtggcccgggcggctgggagaagcggagtgttggtgagtgacgcggcggaggtgtagtttgacgcggtgtgttacgtgggggagagaataaaactccagcgagatccgggccgtgaacgaaagcagtgacggaggagcttgtaccaccggtaactaaatgaccatggaatctggagccgagaaccagcagagtggagatgcagctgtaacagaagctgaaaaccaacaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagtcttcctgtaaggacttaaaaagacttttctccggaacacagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacctgccatcaccactgtaacggtgccaactccaatttaccaaactagcagtggacagtatattgccattacccagggaggagcaatacagctggctaacaatggtaccgatggggtacagggcctgcaaacattaaccatgaccaatgcagcagccactcagccgggtactaccattctacagtatgcacagaccactgatggacagcagatcttagtgcccagcaaccaagttgttgttcaagctgcctctggagacgtacaaacataccagattcgcacagcacccactagcactattgcccctggagttgttatggcatcctccccagcacttcctacacagcctgctgaagaagcagcacgaaagagagaggtccgtctaatgaagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatcagattaatttgggatttaaattttcacctgttaaggtggaaaatggactggcttggccacaacctgaaagacaaaataaacattttattttctaaacatttctttttttctatgcgcaaaactgcctgaaagcaactacagaatttcattcatttgtgcttttgcattaaactgtgaatgttccaacacctgcctccacttctcccctcaagaaattttcaacgccaggaatcatgaagagacttctgcttttcaacccccaccctcctcaagaagtaataatttgtttacttgtaaattgatgggagaaatgaggaaaagaaaatctttttaaaaatgatttcaaggtttgtgctgagctccttgattgccttagggacagaattaccccagcctcttgagctgaagtaatgtgtgggccgcatgcataaagtaagtaaggtgcaatgaagaagtgttgattgccaaattgacatgttgtcacattctcattgtgaattatgtaaagttgttaagagacataccctctaaaaaagaactttagcatggtattgaaggaattagaaatgaatttggagtgctttttatgtatgttgtcttcttcaatactgaaaatttgtccttggttcttaaaagcattctgtactaatacagctcttccatagggcagttgttgcttcttaattcagttctgtatgtgttcaacatttttgaatacattaaaagaagtaaccaactgaacgacaaagcatggtatttgaattttaaattaaagcaaagtaaataaaagtacaaagcatattttagttagtactaaattcttagtaaaatgctgatcagtaaaccaatcccttgagttatataacaagatttttaaataaatgttattgtcctcaccttcaaaaatatttatattgtcactcatttacgtaaaaagatatttctaatttactgttgcccattgcacttacataccaccaccaagaaagccttcaagatgtcaaataaagcaaagtgatatatatttgtttatgaaatgttacatgtagaaaaatactgattttaaatattttccatattaacaatttaacagagaatctctagtgaattttttaaatgaaagaagttgtaaggatataaaaagtacagtgttagatgtgcacaaggaaagttattttcagacatatttgaatgactgctgtactgcaatatttggattgtcattcttacaaaacatttttttgttctcttgtaaaaagagtagttattagttctgctttagctttccaatatgctgtatagcctttgtcattttataattttaattcctgattaaaacagtctgtatttgtgtatatcatacattgttttcaataccacttttaattgttactcattttattcactaagctcgataaatctaacagttactcttaaaaaaaaaaaaaagactaaggtggattttaaaaattggaaactgacataatgttaggttataatttctcatttggagccgggcgcagtggctcacgcctgtaatcccagcactttgggaggccaaggtgggtggatcacctgtggtcaagagttcaagaccagcctggccatcatggtgaaaccccatctctactaaaaatacaaaaattagccaggcgtggtggctggcgcctgtaatcccagctactcaggaggttgaggcagcagaattgcttgaacccaggaggcagagggttgcagtgagccgagatagcaccattgcactccagcctgggcgactccatctcaaaaaataaaaaaaaaaaaaaa

SEQ ID NO: 2 - DNA, 68001 bp, Rattus norvegicus
  misc_feature (1)...(68001): n = A,T,C or G
caaactggcaaacagactggagtgaggcggggacaggtttagaggactccaagccagagatgaaggcagcttaagagtggtggagaacgcatgtggacacagcgaacgccaccagcatttcaggaagcagacaatctaggggatactaactggtatcagaagtgggctcggtttctagcgtatctgaatagacgttctttactgagcgaaggagcgtgggaggaagggtaggaggggatatgaaaactcaaggtgagtaggactccatactctcatcatccagccacatggaccagaggctgctgggaagaggactttagagtctgagggtgaggcctaagactggagagtctgggagaaggcatttgaagcaagtagaagaggatgagaccacttaggggaagagactacggtaggattgtggaggatcagggctcagacgattccagcacaggagttagagaaaggagaatctagttgttggggtttagggttaaaaggaagactggcaagagaagaggggagagtgtgtttggaagagtcaaacagcatcaaatgctctaagatggacttgtaaatgcaagacgcaaaactacacagacaagaatgtgacagttcagagccactgcagatggagagggatgacaagagacgggtggaacttggagaggggcttaaggaggaagtttctctattttatgtattagccacacacactgcttactgtgatgtaagtgctgaggtcaaagggcaacttgtgggagctggttctctccttctaccacgtggattctggcagttgaccttagattatctggcttggtggcaagggcctttatccttctcaccagtgctacagaagtctttttaaagagaggaagaggcattagcatgctaacggttctggttagaaacaaaaggaaagtcaaactactcttcagtgcaatgtccgtgggaaagcaggaacagataggacacacagaatgaaggcaaactcagtgttacaaaaacgtcaataaaggccggagtccaacaggaacaaaactataggaagagggttgaagagctggtggttaggggcgactcagagagcggttgcggtagatttggggctacggaagtcacagtcaagttcgagggtgcaaaaggtggaacctgcgccgctgcagcaagggccgagaaaaggcaacacaggatgtggcgggcaggtggagtgagggtatatttcaagtgttaacagcaccatgggccagagttgatcatttctctggcttcagagggaagacaacgtacccaggggctatctccaaagtagagctgaatcccatgtgccacgtgtgccatagctttcttcccagatagaaccagcttggccccgtgctgatagttctcggcatattcccctggccggcggccggcgtttgcaaccttccaacgccgtccgcagggttctgggcccaggcggcgcgtcaaagttcccgggcggcctgcttccgctgcaggatcagaccgcgcgggggtttccagcaagtccccgccaccccggggaatccccccatctggccacacacaaaccccgcttcctccaaatctcctcagcgggcgttcgaagcctccacccgccatagcgaggataggaccgttcctccagaacgggtcttaatccgccttctcccgccttccccaaccaaccccttcgcagtgcccggaagaagccgaaggctttcggtaggttccgcaagtagcgcgggggcggggcctctcgagcggctccgggtcgagctcggctgtttccgtgagtggccgctgcgcactcggcactgggcggcgctggctggctccctggctgcggctcctcagtcggcggcggctgctgctgcctgtggcccgggcggctgggagaagccgagtgttggtgagtgacgcggcggaggtgtagtttgacgcggtgtgttacgtgggggagagaataaaactccagcgagatccgggccgcgaacgaaagcagtgacggaggagcttgtaccaccggtaagaggagcaggaggaggaggcaggagccggagagagccggggggacggaggggggccggggaggcgccggacacccgcgctcggggccttcccctcgcaggaggggccgcggtaaagatggagcctccgcccgagccccactacaccgccgccgctcgcagcgaacaaagaataatggcggcggagtggagccagcggcggccgggcggcagcgactctgcctgcagccagcggcggcgctcatggcggggggcggccggggcggggtgcgagcgagnnnnnnnnnnnnnnnnnnnnnnnnnnnnnnnnnnnnnnnnnnnnnnnnnnagcggagccgccccggtggaggccgcccgccgccgccccccgttcctcgccgcagccgcccgcctgctcgctcgctcgctcgctctggttgtgccgggctggccgcccgggccgccgcctgggtgcccgtggggggtggaggaggaggaggaagaagaaggagacagttgagggcagaggaggtgaaggtgctgccgccttcgcggcggtgcccgctcggcgagccgcaacgtccgacctgcttttctctccgctctttgtgcaataaagttcgaaacccaaggttttctctggtgctgtattttattcttgcccctctcccttgctcccacgcggttggagaaaatgctgttttctgccctaggctgttttaagggtttgggggggggttgtttgtttttgtttttttactattttcctcattcctcggttttgaagatgaagaacgactcagtagacatgggaatgcattcttttgggaaggaaagaggtgctaagaaaacatttgtaataactttttgcagaaactgttgggatcttgtaaaagggaagctggtgtttgaaaacagtttcggttttaaccctttccaataagataataatcacttgaggtcaatcattctgaaagccttcttagaaacgaatattagcggagatacttttcgttttatatcatttgaggggaggcagccttgccaagcttttttgagtcatccggatctcctcctagccttgctcactcactaccatgcttctcacacgcttctgtctgaactttaatggaggattgataaaattcgcactctgaaaatagtcatcgaaactatccattatctgtttattgcagtgggtaatcccagtctgaggttgatggcagtttaagctagcacaggttttgttccttttttcccttcacctagaacatctgcaaaggggactgtcaaaagtcattgacaccttaaagatcaaaagacacttttaaagggcattgcagtctgacctatttaaacctgcattatcttctcaaagttaagagtttgatcctagcccagtttacatcacatctgccttctaaagatctaacgtaagatacctttgggccatctattgaaatcttgtaccttcatgatccatttcaatttcccaattgaaaagcaaagccccaaattttagttgatttgaaaggtgcacagtttctttctttttttttaaacaaccagttactgtacaattgaatcgtgatcggtccatacagagcttttgattaggaataaatattgattggtaatagaccattgtgctcgatttttctgtttcaggactcaaacttaatggttactttgctagcctttagcacatcaatttgccattgcaaagaaaattaaggttcaatagaaggaatggttccataggattggggagatgactttatttccacacagtgaagaggttatacatggtaatgatgatgataaaatagttttttcaaaagatttcctacaagccaatagaggtgatacatgaagatgattcttcccatgtgagaataaaaagaatttttccttatgtgtcctttcatcagtgttgaccaaaagatattttatttatgggcttgcttttttttttttttttttttttttttttttttttttttggagaatcaaattgattctcagcaactgcaagtgaaactcggagcactttttgtgttcatttacaagcgttcacctggaacccggcactaaataattctctcttgcagaggtcagtgaaagttatggcaccatgtgctttcctcctcttccttctttcttttagttattcattttttttgatggacctgtgcatttagaaagctaatgaattatttaaaataactcacctaatggatactaaatgagcgtttcttttggagtttaaaactaatattaaacacttttgaacagaatttcacacaagggcccgaagaaacagacctaatccataataatctttttttctactttcagacacccatttatcaggtctgaaattagagattttcgttttcttagagaagagaagtagttttgtcactttcaagcccttgatcttatttgttagtatgttttcttgaaacattgttcacttgtgacagtgcttacactatgtcagtaaaaagtaaaatgttaattataaacagataggttggcagataacagccatataaactcccctgttatacattcctgccaacatctttcttgctgtattacttccagtggtagcctcattattttgaataggtagagctcaaatcttgagcttaagcatataatagataatcagaaaatagataattacagcagtttattcctagtgcatgtgtgaacttgactgccctgctgaacacttgacagtagtcactggtgtatgaatatttaacacttacagtgataggcactacagtgaaagagcatctgtctagtttgggaaatcacgagtgtgggtttttttttttttttttttttttttggttctttttttttcggagctggggaccgaacccagggccttgcgcttcctaggtaagcgctctaccactgagctaaatccccagccccacgagtgtgggtttgaatcctaacactgccagtcctgtcacatgcctgcacttggggaattttgtgatcatgggatactgtctgctttctctgcctcaatttcttcatttgtaaagttaaaatagtagcatatactcctggaagttttgagataagtagaaagcacttttcctaaggtgggtttaatgtcatattagcttttaccttttaccatttgggcttcctacaggtgtcataatttctccaaacgttgtgtttaatatagttggccagaaagaccaacaaatccaatgaggaattttactatctttatgatcagggaaccattgggatcagatatttgtcattagaaaacaatcaaaggaaagagaacgctctcagtgagctgaaaagtagtctttgtagtttcagttttgtttaattattcagatggttataatacaaaacaaggtatagcaaaatatcctatattctgtgctatccagtgttgcccacgtgttttccgagatgctctgtccataaacaagtaaacatatatgtgtatctccctctttataccaataatagcatgctctcctcccttacctgtttgacactcatttaccaggctacatataatgactcagcagttggtttgtataagcatgtaaagacctttttaaaaagatctttaaattgtatgtgtatatgtgtatgtttgaatgtatgaatgggtatagtgtctgtataggagcccaagatgatcagaagaggacatcagatcccctggaaccacagttgtgagccagcatgtggatgttggaactaaacatgggtcctctgcaagagcactataatcttaaccactggactgtctctccaacagtagagaagcatgtacatagggatatacttaaatttattaaccttttagtaagctgattctatttctaggtcatgcacgaccttttaaattgtaatgctaccaagcttggtattagttcatgcctttaatcccagcacttggggcaaaagctactggatctctgaattcaaggccagctttgtctgtaaagtaagttccagcaagggctgcatcctgaaacatggtctcaaacacaaacattatttaagtaacactcatttaagtgaaaagtaatggagtagtttatgagaggaaattgaggtttaatttaaactgctttatttttagctccactttataaaagctttagttaaaagattaagatgtatatagggacaagcaagctatgaatttagactgacattgcttcattaaaatttttaccatcctgaactttcacttaactattttgaccaaaaaagtattggaaattttctctgatggtttaaaaaaaatgtttaatttagttcagtgttttgttttgtttgaaatggagaatggttcttttctgtttattttggtggtttgaggagggtcttgtgtagtctagactatcctagaactcatgtagcttaggctggcctcagacttgaagtcattctgccttagcgtgtagccttttatgtgctggtattacagtattttttaaaaaacatttcttggtgtaattaatcagtgaattttgtatttcttgcttaaataggaaacaagtagattgacgcacatctgtctcctgggaatcacactcacactgtactgcttttttaattgttgaaagcccttagtcagctggactatgtaaaatgcatttcaaccccagcacataacaagtctcagtagctctttgtttccttcttggctctttagagcagcttaaagatatagtaggttttaagtatacatattacagattttctaaacaatgtgtcccactgttttcttttagttattaaataaagctgtcatgatggagttatttgtatggttaaaggcttaagctgagaatgacagtgttgtgggtcatgcaggctacagttttacaagactacttttaccgttatttgaaaggttttcttagtattataaaatattaacatgctacttttacctgctgcataattaaggcgtgcacaaagtgtttgtatggatttggtaagaatagggtagatgattgacgtttagaaaggcaggtgactggagggtatctgcaagctctcagtgaggtagatggtttcttggtgtctttaggtcttccttcagtttaatatttctgtttgaatcattggtttttgttttgttttgttttgttttttatcccagactgggcttctctgtgtagccctaactgtcctgaaacttgtgctgtatatagaacaggctggccttgaactcataggttcacttgtttctgcttcccagatgttgggattaaaggcatgactgataatagaataactgtttaatacacacagatgctgagtttattttttcaaataaagtaaatgtaaacttactttatctataaatgcaggttttaaagtttgaatgaggatagccattttgttttccccatcactcaaaatgtattttttcccttaatttgagcacatgcttgtctcaggttctgattttgtttagcttatgttctcttttcttaattcttccagagaagttcatttaaaatatattttattattattttatctagatttttgattgtctagtgaagtacctaaactgtctttcatagtctccttcattttaggagataaacttacctataatttctgcacgtattagatagtctagccatggaaaattgtccagaactaagtacaattcttttctttttctttttctttcttttttttttttttttccggagctggggaccgaacccagggccttgcgcttactaggcaaacgctctaccactgagctaaatcccctaagtactcaaccttgtaagtactcaattcttgaggctatttacacaaaaatcagagcagtagttgggactggtcatgtgacttagtgagcgcacttgcctattgtgaacgaggcaggccctgggtttatttaaatagatcaaagcaaggtttgtcgaggaggtgatctaagggattaatagagaatttttttttctaaaaggagaaaagatacttagttcaagaagagtgagctgagtgggaagataaaatagataaaggtgtatcttaatgctgactaataccagtagtgatggacatttctttcccaagcatcaatgggtcccttttgaaatgtaagatttagtccatcagttttagttgtaccctaacaacctgattatttgcatgtgagaatctgattgtacttatgatatatatgtaacaaacaatgtttaattcagccagcatataatgaaacattttccagaaatttatttctgcttgttttagcagttgacaaaaagacttgaaagtctgttatttttcatttccaaagtgataatttttgcttatttgaagcatttttattcttcttagggtttttcttttttgcgtgattttgtttgttggttttgagagagaaatcttcctttgtagcttggctgtcctggacactttgtggactaggttgaccttagactcaagatatctacctacttctgtgctggagttaaaagtggtatcattttcttgaatcaatagaagcctgtagtttgctgcaaataataatagtaatatcctgctgttgtgtgttgctgtttgtcaacctaatctgttcatggactttgatgatcatcaattcatagttggtagaacaatcacaagaaaatttattcaggaagttaagaaaataattcctggataattgaggatgtttagatgctataagtaaaaaagtcccagtatgtctcctacccataaagaatttgcagtacggtactgtagggtgtggggatgcaaggataagtctctgctatcaggaagagctatagagtttagaaaagagttcacacgggaaatcttgatgggcaggtagcatttgaatggaatgttgaaaattttgtataggatacaaaaggataaccaaaggaggacactgaagtagaatgaacttgatatatacacaaattggtgaagagaatgttagattaactacagattgacattttaaggaatcctgaatgtgaatccacatggcacaagtttaagttttagataagaagcaagtgtgtgtgtatgtgtgtgtctgtgtattatacatatatatgtatgtgaacatatacaatgactgtcttaaaattaatttttttttacttccagtgtgttgtactatcaaggacaccatagcttctctgtcaacttttttgaaatgttatatataatcttcccgtttggtaggtacagttccatgatttttagtgttgttagccaattgtgcatctactgtgacagttagccctaggacacattcaccaccttagaaagaaaccattaccccttaattttcccacacagcatcccaagacaactgctaatctgctttgtgctttattaatttgtttttgaacatttcatatgaataaaagcatacaatgtattatcatggtgaacttcttagcataatgttttcaaagttcatctatgtaatatctattagtattaatttttgttgccaaatagtagtccatattgtactcagtcttttactcattagttcatgaacacagttctctgactgagttatgctatgtaaccattgtcattctctggtataggtaggtatccagggaagcgttgggttgtgtgctgaccctgttacagctgttttctaagctactgcgcatattttatgagcagtgtacaagggtcttaatgtctccatgcccccgctaacactgctgtcatctgtctttgccattatatctgtgatagtcacagtggttttgatttgtattttcctagtggtatatatctttgaaaattatatttatttagataactttgctttaccaggtacatgatttatatttttctcccattttatctattgttatttcattttttgtttcgttttgtttttttttttccagacaagttctcaccatatagctctggctggccttgaacttgctctgtagactatgcttgcctcaaacttacagagatctacctgcctccacctcccaagttctaggattaaaagtgtgtaccaccacacttgagccataaaatttttcatttttatttctagttcatcaatgttatttttcttctattgcttgtacctttagatctttttctaaatgtcacatttttcccaagtaactgaatgcccagcaaacaacattgcttaatgaaaaacagctagctgaatgggtgcaggactatgttctactttcttctgtttttctccattaatctgtgtccttatgccttttttttggagtgacttatataagaaaatgaagatgaaaaaccaagaagtggcaattttgaacataacataataattggctaaattttttggtaaatttatgtcagagtaggtatagtgtattttaaactacttggagcgtgcgcgcgcacgcacgcgcacacacacacacacacacacacacacacacacatacacacacacatacctttgtttaaagaagacgacactggccctgtggagtgtaaatgatgtactcactgtcataggagagtgcttgtttttgtttttgtttttgtttttttgttgttgttgcattatctctgtggaaaaagttttgaaagctcttgttttattaataactccgaaagtttgaccctaagcagtatcaactttagtttataaatagctcacttgaagtaaatattctagtatagatcatggaaaatgcattaatttccttattttattaatcagctctttatcaggagagaggtcatgcccccaacccccactttcttgtgtcccagtcatttccaaatggatgcttttcaacaacttaccaaatagtaatggttgtttagagggaatatttataggtacccaggtcataactattacattatactcgtctcttttgatacagtgtttcatctctaactaggttgttagctctgggagaggctgttcttatgtgttttaatctgttacttcctggacgtccattgtgcaggaaacacagaacatttctttttagggccataattttgaaagaaaggattagcagtattcttttagagttatacagattagaggaagcagacaaaacatgtttactgtacttgttgatttttctttccctgagacaggctttctctgtgtagttctggccttgaactcagagatgagtctgcctctgcctcccatgtgctgggatttaggtgcgagacaccaggcccagcttaattacgggttttgtgttttgtttatttttttttaagataaagaggaagtttatttttgtagcttaagtcttttgttctttggattttcattaatccttatgctttccacagtggtatcttcctctaaagcctaagcatcatgctgagattgtgttgatcaaaataacttcttgagaaatcaagtgtcatggtagtaagaagtatgcctccctgaaggttggacttgatgttgattatttcaagaccaaagataaaaacactgaacttaaaataagtcttatatcaaaactatacataaagaaaaatatttggtctgatactataatagtggatgcatatcattatatatttgtcctgattcagtgtgtaccaccaagaataaaccatgatgtcaaaaataaatacaataaaaatgcataaagtttgtatcattgtttacatgctatttatcagcataccagatagaatatccatggtctgtttaaatattgtctgctatatcttacgtttttttcatttcctgactgactggtaagcaacatattaaaatgacttaaatatttctttctattctgtagagcttcattgttgactaaacagatagttctctactttaataatattgaagacagtgaagatagccctgaagctatccttacagacctctcaacatctccaattgtctggttaaagccagaatgtgcgtcctctgatccttgccctatcatgcagccctacctctaggagctgaatttcactgttccatatctgacctgtaaatatttcatcaagaatttcaagacttagttaaaaatcactaacattactgcttaaacttacaggcctcttctttaaaaaaaaaaaaacatcaataaaatcaaaacacgtaggcatttaatgtccattgaatgcagtctcacacagtcgtttaggatagaatactaaatattaaactgcaggttgtgcataactttgggttccttacagtttagttgaactagtttataaagcagaacttaacagttttgctctctttcagtgtgaaaacttcttttataaaagtaactactcgtcgaaggtgtatagtgctttttatttttaagaaatgagcttctagggctgaagagatggttcatcagcttagagtactggatgctcttccagtattcagaattcagtttccagcaaccacatggtgcttcacaaccagctataacgagatctgatgctctcttctggccagcaccatagatacttacagaatactcattcataaataaaacattttaaaaatgagctttatggttcgttgtgtagcactgtacatatagacattgcttaaagggtcggttttccaaaaaaggctcagtgtaaagaactgtgtgaggtggagttcactgcaaagaggttggataaagatattgccaaaaaagaccagtgccgtggcttgttgggtaaaagtgcttgctgccaagctggatgacctgacttagccccaggaacctgcatggcagaaggtaagaatccactacagaagtccctctacatgcacatgataaataaatcttaaaaaaaaaaagacactaataccatcataaaagatgtatgtatagtgctatatagttgcagtggcttaccaagtagcagaaagcttgtttctagaaccatgtgtagtctcatcttaaagaaaacaaagcaaagaaatgttttcagtactataaaaacaacacagaaacttggatttgataatgtttgacaattttcttgttgaaagaaactatatgtgcgagatattcataaatgtcataagttacaagcgtttacattaaatcacataacatttccctagtttactctcgctgaatatttgctttgagaactgtctttttcttaagctatgctacatgttaaatttgttaaaaccagcattccaataacctggtaattacaaaacgacagtaaaccattaaatactgaaataaactttctctgagtaaagcaaataaatcaggggatttattaaataacattgcagtgttgctatttgctgtctccatttggagtagtggttaggttacactggtgtttactgttggtatatattgtctatgaaagtatgaaaactgaaaagtcttcctttgcatctggaatttacttaaggatttgtgtgtgcacgacacgtatttgggtgcctgcagaggggagaagaatgtgtctgctttcctgaagcagaagtaataagcagttgtaaaccacctgatgagggggctggagagatggctcagcggttaagagcacccgactgctcttccagaggtcctgagttcaattcccagcaaccacatggtggctcacagccatctgtaaagagatctgatgccctcttctggtgtgtctgaagacagctacagtgtacttatatataataaatgaataaatctttaaaaaaaaaaaaactagtaaaccacctgatgtgggtcctgggacctaagtttgatcatggaagagtaacaagcacttttaactgctgagctatctctccagccactagaattaattttagtcattacattacagtcaccttcatattccgtttcttgcctttgctatctagagaagagactttaggtatagattcattctctgtttgaaaaaggccttattaagggctcagaaaatttgagaagaggatgtaataaacttggtatagttaacatttagttgatctcattgtttgctttgttctttcaaaatatacatacagcacttccctttcgttagaacagaatcagtcttaatctcaggtttagacagttgaggttaataagtattaaggtgggtatatatgtaaagtagtatgtatatgatgactgaatatgctgttttcttacctgctttcaatgaagggatgctatatttaccaccaggagactattactcccagaaacttagaacattgctcagtttctgttgattacatacgtttcagttctgatagattagacatacaaaatgtctaaaggcaaacgtttcaaggtgaacaagaaagaactgtgtaaggagataaggtagaggagtacaggctgtgccacaaggttattggctatgaaacaacatctgtcacacagaacccgagctttggccacttgtcggtggcgcttcggcaaaggagcatatatcagaggtgaaaggatgagttgacaaggcctttgaacttcacaaaactaaaacaaaagaactagaaatcattgtacccagtgtaaggctgggtgtgcttctgcagaacttgtgtgtgtctgtgtgctacctgctgaggccgaggtgtcagatccttctggtggtggattatagctgagagtgagctgcttgctagacacatgatgactacatgactgctggaaacaaacacaggtcctccgaatgagcaacacacactctgactgctgacacatctctctaaccctagtcagtttgtaaattacatgctcaaatacgccttttgctcaagttgccctagaaacttgagttctgtttatgtggtttccttaaatccttggaattttaagagttgaggatgagttatgatcttattcttatgctgaaaaatctttctcatttctaacaggttatcttcagtataacaaacagcatatattccattagttagaaaagcattcaaaatcttatctcacatctttaaaagaattaaatttgaatttaagggcttaagtaaaatagccaggttatgaggtggatggatttgttttaagatttatttcatttttaaatatgtatacatgtgtgagactgggcatgcttgttcaggtgctagcagaagccaggagatagcattggatccaggggttggagtttcagtttattgttagctacctctggtgggcgctgggaactaaacttaggtactctgtaggaacagtatgttctgcaggaacaggatacactgctgaaccatatctagcccctgaagatagatttatttctcaaaaacttgtagagtatcgaagatcttaaggactgaggttcttaagaaagtcagaatattttaggaagaagcagcgggggtatacaacctcttttcacttactgattgtggaagtggcagtttggctttctctgatgagctttactttgcagtttgatgacaatttgtgttaacttgctgtagacagggagggaggtagtgctgccaaatgtctttctgatccagcacacttctttgtatgtgctacctagggtgacttaaatgctagcctgttagacaggttcactggtcagccttcataatactggataacagccgcacataatattgccactatttgtacttggctaattttcctgcccactcccaccctctgacttggtgaacattttattttcctgttcttttatcctgttcctgcacagcagaatgcaaaccctataaaagaaagttgtttacagtttcacccaatagcttgaacagtgcttgcatttagaagcataatgcatataatgtagattgatcaaatttaagacttgttagtaaagtatattagtaaaggaaattattattttgtgaattagcccattgcaatttaaaaattctaagtccagtaacttcaagagttatgattttaaaaaatataattatgtgcatgatgggtgtggtagtgatggttagaggacgaccttgtgatgtcagtttggttttctcttctaccattgaggggccttccagggattaaactcgggttgccaggattacatggtaagcacctttacctgctgagccattgcctcagcaccaggaactgatttttttattttcatttgttcaggagcccacaaattgtttctgataggtaattctgtacctggatacgacactggggttaatagaaaaactgaagatcctacacataatgcctcagagtctgttcagcccatactagcagacaaacaggtagaagcttttgaagtgtgacaggaatgtgcctgcttcaaaagtcagctcaggccaacaggcagaatggtgttgtaagaccttcaaggtgaaaaccaaaagtgttgtagttgagtccaagaaccttctcatctgtcgtagtagttattaccagcactgttttcttaggaatggcactatttgtgccattgcccatttggttagcatctatttaagctaaaagttgaccatttaaaactgatttcctacactgagtaaaggtcagtaaccacctgaggtggtgataaaatactagcagtttagttgaagacttgttgctattagaaataccatagtctccaatgagagaggatagtatctagtgtggagaggggatgtgaaaatcatagccctgttttgactgtctgcagctataactacttaaattttccgttcttttattatgatttagtaatcttatttactgaaccaaaaactcatcaagaagccttataccttaaatttaaaaaaacaaaacaaaacaaaacaaaaaaggcaagtagtctcaggctgttcttaatagaaacaaggactacctttgaccaattggtagagtgaaactatgtgtggatggcaacatgtgcataaataagtaaataaacaaaaatttgaaaaggtgtgattggaagaaagtcttttaaaaaatgatcatgtatattttatctgcaaaggataatttattgctcctgaaatgagtactttgttccacatgccattttcactcaagtcaaaggtctaggtatgttttgggatagccaatcatttagttgcttttggtttttttttttttttttttgaactatgtttttcttaaaattttttttagtataacaggtataataactttgtaagctttcataactaggttatttctgtttttccttcagagaattagttcaaccaaaataagcactttctttcacttaaaaaaaaaaaaagtgagggggctggagagagagttcagtgttaagagcagtagctgttcttccagaggacccaggttcgattcccagcacctacatggcaacttacaactgtcagtaactccagttccaggggatctgacactctcacacagacattctatcaaaacaccaatgcatataaaaatatttttttaagttaggtttgattggatgtcagtggatatctccaaatcactgttgaaatacacttttccatgtagctcagaaatagtattcatgacttttgatattgatgagttatttcttaaactatatgaacactcttgaatatgtgttctctagaaggaaaatagtctccaaccagtaccgatattaaagtgtgtaaaaagaataccagacgatcagacttgccgtcctataaaagattgtgtagataccacttccagcttaatgtttaatttcccacacccttttcatgaccgcaccttcacactttgtttttttaattataaatggcattccactgtatttaatgtgattatattgtagacaggtgtaggttgatgtcattttgcaggtgaagccagacacaagataaaataaggcctgtcattggatgagaaggaagggagatgggaacagaggttttaggacagggagaagcagaagagtggaagagacaacatggaggcagatgtaaacgacccccaccttgcatctctacatagatacaggttgataacgaatatttcttaagggatggatttctataggtcaatttatcttatctaggtgagtagtttatgtctttatcaattggttgtgagtttattgtgtggatgtattatggattgagaatttaacatgtaaatctgattgttggtttacagtttgttgagtcttgaatttactgggtggctggaaccaagtgccatatggccccatgggtgctgtcattagaaagagtacaagataccccgcaatgccatgcagccccacgggtgatagtgtgggataaaaggttttactgcaacaagtggcatccaacatggggcaggaatccactaaaaattgaaaatttccagcctgagaaagttttattttctaagtaagagggggccagcgccatgttgagtcacgtgatgttgctactgcattcttttcataaccctcctaaaaatagtgtttgaagttttgtggtatttaaaaaaaaattataaaccttgaactactttgtaaactaactgttgagagtttcatattgattcatagttgcattttgcatgatactgtggtagtcttggcagttacttctcccaatgccttaagaactttatgactgttttaaataaccatgttgtcaagtcttgtgatcttttttactttgccatctatgttttatggatactagtaattttaacttttatattaatttgtgaaatggcattttatcttcaatttaaagtattataatgacatgttaaatacattttgatagtatatagtgaccataaggacgaatttgattagttttagagttgtttggtaaagcagtgagatttctctggtaaatgagagatgagtatagatatcactcatgatagatccatttttgtctggtaatatgtaggcattgagctatgagagggacacacacaccagaagtaatcccttttttcttgtccccaaaattatttagaaaggattgcttccccagttatctgaaaatttaccaaaatctgcatataatttttgcacctaggagctcttctctcttgattcttgctgctgtgaggtgttagaacacttcacagatatgcaaaacaaagtctaaaaagtgctagaatccaatattgacttacccagaattttgcaatcaaacaaaaagttgttttagttttctgtatatgtgtagtgtcttgtactctgccgtgagcgaggcgcggaataccatgaccgtctcagtgttagaagtctctgattaggctctagtcacttcttttttagtttttacctctgtgccccactatgtgggcttttgatttgtcctgttaccagttacctactagggagcagcttgctcttgctctgacaggatgaaatgcctagagggatctgaagccagaatctcagacctgatcactcctgctttctaccttctttctgaacctgactggtgcagcttgggaaaatcagctttgctgccccctttggcctcctagctagtaaaggacggatcaagagagggatcattcctcacttcctattaggaagaaacacccagagaagactttgtatttcttcattgtttttgctattttaagttactagggtttatagctaagcagatttgagcaagttgactaaaaggtcagctgcattctgttctaactagatttctaagtgcatttactaacccagccaccacagagcctgtttataacatcaggaaagcacaaagcactgtagggcttgtaacgctcttccgtgttattgtaggtgactagatgaccatggactctggagcagacaaccagcagagtggagatgctgctgtaacagaagctgaaagtcaacaaatgacagttcaagcccagccacagattgccacattagcccaggtataaaatacatgaagagattccaagctgtatctcttctaagagtaatatgtttctaagagaatttaatttttaataaaaatatacataagatgagtggaaattttcttaagaatgtcagaattcctgttcagtattagcattagcttatgtttagaaatgaaagtaatgattaattaacatgttaataaaatatatcattaaagattaatatagtatgtgtagtaacatgattcagaaataattacttttttattaataaatgaattggcaatgacagctgccctgatttttgttgtttgtttgtttttgttagttttgttttaaataccacacagacttatttttgggacctgaaggtgtagatggtttggaaggctaagaattctaatactaaaaagcagaataaacaaataaattgatgtgtgaatcagtcctaagaacaacagcagtcgttctttcctgtgcattttaaattcattagtctggttttaagtgtcctgtcgttaaggctgtcatctctgtacttgctttattgctctcacctgttcttcattattcatatatttgcttaaatagtagtgaaaacagtgcctaagaaaaaaccaaatatatattaaacctgccagggtaggaatgtggtatttccttctctcatttttggacacagaatgtcactgtatatagccgtgggtggcctcaacctcacagagacccaccgtcctctagattaaaggtgtgtagtatcatgcctggctgtttcactttaaagtgaagggcctcaaaggaaaataatcctggcagagctttgctttgctgcttatctccagtttctacaggagttaaaaatctccagtggaagtggctgattagagtaagtcctgggaaattcagggttcaccgcggctgggctcagggtgggcgtgagttggatactggagctccaggaggagtagtagagtgagcaagctgactgtcctctctcacaggaacagggtttccctgcagatctcggataagacggcaccacatatgcatggagataagttcatgtatcagatgaggcaacacttagtcagggcacgcttagtaaaacaggcacggggaaactggggctcatctgatcatttagctgagcagtctccttgatgatgaccacattctatccagcttgcttgttccctgtgttgactttataaagtcatcttggaaaatctttttgttctcatatctttttcttagggaatttgtattcttggtgaggtagaacatgtaggtaacataaccttgagaataaaagattaactggggtaagaattcttggtaagacattttctttggatgtttcctatggtccacttagtgttgcttataaatttttagaagacttaatttcatttcttatatctgatacattataaaataccttagatctccttacagtgagtactaataaattagttcatcgttcccctctgtatttaaatgatctttttatagtggaaactcaacctctgctttaggaaatttctttcaattagttggtggtggttttgtttttcatgtctgtcaggatccttgtgatccagatgctatgtttctgggactggttttgtagtggtttgattttgtttgtttctccattttccttacattttcctcgaaatttttaatctttttgccctacttcctaggagtgtattcactccatcctgcctgctgaatttttttttttaaataaaaagttcatttacaaaacatgggtttgttttgtttttttttaagaagccatgcaacttttttttagaatttattttattatgtatatagtattctaaaatatattatatatatagtattctagaatttattttattatgtatatagtatactagaatatattatgtgtatagtattctagaatttattttattatgtaatagtgttctaactgcacgaatccttgcagcccagagaggacaccagatctcattacagatggttatgaaccagcttgtggttgctgggaattgaactcaggacctctggaagggcaactagtgctcttaacctctgagccatctccccagcacctgactgctgaattgttatcatgtttccaaggttgttctgtttgttccctgggtagtcttggtattttcatagtatcctgtgttttatagtatcctgtttattctcacaaaagggggtttatttggggggttgtttttgccttatttggtagttattttgagtttctgtttctcatgtggccatatacctcatcatcctcattaacatttgttttaatctccatctttctggttgaggctttcaccaaattcttaaatctttgtgaatgtctgctcaggattaaagtagagggagactagggagaggattgtagcttcaatggtagaacatgtacttagcacatgtaaagccctgggctcatgtcccaacgttaagtggctcataaacaaatataaaatgactaagaagctactttggaagctttgtggatatatttgtaggccaacagctccaaaccaggaacttaactgtaggacagcggtggtcttaactctttgtaccttggatcttgaatttgatcaggtactaacactctgtttatatataaagggcctgagagccaagtgaaagaagagggctaggatgtcatagtgactttcagtgaacttaagacccattaactgtaattctgcactcttctctcatagcaggccagacttctctgcttcaggattaagaacggattcatttgtttttaaagagctttcaatgcaccctttattttcagcccttctcttcatgcttccatttacagttttggagatctcgagcatctgcatctgtgtggtccaaggcagcctttccttcctccatgattgaaccataatataactcagaattcagaagggagcatgtttagttcactgttaggcagcctttcagcatggaagacgtagaatggaatatgtagttgaccgtgaattttaagaaagcttttctcttttcttcttgtccagctacaactatactttttagttattcatctaagttcctaacaaaacaccatacttataaaaattattttatttttgagagtctgatacttatatataatgtatctgtgtcatatttatcacctctgtacccttttattcctgccatactctttccgagataccttctactttattgtcttcttccggtggtggtggtggtggtggtggtggtggtggtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgcttacatgaatgtgagtggggagttacataacaacttgtcagtggctgtctcaataagacaatatctgtccctcttccagcaaccattaactgctaaatattttctcaggagtttatgaagtcctacctccatccgtggtagaatgttacagaccccatcttatacatattttattcaggaaatcagtcgttgtgagtaacagtttgctagtgtaacagcactgtcatgccaagaactcagtttcccaacattccttcccatcctctgacttatattccttccatctcctcttttaaagtgttccctgagccttgagggaggtctatatcatagatgttgacacaacctcttgttaattctgactccagataacattttaatgccagtttttaaatactaggaaaacattgtaaagctaagacaaaatgattatgatttgtcaatgaataggagggggaagaaagcaagagataaaggaagaaagataatcaacaaaaaaatacatcatattttacttttagaatttcaggtttaaaatacctttggttttgaccatgagaatgtaccattttagttcattaggacagtgcatgattaagtaggctgggctcacttgtggagtagtccttcctcagcctcccagatgtttagcaccacacacagacgacagtgtcaaattttaataaatctaagcaaaaagtaatagtagggctttatttagcaataaataattaaaacaaaaataatgaactactatgtagccatattttagtaagactaaagatatttacaatatattgtaaaaccaaaaaaaaaataggttttatggtagtatgttgtaaaagttgaatgtgtctgtgtgtcttaaaaaaaaatctgttaagcatgtcttacttttaaccaggccatacttcccaaggttgttgtgaatatggcccaacacatttgtaagtgacaatattacaatgaaaagataggacacctgtgataggagagtgcttgcctagtaggcacaaggccctgagtttaatccatggcacagaataaaaatagcatgcaagaatagaacgcagcaacagtgtcctggtagttctggtagtaggaaaatatagggtttttagccaggccttatcagccaggcttgtgataacagcttcttgagagtctgagcaggaggatcataagctcaagatctgccctgataatcaagtgagtcagtgtcttaagattggaaaaaaagaggactgaccagtgataacactccatgctagattacttgtctgctacctacccatacatacatgaaagagggcgtatttagtactttatgtttagttcatctgtctgtgtaaacctaagtgtgaagagccttgataagtacgtagggtggaaatcactggattagcttctgtgatcactggattagcttctgtgcaccctgaaaggatgcatgggagacaacactgagtctgtaatggcttcaaatactcagatagcttcctcctgtggacttcagatggcacactcagctcataacaactttatgttatggtgtggctgagtctttcaaacccgaggggacagatccttagttccctcctagatttgtcattaaacagaacaacatgcacggctctgcagacagtcctttcaccacatttatagaatacagtaaaacgaaacatgactaaagtgaccagaaaaggatttcgtgactgtctaaggaaagacacttctagtttgaatctgtgtgttaaagattacctgtcttcaagaggtctttccccgagcttcagtgaagcgtctcgctgggtatgtctttggactcctactcagtgctacactttcacaaccccagctctcccaaaccctgtccctctttcattgtctcatctttaattcctctcttcagatacccattcagaaagcccttgctctgcctctagatgttcatacatgttgcttctctcagacctgtccttaccacctgctaccctatttctggccctcgtcatcctaccctaactatataggctttctcatggcacaccacctccacttttctccctctgaagtatgtatgttattcactaaagtgattgatctagaatgcaaacttcaccatgctatttacttcaaaaagcctttcaacaattcttcctatattttcagaataaatgaagttttgtgtttgaaacatggccttgaatggctcaggctggatttggttggactttacaacaatccttctgcaccaatatctttagtattggaattacaaatatgtgccatttttactgactaaaatataagatttttgtttgagacatgcctgaaactcacagagatctggatacctctgcctcccagtagttctggcttgaaagcttttttaaatctgacatatagagaggtgtggggtctttttgtttgtttgtttgttttattttgtgtgagtacactgtaactgctttcagacacaccaaaagagggcatcatatcccattatagatggttgtgagccaccatgtggttgctgggaattgaactcaggacctctggaaaagcagtcagtgctcttaacctctgaaccatctctccagccccatatagagaggcttttaaaactagaccattaaagccttttagagcctctgtttctttatctctacttatgacataagcagcagtgtgtgtctttcctccattttacatccacacacacatgcatgcacacacaccacacatacacacactcaaacacatctatacttacatgcatgcacacacatacacacacaaacaagtacacaaacacacacacaggtcattaactttttgaaagcagggtcctatatctttctcttttttattataacattgttcttggtacacgagtcctagttactaagttctattgtaaatatgttggagcttaggaataataaatggtattcgatagacatgtcagtcaatttaagaacctttaagatttggatattgaatcattaatattgacttctttgtacttcaagaaaaactagtttctttttaatattctttcagctctgctcctttattcatccaaaatgaaaacatgtcttaaaatccacctgttaccagattagtgttttttatgaataggagaaattgaaaattttaattacaatgcatttgctttcttgttaaaaacatatttgtaattagtctaaatgtgttgttctgaacttagtaagttcttattgcccagactggaaaagttaagattttaaaacatggtttaatatcagggtgttctgaaaacttgtgtgcttaaggccagcatgtctgttgttgacattgggtgaatcagtctcggcatatttttgtctttaagtaaatataatccttattactcagcctttaatttaaaaagaaagaaagaaagcttgaatataaaaagcaggcaggaaagagttttaaattgttggtttcttgcacattgttaaaatatgtattaagtaggaaggtcatagttttgcttattagaagtcactttatgttaaacctgagtagtcccacagaggttgctgtggtggtcattgctttccgtgttttctgtaggtatccatgccagcagctcatgcgacgtcatctgctcccactgtaaccttagtgcagctgcccaatgggcagacagtccaggtccatggggtcatccaggcggcccagccatcagttattcagtctccacaagtccaaacagttcaggtatgtgtgcgacaagctctatatgtacttcaataacttgtttgtagccatttctgtctcctttcattagttgtgaaaataaggacacattagtttagatttattttgcttggtagtaaaggagtatgttgaatctgcatagttcagttatatatgccaagtcatatctaaagaaaattactttttttaagcttcaaaagaaaaagcatttatttagagagatagatattaatacatgtgataagtggaaatgttgggaaacactgtaagactgatgtacctaaattaaggataatcaacttaaaatgagacgaagtttgtatggggaaagggttgctaaagattaattaatagtcttacctgagataagatttgtactagagatcacatcagacatcgaattgtctgttgaacttttaggtagagtatgtaactctgaaataatacagtatctagaaggtacatcatgagggtcattgattgtatcacatatctgattctttggagaggttgctcctttggggtaaagggaataaaggcattctagacatctgaagaggactacttaggtctagggacccaagagcagtaacaggacaattctttgcagatcagctaattgaaggaaaagcgagcaaagactaaacattcatgcaagtaggccataaccccagcctcccgtttcatattttttacttcacagtcttcctgtaaggacttaaaaagacttttctccggaactcaggtgagtcctaggtactagatttgaagaaaattacttttagaaaggaaggtgagaaattatgtatgtctttgtccttctctgtgaacatggaatttcagggtttgagtccaactgtacgcctctgttgcactgctcactgcttagttactgctcaggtctctaacagctctcctccagctgatagccagcccagaatgcacgaacaccttgtgaaaaggaagctgtacctcctgaggactggacagttagacatgaagcctaactgctgaaatactgtgtagttagacagagaatgcctttctaaaatggggacctgctgaccacaggaactaagcaaatctaactgtttctgtcaaacttcttcctttcccttctttccccacactcttcctactatacagaaacatatcaagattcaacaagcttctatttttcttcagcatttattatatggtatatgtttaatttctcttgctcagccttggctgttttctagagtggatctgctgtgtttttcttgtatggtagccagaactgactgatattttcacgaagaggaaaggtgcttgctcctttgtcctggaaccaggctgttcttgtcattaaacccacatcaggtttcgtcagcattatgtcaagctgtagagttgatgttacctcctctacttttttcttccctttgtacttctggtttgctttaacactttcagggtagagaactgttgtccggtgaatttcctgaagatactaggaatggacattactaacaagcagtgacagtattagccaagtcaaagcctcgttttgagaaaccacagtgataagtagtcctgtgacttagcctgtattttagggaatacagtcttgccatcctccatatgatctcagcactaactccatacctgcattcttctctagagcttggcctgctagtcaagagttttagagaccagacccattttggatagtaataggtcaagtgatgctgagcacaaatcacccttacttctctttcacttacagaaagtttttgctattacaaaatacataatacagtttttaattaatagaatatgatatttaattgtgcctttattcttacttttattcttatcttggggcaaaataatttaaactcaagtattaaatgtccttaactttgatagaaatctgggtttgtcttttcaagaataacatttttatcacagtgagctatgcttataactttaaaaatacatgtttgaaaaaggaaacctggactcctgaaagtctggactgtatggagatgtaatgtgtgtttctttttcatgaaatatgctaaaccaggagaatgtgttaaacttgcatggttatttgagccagctaccagtggttagtgttaataactaggaagtagtggcttacaagaggttattaaacagggtgaggtatccttcagacttacagatgatagagtgttagtgttatatataaagtttccaaaacatacaaggtatgtgtctcctagtgagagaataagaatagtaggacttctgctgatgtatttcaatataataatattggagactgagctgtattaattaatattctgtgtacagcttcactttactgccttaatttattcttgtatgtataaaccacatctcacagatgatgtgagaattcttgcaaaaatgacagcagttacagcagtttcacagtgtatacagggttagtagtggcacttaccagtttggaccacatttcaatatagtgctgtctaaatgtaccagaaaagattcatagattttatgagtgtaaattatgacatccatttcttcaacaaatgtttattgatcacgtgctatgtgccagtcattgttctaggtgctatgaaaggcagcagtgagcaaacagtacatctctgttctcatgaagatcacacttgagtgaagagacacactatgagaaaataaaatgtgtcaggtgaggataagtgctgcagagttaataaaacaagggaaagggtagagcaagatgcaaagggaagtatgctgtaattcattcaaggtggttggggagcacagtgagcagaaaccagatagccggagaggcagctaagccaaactaaagatgtggaaatgagggtgtttgttatttgaaacagtagtatgttcctgctggttccctaagtaaagtgagtgagaaaggggaggacagtgatggagactcagtgtcgatagactctttaatactttgatgtgatggagacagcagtaaaggcttgtacaacacagtggttcccctctctggctatcatacagacatagcatctctgtgtctgaattaggaatgggtagtaggtttggtggtgggaaggatatttggtttttgttttgttagcccaagacaagtgttccaaagtttctgatcttggtaatggcaaacaaggactttgaaggggtattttacctaatagaagtccctttgtgctatcattatgggcagggggctttagccataaatgaaaggattattagtttttagaagaaattgtggggagaaattggaaaatgctttcttaacattcatggtgaacggtgtcagtttatctttttaattcaatctttaaagacttagagtttcctcagttgtttccactctcccagccttaggagctgctgaactttgctgttaaatttgagactagctgggcatgtgaccacatctttaacactggcaagggtaggaagagctgagagagaaccagggctggatctctgagttaaaggctggcctgtcctacaaggcaagtttcaggccacccgaaggtttatatagtaatatcttgttgcaaaaaaggaaaaaaaccgtatctttttgctagagtgcagtacaataatcatagaccagcaatcaaaatacattgatatatattgacacttcattgatacatgaaaattaaaataattactttctaagcacagctaaaaggcacttagtttgttagtctttacatttagagttaaaaacaaattggtagatatccagccacatttccaaaaatgcaggcctgccctgggtgggcagggtaagtattactgtaaagacagcctcgaaggcgttgcctcagtgctgggattaaaggcatgcacctccattcttgacttgaatcactggtgaaagatgttaatgatcttttgggctttcaagagtctctgtagccttagctgtcccagaactagctctgtagaccaggctggcccctagcccagagagataacacctacctttacctcctgagtgatgggatgtgtgtcaccactgcctggctcttagtgatcttctaatgaagtacatttaacgatgttgttcttactgttagtaaaatacttaattctatttgaataaattatttaatatttagcctagcctaagaactttttaaatatataacatttaaaatttacaactagacatttttgcccatgagaaattttcttatttgtctgcatgtttgcctgtacaagatgtgcctggtacctactgatgccagaggggggcatcctgaggactggattagggatggtgtgagcttccatatcagtgctgggactgagcccaggtcttttggaagatcagctgttcttacctgagccatttctccaaccccagttttcttatttgtaagggagatagcagatcacgttttaagatctggtcttttttgatccattgagtaaaaagttatctaaaccacaagtcttaatcccagccaagcaaaacacgtttgcaaaggattgtatggttttaataatgctttatcatttcttaatttctagtcactgtatagttcttttaaaattttaagttataaaaaggtaatggtttcggttttggaaatgtccagactgttacaaacagccaaaggtaaaagcctttttaaggactttgggagacagacatggacatggcaagaggtgtgagggcactagggggactctgttactggaatgtggtaattatgaaacctgtattagattttaaattttaaataattgtttttaatttcttatatttatgaatagttataaaattgaatcttgatatttttattagtgtaaaaagcatatatcctctgactttttttttggtaaggctttacttttcaagagcaacttgattcacagcaaaattgaaagttacagggttttcccatgtattcatggcctccacatgcatagctgcctccatgttaacacccccacataccagatgctgtatttgttagcactgctgcatcataaccgtccaagcccgagtgagtgtgaggtggtacacaggcctgtcagtgtcggtttgctctatagccatgtggtttgggtttttcccgttttagttaatcctttctgttccaaatgacaaaatcttttagattagagaaaggcaacaaaatccactgagttggtgtaggaaacaaagttacagctaaaaaagcagaattttatttcaatactcatctatacttagtttatttgtttatatattttgatttttgttgttgttgagtcacagagccctggttgactttggacttgctatatggtatgtataactttgaactactgatcctcttgtctccacctcctagttgctaggtttgtaggcctaaggcacatactcactttcacatggtgctgatgatcaaacccggggttttgttcatgttatgcaagcactttcacctgagctacatccctaaggtgtgatagtacctacctaggcgccagcattgggaagcagggctagggagtctgtctgtatcatagtacacttgccgtagtagtatccactctgaacatatttattggctattgactttttcttgtaagtttttttttcttttcccagtttctgatcttctcattgtccataagaacatttgtgtagagacactagagtgggctgagtttaatggctggagcattccttacttctctctgcagtgagcatatgcatcatatagcactgtcagcaacatgagttagactgcatgccaaattcctttggcatttctcaaatgtgtccaaatatggttttgtatgctgctgttgactgttttgaggtgatgctagcacgcacaggcccttttgtcttgtttaaatctacttctcagtgtgacagcattgcgtcacatggaggaaagcacacgaggcccctgtagtatagactagaaaaacctggtaatataagttttacagactttgagtccagagtaatgtaaaggcctgtgagcttgttgctgtgacagcactgacactggagccaccagggttttaatgcctgtgaagtctgaagtggaagtactgactgcaagcgggggccacactccttagcttggtggcccatagaattgcctgtcctgcttgtgcctcagctctctgtcttcttcagtgaacacaggaccaggactcgtctggtactggatgtttactgtatgaaataatggactacatggaagcgatagtatataatttggcacataatgttattggtaaactctagtttctgccaccccacatagtctttaccccagtctaaggacccttaacggaaaggcatagctcattcattaccttaagttttactcaagaatgccaacttttgaagcatgtttatgtacagtagagattgtttactgacttggaggaaaggcagtgtctatgtcagagctatcattgctgtgatgaatcagcacgaccaaagcaacttggaggaaaaggtttatttggctcacactttcacatcactgttcatcatcaaggaaattcagggcaggaactcaaacagggcaggaacctgaggcaggagctgatacaggggccatggaggaagtgctgcttactggcttgctcctcatgactgctcagcctgcttccttacaaaacccaggaccacctacccctggatggcaccacccccaatagactgagccctcccctgtcaattgctataagaaaatgccctacagacttatctgcagccagatctttctcaattgaggctccctcctctctgatgacgctagcttgtgtcacgttgacataaaaatagccagcagtctactatttggtaaagaatcactgacttaaacaactgtaaatctgtttgtacaacaattataggaaatcatattatcgacttagggctaactcgtgtcagaaaacttagtttgctaggcagttctattctaacaactgtaagaaatcattgccatgatgatatattcagaattgatttcttgggagaagttaaaaacattttctggacagatgtttctgtgtccatggcatcattttaatctgtaggagatgaattttagtggtatggttgtctccattagaaactgaaatatgtataagatttatatatatattctttgtattggacttaaatattttatgggaaataaatttgataatcaagataaagttattctttgttttcagatttcaactattgcagaaagtgaagattcacaggagtctgtggatagtgtaactgattcccaaaaacgaagggaaatcctttcaaggaggccttcctacaggtatgtggtgtaatagagtcagagagcgcaggaaggcatagtacttggcaatagaaatgtcaggacactgataaagctgctccagcaagtactttgcagattatttctcttgagttacatctcaaataagctggcatttgcagtacaaaataagactacttactctaaattagtaaatccctgaaaaccattcaaaaaacagatatttcattatgattcttaataatggaaaaattacagttacgaagtacaacagaagataattttctgtttgaggtcaccacaatatgaggtatgttaaagggttaggaaggctgagaagcactgctctaatatttgtagcacagcatttcccttgttctgtttctgtagctatgctgagggctgctcgggattcctttgagtgttagcgttgtgctcaggtacactgttgatctgcttagctacacatttgcaggccgtttttatgtcatgggccttaacaaagagctggatttgatagttcagggtagcgctttgctatcatcctctagtttgagttcaactgtaatgttacctacctagtctatgccttgccttgaaggaaggtcaaagacaattagtacatatatcaattggttccatattaagtatattgagaaacaacatacctactaatctcatatgcttacttaagaattcagtaaacttcagaattctacagttatcttctctgttagtttaaattatgcctatataaattcctatttatttttagatttattttgttctatgtatatgggtgttttctctgcatatgtctgtattcagctgtgtgcatgatgccaacagaggtcagaagaagatggtggatccactagaactgacttacacatggttgtgagtcactctggattctaggaatcaaaacaggtcttagaatagagcaaccagtgctcttaaccactgagccttctctctagccttgctcctttttttttttttaaaggtagttgatgttttccttacctttctctacatacgtacttagagttaccactaagctctttttctatcctcacccaactataaattctaaatattataagtgaattaattttgagatcttaagaaattttatgttgtaggatatatatcatgggcatttatatgtataatcatttcattttgtttttgttttgtgtgtgtgagtgtttgtatttctgtgcatcatgtgcatgaggtacctgcagaggctggaaatgtcaggtcttctagtactggagttccagtgtgagctgctcatgctgctgggagttgaggctgtatctccagcccttctcttttgttttttatatatatagcgtgtattagagtttgagacattgaagtatatctcatgttattatataatacctttgatcctgaagcaaatccaacaaagtattagaaacaagtcacatgacttctcctaaatcctatgcaaatcataactgccagtcagccagatgactccagcagataaacgtccacgtaaagatgaaaggaaagaaccaactccacacatacacctcctgctaccttcacacttaaaacagtcaaacagacaaaacccacaaaaataccagctttatgtttgccttaatagttttctggccaaaatcctagcagatattatttgaatcaaccatgctttactaatctaatgaaagccctcagaaatcatgcagagagggttataaaggataatgccaccttaccttatagtcagattttcacactgatagactgtttatatgaagtttatgaattcctgaattgtaacctttatagcaagatacttatagcatgcttgcaaggttagaaaaatgaaattgattatacaaaataggtttttattggaacaacctattgttataaagcagttaatttgtatacagcaaaaagatgctccatttggtctcatgtggttttatatttttaccagtaattgttctgaaaaaacagaagtcatgtatatcagatttgcaaatgatagaaccataaggcttaacaaagtgacgtggttctaggagcacgagtaccttgaaagatcacacaggcaaaggaaatgggctgaagaaataggactgtagtgataactgtgaaatctattcaaactgcttgtaaaatgaaccagcttttcagatatctgactctggatttattgatggaagaatttgtttgaaaaaggttatttcttctcattaaccagtatgctctagagcaaacaactagaatgtgaataatgtcttagctgtgtatttggattctcatacaaatgggaactagattagattgggagactaaattgatagtttaaatttcaaccataatcctaaataaggaatatttcttcatgctacaattttatgtagttatctataagtatatatagagagaaccaggcatgcaaggtgtgtaactgtaatcctagtactggggaggctaatgcaggaggattgtgattttcctgagattcataataagacaccttcttaaaaaccaaaataagccaggtggtggcgatgcatgcctttaatcttggcacttaggaagcagaggtaggcagatctcttgagtttgtggccagcctgatctaaatagagtgacagggtgagtttcaagataccaggaccacacagagaaaccctgtctcggggacacacacacacacacacacacacacacacacacacggcttggagtgggtagagggagatgggtcttctaaaagattcaaattcaatccccaggacacatacagcatctcacagccatctgtaactctagtcatacgatatacagtgtcctcctttggattctgcaggcactaaacatgtacaaataaaacaccctagcgcataagataaaaaaaaacaaaacatatgtagttttatttattatccaccaccaccacccccattaccatcacgcaattcttgcatttaaagttcctactttgtttggagtttattcatgtgtttgaatttttgtcctatacattagcagtttctaatcttaatttgtacttagtgacttttttttaattaaaatagctccttatttttctaggaaaattttgaatgacttatcttctgatgcaccaggggtgccaaggattgaagaagaaaaatcagaagaagagacttcagcccctgccatcaccactgtaacagtgccaaccccgatttaccaaactagcagtgggcagtatagtgagtaatacgtttttctattatgaaaagtgagaaggaaaactgttaggttctctttaaggaaatgataaatgccaaagcttttgctggcaatattgtttgccctactgtcagacattattgaagaattgttaaaaggcatttatgggaatgtcactctcttttttcttttacccattcctgccttccaccatgtttcttacatgattcacacacaaattactatttttaattttagcatggtgttggcttgccagcctgtaatttgtattccagctgtatttaaagtggattcctaaagcaaaatttttagtttttccaacacggaaacttttggtattatttataaagaagggtaattttttaatatttgctagttttatttatgagtgtgtgtgtgcctgcatgggtttatatgcatcacatgtgtgcagcagttctgggaagcctttggatcagatcctgtggagatagagtgacagacaattgtgagccacctaatgtgggtgccaagaaccaagcctaagtcctctacaagaggattaagcgttcttaaccactaggtcatctgtctgaacactaggggtggtgttcttgataaattgtctttactgtctgtgataactgagctaaataaacttagatggacttttttccttaaactattggtagctgtaaaggggtacatcttccccaccctggcctggctctgtgtctgtatgtgtgtgtttacatagcttcatacccagattattttaagtgttcaagatggttcttctctgatattgtacaagacttctcctccagcatgaacacacggagggtataacctatccctgtcctaattcactacttagggagtttcgcttgaattaggaaagaaagacacacaggagctgatgaggaggatgaatggattgcctaccattcccttagatgcttcagtttaaacgtactagtactttgagttttcttaaagctttattcaccttatataccatccttttgtacatgcatgtgtgcctttgatgaaagtgatttagcattatgttcttgacttttcttttccactaagaaacaaagtactgaatactcacatgtatggcaaactgcttattttgtaacttgtttatttaaaaaaaaatttttttgttttatgtgtatgagtgctttgcttgcattgtatatcaacagcacatgtatgtggtgcctgtaaaggttagaagagagcattggatcccctgaaactgaagttacaggtggctgttagccaccacgtgggtgctgggaattgaatttgggtcttttggaagagtagtcagtgctcttaacctcttaagtcatctctgcagtcctgtgtaaacctctttctcattctttcctacttgtattcatccctcctttttcattctgcttttttccaaaagctgtcctttacagaatctcagctattacaatttaattgtatttagattatttaagttaaaatttaacataattatatttctttgttcatatttcttggtagcaatttaattatacctagagataatgaatttattttaatgaatttggttttgtcagaactatcttatacttttaaagtaatcaagtgtacaaataaatattacacagttaaaaatgtttaaatcttagatgagacttttattttcaaagaattgtcgtgtgtgtgtgtgtgtgagaccttactgtttaaaacaactatatctgttacataagagcagatgtttttagatttccaggaaaaataacaaaatcaaatgatcttaacagagtagcccatatcttttccttccattcactttgaggttgttgctgtgtaatcaagaaaaagtagaatgttaattaaatacacttgaatttgcaaattaactccaggagaaatgaagatttgattttgataaccttatgattctttaaaagtccttgttccccgaccctcttcgtgtatttttattttatgtgtatggatgtttttgtgcaccagatgtgtgcagtaccagcaaaggccagaagagggtgtcctgtcccctagaactagttgctcatggctgtgagcctccctgttgatgctgggaatagaacctaagttctctgagagagcagccctatctccagacctttccaagtaatcttacaaagttaatcgtagaataattcatagacattttttaacatctaagatagtaaaatctggaattagctaacatagcattatatatgctagtatgttaatttccacaaatttatcaccttgataaagtgactaatagtcatgaactcatagctcttgattgaacagtgaattatccattttctgtgcgcataccactcaggatagtataattacccagaggtcaccgataatcctaaattaatttacttctgttatacaataacttaattaggtacattggtaccacattttttttttaactttccttttaatgcaattataatccattatgaagagctgggaacaaacctcagttgacagggtgtattcctaccatgtgtgaaggtttgattctcagcacttgcccatcatatgcgcgtgcacaccaacacacacacacacacacacacacacacacaatttatgaaattgctcaggtaaaattataatttttaggtttttttttttttttgaaaacttgatccttggatttatctgaggagtcaatcatatttttcagttccagtatccaaagatcatgtatttcaaacaagataagaacttacctttcaagtaactttttctttgcactgttgtgtaaggttgacttggatgagtgctgagtgattttcatttgccagatagtagctattgtgcttcttcatcagcagtcaatagaaggctctgtgaagacccagacaaggagcttagaactagataaggtgcatctaaatgagagtctgtctctagtttttttttttttggttctttttttcggagctggggaccgaacccagggccttgcacttcctaggcaagcgctctaccactgagctaaatccccaaccccctgtctctagttttaaacttcatgagacaatcataataactgagatccagggtatttcacatctttcattgatgagatttttggtggggatgatgggtagtaggtaggtgctagagactgaacccagccagggccttggatattaatgtattaatggtttttctttttctttccttaaatattactaatttcatttatttattttgagatgatgtcttactatttttcccaggctagtcttaaacttgtagctcaagtgactttttttttttttttttttttttttttttttttgccttagctccttgagaagtgtcatggctttctaagtgcactactgacaagtttataaattgctctaaaagttagaaaatataattcactgagagagggccttattaatcatacttttaacaaacatttattgggtgcttcttatattaattaattttctacctgtaaaaatgtatttacaattaaagcagcaggtagtaaaccattatatgagggagttgacggtactgtaaagaaagtattgttgtaaagcaggacctccttggagttgaaataagttcaaaaagagtttgagtttcactttttttttttttttggttctttttttcggagctgggtaccgaacccagggccttgcgcttcctaggtaagcgctctactactgagctaaatccccagccccgagtttcactttttatgttgtttgttaccatgcattatttattgtagtacagttcatagactttatttctcacagaaattggatatggtttcctgagtacacagttatttgaataaaccaagtcttccttggtggtgttattttctagagtaatttctcctagaatatgctttgctaattgcatgggtttagtgtgttttaaaatttttatgtggtattaattaaacttgtaacaataaaattcattgggttttagttgccattacccagggaggagcaatacagctggctaacaatggtaccgatggggtacagggcctgcagacattaaccatgaccaatgcagctgccactcagccgggtactaccattctacaatatgcacagaccactgatggacagcagattctagtgcccagcaaccaagttgttgttcaaggtaatagaattaagaattcatacatacggtacattcttttaaaaattactgtgtatactcatgaaaagtaagtatattgcaaagcactactaaaatagttttcttcagtatagatttagatattaacaatttagatatgtatatattaatacataaataaatattcctcatgaaactttccattatcccacttggacgaaaatgtcatggcataaaaacaactatcttttagctttaaaagggtgatacctgtcttcagtgccttcagtagtcaggggagggagaaccaggatgcgttagaagagaactgaggaagctgtggctaagctacatggcttcaggtaacgcccagcttccttgggcctcttcctttcccacctcattgtttgctacaggtcactatggaagagctttagtggattctgctctctgttttgtttatctgttttgttttttaatttttacttaaaaggaatatagagattttctcaagtcatcagttttgatttcagataaaaacatttcaaaagagcatatgtcattatgcaagtttgctcattggagagtaagttgtccttaagtcctcgacagactagaggattttgggggttttttaccaaaatttcaggagtgtggagagtactaactcttctccattaattcattgaacagttcatagtttataccggattaaaaatttatagagtaagaaaatttttgcttctaaaaatatttttgttaataataactaattgcgtgtgttaatgagagtcgtatcccttcaccatggatattacttttggatcataccaaaacaccaagtaaacatcttttgtttaataaattctaatatatttatattatgtatcttgttactagaccaatgatatttgaaaaaaaatatttgcttattagtgggagtaaaacaccttttcactttacatgcaggtactcaaaaaattgtaaagcaggatgtcagtgactttgaattctcaacgtcagtttgaatatggtaacatgtttagtatataaatctttgtccctcaaacaatacaattggtattttaaactaatacgaatgattttatgaagaccttcaagatctctcacatagtgctagggtacttattagaacatattatcacactgtttctagaagcaaataatggacaaatgatttttttttcacatatggaaattgtatggttaggaaaatccaaagctgagtgaggtgacaacacattttaatcccagcacttgggaggcagagacagacagaggcaggtggatctctgagttctaagccagcctggtctacatagcaagttccaggacagccaaggctacatagtaagaccctatcttagaaaacagaaaagaaaaatatccagctcttacgtatttattatcacttatattaagatatgtttacatataccatgattggcatcaaatgtccagtttttcatactttgagttttgtttgaattagacatttataatagtttaccatatgctcattttctcttctgttttgctattttatgaaaaatcatgtcgtttttatgtcgtggcaagagtctacttgagatttttaatgtggctttatcccaatcaagggaaaacactgagacctttatctagaagaatcatctgtgctgggccccgttttccttctagactaaaggttgacaagcatgtaaaccacataatagatctccattcagtacagcataactataagacccagcagagacactaaggcatccttacctgtacgtgtgctaatagtacagtagattgggatcttctttttagctaggatcatgttccctgtttcccttttaatctaagtatgtttagattgattggagacatagttgaattaaattgttgtgtttctgtgtcttatgacttgttcgctcttctttgcagctgcctctggtgatgtacaaacataccagattcgcacagcacccactagcaccattgcccctggagttgttatggcgtcctccccagcacttcctacacagcctgctgaagaagcagcacgaaagagagaggttcgtctaatgaagaacaggtatagatattccagacacttaaatgctatgggtcaagtatgtgtatgaacgcttctctgcacacctttatatctgctaatagtaggttctatgcattgactttctttttctggtagaattctgatagatttctggtgaatcttgctaatgcctctttgttttaagttgcatttataaagtggctaatgggacttttggtcaatcttcaatcaaaaagtaatttttttcatagtttttccacaagagatttaaaatgcatagctttaatgaatttgtattttattctagccaatagggtaggttaattaaatttttttcaaaagtaatattcctcaaagtgcatatgtatactaacattagactatagtttatttgcattaaatttatttgtaacagatatacggtagtcatttttgttttaatgaatactttcttttgcatttttatgcacttaaagatttattatgcatgtcttatgtatatgtgtggatgcccacatttataccatgaagggcagaagaaagcattagatgtcttggatctagagtgacagagtgacagccacctgtcttgttctttgttttggggtcagaatcttgctctgtgggccaaagtggcctcgaactcttgatcctcctgcctctttgagtgctgggattacaagtttgtgctaccatgcctggccttaggaggcatgtactttcttactcagaataataatttaacatccaaacgcttttaaaatagagtaattctaaaaatatggtattcctgacaaagccttaatgcatagttaatgttggtcttcagaaattaaagtgttgcataggagaagctataaaacataggtaaagcctttcttagaaacttatttgcttttagatcagtttggtatctaaaaacaaatggaagttattaatatggaaatggtcaaaacaaaacaaatactctgaggaggccattggagtgttcaccacacaagcaagaacagtggagttcagatccccagaactcatataaaatgcttggtagggccctataatcccactctgatggcagagatggggttccccatgcaagctggtctgtaactctgggtttaagaaagcgatgtctcaagagtaagctgaaagcaacgtcgaggaagagaggaagatagccttcccttcacacacaaaaacatgggggataccaggggcagtgatgggggagggagggggacaaaaagagaatcgaggtatataaagatatcaactcttcgaaattgacaaaattctttttacctcaagtgtctaaatactatgctaaagtaaatgaaacagaaattgtacttagaagatctaatttcaaatcctgcattcaccctttagctatgattctggaagaacacatattaattttctaaacttgggtttctacctttgagtaaaatcaaatccctccttgcacccaaagctcagctctcagggtcaggtaagagagcccatagtatctgtactgccccttctcttcaaacagtttcatgtaggtcagcattatcgttcctgaaaatagtcttaatgtcacagtcttagtaaattatatctagctatgaaaaaggacaaagccgttctgagatattaggggaaacttgaaaagaatcctggtgacctttgttgtgtaccaaacagctataagtagttaaattataatagaaatgtacttttaactctcatttaaatgtatttggaaatacgatttaaattacacattaatctattcagttttaaactatttgttaagaaatagttgaccttttctaaaataagaaacaatggtgattaagaagtctcaagtaaagacctaattataaaattaaaggcagcagcacttactaaagaaaataattctaaggagacaagtaatcagaaatttttttaaggacttgagattttgtctttgtaatttggctctgtaaaataaaggttcatcttcttacagagaaagacaggagaggctgtgggaccatgtttaggggtggcagcaccagcttcttttttcctttccatgcagtactggggattgtcctgagggccatgttcatgttagacaagcccccctaccactgatttatactccacccagtccttttacaacgtttaaaacttccagacggtctcactgttacccaggctgatgtgaattcactctgcgatgcgatgacacccaggatcccatctcggatcctggagcagagcttggtttcaataagaaagtagccaggctactttctccagtctttctgtctcagcttcctgagtagctgagattacaagctggtgccagccaagcctactagcattatgtcttcttagctgcccagagcactgcccacttacaaactcgtgttaggtttataaccagtagataaatacaataccactttttctccatttctcagtattagagttctgaaagtcactctgacagagtccctgagaaatcaatttaaagaagaaaagatgtattttgactctcagtttcagcagtccatggttgcttggcagcattgattttggtctcatggtgaggcaaaacatcatggccaggagcctgcagttgaacagagtggcccatgttttagcagaccagaggcagagaaagaaatgtgtctggagacggtagtgaatctcacacaatagaaagtgactacccaaaattgtcctctgttccccttctgtgtgccatggcaaatatatattcccccagttacctcccctgcacacccaaaatcagtaaatgtaaaggggtgatcatgtaggaagtgagagataacaatacccatactgacctctggtttccatgagcacatgtacctgacagacatgtacatgtaaacattacatgtgcttataccacactgatgtgaatgagggaatgtaagttgttttaagtgattggaaattaatacatagcctccaggggcataacttaatgacccatttctcttcagctaagttctacctcccaagttttcccgtaacttcctatacaattatgaacctattcgtggattaatctataaatgaggtcaggatcctcatgattgagtaacttctcagaacattgcttttattaggagccaagtcttttcaggagcattccaatgaaaccataatccaccctactacagtcactatctccaacacacaccaggttaggagagcagttcttaagatagttgctacataagatctacaaagctgcctgatttaaaagcagaaattgtaataagatacttctgcttagacttagttatactctctgtcaaagggaagttttccatactctgaagtagttctcttcctagggtactttttaagcttcctagaatctggctcctagaatatcacttacatgtgtctagaaaagtacaggcttcttcatacacctggcaagcctagcgtactttaattttacctctgcagttagccagcgtttcaatgctcaataccctaatcttctctggtttattctaagttacagtgtatattaggcactaaccttcatacctatctcttatagccagataagatcttattccactttgcagaagagaaagaaggtaaataccagagaaaagctaaatatgtagtcagatgtcctgtatttgaagcaacctgtgcctggtgacaaacctcgtgtttgtttcatcctgctgtgcctctaagcagggtaaaacacactgtgtcaggatacatagcaccaagaaataactgtagaatgcttatctgggatatccattttgctcagagtcaagagaaataaactccagttttaagaggaaataaactcaaaaaaaattaatactatagcagttgctttaagccatatcaccagaatcaccatattggtgtcattgttattgtaagttcttctgtaggcaaattagagcatcattcactagaataaacgactctctagtatgtattgaacatagcgtatctcacaccgaactggggagaaaaaggttgaggaaccccaaaactctatggtgtgtgagttgtcaatttttccaaagtgaagataattggcaaacattttgtgttactacagcaaccgtcataaaatttcagcttcttagaacaacacgacctcttcctctcataagttgtacagtgtcagctccaagtcacctgtcatagcttgtcttcatatctcatcctcatagccagctacttcctagatctctaatgagtgagagcagcactttcaagacctagcctttccgtggcaatagggaaaggacaggctagagtgtagagatcagacattttttcaggaattggctctgcttctaccttgctttgaggcatggtctctcatttctgccactgcactgcgttaggagtgctgtagtacagatccagctttctgcgtgggatggaacatagctagacttgttcagcgtgtgcttttatgccctaagctatttcctcagcctactggttggtttttaaatcatatgctttggctctggtgaaaaccatgtccatcggtgtccactggctacaactctgggaaggacaagctaaaggcagtgggctatggatggggattctttccacagagaggggaggattgggagagcaaaacagttaactgcccttaccgttaggtagcctacagtgaggaggggtttagatagggttttcaaagatggaagtaataatccgttaaatacatttcactttattaaatttaaaaactttacttaagtgaagaacaaaagaaaaatagcaaagtaaatattaaactggggttttttaacaatgtcagattagagttaaggatatgaagtggaggctttacagaaaataaatatggttaaaaagtatgaaaaatttcaaactatgaatttgatattaaggtgcatctatttcatatatacaattcactattaaaattaaaatatcttatttttattgagattaaaattttctttgtagagaaaaatcagagtatgtattataattttcccaagtcagtacaagtaaaaaaagcaagtaaagcatgcttgttataaataacttagaaagcataagcaaaattttaattgcctctaactgacccatctaaaatactaaaatccttcagttacccccacacacactccgtgtgtgtgtgtgtgtgtgtgtgtgtgtgtgtgcgtgcgtgcgtgcgtgcgtgcgtgcgcgcgcgcacgtgtctgtacataaatacattttggcttataatttttaatcttttatttgaagtacttttagatttataacagaatgagaaaaattgaaatccaaacctagcactggaaggtaaatgtaggaggatctccaagatcaaagccagcataatctacatagtcagtttcagaacagccagggatacacagagaaaccctgtctccaaaaaaataaataaataaaagaaattattatttattaaataaatagaaagaaaagaaaacaaaaattgtgttccaaatatccttctttcagttttctctcacctgagcgagccctgcatagccagagcttgttgtcaagcctaagaactgcactgccggacttaaggaaggtcttaggcaagtgtctgcagtgctgtgagctcagtaagagcagcacttccttatatacaggacagcagcacttcacagaactcttccccatgctccagctctgactggctttctgccccctcttctgagctttgtgggacttgttatagaggttttatttagagcagggcgctaaacagtcacttattcagagcactttgatcagtttggagcctctgtattcactgctgcacactgcataaaaagcttctcagactcaggttgagaacagtactagccaatgggtagaaacaaatatttggaagacagttttgtactatatgcccgttcagttaaacaacagtactaggttgtcactcataccccagtacctgcctaaggactatatcctttcagtccatgggcattcgaatatgtttaaattaacagacatgaatttcctcctgtggaccaagccttgagttcaatcaaaaagcagtttattccccgcttgtatagcagtaacgccactgtttcacataccttgggcatatcttgccaagtaggatgatgcggctggtgtttgctactttatgtgttctttttcccgccctttatccttcccactcccttacctccatttcacccccatcattagataggagaaaaagcaaagtagaggggagaaaaagagagagggagatctgaatctgatttcttgtttcttctttgaacatgactaccagcaaaccgcaaaccgaaaccagctcccctgaaggaccaccaaaccacacctctcagggccctgtcatttatacatcttctgaaaagttatctgcagctttcaaaaacacacctctgctagaggaagagtcacatcatggtgagctgctactacagtccaaagcagctccacatccctacacctgggagtaaaatgaaagcacattctataatagttctctatcttttaaagaaaccagaattctcactacaggatggtattgtagcatgcaggttctaccctggtaagcccagtatagatttttcttctcagtgatcagcataacaccatcagcatggttggtacaaagcaggcagcggagatgaagtttccagatcagttccagcttgatttccctgtgtccaacaactgaaatgttttgtttttaaaagtagggtgacactgtctagttacattaaggaaataaaagcagtggcagtagcctgtgttgtcttgggaaactttgaggtctatctgacaaataacttgtaggaggtgtctcctcacactgatctgttattattttcatttaataacccacatcttgtggcagtagcgctgtccacctgtgcatgctaataccattctaccagggaagataggctgcaggtgtttgttgtagtgccatgtagatatgaggcctgctttacaaaatggaatttatgccttgtattttatgttgctttgagaaatacctatggaaattttaaagggaggtagactttggtttggatttagagatttaatgacttgtcaactagatccattgctttgggcctaaagtaatacaaaatattatgtcagtacaaacatgtggctgaagctgcttacctcacggcaggtaacaggaaggggtcaggtctatgttatatccttcagaggcacaccagaaacctacttcctccatctatactacagtcccaccacctccctgtagtctgtatgaagccatagtctgtagtttgaatccaactacatgctttgagtaaaagcattaagtcttgagtctttgtgttctaatcatctctggatacatcttcacggacagatgaacatggttaggatatagcaaaccttcctccttgtgccataaaaccagcaacagtacacaattggcagttgctgtgtaagtaaataagatgttatctaacaagctggtatgatggtacatgcctataatgaatgcacttcaaggtgggggaaacaggggcagaagatctggattacatagagagactctgtcttcaaaacaacaccataagaaggacgaatgctttactgtcttcgtcagggtttctacacctgcacaaacatcatgaccaagaagcaagttgggaagaaaagggttggtttattacactgtccacattgctgttcatcaccaaaggaagtcaggactggaactcaagcaggtcaggaagcagaagctaatgcagaggcaggccatagaggggcttgcttcccctggcttgctcagcttgctctcttatagaacccaaggcttccagcacagggatggcatcacccacaatgcgctaggtcctcccccaccttgatcacaagctgagaaaaagccttatagctgggtctcatggaggcatttcctcagctgaggctcctttctctgtgataacttcagcttgtgtcaagttgacacacagaaccagccagtataattgaccccttgtcaacttgacatacacacacatcactattaagcctcaacccttacgctctaattcatccccaagatcaaaataactttaaaagtcccacagtctttacaaattctttcatattaaaatttcaatccctttaaaatatctcttttaaaattcaaagtctttttacaagtctaagtctcttaactagggttccactaaaacactttcttccttcaagagggaaaaatagcagggcacagtcacactcaaaaacaaaatcaaaaagtaaccatccaatatctaggatccacccatgagatcttctgagctcccaagggcttaggtcacttctccagctctgccctttgtaggatacaccttatcttctaggctccagctgcctgttctctgctgctgctgctcttggtggtcatctcatggtagtggtatctttgaaacactgccgtccgccgctgcaactaggcttcactaatagcctctcgtaggctctcttcatggtgtcaagcctcaactcctctgcatgatcccttcagtcctgggccatcaatttcacctggagctgcaccttcactaatggccttccatggcctctaacagtgcccagcctcagctgttcttcatgaccccttcgtgccttcaaaatcagtaccatctgggtgattcttacatgtgaccaagtctagccacagcatgaggtacaaccttgggtatctctggaacacagactctttgcgctctcagaaaacgcttccgaaaagatttcacttcagtgatactggtctcctcttaattgccactaatttattagctctagctgaccagcatcaatagttccagtaatgcaaaagtttcgctttagtagttctggtatctagttaatcacagctgattcttaagccccagctaactaaaaccacaaaatcttcacaataatcgaacaacatggccctaataagagtcttttatcttccctctgaaatttcacaagccaggcctccatcttttgcactgttctcaacattatcttccaagctcctacagaacatcccacagagctcttaacatcccaatagctcttctagctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaccttgtcttagtcagggtactttagagtcaccgaacatataggtattctctatatactaagagaattgtttgatgacttacaacctgttgtccaactccccaacaatgatcagcagcagctgtgaatggaagtccaaggatctagcagtggctcagtccaacaaggcaggtagtcagtcgaaaagagactcttccttcttccaatgtccttatgaaagtctccagcagaagttgtggcccaaattaaggtgtgtaccaccacaactggatctggatcttggtcccaggtgaccttgaactcagatctctttgccctaaactcctgggattcatagctactttgcctcaagatcttcataccaaaatccaggtcagcttcaagatcaggatcaaaggtgagccttccaattctgggttgtagttcattccagatatagtcaagttgacaaccaggaatagccactacactaacctttcctaggtatcttgtaaatccaatctaaatccagtcaagattagatatgcttcttctggtattaattttctatcccagatttcttttgattagtatttagagtatccttcaccagcctttagttcctcacatatacatcctctagagaacatataattaatacattgcctaattttttttaggaattagcagactctgtaccatggatcaaatcttgcttagaatcggtttcgtttatctgtgcctgttgatgtgttggtatggcaacaaaggaacatatttactatctggtattttatagaaaattttgtcttctgatttatctttttaaaaggggtgtgtgtgtgtgtgtatacaggtcctgcttaaatatgtatacaaatgaatgtgaaggccagagcatggcaacaacaggtgtcttcctctaacattcttcaccttagtgtttagacaggttctcttactgacccagagctcattgattggctagactggctggccaatgagcttggaggatctgcctgtttccacttgcccagcataaggattataaaaaccaatatgtgcactcacccatagcttttatgtgggtgtggggacaggcagttcaaatattcagatgtataagtctacattcaaagttaccctgggacacaagcagcctatgggtcagaggttagacagatacctgttttagaatatttaaaaacaactggaaaagtttaatatgtttaccttttagtgctcccctcacctccccccccccttatcatggtttaggctgatgtattgtttttcctttaaaaataattacgtttatttctcagaaatacatacttattggcagtgaattgtttccagctttgagaaaatacttgatcacatttaagatagttttgcctccctctttttctgttctcagttttacttttgatttccagaatactatgttatctctcttcccttcctctactcttttatcccttccacatttatccttatcattaattctgaaaatgtcttaatagctgttttagggttttactgctgtgaagagacaccacgaccaatgcaattcttataaggacaacatttaattgaggctggcttataggtcagaggttcagtccattatcatcaaggtgggaacatggcagcatccaggcaggcatggtacaggaggagctgagagttctatatcttcatctgaaggctgctagcagaatactggctcccagggagctaggatgagggtctcaaagtcacccacagtgacttactccaacaaggccacacctcccaacagtgccactccctgggctaagcatatataaatcattaagtagcatttcagatagttttctgtctgagatttcatttcacatatgttagaacgtttgagactatgacagaccttggtaatttttcccttgtttttcagtatatgtaacttcttgtggcctgtttttagccacactgagctttcttcagctctgtcacttctatttttatccctgttgctattaggtactactttactgctgagctgaccctcagatatgcataacatgcatcctttctgagacattagcctatgttattatagttatctgtgttttatattaaatatttccaacatctgagccatagtttatgaatttgtttgcttatttaaatctgtctagtaattgaaagtccaacacaaggcaaaagactttaaataccacgtcattttttttttaatatttaaagtaatatttaattctgagcacaggagatgacagtaaactgtagattaattttttaaaaaacattagccattgaagttagcacagtcccagtcaccaggattcttggagatgaagcttgatgccttgttgacaaaaaaagttttttaaccatggcttttgggggggggaggtgttaaaggacaaaataaattgaagaacatacaaaatgatgtagtaaacagtgctcacagatcaccaaaatttaaaatgttaaattgttacattcttaagagatgtactaattcaaaacaacctccatcaaaaccacagtggtgttctttgtaatggtaaaaaagttcataaagttcacattaaaacacaaaagatcacaaattctcaaagcaatgccaagccaaaagaataacattaaatattttaacaccagatttcaaattatctgagagagccccagtgacaaagagtgcatggtatggacacaaagtcagggagggttaagagtagagtagaacggagcagctgcacacactagatgtggacaaaggtatcgaaaccatacattgaaaaagagacaagctcttcaacaaataatgcaggaaatcttgtcagttcacatataggaggtgaaactggatccttactttgtgtgttgtacatcaatcaattgaaaatgaactaaagatgtgattgtaagactcagaacacaaaccatgaggaaaaactataggtgcattcagtggctttcctaaaaggactgtaggatctcaggtgattatcacaggacttactcagttgaaacacatgaaatcaaaatgttcctttgcagcaaaagaaataatcagcaggatgaaaatatagactacagaataggaaaaaaatagccaactacatatttaagaaagggttgaaacctaaaattataaagaattcaaaatttaaacttaggaaattggtctagtcaataaatagaaaagtgaattacatcatggtcctcaaagaaagacagatacacatccaataactatatgaaaatttttcactattcaaattagctgaatccaactgaaaattatactggattctatattatttcaaaatggctatcatcaagaataaagaataataatgctagaaacagtagcagaagggtgaaatctactgctagggaaatataaactgttccaagtattgttggaatcattttggaatttcctcagaaaacaaaattgtaactactgctcgatctgttcttcagctcctgaaaatctaaccaaagagattggagccagcaaagaaattctgcccatctaggttcactacagcagaatgcctgatagccacaatgtcattcagggatgagcaattacaaacaaagtagtaaatggataccatgtagttctatataatgtacaggaaaatggatgggactgaggattttgtcaaaataactgagctagagtcaggaagacatgaaacatgtttcatcttctatgtagaacatagattttgagtggggaaggatcatatgagaggaaatgggggaggaaagggaacaacgagaagagaggaggatggcagaacatgagcaaagtcccaatataaaggatgacagtgtcacagggaattcaattatgtagtattccaacaacttaaaaataatatttaaagaatctttagaaaggaattatgattaaagtacaaacaaggtagtgtttctgtagatatctgtttaatctatttggttcataacttctgttagtctcaccttgtctctgtttagtttctgtttccatgatctgtccattgatgagagtggggtgttgaaatctcctactattattgtgtgaggtgcaatgtgtgttttgagctttagtaaggtttcttttacgtatgtaggtgcccttgtatttggagcatagatatttaggattgagcgttcatcttggtggatttttcctttgataaatatgaagtgtcctccatcttttttgatgacttttggttgaaagtcgattttactcgatattagaatggctactccagcttgttttttggaaccatttgcttggaaaattgttttccagccttttactctgtggtagtgtctgtctttgtcctgaggtctgtttcctgtatgcagcaaaaacctgggtcctgtttatgtatgcagtctgttagtctatgcctttttattggggaattgaatccattgatattaagagatattaaggaatagtgattgttgttttgttgttagaggtggaattatgtttgtgcggctaccttcttgtgtttgttgaaagaagattgatttttttttctttttgtaggttgtagtttctctctttgtgtagttctccatctcttatgtagagctggatttgtggaaagatactgtgtaaatttgattttgtcatggaatatcttggtttctccatctaagttaattgagagttttactggataatagtaacctgggctggcattcatgtagagtctgtgtgacatctgcccaggatcttctggctttcagatgtctctggtgagaaggctggtgtaattctgataggtctgcctttatatgttacttgaccttttccccttactgctttcaatattctttgctttgtgaatttggtgttttgattgactattatgtgacgggaggaatttcttttctggtccaatgtatttggagttctgtgggcttcttatatgttcatgggcatatctttctttaggttaaggaagtttacttccataattttggtaattaaggtatttactggccctttaaattgccaatcttcgctctcttctatacctattatccttaggtttgatcttctcattgagtcctggatttcctagttgttttgggttaggagcttctggcattttgcaacttttttgactattgtgtcagggttttctgtgtatcttttgcccttgagattctctcttcaatcttttttattctgttggtgatgcttgcatgtatgactcctgatctctttcctaggttttctatctccagggttgtctctctctttgtgatttctttattctttctatttctgtttttagatcctggatggttttattcaattccttcacctgtttggttgtgttttcctgtaactctttaagggatttttgtgtttcttctttaagggcttctacttgtttacgtgtgttgtcctgtatttctttaagggagttatttatgtccttcttaaagtcctctacatcatcatgagatgtgatttttaaatccaaattttgcttttccagtgtgttaaggtatttagtatttggtgggtgggaggactgggttccgatgatgccaagtagtcttagctacttattctgttgcttagcttcttgtgcttgcctcttgccctctggtcatccctgatgttagctggtcttgctgtgtctgactagagcttgtccctcctctaggtctgtgagcctgtgatcttaggcgtaagagcactcctgggagatcagctctctgtaggcaggctttgggtacagagggctgtaggacagccttagctatgggtgcagatgaagaccaaaatgatcttgtcccaggctgctctgtggttcttgtgtcctgtgtgctcctggcaggtacctctttggacagttattggagtgaaagtggggtctcacttgtgggcttaggagtgagagcactcctgggagacagctttctccgggcaggctttggatctgcagggctgtggaacagccttagctctgggcagagatggagaccagaagctgccaggtcatttttttaagcctcgacataggaacatttttctcctatttggaaggaggacattgctactgtctagggggggagttaggctgttctgttagggtcactgtcgctgaaccacaggcttcaagagtgttgagaactagcttgttttgggggtaaggatcattttccagagcattagctgtttgatttattttggtgtcatttttccacactgtggctcacataggggtttgtatttggggctattttccaggagtatctgttctgattaaggctcggactccggcatctatctctctcttagtctcaagatgatgtaacttttctctaagagctatagtgaatgctggagtaaggcctggaaggttgccctttccttaattgctttaacccttactcagccagggtgtggtgggctcctgtagtcccagcactcagaccagaggattgagattttgaggacaacaaggtctactcagcaggactgagacagaagcacaggctgttgtgcacataacacatccatttcttgatcctcccactcagctgtgctccttcccctgtatctgtaccacagaagattgttaggactctgcattctttccagagaagaaagcattcaagagcatgcaaatttctatagtctccatgctctacagaggttaaaactacccttctcagcagatagcacttaaagtgaaatgacaaaacacgtttactgttatctagttgcctctatcccaaataaggtgtttatctccttaaattttagtaaccttcttccttacataatttgcaaggcacatatatgcttctgttacaaataattcatgttttccaaaactagaatatttttactttttactgctagaattattgaacaatagatatatcttttgaatgtatgaatttgttttgaagcttgctgaaagaatacatttttttattctcttaatttattaagatgtttcaagcctcccttttaatcctggaagaaaaaaaaaaaacagcagaatttaatatcttgaccatttttaagcgtacagttcaggggtactatgtatattatcatcatggtttaatagatttccagaacatttttatcttgcaaaacactcaacctaatggtcatgagtataatgtgtgtgggcactacccatgtaactgaggtaggtttgctatgaatttgagaccaacccatgctgttcacagtaagttccatgctaatctgtgatgtagagcaagatctctcaaaacaagaaccccaaacctctctgtattaaacccccactatccccattcctatcttctggcagatgaataagcgttaatcttagaagcagaattctacagtttcgtcttttatgattagcttacttcacaaagcagggttcacattatagcacataccaaggtgttcttcattttaaaggccaagtaacagtttattgtatgtaagcaccttgttttcttcatccatttgtcatatatgtgtatatatgagattgatgaattgtatgcttaattctattcatagtgttctacacacatacacacaccattctgttggtcagccttggcagaaattgggccatttgtaaatgctatgcattctacaggtttgctcagctatagcactttatattcccaattgtagatagaactgtgctagcttcttacatcctttcgtttaatactttcatttaataatttcttatagtattgattgtcatttttagtatagctctcctaatagttgtcaagtactatcttgtggttctgatttgcattcccctagttattgatattgagcctattgtctatctgtgtatcatctaagtctgtgctaaaattttgtacgtgcgtgtgtgtatgtgtgtgtgcgcgcgcaaccctttgttttcaccttatttttttgtaaaaatgtttctcaatgaacctggagcttctggagcttgacagctagaatagctacctggcaagcctcagggacctacctgtcactgctcatgtgtttctgtacagaaagtctttacctgtcaaaactgtccttcactttctccttaccaggttctgttgtcagattttatgtgaaagtctgtgatccatgtgaagttgagttttgtatagggtgaaaatctgtttggattcttccgcatctagtgatctagttttattggcaccatttgttgaagatgatgtctgtttcagtatgtatttatcagtaatcaggtgtccatagatgtctgggtgcatttgtgtctgggtctttaatttgagtttttgactagtgtgtctcttcttgtgccattaccatgctcgttttattactatagctctgtagtacaagatggcatcagggctagcaatatctcctgcagtccttttattcaggattgttttaactatcctggggtttttatgtttccatatgaagcggaaaattgtcctttcaaattccctggagatttgtgttggaattttgatgggcatatttattacttttgctaggatggccatttttactgtattaatcctaccgatccatgagcatggaaagtctttctatcttctgatatcttcaatttctttcttattgtcttaaagtttttattataaagtctgtcgattacttagatagaatgatcccaaaatactttatattatttaaggctattgtgaaaggaattatatccttgattttagtccacttgtcatttgcatagaagagttactgatttatgtagccatcagctgtgctgactgtcccccagctataggagtttgctggtggagtttttgattcacttatgtataacatcatatcatctctaagtaaagatacgttggcttttcctttcctatttgtatccccttgatctccttcagtcatcttattgctctagctaaattcaagtactgtattgaatagatatagaacaaatggacagccttgtcttgtacctgatttagtagtattgctttgagtttgtctccatttaaattgatgctgggtataggcttagtgttgattgcctttattttattgaggtatgtcccttgtaaccctaatctctccaggactttgtgaagaagtgttgtcaactaatgaaaatattttgtgatttttttttttttgtcttttggtttgtttatatggtagattacatttatctatttacatatgttgagccatccctatatctctggaatgaatcctacttgatcagggttgatgatctctttgatgtgctcatgaattcagtttggaagttttttcttgagaatttttgaagttatgtccataatggggaattggtctgtaattctctttctttgttgggtctttatgtggtttggctatcagggcccacataaaaagagttgggcaatgattctgttttgtagagtattgtgagaattattggcattaaatattctttgaaagtctggtagaattgtatttgctaaaatcatctggccttgggcctgatttttggggggaagacagttaatgactgctttattagaggtcgtgggtctaattaaactgtttatctgatcttggttttcctattgagaaaaattacccatttcttttagatttttacaatttggtggaataatggtttctaagtatgtccttgtagttctctggatttccttgggtctgttgttgtgccacacttttcattttttaattttgttaatttgaatattccctctctgcctgttagttaatttggataagggtttgctaatgttaatgatttttctcaaaggaccaactctgtttcattgattctttgttttgtttctattttaatgatttgggccctgagttttattatttctttccctctactactttgaggtgtgtagtgagaattttgggattttcggtttctttgaccaaaaaaagtgcagacatggggctacttcagaccgtccacagcagctctgatctgcctcgtgctctggtttcttagtgatagtttctgggagggtgtgatgtttggaattctggtgacttcagagggttcagaaaggctaggcccagaggggttgtgggttattgggtggtggtggtggtttgttaattagtcgtgtgcaaagaagaaacaagaaaaagaagaaatccctgagagtgaagataatcttgccccgaggaatttgagcccctaatcagcaggaagtagtctgaagataatgacgtctacttccccctctatgcttttttctcccctatctaatgttagtgggttgaaagggtagaagaaaaagaacctacaaacgtagccaaagtccagaagtatttctgctttttgttctagagctttcgtgtatgttgtcaagttactttgtatgagatctctgcaatgtcttttttcatgcaagcacttaaagctatgaacttgcctcttagaaccaacgtggtagtgttccatgagtttggatgttgtgtattcgttttcgtccaattctaggaagtctttagtttcttactgtctgggtggacccatctttcattccagagtgtgttgttcagtttccacacagtttgtaggatgtcttttgccaggaatttttagatttaacattttctttgatcatttcttgtgtcctgtcttcaaaacctgagattctctcatcatctttgttctctgttggtgtggcttgactctgtagttcctgttccaagttttatctccagattttcccagtttgggtttcctttattgactcttgtttccactttcaggttttgaactgttttatcccttccattccactccttgtttgtgacttcatggatgtctttaagggatttcttcatttcctctatcatattgatgaaggctatttttaagtctttgcttgtgtttcagctctgttacagtactccaggcctgctctggtgggtttgctggggtctagtggagacattgtcctggctattactagttttatgctgacatctaggcatctggtttgagaagaatgaaattctgggtgctggaatctgatcttgtgtttgttttaagggtgatttagtccttggtttctgttgccctctgattcttaggcgagtgtggttgctctatgttgcctagtaggaaattcttctgggatctgacacgtctaggatggtcctagacttggggtgcacagggaggagcaaagcagggtgttccaccaagatctgcttagaccccgggaatggggtcagagtgagaagtcacaagtaatcttctatagggtaggggtgtgtgactgggggactagacttggaggaatttaaggagagttgaggatgggtgcaggaatggggcacagaggcctctatatgatctgcatagctagggattggatttggattagaggaaatgtaagtgaagatcaggaattagtctaccttgttgccctggtcagagtggcatctggattccagggggagttcctgtggagtcagggtctggggtaaggcaatgagttgggggcaggaggcagaggttagcaggctttgaaggaagcctctgcaggtgttctgctgcaaagctgggaatgagactgggaattagatttggagaaacagaggggaaaaggagatctgcagttggcctacatacttcccaataaccttcctttataagagtcttgcattaaatctgaagctcaaggattgagctaggctctatggccagtccattccattcactctgcctctgtcccagcccattgttggcttttatcagggtgcagggcatatgaatccagtctactgctagtgcatctggcactttccctactaagccagttttagggaattgaaagtatgttttctccatagtgcgtgtgcacatgtatgcaagcagataagtaaaggaaaaaagtaggttcttaccttcaaccatgcgggattcaggggttgaactcagggtgtttagctaagcagcaaggggcaacctaaacatcatttactgtattataatatcctttgcatatctcagctgtttctctagaaacattctgttctgaaattagtggaatggagtttatgaacataatgtttttgtgtttttcatttttagggaagcagcaagagaatgtcgtagaaagaagaaagaatatgtgaaatgtttagagaacagagtggcagtgcttgaaaaccaaaacaaaacattgattgaggagctaaaagcacttaaggacctttactgccacaagtcagattaatttgggatttaaattttcacctattgtggtggaaaatggactggattggccacaaccagaaagacaaataaacatttattttctaaacatttcttttttctatgcgcaaaactgcctggaagcaactacagaatctcactcatttctgctttcgcgttaaactgtgaatgtccacaaccgcttccacttctgccctcacgaagtaaatgtgcagcgccaggaaccatgaggagacctctgccctcacctctaccaccctcaagaagtaatcatttgcttatttgtaaattgttgggggaaatgaggaaaatgaaatcttggctttcttatttttgttttaggtttgttttgtttgttttctttttaatgatttcaaggtttgtgctgagctccgtgattgccttagggacaattacccagcctcctgagctgaagtaatgtgtgggacacatgaggaaagtaagtaaggtgcaatgaagaagtgttgattgccaaattgatgtttttacacttcattgtggattatgtaaaactattaaagacatcctttaaataaggatcttagtgtggtgttgaggagtaaacattctatgtatgttcctctcttcagtactgacaacacgctgtccttggttctgggaagcgtttggtggtagttacagctcttccatagggcagttgttgcttcttcagttcttgtgtgttcgccatttgaattaaaagaagtagccagctgagtggaaaatatggtattgaattttgagttcagttaaaatcatagtataaagcttattttggttagtactaaatcttaatgaacaggtgctggccaggaaggcaactccttgaggtataataaggtttttaataaataaaaacttttgtccttgctttcaaattccttaactgtcactcatttacctgaaaagctatttctcattcactggcatttacactgtgcaaccaacaggagcgttttcaagatgcccaggcaaagtgattgccctcagaaagtactaaattgacaacttttcttatattcacatatttttatctgaaaatctcttggaattttctttaactcgaagttacaaggatgtgattctaaaacattgtgttaaatgcacacaagaaaagtaattttcttcaggcattggaatgtgtgctatggtacaatatttggcaaaacacttttttttctaccaaaaaaaggtggttctgctttaactatccaaaagctggtctgtttctaaagatgctccctacttaaatcttaactcatagtctacatttgtatgtaacattgtttctgataagacttaataattcttttttttttttcattaagcctgataaaatctaagagttacctctttaaaagacaaataagaggacagtggaggccagcatgttagattatgtcatgtttgagaaagaactcagactcacaaggaggttttatatttgctttgggatattaagaaagacgatctttatgtcagaatattaagagtgtaaaatgtttggcccatttttaaagtaagactttaggttgctgtctgtaaatgttgtatgttgtttataacaagccattgtcttttttcaaggatgaacagtttgtgaaggaacatcattctaagagttgagtgatgtaataatccttatgtctggacagttcaccagattctccagaaggctttcaaacgactaaagtttgatgtttgtcctgctgagcttactgggaaagagatagcataaaaactgtctgtactagcatgtagatgtatcatttgctaaacttggtgaaacaaaaaatgaagcagaatcacaaatctgtcaacaaactggaattacttgatattaagtcaaaatgtcagattgagcattttaaatgtcacaagtggaaaatttgaggagggtgtagacgttgtaatctctaagtgtctgtcttcaaaagttgaagttttctacatagtgaggaaatggatggtcctcttaacccttagagaccaaggcagcctcagtcatacttcctaattggatggatattaatttacagaggcctgggcctttatgcttgaagcatgactgtactggtttgactccagttacttcatgt

SEQ ID NO: 3 - DNA, 1125 bp, Rattus norvegicus
gaaagcagtgactgaggagcttgtaccaccggtgactagatgaccatggactctggagcagacaaccagcagagtggagatgctgctgtaacagaagctgaaagtcaacaaatgacagttcaagcccagccacagattgccacattagcccaggtatccatgccagcagctcatgcgacgtcatctgctcccactgtaaccttagtgcagctgcccaatgggcagacagtccaggtccatggggtcatccaggcggcccagccatcagttattcagtctccacaagtccaaacagttcagtcttcctgtaaggacttaaaaagacttttctccggaactcagatttcaactattgcagaaagtgaagattcacaggagtctgtggatagtgtaactgattcccaaaaacgaagggaaatcctttcaaggaggccttcctacaggaaaattttgaatgacttatcttctgatgcaccaggggtgccaaggattgaagaagaaaaatcagaagaagagacttcagcccctgccatcaccactgtaacagtgccaaccccgatttaccaaactagcagtgggcagtatattgccattacccagggaggagcaatacagctggctaacaatggtaccgatggggtacagggcctgcagacattaaccatgaccaatgcagctgccactcagccgggtactaccattctacaatatgcacagaccactgatggacagcagattctagtgcccagcaaccaagttgttgttcaagctgcctctggtgatgtacaaacataccagattcgcacagcacccactagcaccattgcccctggagttgttatggcgtcctccccagcacttcctacacagcctgctgaagaagcagcacgaaagagagaggttcgtctaatgaagaacagggaagcagcaagagaatgtcgtagaaagaagaaagaatatgtgaaatgtttagagaacagagtggcagtgcttgaaaaccaaaacaaaacattgattgaggagctaaaagcacttaaggacctttactgccacaagtcagattaatttgggatttaaattttcacctatgtggtggaaaatggactggattggccacaaccagaa

SEQ ID NO: 4 - DNA, 776 bp, Rattus norvegicus
tgggcttagacagttcggtcggattcccgggatgctgctgctgcctgtggcccgggcggctgggagaagccgagtgttggtgagtgacgcggcggaggtgtagtttgacgcggtgtgttacgtgggggagagaataaaactccagcgagatccgggccgcgaacgaaagcagtgacggaggagcttgtaccaccggtgactagatgaccatggactctggagcagacaaccagcagagtggagatgctgctgtaacagaagctgaaagtcaacaaatgacagttcaagcccagccacagattgccacattagcccaggtatccatgccagcagctcatgcgacgtcatctgctcccactgtaaccttagtgcagctgcccaatgggcagacagtccaggtccatggggtcatccaggcggcccagccatcagttattcagtctccacaagtccaaacagttcagtcattgttctaggtgctatgaaaggcagcagtgagcaaacagtacatctctgttctcatgaagatcacacttgagtgaagagacacactatgagaaaataaaatgtgtcagatttcaactattgcagaagtgaagatcacaggatctgtggatagtgtactgatcccaaaacgaaggaaatctttcaggagccttctacggaaatttgatgactttctctgatgcacagggtgccagatgagagaaatcaagagaactcagcctgcatccctgacatgcaccgatacaatacatggcaaatgctaccagaga

SEQ ID NO: 5 - DNA, 486 bp, Rattus norvegicus
atgtcgtagaaagaagaaagaatatgtgaaatgtttagagaacagagtggcagtgcttgaaaaccaaaacaaaacattgattgaggagctaaaagcacttaaggaccttaactgccacaagtcagattaatttgggatttaaattttcacctattgtggtggaaaatggactggattggccacaaccagaaagacaaataaacatttattttctaaacatttcttttttctatgcgcaaaactgcctggaagcaactacagaatctcactcatttctgctttcgcgttaaactgtgaatgtccacaaccgcttccacttctgccctcacgaagtaaatgtgcagcgccaggaaccatgaggagacctctgccctcacctataccaccctcaagaagtaatcatttgcttatttgtaaattgttgggggaaatgaggaaaatgaaatcttggctttcttatttttgttttaaaaaaaaaaaaaaaaa

SEQ ID NO: 6 - DNA, 1023 bp, Rattus norvegicus
atgaccatggactctggagcagacaaccagcagagtggagatgctgctgtaacagaagctgaaagtcaacaaatgacagttcaagcccagccacagattgccacattagcccaggtatccatgccagcagctcatgcgacgtcatctgctcccactgtaaccttagtgcagctgcccaatgggcagacagtccaggtccatggggtcatccaggcggcccagccatcagttattcagtctccacaagtccaaacagttcagatttcaactattgcagaaagtgaagattcacaggagtctgtggatagtgtaactgattcccaaaaacgaagggaaatcctttcaaggaggccttcctacaagaaaatcttgaatgacttatcttctgatgcaccaggggtgccaaggattgaagaagaaaaatcagaagaagagacttcagcccctgccatcaccactgtaacagtgccaaccccgatttaccaaactagcagtgggcagtatattgccattacccagggaggagcaatacagctggctaacaatggtaccgatggggtgcagggcctgcagacattaaccatgaccaatgcagctgccactcagccgggtactaccattctacaatatgcacagaccactgatggacagcagattctagtgcccagcaaccaagttgttgttcaagctgcctctggtgatgtacaaacataccagattcgcacagcacccactagcaccattgcccctggagttgttatggcgtcctccccagcacttcctacacagcctgctgaagaagcagcacgaaagagagaggttcgtctaatgaagaacagggaagcagcaagagaatgtcgtagaaagaagaaagaatatgtgaaatgtttagagaacagagtggcagtgcttaaaaaccaaaacaaaacattgattgaggagctaaaagcacttaaggacctttactgccacaagtcagattaatttgggatttaaattttcacctattgtggtgaaaatgga

SEQ ID NO: 7 - DNA, 2106 bp, Mus musculus
ccacgcgtccgcggacgcgtggggcggagtgttggtgagtgacgcggcggaggtgtagtttgacgcggtgtgttacgtgggggagagaataaaactccagcgagatccgggccgcgaacgaaagcagtgacggaggagcttgtaccaccggtaactaaatgaccatggaatctggagcagacaaccagcagagtggagatgctgctgtaacagaagctgaaaatcaacaaatgacagttcaagcccagccacagattgccacattagcccaggtatccatgccagcagctcatgcaacatcatctgctcccactgtaaccttagtgcagctgcccaatgggcagacagtccaggtccatggcgttatccaggcggcccagccatcagttatccagtctccacaagtccaaacagttcagatttcaactattgcagaaagtgaagattcacaggagtctgtggatagtgtaactgactcccaaaaacgaagggaaatcctttcaaggaggccttcctacaggaaaattttgaatgacttatcttctgatgcaccaggggtgccaaggattgaagaagaaaagtcagaagaggagacttcagcccctgccatcaccactgtaacagtgccaacccccatttaccaaactagcagtgggcagtacattgccattacccagggaggagcaatacagctggctaacaatggtacggatggggtacagggcctgcagacattaaccatgaccaatgcagctgccactcagccgggtactaccattctacagtatgcacagaccactgatggacagcagattctagtgcccagcaaccaagttgttgttcaagctgcctcaggcgatgtacaaacataccagatccgcacagcacccacgagcaccattgcccctggagttgttatggcatcctccccagcacttcctacacagcctgctgaagaagcagcacggaagagagaggtccgtctaatgaagaacagggaggcagcaagagaatgtcgtagaaagaagaaagaatatgtgaaatgtttagagaacagagtggcagtgcttgaaaaccaaaacaaaacattgattgaggagctaaaagcacttaaggaccttcactgccacaaatcagattaatttgggatttaagttctctcctgttacggtggagaatggactggcttggccaaaaccagaaagacaagtaaacatttattttctaaacatttctttttttctatgcgcaaaactgcctgaaagcaactacagaatttcattcatttctgcttttgcattaaactgtgaatgttccaaaaactacttccacttctgccctcaagaaatgtgcagcgccaggaatcatgaagagacttctgccctccgtctccaccaccctcaagaagtaatcatttgcttatttgtaaattgttgggggaaatgaggaaaatgaaatcttggctttcttatttttgttttaggtttgttttgtttgttttcttattaatgacttcaaggtttgttgagctccatgattgccttagggacagttacccagcctgctgagctgacgtaatgtgtgggccacacaggaaagtaaggaaggtgcaatgaagaagtgttgactgccaaattaatgtttttacactttcattgtgaattatgtcgaactattaaaaacatcctttaaataaggatattagtgtggtgttgggagtaaactttgtatgtatgttcctttcttcagcactgacaagttgtccttggtgctgagaagcacggtattggctacagttcctctgtagggcagttgttgcttcttcattcagttctgtgtgttcagcagtttgaatacattaacagaagtaaccaactgaatggaaagcatggttttgaattttgagttaagttaaaatcatactataaagcttattctggtgagtactaagtcttaatgagtaggtgctggccaggaagctaactccttgagttatacaataaggtgtttaataaataaagacttttgtccttgctttccgaaaaaaaaaaaaaaaaaaaaaaaaaaaaaaaaaaaaaaaaaaaaaaaaaaaaaaaaaaaaaaaaa

SEQ ID NO: 8 - DNA, 1205 bp, Mus musculus
gtttgacgcggtgtgttacgtgggggagagaataaaactccagcgagatccgggccgcgaacgaaagcagtgacggaggagcttgtaccaccggtaactaaatgaccatggaatctggagcagacaaccagcagagtggagatgctgctgtaacagaagctgaaaatcaacaaatgacagttcaagcccagccacagattgccacattagcccaggtatccatgccagcagctcatgcaacatcatctgctcccactgtaaccttagtgcagctgcccaatgggcagacagtccaggtccatggcgttatccaggcggcccagccatcagttatccagtctccacaagtccaaacagttcagtcttcctgtaaggacttaaaaagacttttctccggaactcagatttcaactattgcagaaagtgaagattcacaggagtctgtggatagtgtaactgattcccaaaaacgaagggaaatcctttcaaggaggccttcctacaggaaaattttgaatgacttatcttctgatgcaccaggggtgccaaggattgaagaagaaaagtcagaagaggagacttcagcccctgccatcaccactgtaacagtgccaacccccatttaccaaactagcagtgggcagtacattgccattacccagggaggagcaatacagctggctaacaatggtacggatggggtacagggcctgcagacattaaccatgaccaatgcagctgccactcagccgggtactaccattctacagtatgcacagaccactgatggacagcagattctagtgcccagcaaccaagttgttgttcaagctgcctcaggcgatgtacaaacataccagatccgcacagcacccacgagcaccattgcccctggagttgttatggcgtcctccccagcacttcctacacagcctgctgaagaagcagcacggaagagagaggtccgtctaatgaagaacagggaggcagcaagagaatgtcgtagaaagaagaaagaatatgtgaaatgtttagagaacagagtggcagtgcttgaaaaccaaaacaaaacattgattgaggagctaaaagcacttaaggacctttactgccacaaatcagattaatttgggatttaagttctctcctgttacggtgtgaggaatgggactgggctctgggctacaacctgaaagaacaaggaa

SEQ ID NO: 9 - DNA, 76981 bp, Mus musculus
  misc_feature (1)...(76981): n = A,T,C or G
tccaaccttccagcgccgtccgcgcggctctgggccctcgcggcgcgtcaaagttcccgggcggtctgcttccgccgcaggatcagaccgcgcgggggtttccagcaagtccccgccaccccgggaatccccacaccaccccaaatctggctacacacaaaccccgcttcctccaaatctcctcagctggcgtccgaagcctccacccgccacagtgaggctcggatcgtgcctccagagcgggtctcaaaccgcctacaccagcttccccggtcaaccccttcgcagcgccgggaagtagccgaaggccttcggcaggttccgccagtagcgcgggggcggggcctgtcgagcggctccgggtcgagctcggctgtttccgtgagtggccgctgcgcactcggcactgggcggcgctggctggctccctggctgcggctcctcagtcggcggcggctgctgctgcctgtggcccgggcggctgggagaagcggagtgttggtgagtgacgcggcggaggtgtagtttgacgcggtgtgttacgtgggggagagaataaaactccagcgagatccgggccgcgaacgaaagcagtgacggaggagcttgtaccaccggtaagaggagcaggaggaggaggcaggagccggagagagccggggggacagaggggggccggggaggcgccggacacccgcgctcggggccttcccctcgcaggaggggccgcggtaaagatggagcctccgcccgagcccaacaacaccgccgtcgttcgcagcgaacaaagaataatggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngagaagttgagggcagaggaggtgaaggtgctgccgccttcgcggcggtgcccgctcggcgagccgcaacgtccgacctgctttcctctacagcgctctttgtgcaataaagttcgaaacccaaggttttctctggtgctgtattttattcttgcccctctccccggctcccacgcggtcggagaaaatgctgttttctgccctaggctgttttaagggtttggggttttttgtttttgttttgtttggtttgtttttgttttttgttttttttttactatttccctcacccctcggctttgaagatgaagaacgaatcagtagtcctgggaatgcattcttttgggaaggaaagaggtgctaagaaaacatacatgtaataactttttgcagaaactgttgggatcttgtaaaagggaagctggtgcttgaaaacagtttcggttttaaccctttccactaagataatgatcacttgaggtcaatcattctgaaacccttagaaattatcgaaggggaaattagtattagcgaaggtacttttcgttttctgtcatttgaagggaggcagtcttgccaaggttttttgagtcatccgtatctccccttagcctctgccatgctcctcacacgcttctgtctgaactttaatggaggattgatataattctcactctaaaaatagttattgaaactatccattatttgtatattgccgtaggtaatcccagcctgaggttgatggcagtttaagccagcaggggttgtggtgttcctttttttccttcacctagaacatctgcaaaggggactgtcaaaagtcattgacaccttaaagatccaaagacatttttaaagggcattgcagtatgacctatttaaacctgccatatcttctcaaagttaagagtttgatcctagcctcagttttgcaccacatctgccttcaaaagatctagcacaaaatacctttgggccatctattgaaatcttgtgccctgatgatcatttcccaattgaaaagcaaagcccaaaattttagttgatttgaaaggtgcacagttttttatatatttgggggggggggttgcttgtttcttttacaatacaaccagttactgtaccattgaattgtgattggtccatacaaggcttttggttaggaataaatattgattggtaatagaaccattgtgtttgattttttttctatgcgtcaggactctgaaacttgacaagttacattgctagtctttagcactccaatttgcaattgttaagaaaattaagatgcaatagaaggagttgtttccatagggttgagaagatgattttgtttccacacagtgaagactctatacatggtaatgttgatgataaaatagttttttcaaaagattttctacaagccaatagtgatgatacatgaggatgattcttcccctgtgagaataaaaaaaaattcctatgtgtcctttcttcagtgttgaccaaaagatactttaggttttatttatgggatcttttatggagaatcaaattgattctcagcaattgcaagtgaaacttggaacactttttgtgtgcattaattttcaagcattcacctggaacctgacaataaattacagttctctcttgcagaggtcagtgaaagttctagcacggtgtgcttccctcctcttcctccttcctctcctttagttgtttatttttgatggacctatgtattttagaaagctaatgaattatttacaaaaacttacctaatgaatactaagtgagcattcctttggaatttaaaactagtgttaaacacttttgaacagaatttcacacaagggcctgaagaaacagaccataatattcttttttcctaccctcagatactcatagggattttagttttcttagagaagtagttttgtcacttgtaaagcccttgggtttgtttttttaaatctcatatgccttttttaaaacaatagagaacattgtttacttgtgacagtgcttatacgatataaaatgtttattacaaatacgttagcagatacagccatataaactcacctgttatacattcgtgtgagcatctttcttgctgtattacttctagtggtagcttcattattttgaataaatagagctcaaatcttagcttaagaatataatagatatcagaaaatagataattatagcagtttattcctcatgcatgtgtgaacttgactgccccgctgaatacttgacagtagtcactggtgatgtgaatatttaacacatatagtgatgggcagtacattgaaagatcgtctatctggtttgggaaatcacgagtgtgggtttgaaactgctagtcttgtcacatgcctgcacttggggagttttgtgaccatgggacactgtctgctatctctgcctcagtttcttcacttgtaaagttaaaataatagcatatactcctgggggttttgagataaatagaaagtactttttctagggtgagtttaactgttcatcttagtttttacctcttacctttgggacttcccaaagatgtcataatttctccaaacttactgtgtttaatacagttggccagaaagaccaacaaatccaacgaggaattttactggttttatgcttaaggaactgttgggaacagttatttgtcatttgaaaacaatcaaaggaaagagaacactcttggtgagctgaaaaggagtctttgtattttcagttttgtgttttaattatttttgtggttatagtgtaaaacataagttatagcaacatatcctatattctgtgtcatccagtgttgcccacatgttttcagagatgctctgtccatatgcaagtaaacatgtatgtgtatcgtcctctatacaagcagtagcatactatacaatctgtcttgaattctgtcctcccttacctgtttgacactcatttcccaggctacttataatgacttagcaattgatttgtataagcatgtaaagacctttttaaaaagatctttaaattgtatgtgtatatgtgtatgtttgagtgtatgaatgggcacagtgtctgtaggagcccaaaatggtgagaagaggacatcggatcccctggagctgcacttgtgagccaccgtgtggatgttggaacacaacctgggtcttctgcaagagcacttaaccactgggctgtctctccaacctgaaccttttttccttttccattttttattaggtatttagctcatttacatttccaatgctataccaaaagtcccccatacccgcccactcccctacccacccactccccctttttggccctggcgttcccctgtactggggcatataaagtttgcaagtccaatgggcctctctttccagtgatggccgactaggccatcttttgatgcatatgcagctagagtcaagagctccggggtactggttagttcataatgttgttccacccatagggttgcagatctctttagctccttgagtactttctctagctcctccatcaacctgaaccttttaaaagaagcatgtatatagggatatacttaaatttattaaccttttagtaagctgattctatttctaggtcatgcacggccttttaaattgtaatgccaccaagcttggtattagttcatgcctttaatcccagcacttggggcaaaggctgctggatctctaaattcaaggccagccttgttttacaaagtaagttccagcaagggctacatagtaaaacctagtctcaaacacaaactttatttaagtaacactcatctgagtgaaaagtaatggactagttaataagtggaagttgaggtttaatttaaactgccttatttttagctccactttataaaagctttagttaaaagattgagatgtacatagcaacaagcaaactatgagtttagaaggaatgactttgcttcattagaattcttaccaagctgaactttcaattactattttgaccaaaaaaagtcttggaagttttctctactggtaaaaaacaaaaaaagaatggttaattaaatttagtgcagtgttttgtttgagagggagaacggctcttttctgtttatttggttgtttgaggagggtcttggtgtgtaatctagactatcctagaactcattatggagcgtaggctggcctcagacttgaagtcattttgccttagcttgtagccttgtgagtactgaggttacagttttgttttttaaacatttcttggtgtaatttaccagtgagttttgtatttcttgctcaaatagaaaacaagtaggttggcacacgtctatctcctgggagtcacactgtactgctgccttttttaattgttgaaagctcttagtcagtcagctggactatgtaaaatgcatttcagccccagcactgaacaagtctcagtagctctttgtttctttcttggctcttcaaagcagcttaaagctatagtaggttttaagtatacatgctacagattttctaaacagtgcatccccctgttttcttttagttattaaataaagctgtcacaatagagtgaattgtttgtatggttaaaggcttaagttgagaatgacagtgttgtggtcatgcaggttgcagttttacaagactactcttactgctctttggagcttttcttagtattataaatattaacatgctgcttttacctgctgcatagttaaaggactacacaaagcatgtgtatagatttggtaaaaatggggtagataattgaagtttataaaggcaggtgacttgcgggtatttgcaagctatcagcaaggtacatacatggtttcttgatgtctttgggtcttccttcagtttagtattttgttctaatcattggggtttgtttgtttgtttgttttttctagtctgggcttctctgtgttccctaactgtcctggagcttgtgctgtatgtagaacaggctggccttgaattcagagatcacctgtgtctgcttcccagacgttgggattaaaggcatggctgatgtagaataattgttttatatacacacagatgctgagtttattttataaatgtgggttttaaagtttgaatgagggtagccatttcgtcttctccgtcactcagattgtatttcttcccttactatgagcacactcttgtctcaggtcctgactttgtttagcttatggtctcttttcttaattcttccagactagttcatttaaaatatatgttattattattttgtctagatttttggttatctcttgggttaccctaaattgcctttcatattctcctccactttaggagaggaacataatctgtaatttctgcacacattagatagtccagccatgaaaaattgttcagaactaagtacctaattcttgaggctatttacataaatatcaaagcagtagttgggactgatcacgtgactcgatgagagcacttgcctatgatgaacaagacctaaatagatgcaagcaaggtttgtgaggaggtgatctaggggatttatagagcttttttttttctaaaaggagaaaagattgatactttggttcaagaagagtgagctgagtgggaagataaaatagccacttcctgttcctggagtaaagggtgtgtcttaatgctgactaatactagtaggaatggaaatttctttcccaaacattaatggatcctctttgaaactgagatttattcaatcgattgtagttgtatcctaataacctggtgatttgcatgtgaaattctggttgtgattatgatatagatgtagcaagcaatgtttaattcggcaagggtataatgaaacattctccagaaatttatccctgcttgctttagcaattgacaaaaagagacttggaagtctcttatttttcatcaccaaagtaataattattgctcatttgaagcatttttattcttcttagggtttttcttttttgtgtgattttgtttgtttgtcttgagacagaaatcttcccttgtggattggttgtcctgtatctacctacctccgccttctgtgctggggttaaaagtggtatcattttcttgaatcaatagaagcctgcaaataataatagtaatattctgctgttctttgttgctatttatcaacctaatttgatcatggaacatttgatgatcatcaattcaaagttgatgagcaaatcacaagaaaatgtattcaggtagttaagaaaataattcctagccaggcagtggtggtgcacgcctttgatcacagtacttgggaggcagaggcaggcggatttctgagttcgaggccagcctggtctacagagtgagttccaggacagccagggctacacagagaaaccctgtctggaaaaaaacaaaaaacaaaccaaaaaaaaaaaaaaaaaaagaaaaaaaataattcctgaatgttacatgaagatgtttagatgttataagtaaaaaaagtccaaatatgtctcttacccacaaagaatttgcagtacatactgtagggtatggtgatgcaaggacaagtttctgctgtcatgaagagctatagcgtttagaagagaaaagaattcacatggggaacttggtgggaagatagcattcaaatggagtattgaagaatttgtataggtagaagtaaaaagggtgtgtcatagacagtaaaatggaaaaccgtaagaggacgacgctgaggtgcaatgaacttgatatacacacaaattggtgaagagaatgttagattagctacagatcaacactttaaggaatcctgaatatgaatcctcatggcacaggtttaagttttatgtaagtagtgtgtgtgtgtgtgtgtgtgtgtgtgtgtgtgtgtgtgtttgtgttatatgtgtatatgaacaatacaatgactaccttaaaagtgtagttttttttacttccagtgtgttgtattataaaggacacaatagcttctctataaacttttttgaagtgttatatataatcttcccatttggtaggtacagttccatgatttttaatgttgttagccagttgtgcatctactgccacagtcagccctgggacatattcaccacctcagaaagaaactatcaccccttaaacttcctacacaacagcccaagacaacagctaatctgctttgtgctttattaatttgtttttggacatttcatatgaataaaagcatacagtgtattatcatcttagcataattttttcaaagtccacctatgtagtatttatcagtattaatttctgttgccaaataatagtccacattatgctcaatattttactcattcgttcatggacactgggctttttgatttgagttatactaatataaacattttcatttctcttgtatagatatctaggaaagaacgtgttgggttgtatgctaaccctgttaaagctgttttgaggaactgccaggccattttctaaaactactgcacatattttataaggagtgtataagggttttaatgcctccatgtcctcactaatacttgctataatctatcttttccattatatccattcgtgtgggtgacaaggaagctattttgatttgtattttcctaacggtatcaggcatcttttcaaatgtgtttattggtcctttatatatctttgaaaaatgtatttgtttagatcctttgccttaccaggtacatatataatttatatttttccctcccattgtatagattgtcattttatgttttgttttgttttgttttgttttttccagacaagttctcactatatagctctgactggccttggatttgctctgtagactatgcctacctcaagcttacagagatctgcctgcttctacctctcaggttctagaattaaacgtgtgtgccatcatgcttgaggcataaaatttttcatttttatttctggttcatcaattttatttttcttctgttgcttgtacccttagaatctttttctaaacatcacattttttttcccccaagtaactgaaagcccagcaaacaatagcattgcttaatgaaaaacagttaactgaatggatgcaggactatgttctactttcttctgctcttctccattaatctgtgtccttatgccttttttttttttttggagtgacttatgtaaaaaaaaaatgaaaagatgaaaaatcaagaaatggcaattttgaatataacataataattggttatatttttttagttcatttatggtgtcagagtaagtatagtgtattacttataaactaccttacttgggaaatttatatataagtttggaagtttatataaacttagatgtgagggaaattttagtgcctttgtttgtgtgtttgtttgtttgtttgtttgtttaaagaagactaaattggtcctgtggactgtaaatgatgtacttactgtcatagactgctcgttttttggtcacattatctctgtggaaaaagttttcaaggctcttgttttattaataactcctaaagtttgtctctaagcaatatcagctttagtttataaatagctcacttggaggtgattatcctagtataggtcatggaaaatgcattaatttccttattttattaatcagatctttatcagtagagaggttataccctccactcccaatttcttaaatcctagtcatttccaaatggacatgcatacttttcaataacttaccaaacagaaatgcttgtttagagggaatatttataggtacccaagttataactattacattatacttgtctcctttgatacaatatttcatctctaactagattgtaagctcttcaagaggaccgtcttgtgtgttttaatctgttacttcctggagatccatcgtgcaggaaacacagaacatttctttagtgatttattctttttagggccatacattttaaaggaagggttagcagtattcttttagagttgtacagattagaggaagcagaaaaaacatgtttactataatttaattgatttttctttccctgagacagagtttctcagtgtagacctggccttgaactcagagatgagtctgcctctgctttccaagtgctgggatttaggtgtgggccgccaggcccagcgtaattgcaggttttgtcttttgttttttcttttttaaagataaagaggaagtttatttttataccttaaaaaaagtcttctgtgctttggattccctttcatccttatgctttacacagtggcatcttcctctggaacctaagcatagtgctgagattgtgttgctcagaataacttctcgagaagtcggggtcacggtagtaaagaagcgcgcctccctcgaaggctggacttggtgttgattatgtcacaatcaaagaaaaaaacactgaattcaaaataagtcttctcaaaactacacataaagaacaatattcggtctgatactatatagtggatatgcatgtcattctatatgtccagattcagtgcgaagtaccaagataaaccgtgatgtcaaaaaataaatacagtaaaaatacataaagcttgtatcattgtctacatgctatttatcagttttatagactatccatggactgttttaagtataatctgttgcatcttacatttttcatttgctggatgactggtaagcaatgtattaaaatgacttgcatatttctttctattctgtagagctttattgttgacaatacagacagttctccacttttataatattgaagacagtgaggatagccctgaagcctataaattccttacagacttctcagcatctccagttgtctggttaaatgcagaatatgtgttctttgaccttgccttcatgcagtcccacctcagggagctgagttttcactgttccatgtctgacttgtaaatatttcatcaagaattcaagacctaattcaaaatcactcacactgatgcttaaacttacagacctcttaaaaaaatctaccaacaacaatatcaaactgtgtaggccttcagcgtcctttgaatgtagtcttacacagtcatttaggatttgctgaccacctcagatagaatactaaatatttaactgcaggtcatacataactttgtggttctttcagttcaggaattgaactagtttataaagcaaaacttaaagacagcttcactctctttcagtgtgaaaactagttttagaaaagtaactacttgttgaagatgtagtgctttttatttttaagaaatgagcttctggggctgaagagatggttcatcggctaagagtactggatgctcttccagaggtcctgaattcagtttccagcaatcacgtggtccacaacctctatgatgagatctgatgccctcttctggcaagcaccatatatagacacagaacactcatacataaataaaaaaaattttttaaatgaacttcagggtccatttcgtagcactttacacataggcattgcgtgaagggttggctttctaaaaaggctcagtgtagagagctgtgtgaggtgctcatgtctgcgaaggtagttcactgggaagacgtggcataaaagtgttgccaaaaaaagccagtgtgtggtttgttgtgtaaaggtgcttgctgccaagcttggtgacctgagttcagtcccaggaacctgcatggtagaaggtaagaatccgctacataagttgtcctctgatcaccatactcacacatgcacatgataaataaatcttaaaaaaaaataaggcacttaataccatcataaaagatgtataaatagtgctgtgtagttgcaattgcttaccgggtagcaggaagcttgtttttagaaccatgtgtagtctcaccttaaagaaaacaaggcaagaaaatgttgccagtactttaaaaacaagacaaaaaacttggatttgataatgtttgacaactttcttgttgaaagaaactatatatgagatattcataaatgtcataagttacacagcgtttacataaatcacataacattttcctaatttactcttgctgaatatttgctttgagaacagtatttttcttaagctgtgctatatgttaaaatttgttaaaactggcattccaataatctggtaattacaaaattacagtaaaccattaaatactgaaataaactttctctgtgttaagcaaataaatcaggagatttactaaataacattgcagtgttgctatttgctgtttccatttggagtggtggttaggttacactggtgtttactcatggcacatattgtctatgaaaatatgaaaactgaaaagtcttcctttgcatctggaatttactttaggatttgtgtgtgcgcaacacatgtttgggtgcttgcagaggtgagaaatgtgtgtctgatttcctgaagcaggagttacaggcagttgtaaaccacctgatgtgggtgctggaacctaagtttgattacggaagagtaacaaacacttttaactgatgagctgtctctccagcccctagagttctagtcattacattacagtcaccttgatattacctttcttccccctccccctcccccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggccttaataggggctctgaaaatttgagaagagaatttaataaacttagtgtagatagcatttagtgatgtcattgtcttgacttgttctttcaaaatatacaacatttccattttgttagaacatagtctgacttaatctcaggtttagacagttcgggttaataagtattaaattgggtatgtatgtaggagatgactgaatatgctgttttcttgtctggattcgatgaaggggtgctatattgaccaccgggagactattcgcagagatttggaacattgctcagtttctgttgattatatacatttcagttctgatagattagacatacaaaatgtctaatggcaaatatttcaaggtgaacaagaaagaactgtgcaaggataaggtagagaagtacaggcagtgccacaaggtcattggctgtgaaacgtctgtcacacagaacccaagcttttgtccctcgtcagtgacacttcagaaaaggagcatatatatcagcggtgaaaggatgggttggcaaggcttttaaacttcacaaaactaaaaacaagtacaaaagaactagaaatcactgtaccccatgtaaggctggatatacttctgcagaacttgcacaagcgtgtgtgtgtgtgtgtgtgtgtgtgtgtgtgtgtgtgtgtgtgtgtctacatgcaggcacctgctgagaccccatgtaaggctggatatacttctgcagaacttgcacaagcgtgtgtgtgtgtgtgtgtgtgtgtgtgtgtgtctacatgcaggcacctgctgagaccaggggtgtcagatccttctggagctgggttgtagctgagtgtgagtgtgagctgcttgctagacatatgactgctggaaaacagacacaggtcctctgaatgagagatacacacacacttcaactgctgactcatctccctaaccctagtcagtttttaaattacatgcccaaatatgccttttgctgaagttgccctagaaacttcagttctgtttatgtggttgccttaaatccttggaattttaagcgttaaggatgaactaagatcttattcttatgctgaaaaatcgttttctcatttctaacaggttattttctttttaacagacagcatatgtttcactagttagaaaagcatttctttgaaatattatctcatatctttgtaataattaaatttgaatttaagacttaaatagccaggttatgaggagtatggatttgttttgctttaagatttatttcatttttaaatatgaatgcatgtgagactgggcatgtttgtgcaggtgcttgcagaagccaggagttagccttggatccagggggtggagtttcaggttattgtgagctgcctctggtgggtgctgggaattaaacttaggtgctctacaggaacagtatacactgatgaaccatatccagcctcctgaagatagatttatttctcaaaaacttgtagagtgatgaagatcttaaggactgaggttcttaagaaaatcagaatattttaggaaaaagaagtggaagtatacaacctcccttcacttggtgattatgaaagtgacagtttgactttctttgatgagctttaattttcagtttcatgacaatttgtgttaacttggtgtagatagatagggaggtagtgctgccaaatttatttctgatacagcacacttctgtgtaggtgctacttaggggaatttaaatgctagcctgttagacaggttcactggtcagccttcataatactggctagtatgtcacgcataatattgccactgtttgtactaggctaattttcctacacacttccaccctctgacttggtggacattttatatgcctgctcttttgtactgttcctgcacagcagaatgaaagctttataaaagaaagatatttacggtttcactcaatagcttgaatgatgcttagcatgtagaggtaaattgatcaactttaagactattgttagtaaagtatattagtaaaggaaattattttgttgtgaattagcccattgcaatttttaaaattctaagtccagtaacatcaagtgttatgatttaaaatatataattacatgcatgatgggtgcggtagtgatggcagctagaggctgactttgtggtgtcagtttggttttctctttctgccattgagggagcttccagggattaaactcaggttgctaggattgcatggcaagcgtctttacctgcttagccagtctcagccctagggattagtttttatattttcattgttcaggatcctacaaattgtttctgataggtaattctgtacctggatacaacactggggttaatagaaaaatgaagatcctacacgtgatgctccatactctgtccttcccatacgagcagacaagcaggtggaatgggcctgctccaaatgtcagcgcaggccaaccggcagaatggtggtgtaatgtctttaaggtgaaaaccaagagtgttgtagttgagtccaagaaccttctcatctgtcgtagtaatcttactagcactttcttcttaggaaagacactattcgtgtcactgcccatttggttggcatctgtttaaggtaaaaggtaaccatttaaaactggctcctaccttgaataagggccagtagccacctgactaaagtggtaataaaacactagcagtttagttgaggacccgttgctactggaaataccatagtgtccagtgatagagaggattatctagatgcaggaaggggatgtgaaaatcatagccattttcatacatagtctgcagctgtaactacttaaattttctgttccaaaaactcatcaagaagccttataccttaaatttaaattaaaaaaaaaaaaaaaaggcaagtagcctcaggctgttctcaatagaaacaaggaatgggacccatctgtaggctccctttgatcaaatggtaaagtgaaactatgtgtggatggcaacataggtataaataagtaagtagacagaattttgaagaggtatggtttggaagaacgtcttaaaatgatcatttatatttgtcttttctttctttctttcttttttttttttccctttttggtattttgagacagggtttctctgtgtagccctggctgtcctggaactcactttgtagaccaggctggcctggaactcagaaatccacctgcctctgcctcccgagtgctgggattaaaggcatgcaccaccattgccgggcctcacatgtcattttcattcaagtcaaaggtgtagatttgttttgggatagccaacttgttttttaatggtttgggtttttttgttgttgttggttttttttgtttgtttgtttgtttttttacgtactatgtttcttttaaaaaaattagtataactgatatgataattttataaattttcataactacattatttgtttttccttcatttataaaaacagagtaaacagttcagccaaaataagtacttttacttaaaaaagaaaaagtgagggggctggagagatagctcagtggttaagaggacccaggttcaattcccagcacctacatggcagcttacaactgtctgtaactccagtttcaggggatctgacactctcacacagttatacatgcagtcaaaacaccaatgcatataaaaaataattcagtaatttttttttaaaaagtgaggtttgagaaaatgtcagtggatatctccaaatcactgttgaaatacagtttttgtattggtgaattattgaattattgcttaaactatacaaacactcttgaatctatgtttcctagaaggaaaatagtcttcaaccagtgctgatattaaaggatgtaaaaagagtaccagatgatgagacttgccatcctataaaagatttttatagatcccacttccagcttagtgtttaattccccacaccctttttgtggccacaccttcacacttcttttttaattataagtgggattccactgtatttaatgtcattacattgtagacaggcttctttttactgcattaattcttttcatatccctcctaaaaatagtgtttctagttttgtggttttaaaaacaaaagaaaacaaaacaccaaaattctatcccagatacctttgaactactttttttttgtaaactgcctgttgagggtttcatattgactcagagttgcattttgcacgatctcgtggtggtcttggcagttacttctcccagtgcctcatgagcttgatgactgttttaatacccatgctgacaagtctgctgatcttttttctttgtcatctatgttttatggatactggtatttctaactttttatattaatttttgaaatggcattttatcttaaatttaaagtattataaataatgacatgttaaatattggatacatttaatagtatataatgattataaggatgaatgtctgttttgattagttttagagttgtttggtaaagcattgagatttctctggtttatgagagatgagtatagataccactcatggtaagttcatcttgtctggtaatatgtaggcattgagctataagaaggatacactatagaggtattcccttttttcttgtccccaaaatttagaaagggttgcttccccagttatctgaaagcttagtaaaatgtgcatttaacttctgcacctaggagttcttctctcttgaatcttgctgctgagaggtgttagagaactccacaggtatgcaaaacaaggtcttaaaagttcaagagtccaatattgacttatccagaattttacaatcataaaaagttgctttgcttctctctgtgtgtataggtatcgtgttgtactctgccatgagtgaggagtaggacaccatgaccttcgcagtgtttgaagtctctgattaggctctggccatatattttcctttttacctctgtgccctacttgatttgtcctgttaccagttaccttctagggagcagcttacagttgctcttcttgctctgataggaagaaatgcctagagggatctgaagccagacctcaggcctgacttcactcctgctttctacactctttctgaacctggctggtgcagcttgggaaaatcagctttgctgcctcctctggcctcctggctagtaagggaccaatcaagacagggaccattcctcatttcctattaggaagaaacacacagagaagactttgtatttcttcattgtttgtttttgttatttgaagttactaagatttatagctaatcagatttgagcaagttgagtaaaagatcagctgcattctgttttagctagatttttaagtgcatttgctaacttaaccaccacatagcctattcataacttgaagtttgcctttaaagtcactcaagtacaaagcactgtagtgctcgtaacgatcttccatattattgtaggtaactaaatgaccatggaatctggagcagacaaccagcagagtggagatgctgctgtaacagaagctgaaaatcaacaaatgacagttcaagcccagccacagattgccacattagcccaggtataaaaatacatggagagattccaagctgtatctcttttaagagtaatatgtttccaagagaatttaatttttaataaacatatacatagaatgagtggaaattttctttaaaatatcagaattcctgttcagtattagcattagcttatgtttagaaatgaaagtaatgataaattaacatattagtaaaatgtatcattaaacattaatataatgtgtgtagtgacatgattcagaaataattacttttttgttaataaatgaattggccctctcaaggcaatgatgacaactgccttgtttttctttgtttgtttgtttgtttgtttgtttttgttatttttgttttaaataccacacaagcttattttgggaccttgaggtgcagacgatttgaaagtctaaatttatatattctaatactaaaatgcagagtcaaaggtcggtgtggaactcagtcctaaaggtgataacagccgttctttcctgtgcattttaaatgcgttagtcggattttgttttaagagtcctgcccttgccaggtgtggtggcacacacctttaattccagcacttgggaggcagatgcaggcagatttctgagttcgaggccagcctggtctacagagtgagttccaggacagccagggctacacagagaaaccctgtctcagaaagagtcctgcccttgaggctgtcatctctgtgcttgctttattgttcatatctgtttttctttattaacatctttgcttaaatattagtggaaacagtgcttaatataagaaccaaataagtgttaaacctgctagggtaagaatgtggtatttccttctctgatttttggacacagaatgtcactgtgtagccctgagtggcctccacctcagagatccaccttcctctggattaaaggtgcgtagtaccacgcctgtgtgtttttaaagtgaaggctcccaaagaaaaataatcctggcaaagctttgctttgcttatcttcagtttctccaggagttaaaaatctccattggaagtggctaattagaataagtcctgggaaatacagggttaaccacggctggggtgagggtgggaatgagtcagatactggagctccaggccaaatagtcagagtaagcaagctgactgtccttcctcctaagaacagggcttccctgcagatctcacgagtaagacaacaccacatatgcttggagataagatatatcagatgaggtagcacttaataggcttagtaaaacaggcatggagaaattggggcacatctgaccatttagctgagcagtctccttgatgaagacagccttcagcatgttgtctattctggttaccatattctatccagcttggttgttccctgagttgactttatagagtcatcttggaaattctttttgctctcattattttttctttgggaatttgtattcttgatgaagtagaacatgtaggtagcataattttgagaataaaagattaactgagataaaaattcttggtaagatactttctttggaagttttctatggtccacttagtgttgcttacaaatttttagaagacttaatttcttatatctcacatgttataaaataccttagatctcctcacaatgaatgttcattttttttcttgtggaggatggaaactcacaccctctgttttaggaaatttctttcaattagttgttggtggttttgtttttcattgtcagaatccttgtgacccagatgttacgcttctgtgactggttttgtggtggtacgtttttgtttgtttcttccatttccttatattttccttgcaatttttcactttttgctctacttcctaggaatgtattcactccatcctgcctgctgaatttttttttttaaataaacagttcatttacaaaacatgggtttgttttgtttcttttaagaagctgtgcaactgttttttgtgttttgtgttttgttttgttgttttagatttataacatatacagcattctgcctgcacatatccctgcagcacagagagggcaccagatctcattatagatgattatgagccatcttgttactgggaattgaactcaggacctctgggagtgcagctactgctcttaaccactgagccatctctccagcccctgactgatgaattgctatcatgtttccaacgagtgttatgtttgttccctgggtagtcttttggtattttctttttgctttgttttgttttgtttttgttgtttttgtttttcgagacagggtttctttgtatagccctggctgtcctggaactcactttgtgtagaccaggctgacctcgaactcagaaatccgcctgcctttgcctctcgagtgctgggattaaaggcgtgtgccaccacacccggctggtattttcatagtatcctgtgttttataagtattctgtttattttcaggaaaaggggtttgtttacggtgtggttttcgccttattcggtacttattttgagtttctatttctcacatggccacataactcttcatcttcatttccatttgttttagtgtccatctttcctagtagaggctttcaccagattcttaatactctgtgaatgtctgctcaggattaaagtagaggaagactaggagataggattgtagcttaatggtagagcatgtacttagcacttgtaaagccctgggctcgtgtctcaacattaagtgaatcgtaggcagatataaaatgacgaagaagctactttgaaagctttgtgtatatagtagctccaagccaagaacttaactgcaggacagtggttgtcttaacttttagtaccttggatcttgaatttgatcagatccccagaggtttcactaaccctctgtttatatataaaaggcctgagagccaagggaaagaagagggctgggatatcatagtgactttcagtaaacctggtatgacgtggttaagacccattatctgtgtaatgctgcactcttctctcacagcgggccagacttctttcctgcacggattgagtaaggattcatttgtttttaaagagcttccagtgaaccctttacttattttcagcccttctcttcatgcttccgtttgcagtgttgcagaccccaagtacctatctgcatttgtgtggtgcaagtcaggctttccttcctccaagattggaccatctgtgggcagcttagtttgttactgttgacccatctgttccactgcctcctttcctagtccttgcacttatttgattgtgcatatagaagtgaaattaatttcatgaaatataacttggaatccagaagggaacatactcagtttactatcaggcagccttttagcatagaagacttagaatggaatgcgtagttgaccgtggattttaagaaagcttttctttcttcttgtctagttacagttttactttttagttactatctgagttttttttaacaaaaacaccatactaataaaaattatcttttttttttgagagtttgatacatgtatataatgtattgtgtcacatgtatcacctctatactcttttgctcccaccagacttcttctttccaagataccttctactttgttgtcttcttctggtggtagtggtggtggtggtggtggtggtgatgacccactgagtgtaattatgaatgcttacatgaacgtgagtggggagttacataacaccttgtcagtggctatcccactgaagacaatgtctgtccctcttccagtaaccattaactgctaatagttcctaagagagcttatgaaatcctgcctctatccatggtaggatgttacaggccccatcttatacatgttttatgcaggtcatcagagttgttgtagcagtttgttagtgtaacagcagtgtcatgcccagaactcagagtttaccagcattccttcccatcctctgttacattccttccagctgttctcttaaagtgttccctgagccttgagggaggtgtatatcatagatgttgacacacctcctgttaattctgactccagataatgtttgaatgctagtttttaaatactaggaaaacattgtaaagtgaaggcagaatgattgtgcttttgccaatgaataggaggggaggaagcaagtagtgaaggaaagcaacgttaggattgtgtaggctggcctcacccgtgctgtctcctcagtaccacacaggggagaatgttacatccagtaaatctaagcaaaaagtaatcataggactttattaagcaataaagaattaaacagaaataatgaacttctacaatgtagctctatttttagactatgtcaagatattataattaccctaaaaccaaaaaaataggttatggtggtgtgttgtaaaaattgaatgtgtctgtgtgtcttgagagacatctgtcaagcatggctcactttgaccaggccataagcttcccagaatagttgtgaatatggcccaacaacattataatatcaaaagataggacatctgtgataggagagtgcttacctagcaggcgggaggacttgagtttaatctgtggcacagaataaaaaaagctagaacacagcaaaaatgccatggtgattctggtagtagagacagatgggatttttaggcaggccttatcagacagccttgtgagataactgctccttgggagtctgagcaggaggaccataagttcaagatcagcctgagtagtcaagtgagacactgtcttaaaattgaaaaaaaggaggatggtgatacatttccatgttagattacttgtctgctactcacccatacacacacgaaagagggcttacttaatactttatgtttgcttcctgtgtctgtgtaacctaaatgtaaggagctttgataaatagctaggataattcactggagtagcttgttaggtttacccttgaaggatgcaggtaagaagtcactgatactgtaattgcttcaaatactcaaatagctttcctatgaaattaagatgagacgctcagctcataataactttatggctgagccttttaaacctaaggaatcagcattcttagtttcctcttcaatttgtcattacaaacaggacaacatgtgcgactctgcagatagtccttgcaccacatttatagaaccaagtaaaataaaacatgactgaaaataggactagtacagaaggcttttgtggatgtctaaggaaaggaaacttctagttttaatctgtgtgttaaagatcacttgtcttcaagaggccttgctgggtaggtatgtctgcaaactggcattcagtactatacctttcacctcaccccagctctcccaaaccctgtccctctttcattctgtctcgtctttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcctctcagacagcctttcagaaagcccgtggtctgcctcaggatgttcatatgagctgcttctcttggacctgtccttaccacctactaccctacttctggctctcatcagcctaccctaactatataggctttctcatggtacaccacttccacttttctctgtctgaagtgtatatcagtcattcagccactaaagtgattgatctaaaatgcaaacattctatttagttcagcagttcttcctatatattctcagaataaatgaagttttatttttgagacatggccttgaatggctcaggctggccttcctttacagcaatccttctgcccaaatatctttagtattggaattacaaacatgtgacacttttactgactaaaatgcaagatttttgtttgtattgaaacagggtctcactacacctctggtttgcctgaaagtcacagagatctggctatctgcctcccaagtgctgggcttaaacacatgagacaataggtctggcttgaaagcttttttaatctgacatatagagaggtgtggggttttgtttgttttaattattttgtgtatgtgagtacactgttgctgtcttcagacacaccagaagaggacattggatcccattaaagatgtttgtgagccgccatgtggttgctgggaattgaactcaggacctctggaagagcaatcagtgctcttaactgctgagccatctctccaggcccatatagggaggtgtttataactagaccattgaagacttttagaacctctgttactttatctctacttctgacattagcaatgtgtgtgcctttcctccattttacatacacacacattcacgtatacatatgcgtgcatcacatgcgcacacacacatagacacacacaaacaggtgcacacctacacacaggtcaactttttgaaagcagggccctatatctttctcctttttgtggtttttattataacattgctctcagtacatgtgttgtggttactatttatcaaatgcaatgaaagtttcttaaatatgttggtgcttaggaacaataaatactatttgatagacatgtcagtaaactcttaagatctggacattgaatcattaatattgacttctttgtgcttcaagaaaaactagtttccttttaatattcttttagctctgctcctttttcatccaaaattaaaacacttcttaaaattccacctgttaccagattagtgttttttgtgaatgtgaaaaatcgaaatttttaattacaatgcatgtaccttcttgttaatgaaaaaacatacttttttaaaaagtaaatctttttctttctttctttttaattaattaacttatttatttattatatgaaagtacactttagctgttttcagacacaccagaagagggcatcagatttcattatggatggttgtgagccaccatgtggttcctgggatttgaacttaggacctttggaagagcaatcagtgctcttaacagctgagccatctctccagcccagaaacttacttgtaattagtctaaagtagcatactaaagtctttatttagcatgtgttgttctgaacttagcaagtccttgttgcccagattggaaaagctaagattttaaaacatgatttaacagcagggtgttgtgaaaacttttgtacttaagaccagtagcatgtctgtcgttgaccttgagtgaatcagtctcttagcatatttttgtctttacgtaaatataatccttattactcaacctttaaaaagaaggagagaaaatttggatataaaaaagcagacagggaaatgtagacaccgaatgaatggggtattcattagcttcagagaatgactgggttgcatgttttgaagctacaggagatagctggctcttggttgttttgctttgtcacttgggaatggctgaaaagtagagaagatttgtagggtttgaactttttttaagactagtaaattgttggcttcttgtacattgttaaaatatgtatcaagcagaacggtcatagttttgcttattagaagtcactcaacattaaacctgagtagtcccacagagttttctgtggtggtcattgctttctgtgtttctataggtatccatgccagcagctcatgcaacatcatctgctcccactgtaaccttagtgcagctgcccaatgggcagacagtccaggtccatggcgttatccaggcggcccagccatcagttatccagtctccacaagtccaaacagttcaggtatgtgtataaaagttttgtatgtactttaataacttgtttatagccatttctgtctcctttcattagttgtgaaaataaagacacatctagtttagatttattttacttgacagtaaaggagtatgttgtatctgcatagttctgttatatgtaccaagtcagacctcaagaaaattactttttttaagcttcaaaagaaaaagcatttatttagacagataaatagtaaatacatgtgataaaggaaatgttgggaaagactgtaggactgatgtacctcaattaaggaaaattaacttaaaatgaacctaagtttgtatggggaaagggttgataaaaattaattaatagccttacctgagatagaatttgtacttagagatcacatcagacatcatattgtctgttgagcttttaggtagagtatgtaactctgaaataatactatattcagaaggtacatcataaggatcattgattgtatcacatatatctgattctttggagagcttgcttctttggggttttgggaataaaggcattctgaaggtgactacttagatctagggaccaagagcagtaacaggataattctttaccaatcagctaattgaaggaaaaaaacaaagactgaacattcatgcaagtaggccataaccccagcctcccgtttcatatttttttacttcacagtcttcctgtaaggacttaaaaagacttttctccggaactcaggtgagtcctaggtcctagatttgaagaaagttatttttagaaaggaaggtgagaaattatttacgtcttttgtccttctctgtgaacatggagttttggggtttgagtcaaagtctacacctccgttgtactgctcactgcatagttactgctcaggtctctgtaacagctctcctcccccagaactgcatgaatgccttgagaaaaggaagctgaacctcctgaggtctagacaattagacatgaagcctaactgctggcatactgtgtgactagccagagtatactttctaaaattgggacctggtaaccccaagaactcagcaaatctcactcatttctgtcagacttcttcctttcccttctttccccacacccttcccactgtacagcaagttttctagattcaacaagcttctatttttctaaagcacttattatatggtgtatgttgatttctcttgcccagccttggctgtttctgctgtgttttccttgtatggtagccagaactgattagtatttttgcaaaggggaaaggtgcttcctcctttgtccaggaaccattctgttattgttatcaaagcccacataaggttcattagcggtttcttccctttgggcttctgatttgctgtaacatttacagggtagaaaactgttacgtgaaatcctggttactagtttcccgaagatgctgggaatggacattattaacaagccttgacagtattagccagattaaggcctatttttgagaaaccacagtgataagtagtcctgtgacttagtttgtatttcatgaaatgccaccttgccaatctccatatgatcttagcattagtaccgtccctgaattcttctctagggcttacctgctagtcaagagttttagagatcagacctactttggatagtaataggtcaagtgatggtaagcacaaatggcccttactttctctttttcacttacagaaagtttttgttattaaaaaataaagttaagctacaaaatacatattataggatttggggtttgtttgtttgtttgtttgtttgtttgtttgtttgagacagggtttctctgtgtagccctggctgtcctggaactcactttgtagaccaggctggcctcgaactcagaaatccacctgcctctgcctcccaagtgctgggattaaaggcgtgcgccaccacagcccagcccatattacagtttttaattaataaaacatgatatttaattgttgcctgtattcttatttttaatcttatcttggggcaaaataatttgaactcaagtattaaatgtccttaactttgatagaaatctgtgtttgtctttttaagaatacatttttatcacagtgagctatgtttataactttaaaaaaacatgtttgaaaaagaaaactggctccttgaagcctggactatatggagatgtaaatgtgtttcttttgcattaaacactctacaccggaagaatgtgttaaactttgatggttacttgagccagccaacagtggttagttttaataactaggaagtagtggtttacaatagcttatcaaacagggtggaagtatccctcaaacttctagatggtagaatgatagcgttatatataaagtttccaaaatagacaaggtatgactctcctaatggaagagtaggaatagtaggacatcgcttgatatatttcaatatagtaatattggagactgtactaatattttatgtacagcttcactttataattcttacgtgtataaatcacatctcataggtgacatgagaattcttgcaaaatgacagcagtttcacagtgtatatagggttagtagtggcacttaccaatttgggccacatttcaatatagtgctgtctaaatgtaccagaaaggattcatagatttttatgagtgtaaattattacatccatttcttcaacaaatgtttattgatgacatgctatgtgccagtcattgttctaggtgctatcaaaggcagcagtgaacaaacagtacatctctgttctcatgaagatcacatttgagtgaagagagacatactatgagaaaataaaatatgtcaggtgaggataagtgctgcagagttaatcaaacaggggaaagggtagggcaagatgcaaagggaagtgtgctggaactcattgacggtggttgggcagtacagtgagcagaaaccagacagcaggagagggagctaagccaagccaaagatgtggaaatgaggatgtttgtgatttgaaagagtagcatgttcctgctggttaaagtgagtgagaaggggaaggaaaacgatggagactccctcagtgttgatagactcgtcaatgatatgatggagacagtggtaaagccttctacaacataggtagtggttccgttctctggctatcatatagcatcgctgtgtctgaattaagaatgagtagtgggtttggggacctggggggttgggagggatatttggtttttgttttgctagcccaagacaagtgttccaaaatttctgatcttggtgatggcaagcaagggctttgagggatattttacctaatagaaatccctttgtactatctttgtggggagggggcattagccacaagtgaaaggattattagttataagaaattgtcgagagaaatttgaaaatgctttcttaactttttccatggtaaacagtgtaagtttatctgctttaagtcttagagtttcctcagttgtatcactgtcagccttagtggaggtttatatactaagatcttgttgcaaaaaaggaaaaaaagtatcttcttactggagtgcaatatgtaaatgaaaataattattttctaagcacagctaaaaggcactttttttttgttaatctctacatttagagttctcttttaaattggtagatatccagccacatttccaaaaatgtagacctaggcaggcagggtattactgtataggtagccctcaaagtctttgcctcagtgctgggattaaaggcatgtgcctccatacttgacttgaatcaccgttgaaagatgttaatgatcttttgggttttccagatgggctttctctgtgtagccttagctgtcccagaactagctctgtagaccaagctgcctctaacccaaataacgctttcttctacctcctgagtgatgggattaaagatgtatccactatctacctcctgagtgatgggtgtaaagatgtcaccactggctgttagtgatcttttaatgaagtacatttaatgacattgttcttactgttagtgaaaaacttaattctacttgaataatttatttatttagcctatcctaagaacttttaaaataaataacatttaaaatttacaaataaacatatatgtccacgtgaaatttttttatttgtctgcatgtgtgcctgtacaagatgtgcatgcctggtacctgttgatgccagaaggaaggtatcctgaagactggattagaaatggttgtgagcttccatgtaggtgctgggaattaaactcaggatttgtaagaccagctggtgttttttttgttttgttgttttgttgttggtggtggtgttgtttgttttttggttttggtttttcaagacagggtttctctgtatagccctggctggcctcgaactcagaaatccgcctgtctctgcctctgaagtgctgggattaaaggcatgtgccaccacacctggccccagctggtgttattatctgagccatttctccaacccccagttttcttaatgtaagggagatagcagatcaagttttaagatccggccttattttgatccattgagtaaaaagttatctaaaccagccaagcaaaacacttaagtttgcaaaggattgtatagttttaataatgttttagcatttcttaatttctagtcactgtttggttctgttaaaatttcaaattataaaaataagtaatgatttgggttttggcaatgtccagactgttacaaagacccaaaggtaaaaacctgtttaaggaatttggggagatagacattaagagacgtggcaggaggtgtgaggactggggggggggggtgttactggaatgtgataattatgaaacctatattaggtttttaattttacatgattacttttaatttcttacacttatgaagttataaaattgaattttgatatttttattagtgtaaaaagcatgtatcctgtataattttttaagacttcatttttcaagagcagcttgattcacagcaaaattgagagttaaagagttttcccatgtattcctggcccccacatgtgtagcttcttccatgttaacacccctataccagattgctacatttgttagagctgctgcatcataaccatccaagcccaagtgtaaaatgatacacaggctctcagtgttggtatgcttcatagccatgtggtttggatttttcattttagttaatactttctgtttgaaaagacaaaatcttttagattagagaaaggccacaaagtctactgagttggtgtaggaaacaaagttgcaactaaaaaagcagaattttatttcaaaactaatctgtactttatttctttatatattttgatttttgttgttgttgaaacataaagcccaagttgactttggacttactgtatggcatgtataaccttgaactactaatccttctgtctccacctcccaggtgctacgtttgtaggcataaggcaccatactttcttggtcctgagaatcaaacccagggttttgttcatgttaaacaaacactctaccaactgagctacatccccaagcccagtatgtaaggaactagctcatagagcttgctttattgagtttcaacatgaatgaggtattcctctgtcagtgtgtatccttaagatcttgatagttgttaagagtagggcatggcagtacatagctattgctccagcatttagaagcaagactaggaggtctgtctgtctgaggccagcctagctgaccaggaaagactctctgcctcttctttcccctagggaaaaatattaagatggttgtaaggatgggggatatcatatgtgagagtcaaaggtagaaagccaaaggggaagaaagattagccaaaataagtacatttgccatagtggtgcctactatgaatgtatttattggctattgactttttcttgttctgggttttttgtttggtttttttgtttttttgttttcctagcttctgatcttctctttatccgtaagaacatttgtatagagacactagagtgggctaggtatagcagctagagcattccttatttttctctgcaatgagcatatacatcatacagcactgtcaaaagacatgagtgtccaccttgagcttaagtccttgagcttagattgcatgtggaattcctttggaatttctcaaatttgtctgaatatggttttacatgttgctgttgttgactattttgaggcaatgctaacacacactcttgtattgtttaagacactaccgagaagtagtgtgacagcatcatgtcacatggaggaaagcacatgagacttatagtataggctagaaaaacttggtcatataaattttacagatttggagtccagagtgatctaaagtgctgtgagcttgttgctatgacagcactgacactgggaccaccaggattttaatgcgtgtaaagtggaagtgctgagtgcagcaaaggccacactccttagctttgcggccccatagagttgtctatcctgcttgtgccttagctcttctatcttcttcaatgaacataagaccaatggactcatctggtacctgatacttatgaaataatggcaccacccacaatggactggaccttaccccatcaatcactaataagaaaatgtcctacaggctggtctgcagccagatcttacagagaagtattttctcaattgaggcttccacctctctgataactctagcttgtgtcaggttgacataaaactagccaatagtctactacttggtaaagaatcactaacttacttatacaactgtaaatctgtgtttgcacaacagttacaggaaatcatattattattatagactcataactcatgtcggaatccttatttttcttggcagttctgttctaacaactgcaagaaatcactgctgtggtgatatatttaggattgattgcttggtagaaattaaaaacattttctggacaaacatttttgtttccatggcatcatgttagtctgtaggagatgaatcttagtggtgtggttgtctccgttaaaaactgaagtgtgtataagatttatatatacatcttgtgttggacttaaatattttatgggaactaaatttgataaatttgataatcaagataaagttattctttgctttcagatttcaactattgcagaaagtgaagattcacaggagtctgtggatagtgtaactgattcccaaaaacgaagggaaatcctttcaaggaggccttcctacaggtatgtgatttaatagttacagtcagagacgtgcaggaagacttagtacttggcaggtagagatgtcagacaccaataaagctggtccagaaagtactttgcatattatttctcttgagttacttctcaaataagctgatgtttatagtacaaaataagactattactctaaactagtggttctcaaacctgtggtttgcgatccctgaaaaccattggaaaaacaggttacttacattatgattcatagtagtggaaaattatagttacaaagtaacaacgaaaataattttctaggtgagggtcagcacactatgaggaactgtgttaagggttaggagattgggaagcactgctctggcagctgtaggacatttccctcctgtttttgtagctgtgcagactgttgctctccctttaaaaagcttgggttacctttgagtgttagcatcgttcttggtgcactgttgatctgcttagttacacatttataggcgatttttatgtcatggtccttagcaaacagctgtatttaatagttttcagggtaattctttgctaacatcctcaagtttgagtgcaactgtgatgttacctacctagtctatgccttgcctcaaaagaaggtcaaaaccaattactacaatatatcaattggttccatgttaagtatatcaagaaacaacattatctattaatctcatgcttacttaagaattcccttaacagagttaacagagacaaagtttggagctgagacaaaaggatggacaatctagagactgccatatctgggcatccatcccataattagcctccaaacgatgacaccattgcatacactagcaagattttgctgaaaggaccctgatatagctgtctcttgtgagactatgcagggcctagcaaacacataagtagatgctcacagtcagctattggatggatcacagggcccccaatggaggagctagagaaagtacccaaggagctaaagagatctgcaaccctgtaggtgcaacaacattatgaactaaccagtaccccggagctcttgactctagctgcatatgtatcagaagatggcctagtcggccatcactggaaagagaggcccattggacttgcaaagtttatatgccccagtacaggggaatgccagggccaaaaaagggaatgggtgggtagggaaatgggggggagggtatgagggacttttggggtagcattctaaatgtaattgaggaaaatacgtaataataaaaaataaaaaagaaaagaattccgttaacttcagaattttacagtaaccttctgtgttagtttaaattatgcctatttatgtacatttctattcattatttgtttttagatttattttgttttatgttaatggatgttttgtctgcatatgtctgtacaccagttgtgtgcatagtgccaacaaaggtcagaagatgttggatcccctagaactgagttacccatggttgtgaatcactctggatgctgggaatcaaaacaggccttataatagagcagccagtgctcttaacctctgagccgtctatctagccttactcctttttttaaaaagtagttgattgccttatctccctccatgtacttacttagagttactactaagctctttttcaatcctcatccaattataaattccaaatattattagttaattaattttgaggtcctaagaaattttatgttgtaagatatatattataggcatttatatgtatagtctttactatttgttttattttgtgtgtgtgagtatttgtatttctgtacaccatgtgcatgacgtacctgcagaggctagaagtgatgtcaggccctctagtactagagttagaaacagtgtgagctgcttctcttttgttttcaatatagcatgtgttagatcttgagacattgaagtatatcttatgttagtatgtaatacctttgatcctgaagcaaatccaacaaagtattaagaagcaagtcacatgacttctcctaaatcctaagcaaatcatagctgccagatggctccagcagataaacatgctgatgcaagcttggtgacctgagtctcagccacataaggctggaaggcaagaaccaagtccaccaaattgttctatgacctccacacagggacctcctactacattcacacttaaacaaacaaacagacaaaacccacacaaaggccagctttatgtttgtcttaatatttctctggccaaaatcctagcaaaagttatttaaatcaaccatgctttactaatgaaaggaaagctctctaaagaaagcatggagagaaagggttataaagggtactgttaccttaccttagagttaaaccttcacactgagatactatttatatgaaggttatgaattcctgaaatgtagcctttatatctatctagattataacaagctatttaacagatgcttatagtatgcttgcaaggttagaaaaatgaaattgattgattatacaaaataggttaaagcagttaatttgtatacagcagaaaggtgctccattttgtctcttgtggcttgatattttcaccagtaattgttctgaaaaaatagaagccatgcatatcagatttgcaaatgatagaaccatcaggcttaataaagtgccctggtgctaggagcatgagtgccttgaaaggtcacagcaggcaaaggaaatgggctggaaaaataaggtgaaactgtaatgacaactgtaaagtctctgagacagcttacaaaataaacaagttgttcaaatatctgattctggatttattgatgaagaagtaaagttatttatttttatcagccaatataatctagagcaaaaagaactaaagtgtgaataatgtcttagctgtgggtttggattctcatgcaaaggggagctagattagattgtgagactaaattgatgatgtttaaacttccaacgataatcctaaataaggaatatttcttcatgttataatagcgccaggcatacaaggtatgtaactgtaatcccagtactggtgaggctaaggcaggaggattgtgatcttcccaacctgagattcataataagacactgtctggacagtggtggcacacgactttaatcccagcactctggaggcagaggcaggcggatttctgagttccaggacagccagggctacacagagaaaaaaaaacaaaaaaaaagccaggtggtggtggtgcatgccttttatctcagcacttaggaagcagagaggcagatctcttgaatttgggctagcctgttctatacagggtgacaaggtgagtttcagaataaccagggccacacagagaaaccctgtcttggaaaaaaccaaacatacatacattcatacgtatgtatgtgcatatatgtatacacatagacacatggcttgtgaaggctggagtgagtaaggggagatggtctaaaggacccagattcaatccccaggtcacatagtatctcacaaccatctggaactctagtctaagatacacagtggcctcttctggcctctgcaggcaccaggcatgtgcagataaaacacccaaacacatcacaaaaaataaaataaacatatgtagttttattttttatactgccccaccaccaccaccaccaccacgcaattcttgcatttgaagttcctgctttgtttggagtttattcatgtgtttgaatttttgtcctatacgttagtagttttctaatctctatttgtacttagtgaatttttttaaaattaaaatagctctttatttttctaggaaaattttgaatgacttatcttctgatgcaccaggggtgccaaggattgaagaagaaaagtcagaagaggagacttcagcccctgccatcaccactgtaacagtgccaacccccatttaccaaactagcagtgggcagtacagtgagtaatacatttttctattatgaaaagtgagaaggaaaactgttagagtctctttaagaaagtgataaatgccaaggcttttgctgccaatattgtttgccctactgtcagacattattaaagaattgttaagaggcatttattggaaatgtcactttttttttcttttatcctatcttccctttcgccatgtctcttacatgattcatgcacaaattactatttttaattttagaatgatgttggcttgccagcctgtaatttgtattccagctgtatttaaagtagattcctaaaggaaaatttttagcttttccaacactgaaacttttagtattatttataagggagggggttttttttgttgtttttttttttttttttaagatttgctatttttattcatgcgtgtgtgtgctgcatgagtttatatgcatcacctgtgtgcagcagttctgggaaacctttggatcaaatcctttgaaactagagtgacagacacttgtgagccacctaatatgggtgcggggaaccaagcctaagtaccctagaggattaagcattcttagccaccacgtcatcggtctgaaccgtaaggatggtgcttttgataaactgcctttactatctgtgataactgagctaaataagcttagatagatttttttttccttaaattattggtggctgtaaaaaggtatatcttcctcaccctgcctgtgtgtgtctgtgtgtgtgtttagtttgagctaatttcatttccagtttaagtgttcaagatggttcttttctgacattgtacaagacttctttttccaccagcatgaacatactgagggtatatcatattcctgtcctaacttactacctcggggattcttacttgaactagaaaagatgatttttagaaaagacatacaggagctgactgggagaagtattagattactaccatccccttagatactttagtttaaatgtactatactttgagatttttctctaaacctttattcatttatatactgcccctttggtacatgcatgtgtgcctttaatgaaactggtttagcattgtgttcttgatttttcttttccactaagaaacaacataatgaatactcacatgtatggcaaactacttatttttgtaacttatttatttgtaatttttaaaaaaatttatgtgtatgatagctttgtctgcatgtatatcaacaccacatgtatgtggtacctgtaaaaggtagaagagggcgttggattcctctgatactgaagttacaggtggttgttagccaccatgctgatgctgggaattgaatctgggtcttttggaagagtagtcagtgctcttaacctttaatcatctctacagccctgtattttgtaacttgtaaagttaacatggttaccaaacttgaagggttggatttataatatccataaactgtaaacctctttcagcctttcccatgaggaaggattgtatatatctctcctctctcattctatttttcaataagctgtcctgtacagattctcagctaatacagtttaattgtatttagattattaaagttaaaattttacgtaattatatttccttgttcatatttctcagtagtaatttaattaaacctagagataataattttcttaatgaatttggttttgtcaaaactgtcttatacttttaaagtgatcaaatggacaggtaaatattgcatagttaaagacatgtttaaatcttagatgagacgaatatattcaaagaatcgtcatttgtgtgtgtgttagacaccttatttagtgtttaaaacaactatgcatctgttatgtaagagcagatgtttttagatttccaggaaaattacaaagtcgaatgatcttaacagagtagcccatatcttttcttgtgtagttttttccttccattcactttgagcctgttactgtgtaatcaagaaaaaagtagagtgttgattaaatgaagaggttttgttaaccttctgattctttaaagtacccttccccaccctcttggtgtattgttactttatatgcgtggatgttttccttgtgcaccacatgtgtgcagcgcctgcaaaggccaaaagagggtgtccagtcccctggaactagctgcacatggctatgagcctccctgtcggtgctgggaatagaaagagcagccacgtctccaggtcttcccaagtaatcttacagaattagttgtagaacaattcttagacatttttttaacatctaagataaaaaaaatctgtgattagctaacataggaggattatatatgctagtatgttaatttccacaaatttatcaccttgataaagtgactaaataataatgctcttgattgaacagtgaattatcaattttctgtgtacacaccactcaggatggtataattccccagatctcactgatactcccaaactaattcacttctgttagacagtaacatcattagatacattgataccacactttttaaactttccttttaatgcaattataatccattatggagagctgggaacaaacctcaatcaatggagtgcattcctactgtgagtgaaagttttattctcaatactcacccatgcatatacacacacatacacactttatgaaattgctcaggtaaaattaacaatttttagtctttttttggaaaacgtgatccttagatttatctgagaagtcaatcatattttcagttctagtatctaaagattatgtattgcaaacaaggtaagaacttacctttcaagtcacttttcctgtgtactgttgtataagggtgacttgtgtgagtattgattaattttcatttgccagatagtagctagtgtgcttactcatcagtcagtagaaggctctgtgaagacccagacatggagcttagaacaagatgaggtgcatctagctgagggtcttggatctgagtttaaacttgatgagacacacatagtaactgagatccagggtatttcctgtctttcttgatgagatttttggtggagatggtggatggtaggtgctagagactgaacccagggccttgcacattaatgtattactgattcctctttttcttttcttaaatatgactaatttcctggatttattttgagatgctgtcttactgtgtttcccaggctagtcttaaactcctagctcagtgatttttttgttgttgttgttgttgctttagccccctgagaagtatcatgctttcacatggctgtctaagtacactactgacagagtttataaattgttttaagagttagaaaatataattcactgacagatggctttattaaatacacttttaacaaatatttattgggtgcttcttatattaattaattagttttctaccaaaaaatgtatttacaattaaagcagcagatagtaaaaatatctgagggagtttccagtgctgtaaaggaaagtgttgttgtaaagcatgccctccttggagtttaaataagttcaaaaagagtttgtgttttcactttttatattgttaccatgcattatttattatagtacaattcatagagtatatttctctcatagaaattggatatggtttcctgggtacacggttacttgaatataccaagtttttcttggtgatattattttctagaataatttctcctagattatgctttgctaattgcatgggtttagtgtattttaaaagtcttgtgtggtatgaattaaacttgtaacaataaaattcattgggttttagttgccattacccagggaggagcaatacagctggctaacaatggtacggatggggtacagggcctgcagacattaaccatgaccaatgcagctgccactcagccgggtactaccattctacagtatgcacagaccactgatggacagcagattctagtgcccagcaaccaagttgttgttcaaggtaatagaattaagaattcatacatatggtacattcttttaaaaattaatgtgtatactcatgaaaagtaagtatattacaaagcactactaaaatagttttcttcagtatagatttagatattaacagtttagatatgcatgtatttaatacataaatatctctcatgcaactttccactcagatgaaaatgtcatggcataaaaacaactatcttttagcttaaaaggggtttttcctgtctttaatgccttcagtagtcaggattggaagaaccaaaatatgttagaagagaactgaggaagttatagccaggctgaatggcctcaagtaacactagctcccctgggcccctttctttccctcctctgtgtgctatgtcactattaaagagttctagtggattctgctgttttatctgttttgtcttgtttttattatttttaatttaaaaggaatatagagcttttctgaagtcatcaatttggattttggataagaacaagcatcattatgcagatccttgctcattggagaacaagttgtcctttagcctcggtggacgagaggattttgttttgttttgttttgttttgtttcccaaaatttcaagagtctggagagtgttaactattcagcattaattcactgagcagttcatagttcaatattaccagattaaaaatttatagattaaaaaaataatatttgcttctaaaaatattttattaataataactaatgttgcctgtgttaatgagagttatatcccttcaccatggataattacttttggatcataccacaacaccaagtagaacatcttttgtttaacaatgtctaataaatttatactatatatcttgttattagaccaatgatatttaaaaaatattttgcttattagtgggagtaaaacaccttttcactttacatgcaggtactcaaaaaattgtaaagcaggatgtcagtgaatttgaattctcaacgtcagtttgaatatggtaacatgtttagtatataaatctttgtccctcaaacgatacaatcggtgtttttaactgatatgaatgattttataaagacattcaaaatctctcacatagtattagggtacttatgaaagcatatcatgacactgtttctagaagcaaataatggacaaatagtgattttttttttcacatatggagattgtatggtgaggaaaacccaaactacacgcctttaattccagcacttgggaggcagaggcagaggcaggtggatctcttgaattctaagccagcctggtctacataatgagttctagacagccagggctacatagtaagtccctgtcttagaaaactgaaatgaaaacaattcagcttttacttacttagttttagtcacttatattaagatatgtttacatataccatgattggcagcaaatgtccagtttttcatacttggagttttgtttgaattagaaatttataacagtttctatatgcttattttttttcttctattttgctattttatgaaaaatcatggtcgtttttatgtcgtggcaagagtctacttgagttttttgggttttgttttaaatgtgaatttatgtaaagcaagggaagatactgaggcctgtgtgggaggacatctctgctcagctcctttagacttaaagctgacaagcatgcaaaccacagactgcatctccctcccagcacaacatgaataagacccgcagggacgctgaggcaccctgccccgtacctgtactaataaataggacagacaattggagtcttcttcttagatagagtcccattccctgtttcccttttaatctaagtatgtttagattgattgagacacagttgaattaaattgtagtgtttctgtgtcttataccttgttcgctcttctctgcagctgcctcaggcgatgtacaaacataccagatccgcacagcacccacgagcaccattgcccctggagttgttatggcgtcctccccagcacttcctacacagcctgctgaagaagcagcacggaagagagaggtccgtctaatgaagaacaggtacagattccaaacacttaaatgctatgggtcaagtatgtctataaactcttctgtgtgtgcctttacatctgctaacagtaggttctgtgcattgattttctttttctggtagaattagtggatcttgctaaagcttctttgtttattaagttgcatttataagatggctaatggaactttaggtcagtcttcaatcaaaaaagtaatttttcttcatagtttttccacaagagaaatacatagctttaatgaatttgtactttattctagctaataggatatgttaattaaaaaaatttgtaagtaatattcatcaaagtgtcacatatgtatactaacattagactctagtttatttgcattaaatttatttataacaagtatacagtagccatttttgttttaatgaatactttcttttgcatttttatacacttacagatttatttatgcatgttttatgcatatgtgtcaatgcctacatgtatgtatatgtaccacattcataccacaaagggcagaagaatgcattagatctcttggaactagagtgagaggggttggtcagccacctgccttgttctttgtttggggtcagagtcttgctgtgtgggccaaagttgcctggagctcttatcctcccgcctctgggagtgagtgctggaatcacaaccttgggcctggccttaggggcaggggctttctttactcagtaataacttaacatccaaacacttttaaaacagagaaattctaaaaagtatggtattcccgacaaagccttaatgcacagttaatgttagtcttaagaagttaaagtggtacagaagagaagctataaaacacaggtaaagcctttcttagaaatttatttgctttcaaatgtttttgccatcttaaaaaaaaggaagttattaatgtggaaatgatcaaaacaaaacaaaacaaatggtctgaggaggccatcgttggttaatgtgttcaccatccaagcaagaacactggagttcagatccccagaacccatataaaattcttggtagggccctgtaattcctctgatggcacaggtggggttttcagggtaagcttgtctgtaagctctgggttgaagaaaccatgactcaagagtaagcgaaaaagcaacattagggaagatagcctgcacacacataaaacatgtgagtaaacatcaaggggctataatgggggagggaggtgaaaactaaaagagaatcaaggtgtatagagatgtaaactatttaaattcttctttttaccccaagagtgtaagaactatgataaagtaaatgagacagaaattgaacttagcagatctaacttcaattcctgcattcaccctttagctatgactctggaagaatacattaatttcctaaacttgggtttctacctttgagtaaaataagatctctccttgcacccaaagcttggctctcagggtcaggtaagacagcacatggcatctctactgctccttctcttccaacaattttatttttggtaggtcagcatcattgttcctgaaatagtcttaactgtcacagtcttagtaaattatatctagctatgaaaaaggagaaacccattctgagatagtaagggaatcttgaaaagaagccctgtgacctttattgtgcaccaaactgtagtctcccctcccccagcctgaaacctgcttgctcaggggtggagcttcccactcaatcgttctgccacgcccactgctggacctgcggctttgctgtttggagccgtgcacgtggtcaccctgctatttgaccccaagactaattggcgggaatcgggcccccttcccctgcttcataactgcgtgtagaacagtaaaatagaactttgatcagaatgcctgtcttagctgcatttctttatctcgccacctagtcctcttctcttccaggtttccaaaatgcctttccaggctagaacccatgttgtgatctgctggccggacacaacaccaaacaactataagtagttaagatataatagaaatgtactgcctaactcttatttaaatgttatttggaaatacgaattaagttacacattaatctgttcagttttaaagtatttgttaagaaatagttgaccttttctaaaataagaaagaatggtgtttaagatgtctcaagtaaagatctaattgtaaaatttcagacagcagcacttactaaagaaacaattctaaggggacagagtaatcagaatgttttttaaggacttgaaatcatggctgcatactttggctctgtagagtgaaggctcatctcttacagagaacctcagagaggtgtgggaccatgtgcaggggcagcaccagctcctttcttcctcccttccttgcagtactggggattgtcctgagggcctgttcatagtagacaggcgccctaccactgatttagactcaacccagcctttttacaatttttaaaacttccagacagggtgttgctgttacccaggttgatctgaattcactctgagatccggtggcacattacccatccctacctcatctctggaatcctgaagcaaagcttagtttcaataaaaaagagccaggctactttttccagtctttctgtctcagcttcctgagtagctgagattacaacctgtgccagccaagcctactcatattacaccttcttagctgcccagagcattgcctgcttataaacacttgttaggtttaccagtaggcacaatgctgctttttctccacttctcagtgttctgaaagtcactctgacagaatccccgagaaaccaacttaaagaagaaaagttttaaaagacgctcagtttcagcactccatggttactgggcaccattgattttggtctcatggtgaggcagaacatcatggccaggagcctgcagtggaacagagtggctcacgttttagcagactagaggcagagaaagaaaaacatgtctggagagactggtagtggatctcacacaatggaaagaaagtaactctccaaaattgtcctctgttccccttctgtgtgctttggggtgtgtgtgtgtgtgtgtgtgtgtgagtgtgagtgtgtgtgtgttccccctagttatcttccctggacacacaaaaataagtaaatgtaaaaggttggtaatgtaggaatgatagacaacactcatcgacctctggtttccacaagcatatgtacctgtacagacctgcacattacacaagcttagaccacactgataggaatgagtgaatgtaagttgtttttaagtgattggaaattatggcctagtcttcaagggcataacttattgacccacttctcttcagctaagttctatctcccaagttttccgataccttcctatgcaattgtgaacgtatccatggattaacccgtaagtgaggcgaaggtcctcataactcacaacattgcttttattcatagccaagccttttcaggaacactccatttgaaaccataggccactctactactgtcactacctccaaaccacaccaggttaggaaggcaggtcattttttttttttaatcataatcttctcagatggtttatttttgcttggaaacatcatcaaaaggataaatagtgtacataaaatttaaaaacaagatcttccatttaggaagtcaactagtattttatttatgaattttcagggaggaaagtcacattgtaaaatgatattttacatcttcaagtgtcccaggagtagatgaaaaaccacgctgtccaagatggccatccttagtttgcctgctgtgaggatatatactgttgctggccccaactaacagctgtgtatcagcacacaccagatttccacaattgggacaaaataaaagagtacaatatccaaataattttatattaactgtgcattaaaatgataatactttaggataaatgaggttaagtaaaattcatttcaaataaattttgtttctctttaagtgtataatcaaatatatgtatctaatattcagcgtacaaagcaaaaagatactattttattatgtattgttgacatataaatgggcttcagaatctatgctttaaaaaaacaaacatgaacaaagaaaggtcatgaactgcagggcagttccctgggaattcatacaaaaccagtgtttgctctgatggatactgacgcagtattaacttcatcatttactgtgcatgaaggcagggcagatggtgagacagctgtggcagacggtttaaagatcaataaacacacatctaactttgtatctcttatctcttgtccttaatggaaggtaaacagtgatatgcgtgcaagcgtgccgtgggtgcgtgcgtgcgtgcgtgcgtgcgtgcgtgcgtgtgtgtgtgtgtgtgtgtgtgtgtgtgtgtgtgctatgtatgtgcttcttggtgtgttcacatgcacacatacacgcacgcagctcaaggtttccaagaggaaatgaaaagggctaactaaacaggaaggcagttgttaagatagttgcttacaagaggtctacaaagctgcctgattacaagcagggactgtaataagatgcttttgcttagatttagttactgtctgtcaaagggaagttgcccatactctgaagtagttctctttcctctacccagggaactttttaagctttcctagaatctggcttcttaccatatcacttacatgtgcctagaaaagtacaggctttcccatacccctggcaagtctagcatactcttctcagttatttctacctctgtggttagccaacttttgaatgttcagtatcctaagcttccctggtttattctaagttacagtgtgtattagacactagccttcacacgcagtctgtctcttatagccaaataagatctttttccactttgcagaagagaaagaaggtaaataccagagaaaagctaaatagcagtcagatgtccggtatcttaggtaaaaagcctgaaagacagtttgaccctgtgtctaatgacaagtctcctgagcttgtttcaccctgctgtgcctctaagcagagtaaaacacactgttgtcaggatacatagccccaggaaataactgtagaatgctttcctgggatgtccattttgctcagagtcaagagaaacaaactctaattttaagaggaagtaaattcaaaagacattagtactgtagtagttgctttaagctgtatctccagaatcacctaccatattggtgtcattattactgtaagtcttcagtagacaagttagagcatcattgaccacaataagtgaccctcgtgtacattgatcacagcccatctcacactgaacttggggaggtggtttgaggaaccccaaaattctattgtgtgagagttgtcaattattccaaagtgaagataattggcatacattttgtgttactatagcaacagtcataaaatctcagcatcttggaacagcatgacctcttcctctcattataagttgtacattgtcagttccaagtcagttgctgtggcttgtcttcatatctcatcgtcattgccagttgcttcctggatccccactgagtaagagcagcatgttcaagacatagcctttctatggcaaatggggaaggcacagggcctgagcgtaagatccaaccatttttttcaggagctggctctccgcttctaccttgttttgaggcatgtctcatttctgccactgcactgcattagggctgctgtagtacagatccagctttgtgtgtgcaatgaaacagttaggcttgttcagtgtgcgctttcatgccctgagacatctcccaacctacatatgctttggctcactatgtccatttgtgtccactggctacaactctgggaaggcaagcccaagtcagtgggctgtgcatggggattctttccacagagaggattgtgagagtaatacagtttgctgcccttacttttagatagcctacagtgtagaggggccttaaatagggtttccaaagatgaagttaataaaccgttaaatagatttcacactaaatttaaaaaccttacttcagtgaagaacaaaagaaaaatagcaaagtaaacattaaactgggggattagagttgaggatatggattggcagatttacagaaaatgaatatgttcttgagtttcatgtgtttcccaaattgtatcttatatcttgggtattctaagtttctgggctaatatacacttaccactgagaacatatcatatgagttcttttgtgattgggttacctcacacaggatgatgccctccaggtcaaaccatttgcctaggaatttcataaattcattctttttggtagctggtagtactccattgtgtaaatgtaccacattttctgtatccattcctctgttgaggggcatctgggttctttccagcttctggctattataaataaggctgctatgaacatagtggagcatgtgtccttattacatgctggaacatcttctggatatatgcccaggagaggtattgcgggatcctccggtagtactatgtccaattttctgaggaaccgccagacggatttccagagtggttgtacaagcctgcaatcccaccaacaatgggaggagtgttcctctttctccacatcctcgtcagcatctgctgtcacctgaatttttgatcttagccattctcactggtgtgaggtggaatctcagggttgttttgatttgcatttccctgatgattaaggatgttgaacattttttcaggtgcttctctgccattcggtattcctcaggtgagaattctttgttcagttctgagccccaatttttaagggggttatttgattttctgaggtccaccttcttgagttctttatatatgttggatattagtcccctatctgatttaggataggtaaagatcctttcccagtctgttggtggtctttttgtcttatagacagtgtcttttgccttgcagaaactttggagtttcattaggtcccatttgtcaattctcgattcttacagcacaagccattgctgttctgttcaggaatttttcccctgtgcccatatcttcaaggcttttccccactttctcctctataagtttcagtgtctctggttttatgtgaagttccttgatccacttagatttgaccttagtacaaggagataagtatggatcgattcgcattcttctacatgataacaaccagttgtgccagcaccaattgttgaaaatgctgtctttcttccactggatggttttggctcccttgtcgaagatcaagtgcaccataggtgtgtgggttcatttctgggtcttcaattctattccaattggtctatttgtctgtcattataccagtaccatgcagtttttatcacaattgctctgtagtaaagctttaggtcaggcatggtgattccaccagaggttcttttatccttgagaagagttgttgctctcctaggttttttgttattacagatgaatttgcagattgtcctttctaattcgttgaagaattgagttggaattttgatggggattgaattgaatctgtagattgcttttggcaaggtagccatttttactatattgatcctgccaatccatgagcatgtggagatctttccatcttctgaaatcttttttaatttctttcttcagagacttgaagttcttatcatacagatctttcacttccttagttagagtcacgccaaggtattttatattatgttttgactattaagaagggtgttgtttccctaatttctttctcagcctgtttattctttgtgtagagaaaggccattgacttgtttgagttaattttatatccagctacttcaccgaagactgtttatcaggtttaggagttctctggtggaatttttagggtcacttatatatactatcatatcatctgcaaaaagtgatattttgacttcttcctttccaatttgtatccccttgatctccttatattgtcgaattcctctggctaggacttgcaagtacaatgttgaataggtagggagaaagtgggcagccttgtctagtccctgattttagtgggatggcttctagcttctcaccatttactttgatgttggctactggtttactgtagattgcttttataatgtttaggtatgggccttgaattcctgatatttccaagacttttatcatgaatgggtgttggatcttgtcaaatgctttttctgcatctaacgagatgatcatgtggtttttgtctttgagtttgtttatataatggattacgttgatggatttctgtatattaaaccatccctgcatccctagaataaaacctacttggtcaggatggatgattgttttaatgtgttcttggattcggttagcaagaactttattgaggatttttgcatcgatattcataagggaaatcggtctgaagttctctatctttgttagatctttctgtggtttaggtatcagagtaattgtggcttcatagaatgagttgggtagagtaccttctacttctattttgtggaatagtctgtgcagaactggaattagatcttatttgaaggtctgatagaactctgcactaaacccatatggtcctgggctttttttggttgggagactgtaaatgactgcttctattactttaggggatatgggactgtttagatcgttaacttgatcctgatttaactttggtacctgatatttgtctagaaatttgtccatttcgtccaggttttccagttttgttgagtatagccttttgtagaaggatctgatggtgttttggatttcttcaggatctgttgttatgtctcccttttcagttctgattttgttaattaggattttgtccctgtgccctctagtgagtctggctaagggtttatctatcttgttgattttctcaaaagaaccagctcctcgtttggttgattctttgaatagttcttcttgtttccacttggttaatttcgccccgagtttgattatttcctgccgtctactcctcttgggtgaatttgcttactttttttctagagcttttaggtgtgttgtcaagctgctagtgtgtgctctctctagtttctttttggaggcactcagagctatgagtttccctcttagaaatgctttcattgtgtcccataggtttgggtatgttgaggcttcattttcattaaactctaaaaagtctttaatttctttctttattccttccttgaccaacgtatcattgagaagagtgttgttcagtttccacgtgaatgttggctttccattatttgtgttgttattgaagatcagccttagtccatggtggtctgataggatgcatgggacaatttcaatatttttgtatctgttgaggcctgttttgtgaccaattatatggtcagttttggagaatggtaccatgaggtgctgagaagaacggtatatccttttgttttaggataaaatgttctgtagatatctgttaagtccatttgtttcataacttctgttagtttcactgtgtccctgtttagtttctgtttccacgatctgtccattgatgaaagtggggtgttgaagtctcccactattattgtgtgaggtgcaatgtgtgctttgagctttactaaagtttctttaatgaatgtggctatccttgcatttggagcatatatattcagaattgagagttcctcttggaggattttacctttgatgagtatgaagtgtccctccttgtcttttttgataactttgggttggaagtcgattttattcgatattagaatgtctactccagcttatatcttcagaccatttgcttggaaaattgttttccagctttcattctgaggtagtgtctgtcttttgccctgggatgggtttcctgtaagcagcaaaatgttgggtcctgtttgtgtagccagtctgttagtctatgtcttttttattggggaattgagtccattgatattaagagatattaagaaaagtaattgttgcttcctattatttttgttgttagagttggcattctgttcttgtggctgtcttcttttaggtttgttgaaggattactttcttgctttttctagggcgtggtttccgtccttttattggtttttttctgttattatcttttgaaaggctggattcgtggaaagataatgtgtgaatttggttttttcatgaaatactttggtttctccatctatggtaattgagagtttggctgggtatagtagcctgggcttgcgtttgtgttctcttagtgtctgtataacatctgtccaggatcttctggctttcagcgattcattgttccgatcctacgtacgaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgttcgtgggatgtctttctttaggttaggaagtttcttcctaattttgttgaagatatttgctggccctttagttgaaaatcttcattctcatctactcctattgtccgtaggtttggtcttctcattgtgtcctggatttcctggatgttttgagttaggatctttttgcgttttgtattttctttgattgttgtgccgatgttctccatggaatcttctgcacctgagattctctcttccatctcttgtattctgttgctgatgttcgcatctatggttccagatttctttcctagggtttctatctccagcgttgcctcactttgggttttctttattgtgtctacttccctttttaggtcttggatggttttattcagttccatcacctgtttggtcgtgttttcctgcaattctttaaggccaggaatgttgcccttcccttcattgctttaaaccttactcaaccacggtgtggtgggctcctgtaatcccagcactcaggccagacaggactgaaattttgaggccaaccaggtctactcagcaagactttgagacagaacaggctgttgtgcacataatgcccccatttcttgtttctcccagccagctgtgttccttcttccatgtctgtaccacatagtaaggatgtgcaaggaatagatctctgcttctattacaaatggttgcctgtaaaattgctcattttttcatgttttacaaaactagaatatttttactttctattgctaggattattgaactatagatagagcttttgagtttatgagtttgttttgaagcttgccgaataataaatttttattcttttaatttgttaagatttttcaacctcccttttaatccttaaaaaaaaaagcagaatttctatcttgaccatttttaagtgtacagttccgaaatactatatatatccccatcatggtttaatagatttccagaacatttttatcttgcaaaacactcaacctgatagtcatgcttataattcatgtggacactacccacgaaactgagtatgtagtatggaatcatatctcaaaacaagaacccccaaactctctctgtattcgacagttctccactaaccccattccaatctttcggcaaacgcatccagtcctataaaattttattctaacaaactcttagaagcagaattctacagttttgttttttatgactagcttacttcacaaagcaatatccacattatagcatataccaaagttttcttcattttaaaggccatgtaacatttcattgtctgtaagcactttgttttcttttttttctatttttttaagatttatttatttattatatataagtccactatagctgtcttcagacactctagaagagggcatcagatctcattacggatggtgtgagccaccatatggttgctgggatttgaacttgggacctttagaaagtcagtccgtgctcttaacctgaaccatctctccagacccgcactttgttttcttcatccatttgtcaagggacacttggattgcttttgtacatcttgaatattgtgaagagtgctagagacacaggtatacaaagatttcttttaaatctagtgaaattgatgaattgtattgcttaattctattcataatgttctacacacacacacaacacccacccacccacccaccattctgtttggtcaatcttggcagaaattgggccttttgtaaatgtgatgcattctacagatttgctccactgtagcagcaattatagataaaactgtgctagagccgggcagtagtggcacaggcctttagtcccagcacttgggaggcagagacaggtggatttctgagttcaaggctagcctggtctacagagtgagctccaggacagccagggttacttctgaacgtataagccagccccaaataattgttttcctttataagagttgccttggcctctgggtctgttcacagcagtacaacctaaaacagctattaggacatttcagaattaatgataaggataaatgtggtagagtaaccctgtctcgaggggcaggttgggggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgctgggaaataaaacctgggtcctttggaagagcaacaagtgcttctgaccactgcaccatttctctagcccctccaacgttgattgtggattgttgttcttgttgttttcccttttactggaatagatgttttcgcacatatatatccttattagtttcccctccccctactccttccagttccttcccacttcccttcccttccggatccactccctttctgtttctcattagaaaacaagcaggaaactttatatgccccagtacaggggaataccagggccaaaaagggggagtgggtgggcaggggagtggggggtgggtggaatatgggggacttttggtatagcattggaaatgtaaatgagttaaatacctaataaaaaatggaaaaaaaaaagtaaagaaaaaaaaaagaaaacaagcaggattatgaaggattataataaaataaaatataagattaaaaaaataacataccagaattggacaaaacaaatggaaaagagtccaagagaaggcacaataaatctcattcacacattcggggtttctatagactcataacatatatgcagaggacttggaacatagctatgcagaccctgtgcatacagcatcaatctcaatgagtacataccatgtgtgttcttttgtgactgggctaccccactcgggatgatatttctaggtccatccatttgcctgcaaatttcatgaagtcattgtttttaatagctgagtaatattctattgtgtaaatgtaccacattttctgtatccgttcctctgttgagggacatctggttatttccagctactagctattataaataaggctgctatgaatatagtggagcatgtgtcgttgttatatgttggagcatcttttgggtatatgcccaggagtggtaagctgggtcctcaggaagtgctatgtccaattttctgaggaactgccagactgatttccagagtgattgtaacagcttgcaatcccaccaacaatggaggagtgttcctttttctccacatcctcgccagcatctgctgtcacctgaatttttgatcttagccattctgagtggtgtgaggtggaatctcagggttgttttgatttgcatttccctgatgactaaggatgttgaacatttctttaggtgcttcttgaccattcgagattctttagttagagaattctttgtttagctctgtacctgcattttttaatagggttatttggttctccagagtctaacttattgagttctttatatatattggacattagccttctgtcagatgtagaataggtaaaaacctttccccaatctgtacgttgccattttgttctgacagtgtcctttgccttacagaagctttgcaattttatgaggtcccatttgtctataattcattttagagcctgggccattagtgttctattgagaaattcccccctgtgccgatatattcaaggctctttcctgctttctcttttaatagattcagtatatctggttttatgtggaggtctttgattcacttggacttgaactttgtacaaggacataacagtgggtcaatttgcattcttctacatgttgactgccagttggaccagcaccattccatttgttgaaaatgctgtcttttttccactgaatggctttagcttctttgtcaaatatcaaggtctatgggttcatttctgggtcttcaattctattccattgatctacctgcctgtcactgtaccaatactatgaggtttttatcactgttgctctgtagtagagcttgaggtcagggatagtgatttccccagaagttttcttattgttgagaatgttttttcactatcctgggtttcttgttattccaaatgaatttgagaattgctctctctatctcagaaaaattgaattggaattttcatagggattgcattgaatctatagatgacttttggtaaggtggccatttttactatgttaatcctgccaatgcatgagcataggagatctttccatcttctgaggtcttcattttctttcttcagagacttgaagtttttgtcatacagatctttcacttgcttggttagagtcccaccagtatattttatattatttgtgactattgtgaagggtgttatttccctaatttcttgctcagccagcttattctttgagtagaaaaaggctactgatttgtttgagtaaattttatatccagacactttgctgaagtagtttatcagctgaaggagttttctggtgcaatttttgaggtcacttaagtatactatctgcaaatagtgatgtgttgacttcttcctttctaatttgtatccctttgacatccttttgttgcttgattgctctggctagaacttcaagtactatattgagtacatagggagagagtgggcagctttgtctagtccctgattttaatggaattgcttcaagtttctctccatttagtttgatgttggctactagtttgctgtatgttgcttttactatctttaggtatggaccttgaattcctgatctttccaagacttttaacatgaagggctgtcaaattttgtaaaatcctttttcagcatctaatgagatttttttctttgagttagtttatatagtgtattatattgatgaatttctgtttattgaaccatgcttacatcccattaaaatgaagcctacttgatcatggtgagtgatcattttgatgtgttcttggattcggtttgtgagaattttattgagtatttttgcattgttatgcataaaggaaattggtctgaagttctctttctttgttgggtctttgtgtggttttggtatcagtgtgtaactgtggcttcatagaatgaattaggtagtgttccttctgtttctattttgtggaaaagtttgaaaagtataggtagtaggtcttctttgaagatctgatagaactctgcactaaaaccatctggtcttgggttcattttttggttgggagacttttaatgactgtttctattaggggttatgggactgtttagatggtttatctgatcctgatttaactttggcacctggtatctgtctagaaaattgtccatttcacccaggttttccagttttgttgagtataggcttttgtaataggatctgatttttttttttaatttctttggtttctgttgttatgtctccttttcatttctgattttgttaatttagatactgtctctgtgccctctggttagtctggctagggtttattacctatcttgttgatttttctcaaagaacacagctcctggtttggctgattctttgtatagttctttttgtttccacttggttgatttcagccctgagtttgattattttctgccagattatttcctgccttctacttcctcttgggtgaatttgcttccttttcttctagaccttttaggtgttgttgtcaagctgcgaatgtgtgctctctctagtttctttttggaggcactcagagctatgagttttcctcttagtactgctttcattgtgtcccataggtttgggtatgttgtgccttcattttcattaaattctagaaagtcttttatttctgcctttatttctctcctgaccaaattgtgattgaatagagagttgttcagcttccatgtgtatgtgggctttctggtgttttgttttgttttgttttgttttgttttgttttgttttgttattgaagaccaaccttagtccctggcaatctgatgggatgcatgggattatttcagtatttttatatctgttgaggcctgttttgtgaccaattatatggtcagttttggataaggtaccataagatgctgagaagacagtatattgattgtttgttttaggatgaaatgtccaatagatatctattaaacccattcagttcataacttctgttcattgcattgtttctctatgtagtttcttagacatgtcatagatgatagtttcatcatctatggtaattgagagttttgttgggtatagtagtctgggctgacattttgttctgttagggtctgtatgacaactgctcaagatcttctgactttcatagtctctggtgagaagtctggtgtaattctgataggtctgccttcatatgttacttgacctttttcccttattgcttttaatattctttctttgttttgtgcatttggtgttttgattattatctgacaggaggaatttttttctgatccaatctatttaggactctatcatcttcttatatgtttaggtctatctctttctttaggaaagttttattctataattttgttgaacttgtttactggcccttttgacttgggaatctttgctttcttctatgcctgttatccttcggtttggtcttctcattgtgtcctggatttcctggatgtttttggttaggatctttttgcattttctttgactgttgtgtcaaggttgagactctttcttctatctcttgtcttctgtcggtgatgcttgcatctatgaatcctgatctctttcctaggtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttggttttggaggagagaaaatataggtgaagatcggaaattatctgcctgtttccctggtgagagtggcatgcagattcctagggagtccctgttggagttggggtctgagataaggcaacgagttgggggggccggaggagggaggttagaggtgttcaaggaagaaacctctgcaggtgacacgggattggatttggagggacagagggaaaaaaagagaagatctccacttagcctccatccttcccaccaacttccatttttgaaagtctttcattaaatctggagctcaaggattgaggtaggctttatggccagttcattccatggagctttctgccttctgtctgagcccgttgtgggcttttatcagggtgctaagcatctgaatccagtccactgcttgtgcgttggtactttacctactgagctatttttaggtagttggaaaagatacattttttacctagtgcgtgtacacatgcatgggagcagataagtaaagaagaagcagtaggttctcaccatcagccatgtggggttcaggggctgcgctcagattgtccagcttagcagcaaggggcattaccagtgtgctgccgcatcaggcaatgccagtatcatttcctgagcatttcttgtataatactgaacgtttgcttatttcagctgtttctctagaaaacattctgttctaaaattagtggaatggagtttatgaacataaatttttttgtgtttttcatttttagggaggcagcaagagaatgtcgtagaaagaagaaagaatatgtgaaatgtttagagaacagagtggcagtgcttgaaaaccaaaacaaaacattgattgaggagctaaaagcacttaaggacctttactgccacaaatcagattaatttgggatttaagttctctcctgttacggtggagaatggactggcttggccacaaccagaaagacaagtaaacatttattttctaaacatttctttttttctatgcgcaaaactgcctgaaagcaactacagaatttcattcatttctgcttttgcattaaactgtgaatgttccaaaaactacttccacttctgccctcaagaaatgtgcagcgccaggaatcatgaagagacttctgccctccgtctccaccaccctcaagaagtaatcatttgcttatttgtaaattgttgggggaaatgaggaaaatgaaatcttggctttcttatttttgttttaggtttgttttgtttgttttcttattaatgacttcaaggtttgttgagctccatgattgccttagggacagttacccagcctgctgagctgacgtaatgtgtgggccacacaggaaagtaaggaaggtgcaatgaagaagtgttgactgccaaattaatgtttttacactttcattgtgaattatgtcgaactattaaaaacatcctttaaataaggatattagtgtggtgttgggagtaaactttatatgtatgttcctttcttcagcactgacaagttgtccttggtgctgagaagcacggtattggctacagttcctctgtagggcagttgttgcttcttcattcagttctgtgtgttcagcagtttgaatacattaacagaagtaaccaactgaatggaaagcatggttttgaattttgagttaagttaaaatcatactataaagcttattctggtgagtactaagtcttaatgagtaggtgctggccaggaagctaactccttgagttatacaataaggtgtttaataaataaagacttttgtccttgctttccgaatcctcatactgtcactcatttacctagtccactgctgcatttacactgtgcagccaacaggagcgttttcaagatgtccagccaaagtgatggaaagtactaaatagacaaccttttctatagtcatatttttatcagagaatctcttgggatttttttttttaaatcaaagaagttacaaggatgtgattctaaaacattgtgttaaatgcacacaagaaaagtaatttccttcagacattggagtgtgtgctatggtacaatatttggcaaacacttttttttttctcctaaaaaaggtggtactgctttaactatccaaaagctgttctgtttctgaagatgtttctacttacatccttacctatagtctacatttatgtgtaacattgtttctgataagacttagtaataactcattttttttttattaaacctaataaaatctaagagttacctctttaaaagacaaatatgaggacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaataaccatgttcaattcctttacctgtttggttatgttttcttgtgtttctttatgggatttatgtgtttcctctttaagagcttctccctgtttgcttgtgttctcctgtatttctttaagagagttatttctgtccatcttaaagtccactatcattatcatgagatgtgattttaaatcagaatcttgctttttcaggtgtgttggggtatccaaggcttgctgtggtgggagaactgggttctaataatatcaagtcgtcttggtttctgttgcttatgttcttgtacttgcctcttgccatctggttatctctggtgttagtggtcttgctgtctctgactggagcttgttctcctggcttgttagcactcctgggagatgaggggcaggatttgggtgcagatagctctgtggcacagggtcagctccagggcgcagatggaaacctctagaaggatcctgtccccagctgcaccatggttcctgggtcctgtgggctctggttgggtctcataggtctcagctgtgatcttatgtgtgtcagcactcgtgggagattcgctttctcgggtcgggactggggagcagatagttttggcacagggtcaactccaggtcccatatggaaaccaatgatctgggtttcttatccttcctgtccgactgttatttttagattttgtggtgtcccgtatttctaggatgtcttttgccaggaatttttagatttaacattttccttgattgatcaatttcttctgtcctgtcttcaaaaccagagattacctcatcatctttgcattctgttcatgtgacttgcttctgtgattcctgttcaagttttaaaggttttatttgcagagtgtcctcagcttgggttcctttattctgtttgcactttcagactttgagttgttttatcccttccatcccactgcttgttgtgacttcatgggagtctttaagggatttgttcatttcttctttaaggccctctatcatattaataaaggctattttaaaatcggtttcttgttttcggctctgttgcaatactcaaggcctgctctggtaagtttgctgagccctgctggatacattgtcctagctgttactatttttatgctgacatctaggcatctggtttgagaagaatgtaattctaggtgttgatatcagatcttgtgtttgtgagcatgatttattccttggtttctgttgctctctggttctttggagaatgtggttactctctgttgttactctttgttgtctggtgggacattcttctgggatctcacacacagttgatctatgatggtcctggaggtggggtgcacagggaagagcaaagcagggtgttccaccaggatctgctcagacccctgggagtgaggccagagtgagaagttacaagtagtcttctacagagttggggatgagactcggggacagacttggaggaatacaggaagaggtgaggagatgggcgcaggaggtggcacagaggcctcagaggtaatctacatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaggacagtggaggctggcatgttaggttacttcacgtttgaggaagaactcagactcagaagagtgtcttatatttgctttgggatattaagaaagttaatattaatatcagtatacgtatgagcgggaactgtttggcccatttctaagctaagactaggttgctatctgtaaatgttctacgttgtttataacaaaccattgtcttttttcaaggatgaacagtttgtgaaggacatgattctaggagttgagtgatgtaataatccttctgtctggacagttcaccagattctccagaaggctttcaaacggctaaagtttgatctttgtcctgctgagcttgctgggaaggagatagcataaaagctgtctgtgctagcaagtagatgtatcatttgctaaacgtggtgaaacaaaaaggaagcagaatcacaaatctgtcaacaaactggaattacttcacattaagtcagaatgtcagattgaacattttaaatgtcacaagtggaaaatttgcctagcgtgcagaagttgtaatctctaagtgtctatcttcaaaagtcgaggttttctacataatgaggaaacaggtggtctccttaaccctttagagaccaaggcagtctcagccatacttcctagttggatggatattaatttacagaggcctgggtctttatgcttggagcatgactgtactagtttgacaccagttacttgataatgtgagaaatatatggtgttgtgaaacctgactttgaaatttcagttcatacagaaagaaatcaaagcctctttaatgcaagctgg

SEQ ID NO: 10 - DNA, 1161 bp, Mus musculus
gctccctggctgcggctcctcagtcggcggcggctgctgctgcctgtggcccgggcggctgggagaagcggagtgttggtgagtgacgcggcggaggtgtagtttgacgcggtgtgttacgtgggggagagaataaaactccagcgagatccgggccgcgaacgaaagcagtgacggaggagcttgtaccaccggtatccatgccagcagctcatgcaacatcatctgctcccactgtaaccttagtgcagctgcccaatgggcagacagtccaggtccatggcgttatccaggcggcccagccatcagttatccagtctccacaagtccaaacagttcagatttcaactattgcagaaagtgaagattcacaggagtctgtggatagtgtaactgattcccaaaaacgaagggaaatcctttcaaggaggccttcctacaggaaaattttgaatgacttatcttctgatgcaccaggggtgccaaggattgaagaagaaaagtcagaagaggagacttcagcccctgccatcaccactgtaacagtgccaacccccatttaccaaactagcagtgggcagtacattgccattacccagggaggagcaatacagctggctaacaatggtacggatggggtacagggcctgcagacattaaccatgaccaatgcagctgccactcagccgggtactaccattctacagtatgcacagaccactgatggacagcagattctagtgcccagcaaccaagttgttgttcaagctgcctcaggcgatgtacaaacataccagatccgcacagcacccacgagcaccattgcccctggagttgttatggcgtcctccccagcacttcctacacagcctgctgaagaagcagcacggaagagagaggtccgtctaatgaagaacagggaggcagcaagagaatgtcgtagaaagaagaaagaatatgtgaaatgtttagagaacagagtggcagtgcttgaaaaccaaaacaaaacattgattgaggagctaaaagcacttaaggacctttactgccacaaatcagattaatttgggatttaagttctctcctgtacggtggagaatggactggcttggcacaaccagaaagacaagtaaacatttattttctaaacatttctttttt

SEQ ID NO: 11 - DNA, 1007 bp, Mus musculus
gaattcggcacgagtgctgctgtaacagaagctgaaaatcaacaaatgacagttcaagcccagccacagattgccacattagcccaggtatccatgccagcagctcatgcaacatcatctgctcccactgtaaccttagtgcagctgcccaatgggagacagtccatgggtcatggcgttatccaggcggcccagccatcagttatccagtctccacaagtccaaacagttcagatttcaactattgcagaaagtgaagattcacaggagtctgtggatagtgtaactgattcccaaaaacgaagggaaatcctttcaaggaggccttcctacaggaaaattttgaatgacttatcttctgatgcaccaggggtcgcaaggattgaagaagaaaagtcagaagaggagacttcatctcctgccatcaccactgtaacagtgccaacccccatttaccaaactagcagtgggcagtacactgcctcaggcgatgtacaaacataccagatccgcacagcacccacgagcaccattgcccctggagttgttatggcgtcctccccagcacttcctacacagcctgctgaagaagcagcacggaagagagaggtccgtctaatgaagaacagggaggcagcaagagaatgtcgtagaaagaagaaagaatatgtgaaatgtttagagaacagagtggcagtgcttgaaaaccaaaacaaaacattgattgaggagctaaaagcacttaaggacctttactgccacaaatcagattaatttgggatttaagttctctcctgttacggtggagaatggactggcttggccacaaccagaaagacaagtaaacatttattttctaaacatttcttttttttctatgcgcaaaactgcctgaaagcaactacagaatttcattcatttctgcttttgcattaaactgtgaatgttccaaaaactacttccacttctgccctcaagaaatgtgcagcgccactcgtgccgaattc

SEQ ID NO: 12 - DNA, 1289 bp, Mus musculus
tctggctgcggctcctcagtcggcggcggctgctgctgcctgtggcccgggcggctgggagaagcggagtgttggtgagtgacgcggcggaggtgtagtttgacgcggtgtgttacgtgggggagagaataaaactccagcgagatccgggccgcgaacgaaagcagtgacggaggagcttgtaccaccggtaactaaatgaccatggaatctggagcagacaaccagcagagtggagatgctgctgtaacagaagctgaaaatcaacaaatgacagttcaagcccagccacagattgccacattagcccaggtatccatgccagcagctcatgcaacatcatctgctcccactgtaaccttagtgcagctgcccaatgggcagacagtccaggtccatggcgttatccaggcggcccagccatcagttatccagtctccacaagtccaaacagttcagatttcaactattgcagaaagtgaagattcacaggagtctgtggatagtgtaactgattcccaaaaacgaagggaaatcctttcaaggaggccttcctacaggaaaattttgaatgacttatcttctgatgcaccaggggtgccaaggattgaagaagaaaagtcagaagaggagacttcagcccctgccatcaccactgtaacagtgccaacccccatttaccaaactagcagtgggcagtacattgccattacccagggaggagcaatacagctggctaacaatggtacggatggggtacagggcctgcagacattaaccatgaccaatgcagctgccactcagccgggtactaccattctacagtatgcacagaccactgatggacagcagattctagtgcccagcaaccaagttgttgttcaaggaatatagagcttttctgaagtcatcaatttggattttggataagaacaagcatcattatgcagatccttgctcattggagaacaagttgtcctttagcctcggtggacgagaggattttgttttgttttgttttgttttgtttcccaaaatttcaagagtctggagagtgttaactattcagcattaattcactgagcagttcatagttcaatattaccagattaaaaatttatagattaaaaaaataatatttgcttctaaaaatattttattaataataactaatgttgcctgtgttaatgagagttatatcccttcaccatggataattacttttggatcataccacaacaccaagtagaacatcttttgtttaacaatgtctaataaatttatactat

SEQ ID NO: 13 - DNA, 20 bp, Artificial Sequence
  Synthetic oligonucleotide
acctgggctaatgtggcaat

SEQ ID NO: 14 - DNA, 20 bp, Artificial Sequence
  Synthetic oligonucleotide
gtctgcccattgggcagctg

SEQ ID NO: 15 - DNA, 20 bp, Artificial Sequence
  Synthetic oligonucleotide
gtttggacttgtggagactg

SEQ ID NO: 16 - DNA, 20 bp, Artificial Sequence
  Synthetic oligonucleotide
ctgcaatagttgaaatctga

SEQ ID NO: 17 - DNA, 20 bp, Artificial Sequence
  Synthetic oligonucleotide
actttctgcaatagttgaaa

SEQ ID NO: 18 - DNA, 20 bp, Artificial Sequence
  Synthetic oligonucleotide
atcagaagataagtcattca

SEQ ID NO: 19 - DNA, 20 bp, Artificial Sequence
  Synthetic oligonucleotide
ggtgcatcagaagataagtc

SEQ ID NO: 20 - DNA, 20 bp, Artificial Sequence
  Synthetic oligonucleotide
gttacagtggtgatggcagg

SEQ ID NO: 21 - DNA, 20 bp, Artificial Sequence
  Synthetic oligonucleotide
ccactgctagtttggtaaat

SEQ ID NO: 22 - DNA, 20 bp, Artificial Sequence
  Synthetic oligonucleotide
gctgcattggtcatggttaa

SEQ ID NO: 23 - DNA, 20 bp, Artificial Sequence
  Synthetic oligonucleotide
aacaacaacttggttgctgg

SEQ ID NO: 24 - DNA, 20 bp, Artificial Sequence
  Synthetic oligonucleotide
gcttgaacaacaacttggtt

SEQ ID NO: 25 - DNA, 20 bp, Artificial Sequence
  Synthetic oligonucleotide
aggcagcttgaacaacaact

SEQ ID NO: 26 - DNA, 20 bp, Artificial Sequence
  Synthetic oligonucleotide
aggtccttaagtgcttttag

SEQ ID NO: 27 - DNA, 20 bp, Artificial Sequence
  Synthetic oligonucleotide
taaatcccaaattaatctga

SEQ ID NO: 28 - DNA, 20 bp, Artificial Sequence
  Synthetic oligonucleotide
aagaagcaacaactgcccta

SEQ ID NO: 29 - DNA, 20 bp, Artificial Sequence
  Synthetic oligonucleotide
tttttaagtccttacaggaa

SEQ ID NO: 30 - DNA, 20 bp, Artificial Sequence
  Synthetic oligonucleotide
atgaattttattgttacaag

SEQ ID NO: 31 - DNA, 20 bp, Artificial Sequence
  Synthetic oligonucleotide
agattttcttgtaggaaggc

SEQ ID NO: 32 - DNA, 20 bp, Artificial Sequence
  Synthetic oligonucleotide
ccattttcaccacaataggt

SEQ ID NO: 33 - DNA, 20 bp, Artificial Sequence
  Synthetic oligonucleotide
gtccatggtcatctagtcac

SEQ ID NO: 34 - DNA, 20 bp, Artificial Sequence
  Synthetic oligonucleotide
atggatacctgggctaatgt

SEQ ID NO: 35 - DNA, 20 bp, Artificial Sequence
  Synthetic oligonucleotide
tgctggcatggatacctggg

SEQ ID NO: 36 - DNA, 20 bp, Artificial Sequence
  Synthetic oligonucleotide
gcatgagctgctggcatgga

SEQ ID NO: 37 - DNA, 20 bp, Artificial Sequence
  Synthetic oligonucleotide
gttacagtgggagcagatga

SEQ ID NO: 38 - DNA, 20 bp, Artificial Sequence
  Synthetic oligonucleotide
tgcactaaggttacagtggg

SEQ ID NO: 39 - DNA, 20 bp, Artificial Sequence
  Synthetic oligonucleotide
attgggcagctgcactaagg

SEQ ID NO: 40 - DNA, 20 bp, Artificial Sequence
  Synthetic oligonucleotide
tgaataactgatggctgggc

SEQ ID NO: 41 - DNA, 20 bp, Artificial Sequence
  Synthetic oligonucleotide
gacttgtggagactgaataa

SEQ ID NO: 42 - DNA, 20 bp, Artificial Sequence
  Synthetic oligonucleotide
gtccttacaggaagactgaa

SEQ ID NO: 43 - DNA, 20 bp, Artificial Sequence
  Synthetic oligonucleotide
aagtctttttaagtccttac

SEQ ID NO: 44 - DNA, 20 bp, Artificial Sequence
  Synthetic oligonucleotide
ggagaaaagtctttttaagt

SEQ ID NO: 45 - DNA, 20 bp, Artificial Sequence
  Synthetic oligonucleotide
gaatcagttacactatccac

SEQ ID NO: 46 - DNA, 20 bp, Artificial Sequence
  Synthetic oligonucleotide
ttttgggaatcagttacact

SEQ ID NO: 47 - DNA, 20 bp, Artificial Sequence
  Synthetic oligonucleotide
agtcattcaaaattttcctg

SEQ ID NO: 48 - DNA, 20 bp, Artificial Sequence
  Synthetic oligonucleotide
agataagtcattcaaaattt

SEQ ID NO: 49 - DNA, 20 bp, Artificial Sequence
  Synthetic oligonucleotide
acccctggtgcatcagaaga

SEQ ID NO: 50 - DNA, 20 bp, Artificial Sequence
  Synthetic oligonucleotide
aatccttggcacccctggtg

SEQ ID NO: 51 - DNA, 20 bp, Artificial Sequence
  Synthetic oligonucleotide
ctgggtaatggcaatatact

SEQ ID NO: 52 - DNA, 20 bp, Artificial Sequence
  Synthetic oligonucleotide
gttaatgtctgcaggccctg

SEQ ID NO: 53 - DNA, 20 bp, Artificial Sequence
  Synthetic oligonucleotide
tggtcatggttaatgtctgc

SEQ ID NO: 54 - DNA, 20 bp, Artificial Sequence
  Synthetic oligonucleotide
tggcagctgcattggtcatg

SEQ ID NO: 55 - DNA, 20 bp, Artificial Sequence
  Synthetic oligonucleotide
ggctgagtggcagctgcatt

SEQ ID NO: 56 - DNA, 20 bp, Artificial Sequence
  Synthetic oligonucleotide
ggttgctgggcactagaatc

SEQ ID NO: 57 - DNA, 20 bp, Artificial Sequence
  Synthetic oligonucleotide
atctggtatgtttgtacatc

SEQ ID NO: 58 - DNA, 20 bp, Artificial Sequence
  Synthetic oligonucleotide
tttggttttcaagcactgcc

SEQ ID NO: 59 - DNA, 20 bp, Artificial Sequence
  Synthetic oligonucleotide
agtaaaggtccttaagtgct

SEQ ID NO: 60 - DNA, 20 bp, Artificial Sequence
  Synthetic oligonucleotide
ttgtggcagtaaaggtcctt

SEQ ID NO: 61 - DNA, 20 bp, Artificial Sequence
  Synthetic oligonucleotide
cccaaattaatctgacttgt

SEQ ID NO: 62 - DNA, 20 bp, Artificial Sequence
  Synthetic oligonucleotide
ggtgaaaatttaaatcccaa

SEQ ID NO: 63 - DNA, 20 bp, Artificial Sequence
  Synthetic oligonucleotide
caagatttcattttcctcat

SEQ ID NO: 64 - DNA, 20 bp, Artificial Sequence
  Synthetic oligonucleotide
taagaaagccaagatttcat

SEQ ID NO: 65 - DNA, 20 bp, Artificial Sequence
  Synthetic oligonucleotide
gcacaaaccttgaaatcatt

SEQ ID NO: 66 - DNA, 20 bp, Artificial Sequence
  Synthetic oligonucleotide
ggagctcagcacaaaccttg

SEQ ID NO: 67 - DNA, 20 bp, Artificial Sequence
  Synthetic oligonucleotide
ccacacattacttcagctca

SEQ ID NO: 68 - DNA, 20 bp, Artificial Sequence
  Synthetic oligonucleotide
caatcaacacttcttcattg

SEQ ID NO: 69 - DNA, 20 bp, Artificial Sequence
  Synthetic oligonucleotide
tcaatttggcaatcaacact

SEQ ID NO: 70 - DNA, 20 bp, Artificial Sequence
  Synthetic oligonucleotide
cataatccacaatgaagtgt

SEQ ID NO: 71 - DNA, 20 bp, Artificial Sequence
  Synthetic oligonucleotide
aatagttttacataatccac

SEQ ID NO: 72 - DNA, 20 bp, Artificial Sequence
  Synthetic oligonucleotide
gttctctaaacatttcacat

SEQ ID NO: 73 - DNA, 20 bp, Artificial Sequence
  Synthetic oligonucleotide
cagttaaggtccttaagtgc

SEQ ID NO: 74 - DNA, 20 bp, Artificial Sequence
  Synthetic oligonucleotide
cagtccattttccaccacaa

SEQ ID NO: 75 - DNA, 20 bp, Artificial Sequence
  Synthetic oligonucleotide
gttgcttccaggcagttttg

SEQ ID NO: 76 - DNA, 20 bp, Artificial Sequence
  Synthetic oligonucleotide
gttgtggacattcacagttt

SEQ ID NO: 77 - DNA, 20 bp, Artificial Sequence
  Synthetic oligonucleotide
gattacttcttgagggtggt

SEQ ID NO: 78 - DNA, 20 bp, Artificial Sequence
  Synthetic oligonucleotide
ataagcaaatgattacttct

SEQ ID NO: 79 - DNA, 20 bp, Artificial Sequence
  Synthetic oligonucleotide
acaggcagcagcagcatccc

SEQ ID NO: 80 - DNA, 20 bp, Artificial Sequence
  Synthetic oligonucleotide
acctagaacaatgactgaac

SEQ ID NO: 81 - DNA, 20 bp, Artificial Sequence
  Synthetic oligonucleotide
agcatttgccatgtattgta

SEQ ID NO: 82 - DNA, 20 bp, Artificial Sequence
  Synthetic oligonucleotide
tattttatacctgggctaat

SEQ ID NO: 83 - DNA, 20 bp, Artificial Sequence
  Synthetic oligonucleotide
gcatggatacctacagaaaa

SEQ ID NO: 84 - DNA, 20 bp, Artificial Sequence
  Synthetic oligonucleotide
ttatcctcacctgacacatt

SEQ ID NO: 85 - DNA, 20 bp, Artificial Sequence
  Synthetic oligonucleotide
ttcccagctcttcataatgg

SEQ ID NO: 86 - DNA, 20 bp, Artificial Sequence
  Synthetic oligonucleotide
ctcagataaatccaaggatc

SEQ ID NO: 87 - DNA, 20 bp, Artificial Sequence
  Synthetic oligonucleotide
gtaatggcaactaaaaccca

SEQ ID NO: 88 - DNA, 20 bp, Artificial Sequence
  Synthetic oligonucleotide
attctattaccttgaacaac

SEQ ID NO: 89 - DNA, 20 bp, Artificial Sequence
  Synthetic oligonucleotide
caattttgggtagtcacttt

SEQ ID NO: 90 - DNA, 20 bp, Artificial Sequence
  Synthetic oligonucleotide
aaaacttgggaggtagaact

SEQ ID NO: 91 - DNA, 20 bp, Artificial Sequence
  Synthetic oligonucleotide
ctgcccattgggcagctgta

SEQ ID NO: 92 - DNA, 20 bp, Artificial Sequence
  Synthetic oligonucleotide
tttgtggcagtaaaggtcct

SEQ ID NO: 93 - DNA, 20 bp, Artificial Sequence
  Synthetic oligonucleotide
atcccaaattaatctgattt

SEQ ID NO: 94 - DNA, 20 bp, Artificial Sequence
  Synthetic oligonucleotide
ccttccctgaaggttcctcc

SEQ ID NO: 95 - DNA, 20 bp, Artificial Sequence
  Synthetic oligonucleotide
acacaccgcgtcaaactaca

SEQ ID NO: 96 - DNA, 20 bp, Artificial Sequence
  Synthetic oligonucleotide
ccgtcactgctttcgttcac

SEQ ID NO: 97 - DNA, 20 bp, Artificial Sequence
  Synthetic oligonucleotide
tttagttaccggtggtacaa

SEQ ID NO: 98 - DNA, 20 bp, Artificial Sequence
  Synthetic oligonucleotide
catttagttaccggtggtac

SEQ ID NO: 99 - DNA, 20 bp, Artificial Sequence
  Synthetic oligonucleotide
gtcatttagttaccggtggt

SEQ ID NO: 100 - DNA, 20 bp, Artificial Sequence
  Synthetic oligonucleotide
tggtcatttagttaccggtg

SEQ ID NO: 101 - DNA, 20 bp, Artificial Sequence
  Synthetic oligonucleotide
catggtcatttagttaccgg

SEQ ID NO: 102 - DNA, 20 bp, Artificial Sequence
  Synthetic oligonucleotide
tccatggtcatttagttacc

SEQ ID NO: 103 - DNA, 20 bp, Artificial Sequence
  Synthetic oligonucleotide
attccatggtcatttagtta

SEQ ID NO: 104 - DNA, 20 bp, Artificial Sequence
  Synthetic oligonucleotide
agattccatggtcatttagt

SEQ ID NO: 105 - DNA, 20 bp, Artificial Sequence
  Synthetic oligonucleotide
ccagattccatggtcattta

SEQ ID NO: 106 - DNA, 20 bp, Artificial Sequence
  Synthetic oligonucleotide
ctccagattccatggtcatt

SEQ ID NO: 107 - DNA, 20 bp, Artificial Sequence
  Synthetic oligonucleotide
ggctccagattccatggtca

SEQ ID NO: 108 - DNA, 20 bp, Artificial Sequence
  Synthetic oligonucleotide
tgcatctccactctgctggt

SEQ ID NO: 109 - DNA, 20 bp, Artificial Sequence
  Synthetic oligonucleotide
cttgaactgtcatttgttgg

SEQ ID NO: 110 - DNA, 20 bp, Artificial Sequence
  Synthetic oligonucleotide
agttacggtgggagcagatg

SEQ ID NO: 111 - DNA, 20 bp, Artificial Sequence
  Synthetic oligonucleotide
actgatggctgggccgcctg

SEQ ID NO: 112 - DNA, 20 bp, Artificial Sequence
  Synthetic oligonucleotide
tggcaggtgctgaagtctcc

SEQ ID NO: 113 - DNA, 20 bp, Artificial Sequence
  Synthetic oligonucleotide
ctgtccactgctagtttggt

SEQ ID NO: 114 - DNA, 20 bp, Artificial Sequence
  Synthetic oligonucleotide
aggccctgtaccccatcggt

SEQ ID NO: 115 - DNA, 20 bp, Artificial Sequence
  Synthetic oligonucleotide
attggtcatggttaatgttt

SEQ ID NO: 116 - DNA, 20 bp, Artificial Sequence
  Synthetic oligonucleotide
gtgcatactgtagaatggta

SEQ ID NO: 117 - DNA, 20 bp, Artificial Sequence
  Synthetic oligonucleotide
gctgtgtaggaagtgctggg

SEQ ID NO: 118 - DNA, 20 bp, Artificial Sequence
  Synthetic oligonucleotide
ctctctctttcgtgctgctt

SEQ ID NO: 119 - DNA, 20 bp, Artificial Sequence
  Synthetic oligonucleotide
ttgtggccaagccagtccat

SEQ ID NO: 120 - DNA, 20 bp, Artificial Sequence
  Synthetic oligonucleotide
ctgtagttgctttcaggcag

SEQ ID NO: 121 - DNA, 20 bp, Artificial Sequence
  Synthetic oligonucleotide
ggcgttgaaaatttcttgag

SEQ ID NO: 122 - DNA, 20 bp, Artificial Sequence
  Synthetic oligonucleotide
ttttcttttcctcatttctc

SEQ ID NO: 123 - DNA, 20 bp, Artificial Sequence
  Synthetic oligonucleotide
ttatgcatgcggcccacaca

SEQ ID NO: 124 - DNA, 20 bp, Artificial Sequence
  Synthetic oligonucleotide
tccttcaataccatgctaaa

SEQ ID NO: 125 - DNA, 20 bp, Artificial Sequence
  Synthetic oligonucleotide
agctgtattagtacagaatg

SEQ ID NO: 126 - DNA, 20 bp, Artificial Sequence
  Synthetic oligonucleotide
ggttacttcttttaatgtat

SEQ ID NO: 127 - DNA, 20 bp, Artificial Sequence
  Synthetic oligonucleotide
gctttgtacttttatttact

SEQ ID NO: 128 - DNA, 20 bp, Artificial Sequence
  Synthetic oligonucleotide
gtggtatgtaagtgcaatgg

SEQ ID NO: 129 - DNA, 20 bp, Artificial Sequence
  Synthetic oligonucleotide
ttctctgttaaattgttaat

SEQ ID NO: 130 - DNA, 20 bp, Artificial Sequence
  Synthetic oligonucleotide
tgcagtacagcagtcattca

SEQ ID NO: 131 - DNA, 20 bp, Artificial Sequence
  Synthetic oligonucleotide
caggaattaaaattataaaa

SEQ ID NO: 132 - DNA, 20 bp, Artificial Sequence
  Synthetic oligonucleotide
gtcaaactacacctccgccg

SEQ ID NO: 133 - DNA, 20 bp, Artificial Sequence
  Synthetic oligonucleotide
aacacaccgcgtcaaactac

SEQ ID NO: 134 - DNA, 20 bp, Artificial Sequence
  Synthetic oligonucleotide
attctctcccccacgtaaca

SEQ ID NO: 135 - DNA, 20 bp, Artificial Sequence
  Synthetic oligonucleotide
ctggagttttattctctccc

SEQ ID NO: 136 - DNA, 20 bp, Artificial Sequence
  Synthetic oligonucleotide
ctccgtcactgctttcgttc

SEQ ID NO: 137 - DNA, 20 bp, Artificial Sequence
  Synthetic oligonucleotide
agctcctccgtcactgcttt

SEQ ID NO: 138 - DNA, 20 bp, Artificial Sequence
  Synthetic oligonucleotide
accggtggtacaagctcctc

SEQ ID NO: 139 - DNA, 20 bp, Artificial Sequence
  Synthetic oligonucleotide
atttagttaccggtggtaca

SEQ ID NO: 140 - DNA, 20 bp, Artificial Sequence
  Synthetic oligonucleotide
gcatctccactctgctggtt

SEQ ID NO: 141 - DNA, 20 bp, Artificial Sequence
  Synthetic oligonucleotide
gcttgaactgtcatttgttg

SEQ ID NO: 142 - DNA, 20 bp, Artificial Sequence
  Synthetic oligonucleotide
gctgggcttgaactgtcatt

SEQ ID NO: 143 - DNA, 20 bp, Artificial Sequence
  Synthetic oligonucleotide
ctgtggctgggcttgaactg

SEQ ID NO: 144 - DNA, 20 bp, Artificial Sequence
  Synthetic oligonucleotide
gcaatctgtggctgggcttg

SEQ ID NO: 145 - DNA, 20 bp, Artificial Sequence
  Synthetic oligonucleotide
atgtggcaatctgtggctgg

SEQ ID NO: 146 - DNA, 20 bp, Artificial Sequence
  Synthetic oligonucleotide
ggctaatgtggcaatctgtg

SEQ ID NO: 147 - DNA, 20 bp, Artificial Sequence
  Synthetic oligonucleotide
agcagatgatgttgcatgag

SEQ ID NO: 148 - DNA, 20 bp, Artificial Sequence
  Synthetic oligonucleotide
gaactgtttggacttgtgga

SEQ ID NO: 149 - DNA, 20 bp, Artificial Sequence
  Synthetic oligonucleotide
tcttcactttctgcaatagt

SEQ ID NO: 150 - DNA, 20 bp, Artificial Sequence
  Synthetic oligonucleotide
gtgaatcttcactttctgca

SEQ ID NO: 151 - DNA, 20 bp, Artificial Sequence
  Synthetic oligonucleotide
ctcctgtgaatcttcacttt

SEQ ID NO: 152 - DNA, 20 bp, Artificial Sequence
  Synthetic oligonucleotide
taggaaggcctccttgaaag

SEQ ID NO: 153 - DNA, 20 bp, Artificial Sequence
  Synthetic oligonucleotide
cctccctgggtaatggcaat

SEQ ID NO: 154 - DNA, 20 bp, Artificial Sequence
  Synthetic oligonucleotide
ttgctcctccctgggtaatg

SEQ ID NO: 155 - DNA, 20 bp, Artificial Sequence
  Synthetic oligonucleotide
ctgtattgctcctccctggg

SEQ ID NO: 156 - DNA, 20 bp, Artificial Sequence
  Synthetic oligonucleotide
gccagctgtattgctcctcc

SEQ ID NO: 157 - DNA, 20 bp, Artificial Sequence
  Synthetic oligonucleotide
tgttagccagctgtattgct

SEQ ID NO: 158 - DNA, 20 bp, Artificial Sequence
  Synthetic oligonucleotide
accattgttagccagctgta

SEQ ID NO: 159 - DNA, 20 bp, Artificial Sequence
  Synthetic oligonucleotide
tgcaggccctgtaccccatc

SEQ ID NO: 160 - DNA, 20 bp, Artificial Sequence
  Synthetic oligonucleotide
gtagtacccggctgagtggc

SEQ ID NO: 161 - DNA, 20 bp, Artificial Sequence
  Synthetic oligonucleotide
gaatggtagtacccggctga

SEQ ID NO: 162 - DNA, 20 bp, Artificial Sequence
  Synthetic oligonucleotide
aacttggttgctgggcacta

SEQ ID NO: 163 - DNA, 20 bp, Artificial Sequence
  Synthetic oligonucleotide
aggctgtgtaggaagtgctg

SEQ ID NO: 164 - DNA, 20 bp, Artificial Sequence
  Synthetic oligonucleotide
tcagcaggctgtgtaggaag

SEQ ID NO: 165 - DNA, 20 bp, Artificial Sequence
  Synthetic oligonucleotide
cttcttcagcaggctgtgta

SEQ ID NO: 166 - DNA, 20 bp, Artificial Sequence
  Synthetic oligonucleotide
tgctgcttcttcagcaggct

SEQ ID NO: 167 - DNA, 20 bp, Artificial Sequence
  Synthetic oligonucleotide
attagacggacctctctctt

SEQ ID NO: 168 - DNA, 20 bp, Artificial Sequence
  Synthetic oligonucleotide
cctgttcttcattagacgga

SEQ ID NO: 169 - DNA, 20 bp, Artificial Sequence
  Synthetic oligonucleotide
tcaagcactgccactctgtt

SEQ ID NO: 170 - DNA, 20 bp, Artificial Sequence
  Synthetic oligonucleotide
gtgcttttagctcctcaatc

SEQ ID NO: 171 - DNA, 20 bp, Artificial Sequence
  Synthetic oligonucleotide
ccaaattaatctgatttgtg

SEQ ID NO: 172 - DNA, 20 bp, Artificial Sequence
  Synthetic oligonucleotide
caggcagttttgcgcataga

SEQ ID NO: 173 - DNA, 20 bp, Artificial Sequence
  Synthetic oligonucleotide
atgaaattctgtagttgctt

SEQ ID NO: 174 - DNA, 20 bp, Artificial Sequence
  Synthetic oligonucleotide
cacagtttaatgcaaaagca

SEQ ID NO: 175 - DNA, 20 bp, Artificial Sequence
  Synthetic oligonucleotide
ttggaacattcacagtttaa

SEQ ID NO: 176 - DNA, 20 bp, Artificial Sequence
  Synthetic oligonucleotide
tctcttcatgattcctggcg

SEQ ID NO: 177 - DNA, 20 bp, Artificial Sequence
  Synthetic oligonucleotide
acacttcttcattgcacctt

SEQ ID NO: 178 - DNA, 20 bp, Artificial Sequence
  Synthetic oligonucleotide
tacaggaagactgaactgtt

SEQ ID NO: 179 - DNA, 20 bp, Artificial Sequence
  Synthetic oligonucleotide
tagttgaaatctgagttccg

SEQ ID NO: 180 - DNA, 20 bp, Artificial Sequence
  Synthetic oligonucleotide
tattactcactgtactgccc

SEQ ID NO: 181 - DNA, 20 bp, Artificial Sequence
  Synthetic oligonucleotide
agctctatattccttttaaa

SEQ ID NO: 182 - DNA, 20 bp, Artificial Sequence
  Synthetic oligonucleotide
gcatggataccggtggtaca

SEQ ID NO: 183 - DNA, 20 bp, Artificial Sequence
  Synthetic oligonucleotide
tttagttaccaacactccgc

SEQ ID NO: 184 - DNA, 20 bp, Artificial Sequence
  Synthetic oligonucleotide
cctgaggcagtgtactgccc

SEQ ID NO: 185 - DNA, 20 bp, Artificial Sequence
  Synthetic oligonucleotide
aactgtccagacagaaggat

SEQ ID NO: 186 - DNA, 20 bp, Artificial Sequence
  Synthetic oligonucleotide
ctctatattccttgaacaac

SEQ ID NO: 187 - DNA, 20 bp, Artificial Sequence
  Synthetic oligonucleotide
acaaaagatgttctacttgg

(19) United States
(12) Patent Application Publication
(10) Pub. No.: US 2011/0177097 A1
(43) Pub. Date: Jul. 21, 2011

(54) METHODS FOR MODULATING EXPRESSION OF CREB

(76) Inventors: Gerald Shulman, East Haven, CT (US); Sanjay Bhanot, Carlsbad, CA (US); Xing-Xian Yu, San Diego, CA (US); Brett P. Monia, Encinitas, CA (US)

(21) Appl. No.: 12/993,435

(22) PCT Filed: May 22, 2009
(86) PCT No.: PCT/US09/45043

§ 371 Date: Apr. 8, 2011

Related U.S. Application Data
(60) Provisional application No. 61/128,627, filed on May 22, 2008.

(51) Int. Cl. A61K 39/395; A61K 31/7088; A61K 38/02; A61K 31/7125; A61K 31/712; A61K 31/7115; A61P 3/00; A61P 9/00; A61P 3/06; A61P 3/04; A61P 3/10; A61P 9/10; A61P 1/16; A61P 5/50

(52) U.S. Cl. 424/172.1; 514/44 R; 514/7.4; 514/6.8; 514/1.1; 514/4.8

(57) ABSTRACT

Methods are provided for modulating CREB by administering a CREB-specific modulator. Also provided are methods for treating cardiovascular and metabolic disorders in a subject or delaying or preventing risk factors thereof through the modulation of CREB. The present invention is also directed to methods of decreasing lipid levels in a subject or for preventing or delaying the onset of a rise in lipid levels in a subject, comprising administering to said subject a CREB-specific inhibitor.

### CROSS REFERENCE TO RELATED APPLICATIONS

This application claims benefit under 35 USC 119(e) to U.S. Provisional Application No. 61/128,627, filed May 22, 2008, which is incorporated herein by reference.

### SEQUENCE LISTING

The present application is being filed along with a Sequence Listing in electronic format. The Sequence Listing is provided as a file entitled BIOL0101WO2SEQ.txt, created on May 21, 2009, which is 224 Kb in size. The information in the electronic format of the sequence listing is incorporated herein by reference in its entirety.

### FIELD OF THE INVENTION

This invention is related generally to methods and agents for modulating cAMP Response Element Binding protein (CREB). More particularly, the present invention including and can relate to methods of inhibiting CREB with a CREB-specific inhibitor to treat diseases associated with metabolic and cardiovascular-related including disorders, particularly disorders associated with diabetes, dyslipidemia and body fat.

### BACKGROUND OF THE INVENTION

Obesity is considered a long-term disease and is characterized by the accumulation of excess adipose tissue. A large number of medical conditions have been associated with obesity. Certain such medical conditions, including diabetes, cancer, cardiovascular disease, non-alcoholic fatty liver disease (NALFD), are thought to be the result of increased number of fat cells. Obesity often results in an altered response to insulin (insulin resistance), and an increased tendency to thrombosis (prothrombotic state) (Bray, G. A. J. Clin. Endocrinol. Metab., 2004, 89 (6), 2583-9). Mortality is increased in obesity, with a body mass index (BMI) of over 32 being associated with a doubled risk of death (Manson et al N. Engl. J. Med., 1995, 333 (11), 677-85). Central obesity (male-type or waist-predominant obesity, characterized by a high waist-hip ratio), is an important risk factor for metabolic syndrome. Despite attempts to control weight gain, obesity remains a serious health concern in the United States and other industrialized countries.

Diabetes affects over 18.2 million people in the United States, representing over 6% of the population. Diabetes is characterized by the inability to produce or properly use insulin. Type 2 diabetes (T2DM; also called non-insulin-dependent diabetes or NIDDM) accounts for 80-90% of the diagnosed cases of diabetes and usually begins as insulin resistance. Type 2 diabetes is often a disease of over-nutrition; the onset and progression of which is generally associated with excess fat accumulation in the abdomen, muscles and liver (Gagliardi, L. and Wittert, G., 2007, Curr Diabetes Rev., 3, 95-101). Modest weight loss (˜7%) achieved by diet and exercise can prevent, or delay, the onset of T2DM. In individuals with established T2DM, weight loss, meal replacements and pharmacology for obesity, as part of an integrated management plan is useful for optimizing glycaemic control. Insulin resistance in diabetes type 2 prevents maintenance of blood glucose within desirable ranges, despite normal to elevated plasma levels of insulin. Insulin resistance is also an important factor for the metabolic syndrome (Matfin, G., Curr. Dian. Rep., 2008, 8, 31-36). Additionally, glucotoxicity, which results from long-term hyperglycemia, induces tissue-dependent insulin resistance (Nawano et al., Am. J. Physiol. Endocrinol. Metab., 2000, 278, E535-543) exacerbating the disease. Such chronic hyperglycemia is also a major risk factor for diabetes-associated complications, including heart disease, retinopathy, nephropathy and neuropathy.

Diabetes and obesity (sometimes collectively referred to as “diabesity”) are interrelated in that obesity is known to exacerbate the pathology of diabetes and greater than 60% of diabetics are obese. Most human obesity is associated with insulin resistance and leptin resistance. In fact, it has been suggested that obesity may have an even greater impact on insulin action than diabetes itself (Sindelka et al., Physiol Res., 2002, 51, 85-91). Additionally, several compounds on the market for the treatment of diabetes are known to induce weight gain, a very undesirable side effect to the treatment of this disease.

Cardiovascular disease is also interrelated to obesity and diabetes. Cardiovascular disease encompasses a wide variety of etiologies and has an equally wide variety of causative agents and interrelated players. Many causative agents contribute to symptoms such as elevated plasma levels of cholesterol, including non-HDL cholesterol, as well as other lipid-related disorders. Such lipid-related disorders, generally referred to as dyslipidemia, include hypercholesterolemia and hypertriglyceridemia among other indications. Non-HDL cholesterol is firmly associated with atherogenesis and its sequalea, including cardiovascular diseases such as arteriosclerosis, coronary artery disease, myocardial infarction, ischemic stroke, and other forms of heart disease. These rank as the most prevalent types of illnesses in industrialized countries. Indeed, an estimated 12 million people in the United States suffer with coronary artery disease and about 36 million require treatment for elevated cholesterol levels.

Metabolic syndrome is a combination of medical disorders that increase one's risk for cardiovascular disease. The risk factors include obesity, diabetes, hypertension and dyslipidemia (Grundy, S. M., J. Clin. Endocrinol. Metab., 2004, 89 (6), 2595-600). In some studies, the prevalence in the USA is calculated as being up to 25% of the population. Metabolic syndrome is known under various other names, such as (metabolic) syndrome X, insulin resistance syndrome, Reaven's syndrome or CHAOS.

Because of their interrelatedness, an agent or method that has the potential to treat diabetes, obesity, other metabolic-related disorders as well as cardiovascular-related disorders would provide a significant improvement over current treatments.

The regulation of gene expression by specific signal transduction pathways is tightly connected to the cell phenotype and the response elicited by a given pathway varies depending on the cell type. One of the most common secondary messenger signaling molecules in cells is cyclic adenosine monophosphate (cAMP). The binding of specific ligands to their receptors results in the activation or inhibition of the cAMP-dependent pathway, ultimately affecting the transcriptional regulation of various genes through distinct promoter-responsive sites. All cAMP responsive gene promoters have in common an 8-base enhancer known as the cAMP response element (CRE). One of the best characterized stimulus-induced transcription factors is CREB (cAMP response element binding protein or CREB1), a central transcription factor that mediates cAMP and calcium-dependent gene expression.

With the high prevalence of diabetes, obesity and other metabolic disorders as well as cardiovascular-related disorders such as cholesterol-related conditions (including lipid disorders, generally), there remains a need for improved approaches to treat one or more of these conditions. Modulation of CREB expression may prove to be a useful method for treating a wide range of metabolic and cardiovascular conditions, including but not limited to the interrelated conditions of diabetes, obesity, metabolic syndrome and there associated etiology and sequalea as provided herein.

### SUMMARY OF THE INVENTION

Provided herein are methods, agents and compositions for modulating CREB. The agents and compositions include CREB-specific modulators. CREB-specific modulators include proteins, peptides, polypeptides, antibodies, antisense compounds, including oligonucleotides and antisense oligonucleotides, ssRNA, dsRNA molecules, ribozymes, triple helix molecules, siRNA and other RNAi compounds, and small molecule modulators. Any of the listed CREB-specific modulators can be CREB-specific inhibitors.

Also provided are methods of treating diseases and disorders. Included are methods of treating cardiovascular and metabolic diseases and disorders. The diseases and disorders include, but are not limited to, those associated with lipid dysregulation, fat dysregulation, adipocyte dysregulation, and glucose dysregulation.

Also provided are methods of treating multiple disease or disorders. The multiple diseases or disorders can include any of the disease and disorders provided herein. The multiple diseases and disorder can have one or more risk factors, causes or outcomes in common.

The present invention is also directed to methods of reducing risk factors associated with disease and causes of disease. Such diseases include cardiovascular and metabolic diseases such as, but not limited to diabetes, metabolic syndrome and atherosclerosis. Risk factors include, but are not limited to, lipid level, adiposity, glucose level and insulin responsiveness.

In particular embodiments, methods of treatment include administering to a subject a CREB-specific modulator, as described herein. In particular embodiments, a CREB-specific inhibitor is administered, as described herein.

Methods of modulating CREB include methods of modulating levels of CREB. The levels can include but are not limited to CREB mRNA levels and CREB protein levels. Modulation can occur in a cell or tissue. In a certain embodiment the cell or tissue is in an animal. In certain embodiments, the animal is a human, as described herein. In certain embodiments, CREB levels are reduced, as described herein. Such reduction can occur in a time-dependent manner or in a dose-dependent manner or both.

### BRIEF DESCRIPTION OF THE DRAWINGS

FIG. 1

Is a graphical representation showing that CREB antisense oligonucleotide, ISIS 385915, decreases CREB expression in Liver and white adipose tissue (WAT). Total RNA was isolated from fasted and fed livers (A) and fasted WAT (B), and quantified using RT-PCR (n=4-6 rats per treatment group). (C) Immunoblots of liver homogenates from control, ISIS 141923, and CREB antisense oligonucleotide rats in the fasted state. * P<0.05.

FIG. 2

Is a graphical representation showing that CREB antisense oligonucleotide, therapy improves whole body insulin sensitivity. Plasma glucose (A), insulin (B), and glucagon (C) concentrations. Liver gene expression of key gluconeogenic transcription factors (D) (n=4-8 per treatment group).

FIG. 3

Is a graphical representation showing that CREB antisense oligonucleotide therapy decreases plasma triglycerides and cholesterol. Plasma triglycerides were decreased with CREB antisense oligonucleotide (A). The lipoprotein profile showed significant reductions in VLDL, LDL, and HDL with CREB antisense oligonucleotide (B). Synthesis of cholesterol in rat hepatocytes (C) (n=3-7 per treatment group). Liver gene expression of key enzymes in cholesterol metabolism (D) (n=4-8 per treatment group). Amount of bile acids extracted from feces during a 12 hour fast (E) (n=7 per treatment group). * P<0.05.

FIG. 4

Is a graphical representation showing that CREB antisense oligonucleotide treatment, ISIS 385915, improves hepatic lipid content. Liver triglycerides (A), Liver DAGs (B), and long chain acyl CoAs (C) are all significantly decreased with CREB antisense oligonucleotide treatment (n=8-13 per treatment group). The incorporation of glycerol into triglycerides did not differ (D) but the rate of fat oxidation (E) was increased (n=3-7 per treatment group). Gene expression of major lipid transcription factors and enzymes (F) (n=5-8 per treatment group). * P<0.05, ** P<0.05.

FIG. 5

Is a graphical representation showing CREB antisense oligonucleotide treatment improves hepatic insulin sensitivity in T2DM rats. Glucose basal endogenous glucose production (A), insulin-stimulated whole-body glucose metabolism (B), and suppression of hepatic glucose production (C) were assessed using the hyperinsulemic-euglycemic clamp (n=4-17 per treatment group). In the T2DM rat model CREB antisense oligonucleotide reduced membrane PKCε (D) (n=4 per treatment group), improved Akt2 activity (E) (n=5 per treatment group). CREB antisense oligonucleotide decreased the putative Akt inhibitor TRB3 (F) but did not effect IRS-2 expression (G)* P<0.05.

### DETAILED DESCRIPTION OF THE PREFERRED EMBODIMENTS

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methylcytosine” means a cytosine modified with a methyl group attached to the 5′ position. A 5-methylcytosine is a modified nucleobase.

“3′ target site” refers to the nucleotide of a target nucleic acid which is complementary to the 3′-most nucleotide of a particular antisense compound.

‘“5’ target site” refers to the nucleotide of a target nucleic acid which is complementary to the 5′-most nucleotide of a particular antisense compound.

“Active pharmaceutical ingredient” means the substance in a pharmaceutical composition that provides a desired effect.

“Adipogenesis” means the development of fat cells from preadipocytes. “Lipogenesis” means the production or formation of fat, either fatty degeneration or fatty infiltration.

“Adiposity” or “Obesity” refers to the state of being obese or an excessively high amount of body fat or adipose tissue in relation to lean body mass. The amount of body fat includes concern for both the distribution of fat throughout the body and the size of the adipose tissue deposits. Body fat distribution can be estimated by skin-fold measures, waist-to-hip circumference ratios, or techniques such as ultrasound, computed tomography, or magnetic resonance imaging. According to the Center for Disease Control and Prevention, individuals with a body mass index (BMI) of 30 or more are considered obese. The term “Obesity” as used herein includes conditions where there is an increase in body fat beyond the physical requirement as a result of excess accumulation of adipose tissue in the body. The term “obesity” includes, but is not limited to, the following conditions: adult-onset obesity; alimentary obesity; endogenous or metabolic obesity; endocrine obesity; familial obesity; hyperinsulinar obesity; hyperplastic-hypertrophic obesity; hypogonadal obesity; hypothyroid obesity; lifelong obesity; morbid obesity and exogenous obesity.

“Administering” means providing a pharmaceutical agent to an individual, and includes, but is not limited to administering by a medical professional and self-administering.

“Administered concomitantly” refers to the administration of two agents within the same therapeutic time frame; which means, in any manner in which the pharmacological effects of both are manifest in the patient at the same time or during the same time period. Concomitant administration does not require that both agents be administered in a single pharmaceutical composition, in the same dosage form, or by the same route of administration.

“Amelioration” refers to a lessening of at least one indicator, sign or symptom of an associated condition or disease. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antibody” refers to an immunoglobulin molecule or immunologically active portion thereof characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region. The antibody can be a polyclonal, monoclonal, recombinant; e.g., a chimeric or humanized; fully human, non-human; e.g., murine; or single chain antibody.

“Antisense compound” refers to an oligomeric compound that is at least partially complementary to a target nucleic acid molecule to which it hybridizes. For example, “antisense compound targeted to CREB” refers to an oligomeric compound at least partially complementary to the CREB nucleic acid molecule. In certain embodiments, an antisense compound modulates (increases or decreases) levels and/or expression of a target nucleic acid, as described herein. Antisense compounds include, but are not limited to, compounds that are oligonucleotides, oligonucleotides, oligonucleotide analogs, oligonucleotide mimetics, and chimeric combinations of these. Consequently, while all antisense compounds are oligomeric compounds, not all oligomeric compounds are antisense compounds.

“Antisense inhibition” means reduction of target nucleic acid levels, in the presence of an antisense compound complementary to a target nucleic acid, compared to target nucleic acid levels in the absence of the antisense compound.

“Antisense oligonucleotide” means a single-stranded oligonucleotide having a nucleobase sequence that will permits hybridization to a corresponding region of a target nucleic acid.

“ApoB-containing lipoprotein” means any lipoprotein that has apolipoprotein B as its protein component, and is understood to include LDL, VLDL, IDL, and lipoprotein(a) and can be generally targeted by lipid lowering agent and therapies.

“Atherosclerosis” means a hardening of the arteries affecting large and medium-sized arteries and is characterized by the presence of fatty deposits. The fatty deposits are called “atheromas” or “plaques,” which consist mainly of cholesterol and other fats, calcium and scar tissue, and damage the lining of arteries.

“Bicyclic nucleic acid sugar moiety” means a furanosyl ring modified by the bridging of two non-geminal ring atoms. A bicyclic sugar is a modified sugar.

“Body weight” refers to an animal's total weight, inclusive of all tissues including adipose tissue.

“Body fat content” refers to an animal's total amount of adipose tissue mass or weight.

“Cap structure” or “terminal cap moiety” means chemical modifications, which have been incorporated at either terminus of an antisense compound.

“Cardiovascular disease” or “cardiovascular disorder” refers to a group of conditions related to the heart, blood vessels, or the circulation. Examples of cardiovascular diseases include, but are not limited to, aneurysm, angina, arrhythmia, atherosclerosis, cerebrovascular disease (stroke), coronary heart disease, hypertension, dyslipidemia, hyperlipidemia, and hypercholesterolemia.

“Chimeric antisense compounds” means antisense compounds that have at least 2 chemically distinct regions, each region having a plurality of subunits.

“Cholesterol” is a sterol molecule found in the cell membranes of all animal tissues. Cholesterol may be transported in an animal's blood plasma by lipoproteins including very low density lipoprotein (VLDL), intermediate density lipoprotein (IDL), low density lipoprotein (LDL), and high density lipoprotein (HDL). “Plasma cholesterol” refers to cholesterol present in the plasma.

“Cholesterol absorption inhibitor” means an agent that inhibits the absorption of exogenous cholesterol obtained from diet.

“Co-administration” refers to administration of two or more agents to an animal. The two or more agents may be in a single pharmaceutical composition, or may be in separate pharmaceutical compositions. Both agents may be administered through the same or different routes of administration. Co-administration encompasses administration in parallel or sequentially.

“Complementarity” means the capacity for pairing between nucleobases of a first nucleic acid and a second nucleic acid.

“Contiguous nucleobases” means nucleobases immediately adjacent to each other.

“Coronary heart disease (CHD)” means a narrowing of the small blood vessels that supply blood and oxygen to the heart, which is often a result of atherosclerosis.

“CREB” means any nucleic acid encoding CREB or CREB protein. An exemplary CREB includes CREB having the amino acid sequence encoded by a nucleic acid sequence, e.g. SEQ ID NO: 1. For example, in certain embodiments, a CREB nucleic acid includes, without limitation, a DNA sequence encoding CREB, an RNA sequence transcribed from DNA encoding CREB, and an mRNA sequence encoding CREB. “CREB mRNA” means an mRNA encoding a CREB protein.

“CREB specific-inhibitor” refers to an agent that inhibits, reduces, impairs or decreases the expression, activity or processing of CREB. A CREB-specific inhibitor can also refer to any agent that inhibits targets up-stream or downstream of CREB resulting in the inhibition of CREB expression. A CREB-specific inhibitor can also refer to an agent that inhibits the differentiation potential or proliferation of a CREB-expressing cell. For example, a CREB specific-inhibitor can include, but is not limited to, proteins, peptides, polypeptides, antibodies, antisense compounds, including oligonucleotides and antisense oligonucleotides, ssRNA, dsRNA molecules, ribozymes, triple helix molecules, siRNA and other RNAi compounds, and small molecule modulators. The antisense compounds included herein, can operate by an RNaseH or RNAi mechanism or by other known mechanism, such as splicing. A CREB-specific modulator can be a CREB-specific inhibitor.

“CREB-specific modulator” as used herein, refers to an agent that modulates the expression, activity, or processing of CREB. A CREB-specific modulator can also refer to an agent that modulates the differentiation potential or proliferation of a CREB-expressing cell. A CREB-specific modulator can also refer to any agent that modulates targets up-stream or downstream of CREB resulting in the modulation of CREB expression. For example, a CREB-specific modulator can include, but is not limited to proteins, peptides, polypeptides, antibodies, antisense compounds, including oligonucleotides and antisense oligonucleotides, ssRNA, dsRNA molecules, ribozymes, triple helix molecules, siRNA and other RNAi compounds and small molecule modulators. The antisense compounds included herein, can operate by an RNaseH or RNAi mechanism or by other known mechanism, such as splicing. As used herein, CREB-specific modulator also can provide a therapeutic benefit when administered to a subject

“Deoxyribonucleotide” means a nucleotide having a hydrogen at the 2′ position of the sugar portion of the nucleotide. Deoxyribonucleotides may be modified with any of a variety of substituents.

“Diabetes mellitus” or “diabetes” is a syndrome characterized by disordered metabolism and abnormally high blood sugar (hyperglycemia) resulting from insufficient levels of insulin or reduced insulin sensitivity. The characteristic symptoms are excessive urine production (polyuria) due to high blood glucose levels, excessive thirst and increased fluid intake (polydipsia) attempting to compensate for increased urination, blurred vision due to high blood glucose effects on the eye's optics, unexplained weight loss, and lethargy.

“Diabetic dyslipidemia” or “type 2 diabetes with dyslipidemia” means a condition characterized by Type 2 diabetes, reduced HDL-C, elevated triglycerides, and elevated small, dense LDL particles.

“Diluent” means an ingredient in a composition that lacks pharmacological activity, but is pharmaceutically necessary or desirable. For example, in agents that are injected, the diluent may be a liquid, e.g. saline solution.

“Dose” means a specified quantity of a CREB-specific modulator or agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in a subject.

“Dyslipidemia” refers to a disorder of lipid and/or lipoprotein metabolism, including lipid and/or lipoprotein overproduction or deficiency. Dyslipidemias may be manifested by elevation of lipids such as cholesterol and triglycerides as well as lipoproteins such as low-density lipoprotein (LDL) cholesterol.

“Effective amount” in the context of modulating an activity or of treating or preventing a condition means the administration of that amount of active ingredient to a subject in need of such modulation, treatment or prophylaxis, either in a single dose or as part of a series, that is effective for modulation of that effect, or for treatment or prophylaxis or improvement of that condition. The effective amount will vary depending upon the health and physical condition of the subject to be treated, the taxonomic group of subjects to be treated, the formulation of the composition, the assessment of the medical situation, and other relevant factors.

“Elevated apoB-levels” means a subject who has been identified as having apoB levels near or above the level at which therapeutic intervention is recommended, according to guidelines recognized by medical professionals. Such a subject may also be considered “in need of treatment” to decrease apoB levels.

“Elevated cholesterol” means cholesterol at a concentration in a subject at which lipid-lowering therapy is recommended, and includes, without limitation, elevated LDL-C″, “elevated VLDL-C,” “elevated IDL-C” and “elevated non-HDL-C.” In certain embodiments, cholesterol concentrations of less than 200 mg/dL, 200-239 mg/dL, and greater than 240 mg/dL are considered desirable, borderline high, and high, respectively. In certain embodiments, LDL-C concentrations of 100 mg/dL, 100-129 mg/dL, 130-159 mg/dL, 160-189 mg/dL, and greater than 190 mg/dL are considered optimal, near optimal/above optimal, borderline high, high, and very high, respectively.

“Elevated lipoprotein” means a concentration of lipoprotein in a subject at which lipid-lowering therapy is recommended.

“Elevated triglyceride” means a concentration of triglyceride in the serum or liver at which lipid-lowering therapy is recommended, and includes “elevated triglyceride” and “elevated liver triglyceride.” In certain embodiments, triglyceride concentration of 150-199 mg/dL, 200-499 mg/dL, and greater than or equal to 500 mg/dL is considered borderline high, high, and very high, respectively.

“Fully complementary” means each nucleobase of a first nucleic acid has a complementary nucleobase in a second nucleic acid. In certain embodiments, a first nucleic acid is an antisense compound and a target nucleic acid is a second nucleic acid.

“Gapmer” refers to a chimeric oligomeric compound comprising a central region (“gap”) and a region on either side of the central region (the “wings”), wherein, the gap comprises at least one modification that is different from that of each wing. Such modifications include nucleobase, monomeric linkage, and sugar modifications, as well as the absence of modification (unmodified). The gap region generally supports RNaseH cleavage.

“Gap-widened” means an antisense compound having a gap segment of 12 or more contiguous 2′-deoxyribonucleotides positioned between 5′ and 3′ wing segments having from one to six nucleotides having modified sugar moieties.

“Glucose” is a monosaccharide used by cells as a source of energy and metabolic intermediate. “Plasma glucose” refers to glucose present in the plasma.

“High density lipoprotein-C(HDL-C)” means cholesterol associated with high density lipoprotein particles. Concentration of HDL-C in serum (or plasma) is typically quantified in mg/dL or nmol/L. “Serum HDL-C” and “plasma HDL-C” mean HDL-C in the serum and plasma, respectively.

“HMG-CoA reductase inhibitor” means an agent that acts through the inhibition of the enzyme HMG-CoA reductase, such as atorvastatin, rosuvastatin, fluvastatin, lovastatin, pravastatin, and simvastatin.

“Hybridization” means the annealing of complementary nucleic acid molecules. In certain embodiments, complementary nucleic acid molecules include, but are not limited to, an antisense compound and a nucleic acid target. In certain such embodiments, complementary nucleic acid molecules include, but are not limited to, an antisense oligonucleotide and a nucleic acid target.

“Hypercholesterolemia” means a condition characterized by elevated cholesterol or circulating (plasma) cholesterol, LDL-cholesterol and VLDL-cholesterol, as per the guidelines of the Expert Panel Report of the National Cholesterol Educational Program (NCEP) of Detection, Evaluation of Treatment of high cholesterol in adults (see, Arch. Int. Med. (1988) 148, 36-39).

“Hyperlipidemia” or “hyperlipemia” is a condition characterized by elevated serum lipids or circulating (plasma) lipids. This condition manifests an abnormally high concentration of fats. The lipid fractions in the circulating blood are cholesterol, low density lipoproteins, very low density lipoproteins and triglycerides.

“Hypertriglyceridemia” means a condition characterized by elevated triglyceride or circulating (plasma) triglyceride levels.

“Identifying” or “selecting a subject having a metabolic or cardiovascular disease” means identifying or selecting a subject having been diagnosed with a metabolic disease, a cardiovascular disease, or a metabolic syndrome; or, identifying or selecting a subject having any symptom of a metabolic disease, cardiovascular disease, or metabolic syndrome including, but not limited to, hypercholesterolemia, hyperglycemia, hyperlipidemia, hypertriglyceridemia, hypertension increased insulin resistance, decreased insulin sensitivity, above normal body weight, and/or above normal body fat content or any combination thereof. Such identification may be accomplished by any method, including but not limited to, standard clinical tests or assessments, such as measuring serum or circulating (plasma) cholesterol, measuring serum or circulating (plasma) blood-glucose, measuring serum or circulating (plasma) triglycerides, measuring blood-pressure, measuring body fat content, measuring body weight, and the like.

“Identifying” or “selecting a diabetic subject” means identifying or selecting a subject having been identified as diabetic or identifying or selecting a subject having any symptom of diabetes (type 1 or type 2) such as, but not limited to, having a fasting glucose of at least 110 mg/dL, glycosuria, polyuria, polydipsia, increased insulin resistance, and/or decreased insulin sensitivity.

“Identifying” or ‘selecting an obese subject” means identifying or selecting a subject having been diagnosed as obese or identifying or selecting a subject with a BMI over 30 and/or a waist circumference of greater than 102 cm in men or greater than 88 cm in women.

“Identifying” or “selecting a subject having dyslipidemia” means identifying or selecting a subject diagnosed with a disorder of lipid and/or lipoprotein metabolism, including lipid and/or lipoprotein overproduction or deficiency. Dyslipidemias may be manifested by elevation of lipids such as cholesterol and triglycerides as well as lipoproteins such as low-density lipoprotein (LDL) cholesterol.

“Identifying” or “selecting a subject having increased adiposity” means identifying or selecting a subject having an increased amount of body fat (or adiposity) that includes concern for one or both the distribution of fat throughout the body and the size of the adipose tissue deposits. Body fat distribution can be estimated by skin-fold measures, waist-to-hip circumference ratios, or techniques such as ultrasound, computed tomography, or magnetic resonance imaging. According to the Center for Disease Control and Prevention, individuals with a body mass index (BMI) of 30 or more are considered obese.

“Improved cardiovascular outcome” means a reduction in the occurrence of adverse cardiovascular events, or the risk thereof. Examples of adverse cardiovascular events include, without limitation, death, reinfarction, stroke, cardiogenic shock, pulmonary edema, cardiac arrest, and atrial dysrhythmia.

“Individual” means a human or non-human animal selected for treatment or therapy.

“Inhibiting the expression or activity” refers to a reduction or blockade of the expression or activity and does not necessarily indicate a total elimination of expression or activity.

“Insulin resistance” is defined as the condition in which normal amounts of insulin are inadequate to produce a normal insulin response from fat, muscle and liver cells. Insulin resistance in fat cells results in hydrolysis of stored triglycerides, which elevates free fatty acids in the blood plasma. Insulin resistance in muscle reduces glucose uptake whereas insulin resistance in liver reduces glucose storage, with both effects serving to elevate blood glucose. High plasma levels of insulin and glucose due to insulin resistance often leads to metabolic syndrome and type 2 diabetes.

“Insulin sensitivity” is a measure of how effectively an individual processes glucose. An individual having high insulin sensitivity effectively processes glucose whereas an individual with low insulin sensitivity does not effectively process glucose.

“Intermediate low density lipoprotein-cholesterol (IDL-C)” means cholesterol associated with intermediate density lipoprotein. Concentration of IDL-C in serum (or plasma) is typically quantified in mg/mL or nmol/L. “Serum IDL-C” and “plasma IDL-C” mean IDL-C in the serum or plasma, respectively.

“Internucleoside linkage” means a covalent linkage between adjacent nucleosides.

“Intravenous administration” means administration into a vein.

“Linked nucleosides” means adjacent nucleosides which are bonded together.

“Lipid-lowering” means a reduction in one or more serum lipids in a subject. Lipid-lowering can occur with one or more doses over time.

“Lipid-lowering agent” means an agent; for example, a CREB-specific modulator; provided to a subject to achieve a lowering of lipids in the subject. For example, in certain embodiments, a lipid-lowering agent is provided to a subject to reduce one or more of ApoB, LDL-C, cholesterol, and triglycerides.

“Lipid-lowering therapy” means a therapeutic regimen provided to a subject to reduce one or more lipids in a subject. In certain embodiments, a lipid-lowering therapy is provided to reduce one or more of ApoB, cholesterol, LDL-C, VLDL-C, IDL-C, non-HDL-C, triglycerides, small dense LDL particles, and Lp(a) in a subject.

“Lipoprotein”, such as VLDL, LDL and HDL, refers to a group of proteins found in the serum, plasma and lymph and are important for lipid transport. The chemical composition of each lipoprotein differs in that the HDL has a higher proportion of protein versus lipid, whereas the VLDL has a lower proportion of protein versus lipid.

“Low density lipoprotein-cholesterol (LDL-C)” means cholesterol carried in low density lipoprotein particles. Concentration of LDL-C in serum (or plasma) is typically quantified in mg/dL or nmol/L. “Serum LDL-C” and “plasma LDL-C” mean LDL-C in the serum and plasma, respectively.

“Low HDL-C” means a concentration of HDL-C in a subject at which lipid-lowering therapy to increase HDL-C is recommended. In certain embodiments, lipid-lowering therapy is recommended when low HDL-C is accompanied by elevations in non-HDL-C and/or elevations in triglyceride. In certain embodiments, HDL-C concentrations of less than 40 mg/dL are considered low. In certain embodiments, HDL-C concentrations of less than 50 mg/dL are considered low.

“Major risk factors” refers to factors that contribute to a high risk for a particular disease or condition. In certain embodiments, major risk factors for coronary heart disease include, without limitation, cigarette smoking, hypertension, low HDL-C, family history of coronary heart disease, age, and other factors disclosed herein.

“Metabolic disorder” refers to a condition characterized by an alteration or disturbance in metabolic function. “Metabolic” and “metabolism” are terms well known in the art and generally include the whole range of biochemical processes that occur within a living organism. Metabolic disorders include, but are not limited to, hyperglycemia, prediabetes, diabetes (type I and type 2), obesity, insulin resistance, metabolic syndrome and dyslipidemia related to metabolic conditions.

“Metabolic syndrome” means a condition characterized by a clustering of lipid and non-lipid cardiovascular risk factors of metabolic origin. In certain embodiments, metabolic syndrome is identified by the presence of any 3 of the following factors: waist circumference of greater than 102 cm in men or greater than 88 cm in women; triglyceride of at least 150 mg/dL; HDL-C less than 40 mg/dL in men or less than 50 mg/dL in women; blood pressure of at least 130/85 mmHg; and fasting glucose of at least 110 mg/dL. These determinants can be readily measured in clinical practice (JAMA, 2001, 285: 2486-2497).

“Mismatch” refers to a non-complementary nucleobase within a complementary oligomeric compound.

“Mixed dyslipidemia” means a condition characterized by elevated cholesterol and elevated triglycerides.

“Modified internucleoside linkage” means substitution and/or any change from a naturally occurring internucleoside linkage.

“Modified nucleobase” means any nucleobase other than adenine, cytosine, guanine, thymidine, or uracil. An “unmodified nucleobase” means the purine bases, adenine (A) and guanine (G), and the pyrimidine bases, thymine (T), cytosine (C) and uracil (U).

“Modified nucleoside” means a nucleoside having, independently, a modified sugar moiety or modified nucleobase.

“Modified nucleotide” means a nucleotide having, independently, a modified sugar moiety, modified internucleoside linkage, or modified nucleobase.

“Modified sugar” means substitution and/or any change from a natural sugar moiety.

“Modified oligonucleotide” means an oligonucleotide comprising a modification such as a modified internucleoside linkage, a modified sugar, and/or a modified nucleobase.

“Motif” means the pattern of unmodified and modified nucleosides in an antisense compound.

“MTP inhibitor” means an agent inhibits the enzyme, microsomal triglyceride transfer protein.

“Naturally occurring internucleoside linkage” means a 3′ to 5′ phosphodiester linkage.

“Natural sugar moiety” means a sugar moiety found in DNA (2′-H) or RNA (2′-OH).

“Non-alcoholic fatty liver disease” or “NAFLD” means a condition characterized by fatty inflammation of the liver that is not due to excessive alcohol use (for example, alcohol consumption of over 20 g/day). In certain embodiments, NAFLD is related to insulin resistance and the metabolic syndrome. NAFLD encompasses a disease spectrum ranging from simple triglyceride accumulation in hepatocytes (hepatic steatosis) to hepatic steatosis with inflammation (steatohepatitis), fibrosis, and cirrhosis.

“Nonalcoholic steatohepatitis (NASH)” occurs from progression of NAFLD beyond deposition of triglycerides. A second-hit capable of inducing necrosis, inflammation, and fibrosis is required for development of NASH. Candidates for the second-hit can be grouped into broad categories: factors causing an increase in oxidative stress and factors promoting expression of proinflammatory cytokines. It has been suggested that increased liver triglycerides lead to increased oxidative stress in hepatocytes of animals and humans, indicating a potential cause-and-effect relationship between hepatic triglyceride accumulation, oxidative stress, and the progression of hepatic steatosis to NASH (Browning and Horton, J. Clin. Invest., 2004, 114, 147-152). Hypertriglyceridemia and hyperfattyacidemia can cause triglyceride accumulation in peripheral tissues (Shimamura et al., Biochem. Biophys. Res. Commun., 2004, 322, 1080-1085). In some embodiments, the steatosis is steatohepatitis. In some embodiments, the steatosis is NASH.

“Non-complementary nucleobase” refers to a pair of nucleobases that do not form hydrogen bonds with one another or otherwise support hybridization.

“Non-familial hypercholesterolemia” means a condition characterized by elevated cholesterol that is not the result of a single gene mutation.

“Non-high density lipoprotein-cholesterol (Non-HDL-C)” means cholesterol associated with lipoproteins other than high density lipoproteins, and includes, without limitation, LDL-C, VLDL-C, and IDL-C.

“Non-specific CREB inhibitor” or “additional therapy” means an agent that is not specifically targeted to CREB. For example, a non-specific CREB inhibitor is not exclusively directed to the modulation of CREB. The Non-specific CREB inhibitor or additional therapy can be an agent that can be administered in combination with a CREB-specific modulator or inhibitor. In some instances, an additional therapy can be a cholesterol-lowering agent and/or glucose-lowering agent and/or a lipid-lowering agent and/or fat/adipose tissue-lowering agent.

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleoside” means a nucleobase linked to a sugar.

As used herein the term “nucleoside mimetic” is intended to include those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics e.g. non furanose sugar units.

The term “nucleotide mimetic” is intended to include those structures used to replace the nucleoside and the linkage at one or more positions of an oligomeric compound such as for example peptide nucleic acids or morpholinos (morpholinos linked by —N(H)—C(═O)—O— or other non-phosphodiester linkage).

“Nucleobase sequence” means the order of contiguous nucleobases independent of any sugar, linkage, and/or nucleobase modification.

“Nucleotide” means a nucleoside having a phosphate group covalently linked to the sugar portion of the nucleoside.

“Oligomeric compound” refers to a polymeric structure comprising two or more sub-structures and capable of hybridizing to a region of a nucleic acid molecule. In certain embodiments, oligomeric compounds are oligonucleosides. In certain embodiments, oligomeric compounds are oligonucleotides. In certain embodiments, oligomeric compounds are antisense compounds. In certain embodiments, oligomeric compounds are antisense oligonucleotides. In certain embodiments, oligomeric compounds are chimeric oligonucleotides.

“Oligonucleoside” means an oligonucleotide in which the internucleoside linkages do not contain a phosphorus atom.

“Oligonucleotide” refers to an oligomeric compound comprising a plurality of linked nucleotides. In certain embodiment, one or more nucleotides of an oligonucleotide is modified. In certain embodiments, an oligonucleotide contains ribonucleic acid (RNA) or deoxyribonucleic acid (DNA). In certain embodiments, oligonucleotides are composed of naturally- and/or non-naturally-occurring nucleobases, sugars and covalent internucleotide linkages, and may further include non-nucleic acid conjugates.

“Parenteral administration” means administration through injection or infusion. Parenteral administration includes, but is not limited to, subcutaneous administration, intravenous administration, or intramuscular administration.

“Peptide” means a molecule formed by linking at least two amino acids by amide bonds. Without limitation, as used herein, “peptide” refers to polypeptides and proteins.

“Pharmaceutical agent” means a substance that provides a therapeutic benefit when administered to an individual. For example, in certain embodiments, an antisense oligonucleotide targeted to CREB is pharmaceutical agent.

“Pharmaceutical composition” means a mixture of substances suitable for administering to a subject. For example, a pharmaceutical composition may comprise an agent, for example a CREB-specific modulator like an antisense oligonucleotide; and a sterile aqueous solution.

“Pharmaceutically acceptable carrier” means a medium or diluent that does not interfere with the structure of the oligonucleotide. Certain such carriers enable pharmaceutical compositions to be formulated as, for example, tablets, pills, dragees, capsules, liquids, gels, syrups, slurries, suspension and lozenges for the oral ingestion by a subject.

“Polygenic hypercholesterolemia” means a condition characterized by elevated cholesterol that results from the influence of a variety of genetic factors. In certain embodiments, polygenic hypercholesterolemia may be exacerbated by dietary intake of lipids.

“Phosphorothioate internucleoside linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a target nucleic acid. In certain embodiments, a portion is a defined number of contiguous nucleobases of a target nucleic acid. In certain embodiments, a portion is a defined number of contiguous nucleobases of an antisense compound.

“Prevention” or “preventing” refers to delaying or forestalling the onset or development of a condition or disease for a period of time from hours to days, preferably weeks to months.

“Prodrug” means a therapeutic agent that is prepared in an inactive or less active form that is converted to an active or more active form (i.e., drug) within the body or cells thereof by the action of endogenous enzymes or other chemicals and/or conditions. In certain embodiments, a shortened oligonucleotide or oligonucleotide metabolite may be more active than it's parent (e.g. 20mer) oligonucleotide.

“Ribonucleotide” means a nucleotide having a hydroxy at the 2′ position of the sugar portion of the nucleotide. Ribonucleotides may be modified with any of a variety of substituents.

“Ribozymes” refers to enzymatic RNA molecules capable of self-catalyzing the specific cleavage of RNA.

“Single-stranded oligonucleotide” means an oligonucleotide which is not hybridized to a complementary strand.

“Statin” means an agent that inhibits the activity of HMG-CoA reductase.

“Statin-intolerant subject” means a subject who, as a result of statin therapy, experiences one or more symptoms, such as, creatine kinase increases, liver function test abnormalities, muscle aches, or central nervous system side effects.

“Subcutaneous administration” means administration just below the skin.

“Subject” means a human or non-human animal selected for treatment or therapy.

“Target” refers to a molecule, the modulation of which is desired.

“Target gene” or “target nucleic acid” refers to a gene or nucleic acid encoding a target molecule.

“Targeting” or “targeted to” refers to the association of an antisense compound to a particular target nucleic acid molecule or a particular region of nucleobases within a target nucleic acid molecule to induce a desired effect. In certain embodiments, “targeted” means having a nucleobase sequence that will allow hybridization of an antisense compound to a target nucleic acid to induce a desired effect. In certain embodiments, a desired effect is reduction of a target nucleic acid or target molecule. In certain such embodiments, a desired effect is reduction of CREB, including CREB mRNA or CREB protein.

“Target nucleic acid,” “target RNA,” “target RNA transcript” and “nucleic acid target” all mean any nucleic acid capable of being targeted by an antisense compound.

“Target region,” refers to a portion of a target nucleic acid to which one or more antisense compounds is targeted.

“Target segment” refers to a smaller or sub-portion of a region within a target nucleic acid.

“Therapeutically effective amount” refers to an amount of an agent that provides therapeutic benefit to an animal.

“Therapeutic lifestyle change” means dietary and lifestyle changes intended to lower fat/adipose tissue and/or cholesterol. Such change can reduce the risk of developing heart disease, and may includes recommendations for dietary intake of total daily calories, total fat, saturated fat, polyunsaturated fat, monounsaturated fat, carbohydrate, protein, cholesterol, insoluble fiber, as well as recommendations for physical activity.

“Triglyceride” means a lipid or neutral fat consisting of glycerol combined with three fatty acid molecules.

“Type 2 diabetes,” (also known as “type 2 diabetes mellitus” or “diabetes mellitus, type 2”, and formerly called “diabetes mellitus type 2”, “non-insulin-dependent diabetes (NIDDM)”, “obesity related diabetes”, or “adult-onset diabetes”) is a metabolic disorder that is primarily characterized by insulin resistance, relative insulin deficiency, and hyperglycemia.

“Very low density lipoprotein-cholesterol (VLDL-C)” means cholesterol associated with very low density lipoprotein particles. Concentration of VLDL-C in serum (or plasma) is typically quantified in mg/dL or nmol/L. “Serum VLDL-C” and “plasma VLDL-C” mean VLDL-C in the serum or plasma, respectively.

### Certain Embodiments, as Described Herein

The present invention relates generally to treatment of diseases associated with lipid dysregulation. Significantly, as presented herein, treatment with CREB specific-inhibitors reduces plasma cholesterol and plasma triglycerides in vivo. This finding is bolstered by a concomitant reduction in fatty acid synthesis, increased fatty acid oxidation and changes in gene expression related to cholesterol metabolism. Additionally, white adipose tissue (WAT) weight was reduced by treatment with a CREB-specific inhibitor.

It is therefore an objective herein to treat dyslipidemia and obesity. As described herein, lipid deregulation and obesity are significant factors associated with metabolic and/or cardiovascular disease. As such, it is an objective herein to treat metabolic and cardiovascular diseases, having as a component lipid dysregulation and/or obesity, by administering a CREB-specific modulator.

As identified herein, treatment with a CREB-specific inhibitor also reduces plasma glucose levels and increases insulin sensitivity. This finding is confirmed by a concomitant reduction in gluconeogenesis as further indicated by expression levels of key gluconeogenic genes.

It is therefore also an objective herein to treat metabolic and/or cardiovascular diseases that have as a component or are characterized by combined lipid and glucose dysregulation and/or insulin resistance.

Another significant finding provided herein is improved hepatic insulin sensitivity achieved by administering a CREB-specific inhibitor. CREB-specific inhibition by a dominant negative polypeptide or CREB RNAi has previously been shown to reduce expression of insulin receptor substrate 2(IRS-2) (a key glucose regulator) and specifically decrease hepatic insulin sensitivity. As described herein, antisense oligonucleotide reduction of CREB expression results in no significant differences in IRS-2 mRNA expression in liver and significantly improves hepatic insulin responsiveness. Reducing CREB by a CREB-specific inhibitor, particularly an antisense oligonucleotide that works by an RNase H based mechanism results in the benefit of improved hepatic insulin sensitivity. Reduction of CREB also resulted in a reduction of hepatic lipids.

It is therefore an objective herein to treat hepatic insulin resistance or disease characterized by hepatic insulin resistance and/or hepatic lipid content such as NAFLD and NASH. Also, because NAFLD associated hepatic insulin resistance is a major factor contributing to hyperglycemia in Type 2 diabetes; it is a specific objective herein to treat type 2 diabetes and/or type 2 diabetes with dyslipidemia with a CREB-specific inhibitor such as, for example, an antisense oligonucleotide targeting CREB.

The CREB-specific inhibition with an antisense oligonucleotide reduced CREB mRNA and protein levels including specifically in both adipose tissue and liver tissue. Thus, antisense oligonucleotide inhibitors of CREB are useful agents for the treatment of disorders characterized by CREB expression in adipose (such as adipogenesis and obesity) and liver tissues (such as hepatic steatosis, NAFLD and NASH). Additionally, unlike other CREB-specific inhibitors, specifically small molecule inhibitors, the added benefit of using antisense compounds, for example antisense oligonucleotide inhibitors of CREB includes the ability to target both adipose and liver tissues simultaneously, both of which play key roles in metabolic disorders like obesity and diabetes.

The present invention also provides herein, methods to decrease total body adiposity. This method includes the step of administering a CREB-specific inhibitor, such as a modified antisense oligonucleotide encoding CREB, to said patient wherein CREB expression is reduced by said CREB-specific inhibitor in adipocytes of said patient, and the inhibition of CREB expression in said adipocytes is sufficient to inhibit differentiation of said adipocytes, resulting in a decrease in total body adiposity in said patient.

The present invention also provides herein, methods of modulating the levels of CREB in a cell, or tissue by contacting the cell or tissue with a CREB specific modulator. The levels can include but are not limited to CREB mRNA levels and CREB protein levels. In a certain embodiment the cell or tissue is in an animal. In certain embodiments, as described herein the animal is a human. In some aspects, CREB levels are reduced. Such reduction can occur in a time-dependent manner or in a dose-dependent manner or both.

Another aspect of the invention provides methods of treating an animal having a disease or disorder comprising administering to said animal a therapeutically effective amount of a CREB-specific modulator, including, more specifically, wherein the CREB specific modulator is a CREB-specific inhibitor, as described herein. In various aspects, the disease or disorder is a cardiovascular and/or metabolic disease or disorder, as described herein. In particular embodiments, the disease or disorder is characterized by dyslipidemia, including, more specifically hyperlipidemia, including, even more specifically hypercholesterolemia and/or hypertriglyceridemia, as described herein. In particular embodiments, the disease or disorder is atherosclerosis. In another embodiment, the disease or disorder is diabetes, including, more specifically Type 2 diabetes, as described herein. In a further embodiment, the disease or disorder is obesity, as described herein. As many of these diseases and disorders are interrelated and as the CREB-specific modulators demonstrate therapeutic benefit with regard to such diseases and disorders, it is also an object herein, to treat a combination of the above diseases and disorder by administering a CREB-specific modulator, as described herein.

In some embodiments, the CREB-specific modulator is a CREB-specific inhibitor, as described herein, which reduces lipid accumulation or lipid levels, as described herein. The lipid levels can be cholesterol levels or triglyceride levels or both. In a particular embodiment such inhibitor is useful for treating dyslipidemia or conditions characterized by dyslipidema, as described herein, such as cardiovascular diseases, such as atherosclerosis and coronary heart disease, obesity, lipoma, non-alcoholic fatty liver disease (NAFLD), hyperfattyacidemia, as described herein. The reduction in lipid levels can be in combination with a reduction in glucose levels and/or insulin resistance, as described herein. In a particular embodiment, such inhibitor is useful for treating conditions characterized by both dyslipidemia and glucose dysregulation such as metabolic disorder including diabetes and metabolic syndrome, as described herein.

In other embodiments, the CREB-specific inhibitor reduces adiposity, lipogenesis, lipogenic genes, adipose tissue mass, body weight and/or body fat, as described herein. In a particular embodiment such inhibitor is useful for treating obesity and/or obesity related diseases and disorders. Such reduction can be in combination with a reduction in glucose levels and/or insulin resistance. In a particular embodiment, such inhibitor is useful for treating metabolic syndrome and other disorders associated with diabesity, as described herein.

In a particular embodiment, the CREB-specific inhibitor reduces lipid levels, adipose tissue mass or weight, and glucose levels. In a particular embodiment such inhibitor is useful for treating any number of cardiovascular, metabolic and obesity related diseases and disorders as further provided herein.

In some embodiments, are methods of identifying or selecting a subject having dyslipidemia and administering to the subject a CREB-specific modulator, as described herein.

In other embodiments, are methods of identifying or selecting a subject having obesity or a condition of localized increase in adipogenesis and administering to the subject a CREB-specific inhibitor, as described herein.

In another embodiment provided herein, are methods of identifying or selecting a subject having or at risk of having a cardiovascular disorder and administering to the subject a CREB-specific modulator.

In particular embodiments are methods of identifying or selecting a subject having a metabolic disease characterized by dyslipidemia or a change in fat accumulation and administering to the subject a CREB-specific modulator.

In another embodiment provided herein, are methods of identifying or selecting a subject having elevated cholesterol levels and administering to the subject a CREB-specific inhibitor, thereby reducing cholesterol levels.

In a particular embodiment provided herein, are methods of identifying or selecting a subject having elevated triglyceride levels and administering to the subject a CREB-specific inhibitor, thereby reducing triglyceride levels.

In another embodiment provided herein, are methods of identifying or selecting a subject having reduced hepatic insulin sensitivity and administering to the subject a CREB-specific inhibitor, thereby improving hepatic insulin sensitivity.

Further provided herein are methods for treating or preventing metabolic or cardiovascular disorder in a subject comprising selecting a subject having elevated lipid levels, increased fat accumulation, reduced hepatic insulin sensitivity or a combination thereof; and administering to the subject a CREB-specific modulator.

The invention also provides methods of preventing or delaying the onset of or reducing the risk-factors for a cardiovascular-related or metabolic-related disease or disorder in an animal comprising administering a therapeutically or prophylactically effective amount of a CREB-specific modulator. In one aspect, the animal is a human. In other aspects, the metabolic and cardiovascular-related disease or disorder includes, but is not limited to obesity, lipoma, lipomatosis, diabetes (including Type 1 diabetes, Type 2 diabetes and Type 2 diabetes with dyslipidemia), dyslipidemia (including hyperlipidemia, hypertriglyceridemia, and mixed dyslipidemia), non-alcoholic fatty liver disease (NAFLD) (including hepatic steatosis and steatohepatitis), hyperfattyacidemia, metabolic syndrome, hyperglycemia, insulin resistance, hypercholesterolemia (including polygenic hypercholesterolemia), coronary heart disease (early onset coronary heart disease), elevated ApoB, or elevated cholesterol (including elevated LDL-cholesterol, elevated VLDL-cholesterol, elevated IDL-cholesterol, and elevated non-HDL cholesterol).

Methods of administration of the CREB-specific modulators of the invention to a subject are intravenously, subcutaneously, or orally. Administrations can be repeated.

In a further embodiment, the CREB-specific modulator is a CREB-specific inhibitor, for example an antisense compound targeted to a nucleic acid encoding CREB to inhibit CREB mRNA levels or protein expression.

In a further embodiment, the CREB-specific antisense compound is selected from: an oligonucleotide and antisense oligonucleotide, a ssRNA, a dsRNA, a ribozyme, a triple helix molecule and an siRNA or other RNAi compounds.

In some embodiments, a CREB-specific modulator can be co-administered with at least one or more additional therapies, as described herein. In related embodiments, the CREB-specific modulator and additional therapy are administered concomitantly. In a separate embodiment, the CREB-specific modulator and additional therapy are administered in a single formulation. In some embodiments, the CREB-specific modulator is administered in combination with one or more of a non-specific modulator of CREB or additional therapy that does not modulate CREB activity. In some embodiments, the CREB-specific modulator is administered in combination with a cholesterol-lowering agent and/or glucose-lowering agent and/or a lipid-lowering agent and/or a anti-obesity agent.

The present invention also provides a CREB-specific modulator as described herein for use in treating or preventing a cardiovascular and/or metabolic disease or disorder as described herein. For example, the invention provides a CREB-specific modulator as described herein for use in treating or preventing dyslipidemia, atherosclerosis, coronary heart disease, hyperfattyacidemia, or hyperlipoprotenemia, obesity, lipoma, diabetes, atherosclerosis, coronary heart disease, type 2 diabetes, non-alcoholic fatty liver disease (NAFLD), hyperfattyacidemia, metabolic syndrome.

The present invention also provides the use of a CREB-specific modulator as described herein in the manufacture of a medicament for treating or preventing a cardiovascular and/or metabolic disease or disorder as described herein. For example, the invention provides the use of a CREB-specific modulator as described herein in the manufacture of a medicament for treating or preventing dyslipidemia, atherosclerosis, coronary heart disease, hyperfattyacidemia, or hyperlipoprotenemia, obesity, lipoma, diabetes, atherosclerosis, coronary heart disease, type 2 diabetes, non-alcoholic fatty liver disease (NAFLD), hyperfattyacidemia, metabolic syndrome.

The invention also provides a CREB-specific modulator as described herein for reducing serum lipid levels, e.g. for reducing serum lipid levels in a subject having elevated serum lipid levels. The present invention also provides the use of a CREB-specific modulator as described herein in the manufacture of a medicament for reducing serum lipid levels, e.g. for reducing serum lipid levels in a subject having elevated serum lipid levels.

The invention also provides a CREB-specific modulator as described herein for reducing cholesterol levels, e.g. for reducing cholesterol levels in a subject having elevated cholesterol levels. The present invention also provides the use of a CREB-specific modulator as described herein in the manufacture of a medicament for reducing cholesterol levels, e.g. for reducing cholesterol levels in a subject having elevated cholesterol levels.

The invention also provides a CREB-specific modulator as described herein for reducing triglyceride levels, e.g. for reducing triglyceride levels in a subject having elevated triglyceride levels. The present invention also provides the use of a CREB-specific modulator as described herein in the manufacture of a medicament for reducing triglyceride levels, e.g. for reducing triglyceride levels in a subject having elevated triglyceride levels.

The invention also provides a CREB-specific modulator as described herein for improving hepatic insulin sensitivity, e.g. for improving hepatic insulin sensitivity in a subject having reduced hepatic insulin sensitivity. The present invention also provides the use of a CREB-specific modulator as described herein in the manufacture of a medicament for improving hepatic insulin sensitivity, e.g. for improving hepatic insulin sensitivity in a subject having reduced hepatic insulin sensitivity.

The invention also provides a CREB-specific modulator as described herein for reducing adipogenesis, e.g. for reducing adipogenesis in a subject having elevated adipose tissue mass or weight. The present invention also provides the use of a CREB-specific modulator as described herein in the manufacture of a medicament for reducing adipogenesis, e.g. for reducing adipogenesis in a subject having elevated adipose tissue mass or weight.

The invention also provides a CREB-specific modulator as described herein for treating diabetes, e.g. for treating diabetes in a subject having type 2 diabetes with dyslipidemia. The present invention also provides the use of a CREB-specific modulator as described herein in the manufacture of a medicament for treating diabetes, e.g. for treating diabetes in a subject having type 2 diabetes with dyslipidemia.

The invention also provides a CREB-specific modulator as described herein for treating metabolic syndrome, e.g. for treating metabolic syndrome in a subject having metabolic syndrome or one or more risk factors of metabolic syndrome. The present invention also provides the use of a CREB-specific modulator as described herein in the manufacture of a medicament for treating metabolic syndrome, e.g. for treating metabolic syndrome in a subject having metabolic syndrome or one or more risk factors of metabolic syndrome.

The invention also provides a CREB-specific modulator as described herein for use in treating or preventing a cardiovascular and/or metabolic disease or disorder as described herein by combination therapy with an additional therapy as described herein.

The invention also provides a pharmaceutical composition comprising a CREB-specific modulator as described herein in combination with an additional therapy as described herein. The invention also provides the use of a CREB-specific modulator as described herein in the manufacture of a medicament for treating or preventing a cardiovascular and/or metabolic disease or disorder as described herein by combination therapy with an additional therapy as described herein.

The invention also provides the use of a CREB-specific modulator as described herein in the manufacture of a medicament for treating or preventing a cardiovascular and/or metabolic disease or disorder as described herein in a patient who has previously been administered an additional therapy as described herein.

The invention also provides the use of a CREB-specific modulator as described herein in the manufacture of a medicament for treating or preventing a cardiovascular and/or metabolic disease or disorder as described herein in a patient who is subsequently to be administered an additional therapy as described herein.

The invention also provides a kit for treating or preventing a cardiovascular and/or metabolic disease or disorder said kit comprising:

(i) a CREB-specific modulator as described herein; and optionally
(ii) an additional therapy as described herein.

A kit of the invention may further include instructions for using the kit to treat or prevent a cardiovascular and/or metabolic disease or disorder as described herein by combination therapy as described herein.

The antisense compounds targeting CREB may have the nucleobase sequence of any of SEQ ID NOs: 13-187.

In another embodiment, the method comprises identifying or selecting an animal having a metabolic or cardiovascular disease and administering to the animal having a metabolic or cardiovascular disease a therapeutically effective amount of a compound comprising a modified oligonucleotide consisting of 12 to 30 linked nucleosides, wherein the modified oligonucleotide is complementary to human CREB.

In one such embodiment, the metabolic or cardiovascular disease is obesity, diabetes, atherosclerosis, coronary heart disease, non-alcoholic fatty liver disease (NAFLD), hyperfattyacidemia or metabolic syndrome, or a combination thereof.

In another such embodiment, the administering results in a reduction of lipid levels, including triglyceride levels, cholesterol levels; insulin resistance; body fat, body weight, adipose tissue mass, glucose levels or a combination thereof.

In another such embodiment, the disease wherein the levels are reduced by 5%, 10%, 20%, 30%, 35%, or 40%.

In one such embodiment, the disease is dyslipidemia.

In another such embodiment, the disease of dyslipidemia is hyperlipidemia.

In yet another such embodiment, the hyperlipidemia is hypercholesterolemia, hypertriglyceridemia, or both hypercholesterolemia and hypertriglyceridemia.

In one such embodiment, the disease of NAFLD is hepatic steatosis or steatohepatitis.

In another such embodiment, the disease the diabetes is type 2 diabetes or type 2 diabetes with dyslipidemia.

In one such embodiment, the method results in a reduction of triglyceride levels of at least 20, 30, 35, or 40%.

In another embodiment, the method results in a reduction of cholesterol levels.

In one such embodiment, the method results in a reduction of cholesterol levels by at least 10, 20, 30, 35 or 40%.

In another embodiment, the method results in a reduction of glucose levels.

In one such embodiment, the method results in a reduction of glucose levels by at least 5 or 10%.

In another embodiment, the method results in a reduction of body weight.

In one such embodiment, the method results in a reduction of body weight by at least 10 or 15%.

In another embodiment, the method results in a reduction of body fat.

In one such embodiment, the method results in a reduction of body fat by at least 10, 20, 30, or 40%.

In another embodiment, the method results in a reduction of triglyceride levels, cholesterol levels, glucose levels, body weight, fat content, insulin resistance, or any combination thereof, wherein levels are independently reduced by 5%, 10%, or 15%.

In another embodiment, the method comprises identifying or selecting an obese animal and administering to the obese animal a therapeutically effective amount of a CREB inhibitor.

In one such embodiment, the method results in a reduction of body fat.

In another such embodiment, the method results in a reduction of body fat by at least 10, 20, 30, or 40%.

In another embodiment, the method comprises identifying or selecting a diabetic animal and administering to the diabetic animal a therapeutically effective amount of a CREB inhibitor.

In one such embodiment, the method results in a reduction of glucose levels.

In another such embodiment, the method results in a reduction of glucose level by at least 10 or 15%.

### Certain Indications

In certain embodiments, the invention provides methods of treating an individual comprising administering one or more pharmaceutical compositions of the present invention. In certain embodiments, the individual has a metabolic disorder or cardiovascular disorder. In certain embodiments, the disorder is dyslipidemia, atherosclerosis, coronary heart disease, hyperfattyacidemia, or hyperlipoprotenemia, obesity, lipoma, diabetes, atherosclerosis, coronary heart disease, type 2 diabetes, non-alcoholic fatty liver disease (NAFLD), hyperfattyacidemia, or metabolic syndrome.

In one embodiment, administration of a therapeutically effective amount of an antisense compound targeted to a CREB nucleic acid is accompanied by monitoring plasma glucose, plasma triglycerides, and plasma cholesterol levels in the serum of an individual, to determine an individual's response to administration of the antisense compound. In another embodiment, body weight is monitored. An individual's response to administration of the antisense compound is used by a physician to determine the amount and duration of therapeutic intervention.

In one embodiment, administration of an antisense compound targeted to a CREB nucleic acid results in reduction of CREB expression by at least 15, 20, 25, 30, 35, 40, 45, 50, 55, 60, 65, 70, 75, 80, 85, 90, 95 or 99%, or a range defined by any two of these values. In one embodiment, administration of an antisense compound targeted to a CREB nucleic acid results in a change in plasma glucose, plasma triglycerides, plasma cholesterol, and/or body weight. In some embodiments, administration of a CREB antisense compound decreases plasma glucose, plasma triglycerides, plasma cholesterol, and/or body weight by at least 15, 20, 25, 30, 35, 40, 45, or 50%, or a range defined by any two of these values.

In certain embodiments, as described herein a pharmaceutical composition comprising an antisense compound targeted to CREB is used for the preparation of a medicament for treating a patient suffering or susceptible to a metabolic disorder.

### Metabolic Disorders

Conditions associated with and included in metabolic disorders encompass, but are not limited to obesity, lipoma, lipomatosis, diabetes (including Type 1 diabetes, Type 2 diabetes and Type 2 diabetes with dyslipidemia), dyslipidemia (including hyperlipidemia, hypertriglyceridemia, and mixed dyslipidemia), non-alcoholic fatty liver disease (NAFLD) (including hepatic steatosis and steatohepatitis), hyperfattyacidemia, metabolic syndrome, hyperglycemia, and insulin resistance.

Blood sugar regulation is the process by which the levels of blood sugar, primarily glucose, are maintained by the body. Blood sugar levels are regulated by negative feedback in order to keep the body in homeostasis. If the blood glucose level falls glucagon is released. Glucagon is a hormone whose effects on liver cells act to increase blood glucose levels. They convert glycogen storage into glucose, through a process is called glycogenolysis. The glucose is released into the bloodstream, increasing blood sugar levels. When levels of blood sugar rise, whether as a result of glycogen conversion, or from digestion of a meal, insulin is released, and causes the liver to convert more glucose into glycogen (glycogenesis), and forces about ⅔ of body cells to take up glucose from the blood, thus decreasing blood sugar levels. Insulin also provides signals to several other body systems, and is the chief regulatory metabolic control in humans.

Diabetes mellitus type 2 is caused by insulin resistance which, if untreated, results in hyperglycemia. Insulin resistance is the condition in which normal amounts of insulin are inadequate to produce a normal insulin response from fat, muscle and liver cells. Insulin resistance in fat cells reduces the effects of insulin and results in elevated hydrolysis of stored triglycerides in the absence of measures which either increase insulin sensitivity or which provide additional insulin. Increased mobilization of stored lipids in these cells elevates free fatty acids in the blood plasma. Insulin resistance in muscle cells reduces glucose uptake and causes local storage of glucose as glycogen, whereas insulin resistance in liver cells reduces storage of glycogen, making it unavailable for release into the blood when blood insulin levels fall. High plasma levels of insulin and glucose due to insulin resistance often lead to metabolic syndrome and type 2 diabetes, and other related complications.

CREB-specific inhibitors are shown herein to increase insulin sensitivity, reduce glucose and generally improve diabetic state indicated, for, example, by a reduction in ketogenesis and plasma free fatty acids. These studies support the use of CREB-specific inhibitors for the treatment of diabetes, metabolic syndrome and other disorders characterized by glucose deregulation.

Nonalcoholic fatty liver disease (NAFLD) is strongly associated with hepatic insulin resistance in patients with poorly controlled type 2 diabetes mellitus (OB/OB) (Petersen, K. F., et al., 2005, Diabetes 54:603-608; Petersen, K. F., et al. 2002, J Clin Invest 109:1345-1350; Yki-Jarvinen, H., Helve, E., et al., 1989. Am J Physiol 256:E732-739). Modest weight reduction in these subjects has been described to reduce hepatic steatosis and normalize fasting plasma glucose concentrations by decreasing hepatic gluconeogenesis and improving hepatic insulin sensitivity (Petersen, K. F., et al., 2005, Diabetes 54:603-608). Furthermore, preventing hepatic steatosis in high-fat fed rodents by either increasing mitochondrial oxidation by knockdown of acetyl-CoA carboxylase (ACC) (Savage, D. B., et al. 2006. J Clin Invest 116:817-824.), or decreasing lipid synthesis by decreasing the expression of key lipogenic enzymes mitochondrial acyl-CoA glycerol-sn-3-phosphate transferase 1 (mtGPAT) (Neschen, S., et al. 2005, Cell Metab 2:55-65), stearoyl-CoA desaturase-1 (SCD1) (Gutierrez-Juarez, R., et al., 2006. J Clin Invest 116:1686-1695) or diacylglycerol acyltransferase-2 (DGAT2) (Choi, C. S., et al. 2007. J Biol Chem 282:22678-22688) has been described to prevent hepatic insulin resistance.

As seen in FIGS. 4A, 4B, and 4C, intrahepatic lipids (triglycerides, diacylglycerols and long chain CoAs) were lowered with CREB antisense oligonucleotide treatment respectively over that in the control antisense oligonucleotide treated Type 2 Diabetic rats, indicating that inhibition of CREB expression could have therapeutic benefit in subjects having disorders or conditions characterized by elevated liver triglycerides, diacylglycerols and long chain CoAs including metabolic disorders such as NAFLD and NASH. Because NAFLD is strongly associated with hepatic insulin resistance and increased glucose, the ability of CREB inhibitors to reduce glucose concentrations and improve hepatic insulin sensitivity provide additional therapeutic benefits. These studies also support the use of CREB inhibitors for more general metabolic disorders including Type 2 diabetes and obesity because insulin resistance, which is also associated with NAFLD, is a major factor contributing to hyperglycemia in Type 2 diabetes.

Metabolic syndrome is the clustering of lipid and non-lipid cardiovascular risk factors of metabolic origin. It has been closely linked to the generalized metabolic disorder known as insulin resistance. The National Cholesterol Education Program (NCEP) Adult Treatment Panel III (ATPIII) established criteria for diagnosis of metabolic syndrome when three or more of five risk determinants are present. The five risk determinants are abdominal obesity defined as waist circumference of greater than 102 cm for men or greater than 88 cm for women, triglyceride levels greater than or equal to 150 mg/dL, HDL cholesterol levels of less than 40 mg/dL for men and less than 50 mg/dL for women, blood pressure greater than or equal to 130/85 mm Hg and fasting glucose levels greater than or equal to 110 mg/dL. These determinants can be readily measured in clinical practice (JAMA, 2001, 285: 2486-2497).

The World Health Organization definition of metabolic syndrome is diabetes, impaired fasting glucose, impaired glucose tolerance, or insulin resistance (assessed by clamp studies) and at least two of the following criteria: waist-to-hip ratio greater than 0.90 in men or greater than 0.85 in women, triglycerides greater than or equal to 1.7 mmol/l or HDL cholesterol less than 0.9 mmol in men and less then 1.0 mmol in women, blood pressure greater than or equal to 140/90 mmHg, urinary albumin excretion rate greater than 20 .mu.g/min or albumin-to-creatinine ratio greater than or equal to 30 mg/g (Diabetes Care, 2005, 28(9): 2289-2304).

A statement from the American Diabetes Association and the European Association for the Study of Diabetes comments on the construct of metabolic syndrome to denote risk factor clustering. In addition to suggestions for research of the underlying pathophysiology, the recommendations include separately and aggressively treating all cardiovascular disease risk factors (Diabetes Care, 2005, 28(9): 2289-2304).

Significantly, it has been described herein that an increase of insulin sensitivity and decrease in glucose levels as well as a decrease in lipid levels can be achieved through the modulation of CREB expression. Therefore, another embodiment is a method of treating metabolic and cardiovascular disease or disorders or risks thereof, with CREB-specific modulating agents.

Unlike the understanding of the mechanism of fat-induced hepatic insulin resistance, the pathogenesis of increased hepatic gluconeogenesis in ob/ob is less well understood. Gluconeogenesis is a metabolic pathway that results in the generation of glucose from non-carbohydrate carbon substrates such as pyruvate, lactate, glycerol, and glucogenic amino acids. The vast majority of gluconeogenesis takes place in the liver and, to a smaller extent, in the cortex of kidneys, and is triggered by the action of insulin. Gluconeogenesis is a target of therapy for metabolic disorders such as hyperglycemia and type 2 diabetes.

Progressive declines in insulin secretion as well as inappropriately increased glucagon secretion have both been viewed as critical factors responsible for increased rates of hepatic gluconeogenesis (Unger, R. H., et al., 1977. Annu Rev Med 28:119-130; Reaven, G. M., et al., 1987, J Clin Endocrinol Metab 64:106-110; Del Prato, S., et al., 2004, Horm Metab Res 36:775-781; Cherrington, A. D., et al., 1987. Diabetes Metab Rev 3:307-332). Glucagon regulates hepatic glucose metabolism by binding to its receptor, a heterotrimeric G protein. This results in activation of adenylate cylase leading to increased intracellular cAMP production (Jelinek, L. J., Lok, et al. 1993, Science 259:1614-1616). Consequently cAMP activates cAMP-dependent protein kinase resulting in the phosphorylation of Ser133 on cAMP response element-binding protein (CREB) and subsequent translocation to the nucleus (Gonzalez, G. A., et al., 1989, Cell 59:675-680). CREB is a leucine BH/zipper transcription factor that promotes gene transcription by binding to conserved sequences known as a cAMP responsive element (CRE) (Mayr, B., et al., 2001, Nat Rev Mol Cell Biol 2:599-609). CREB is a well known activator of gluconeogenic gene transcription through CRE elements located on key gluconeogenic genes. Insulin antagonizes the induction of gluconeogenic enzymes by phosphorylating CREB-binding protein (CBP) (Zhou, X. Y., et al., 2004, Nat Med 10:633-637) and transducer of regulated CREB activity 2 (TORC2). Phosphorylation of CBP prevents complex formation with CREB to activate transcription. Phosphorylation of TORC2 results in nuclear exclusion and subsequent ubiquitin-dependent degradation (Dentin, R., et al., 2007, Nature 449:366-369). Thus, CREB plays a role in the regulation of hepatic gluconeogenesis.

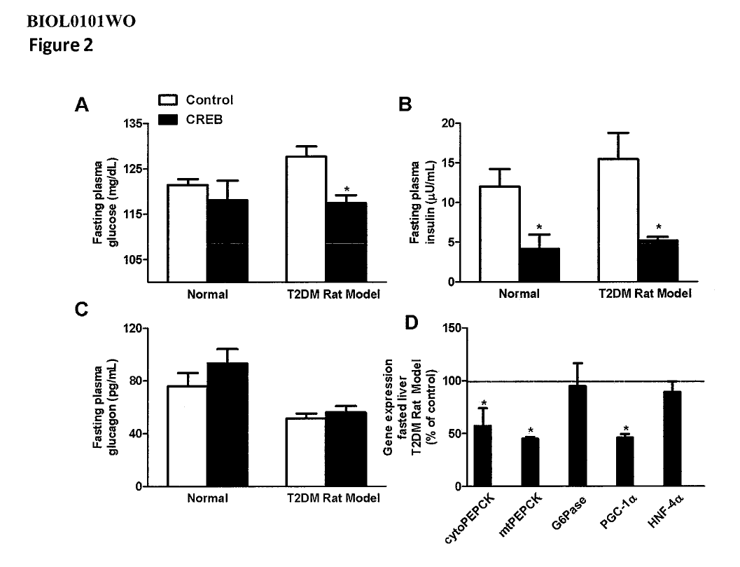

As described herein, treatment with the CREB antisense oligonucleotide in the T2DM models showed a reduction in gluconeogenesis, through a reduction in gluconeogenic mRNA expression. Gluconeogenesis is a major factor contributing to hyperglycemia in subjects with Type 2 diabetes. These results further indicate that inhibition of CREB expression could have therapeutic benefit in subjects having metabolic disorders, such as Type 2 diabetes. The decreased expression of CREB mRNA led to decreased expression of the key gluconeogenic enzyme cytosolic phosphoenolpyruvate carboxykinase (PEPCK), which may partly explain the mechanism of the improved hepatic insulin sensitivity observed in the CREB antisense oligonucleotide treated rats. The gluconeogenic enzymes, cytosolic phosphoenolpyruvate carboxykinase (PEPCK), mitochondrial PEPCK and the transcriptional co-activator peroxisomal proliferator activated receptor gamma coactivator-1-alpha (PGC-1β) mRNA levels were decreased by 43%, 55% and 54% respectively in the liver of the CREB antisense oligonucleotide groups (FIG. 2D).

CREB-specific inhibitors are shown herein to reduce hepatic gluconeogenesis. Accordingly, for therapeutics, a subject, preferably an animal, even more preferably a human, suspected of having a metabolic disorder associated with gluconeogenesis which can be treated by modulating the expression of CREB. A subject is treated by administering a CREB-specific modulator, preferably a CREB-specific inhibitor, an antisense compounds targeting CREB.

The administration of CREB-specific modulators herein, include, but are not limited to proteins, peptides, polypeptides, antibodies, antisense compounds including oligonucleotides and antisense oligonucleotides, ssRNA, dsRNA molecules, ribozymes, triple helix molecules, siRNA and other RNAi compounds, and small molecule modulators. The antisense compounds included herein, can operate by an RNaseH or RNAi mechanism or by other known mechanism such as splicing.

Further described herein, are CREB-specific inhibitors, for example antisense compounds targeting CREB that reduce CREB mRNA and protein.

Also described herein, are CREB-specific inhibitors, for example antisense compounds targeting CREB that reduce liver CREB mRNA.

Also described herein, are CREB-specific inhibitors, for example antisense compounds targeting CREB that reduce white adipose tissue (WAT) CREB mRNA. Included herein, are examples of antisense compounds targeting CREB and methods of their use prophylactically, for example, to prevent or delay the progression or development of metabolic disorders such as diabetes or elevated blood glucose levels.

Also included herein, are methods for treating or preventing a metabolic disorder, in a subject, comprising administering one or more CREB-specific modulators. In certain embodiments, the subject has metabolic disorders or conditions including, but not limited obesity, lipoma, lipomatosis, diabetes (including Type 1 diabetes, Type 2 diabetes and Type 2 diabetes with dyslipidemia), dyslipidemia (including hyperlipidemia, hypertriglyceridemia, and mixed dyslipidemia), non-alcoholic fatty liver disease (NAFLD) (including hepatic steatosis and steatohepatitis), hyperfattyacidemia, metabolic syndrome, hyperglycemia, and insulin resistance.

In one embodiment are methods for decreasing blood glucose levels and/or increasing insulin sensitivity, or alternatively methods for treating type 2 diabetes or metabolic syndrome, by administering to a subject suffering from elevated glucose levels or insulin resistance a therapeutically effective amount of a CREB-specific modulator. In another embodiment, a method of decreasing blood glucose levels and/or increasing insulin sensitivity comprises selecting a subject in need of a decrease in blood glucose or increase in insulin sensitivity, and administering to the subject a therapeutically effective amount of a CREB-specific modulator. In a further embodiment, a method of reducing risk of development of type 2 diabetes and metabolic syndrome includes selecting a subject having elevated blood glucose levels or reduced insulin sensitivity and one or more additional indicators risk of development of type 2 diabetes or metabolic syndrome, and administering to the subject a therapeutically effective amount of a CREB-specific modulator, for example a antisense compound.

In one embodiment, administration of a therapeutically effective amount of a CREB-specific modulator targeted a CREB nucleic acid is accompanied by monitoring of glucose levels in the serum of a subject, to determine a subject's response to administration of the CREB-specific modulator. A subject's response to administration of the CREB-specific modulator is used by a physician to determine the amount and duration of therapeutic intervention.

In one embodiment, administration of a therapeutically effective amount of an antisense compound targeted a CREB nucleic acid is accompanied by monitoring of glucose levels in the serum or insulin sensitivity of a subject, to determine a subject's response to administration of the antisense compound. A subject's response to administration of the antisense compound is used by a physician to determine the amount and duration of therapeutic intervention.

Further described herein, are antisense compounds targeting CREB that reduce diet induced obesity in animals. Thus, antisense compounds targeting CREB are useful in treating, preventing or delaying obesity. These results are consistent with previous studies demonstrating that CREB promotes adipocyte differentiation (Zhang, J. W., Klemm, D. J., Vinson, C., and Lane, M. D. 2004, J Biol Chem 279:4471-4478) and the observations that glucagon receptor knockout mice are leaner than wild type control mice (Gelling, R. W., Du, X. Q., Dichmann, D. S., Romer, J., Huang, H., Cui, L., Obici, S., Tang, B., Holst, J. J., Fledelius, C., et al. 2003, Proc Natl Acad Sci USA 100:1438-1443).

Further described herein, are antisense compounds targeting CREB that reduce white adipose tissue mass or weight in Type 2 diabetic animals.

Further described herein, are antisense compounds targeting CREB that reduce fasting plasma leptin concentrations in Type 2 diabetic animals.

Further described herein, are antisense compounds targeting that reduce plasma insulin in Type 2 diabetic animals

Further described herein, are antisense compounds targeting CREB that reduce plasma glucose in Type 2 diabetic animals.

Further described herein, are antisense compounds targeting CREB that improve insulin sensitivity.

Further described herein, are antisense compounds targeting CREB that reduce fasting plasma insulin concentrations.

Further described herein, are antisense compounds targeting CREB that reduce fasting plasma glucose concentrations in Type 2 diabetic animals.

Further described herein, are antisense compounds targeting CREB that reduce the gluconeogenic enzymes, for example, cytosolic phosphoenolpyruvate carboxykinase (PEPCK), mitochondrial PEPCK, and the transcriptional co-activator peroxisomal proliferator activated receptor gamma coactivator-1 alpha (PGC-1α) mRNA. Because antisense compounds targeting CREB are described herein, to increase insulin sensitivity in normal animals fed a high-fat diet, and to reduce weight gain of these animals, antisense compounds targeting CREB is useful in treating, preventing or delaying insulin resistance and weight gain and are therefore useful for the treatment of metabolic disorders such as Type 2 diabetes and obesity.

A physician may determine the need for therapeutic intervention for subjects in cases where more or less aggressive blood glucose or triglyceride-lowering therapy is needed. The practice of the methods herein may be applied to any altered guidelines provided by the NCEP, or other entities that establish guidelines for physicians used in treating any of the diseases or conditions listed herein, for determining coronary heart disease risk and diagnosing metabolic syndrome.

Various CREB-specific modulators targeting CREB, such as antisense compounds, can be utilized in pharmaceutical compositions by adding an effective amount of a compound to a suitable pharmaceutically acceptable diluent or carrier. Use of the compounds and methods of the invention may also be useful prophylactically to prevent such diseases or disorders, e.g., to prevent or delay undue weight gain, diabetes, other metabolic disorders, or cardiovascular disorders.

### Cardiovascular Disorders

Conditions associated with risk of developing a cardiovascular disorders include, but are not limited to: history of myocardial infarction, unstable angina, stable angina, coronary artery procedures (angioplasty or bypass surgery), evidence of clinically significant myocardial ischemia, noncoronary forms of atherosclerotic disease (peripheral arterial disease, abdominal aortic aneurysm, carotid artery disease), diabetes, cigarette smoking, hypertension, low HDL cholesterol, family history of premature coronary heart disease, obesity, physical inactivity, elevated triglyceride (hypertriglyceridemia), hypercholesterolemia (including polygenic hypercholesterolemia), coronary heart disease (early onset coronary heart disease), elevated ApoB, or elevated cholesterol (including elevated LDL-cholesterol, elevated VLDL-cholesterol, elevated IDL-cholesterol, and elevated non-HDL cholesterol). (Jama, 2001, 285, 2486-2497; Grundy et al., Circulation, 2004, 110, 227-239).

Hypertriglyceridemia (or “hypertriglyceridaemia”) denotes high blood levels of triglycerides. A triglyceride is glyceride in which the glycerol is esterified with three fatty acids. Elevated triglyceride levels have been associated with atherosclerosis, even in the absence of hypercholesterolemia (high cholesterol levels). It can also lead to pancreatitis in excessive concentrations. A related term is “hyperglyceridemia” or “hyperglyceridaemia”, which refers to a high level of all glycerides, including monoglycerides, diglycerides and triglycerides

Triglycerides, as major components of very low density lipoprotein (VLDL) and chylomicrons, play an important role in metabolism as energy sources and transporters of dietary fat. Fat and liver cells can synthesize and store triglycerides. When the body requires fatty acids as an energy source, the hormone glucagon signals the breakdown of the triglycerides by hormone-sensitive lipase to release free fatty acids. The glycerol component of triglycerides can be converted into glucose, via gluconeogenesis, for brain fuel when it is broken down.

Further described herein, are antisense compounds targeting CREB that reduce plasma triglycerides in Type 2 diabetic animals. The studies show a significant reduction in plasma triglyceride levels after treatment with the CREB antisense oligonucleotide. These studies indicate that inhibition of CREB expression can provide therapeutic benefit in subjects having metabolic disorders like obesity and Type 2 Diabetes, with the added benefit of preventing or reducing associated dyslipidemia that can also lead to the risk of cardiovascular disorders characterized by hypercholesterolemia and hypertriglyceridemia. Thus, antisense inhibitors of CREB could be candidate therapeutic agents for the treatment of conditions characterized by hypercholesterolemia, and hypertriglyceridemia, or conditions of dyslipidemia associated with NAFLD, Type 2 diabetes, obesity and other metabolic disorders.

Hypercholesterolemia (elevated blood cholesterol) is the presence of high levels of cholesterol in the blood. It is not a disease but a metabolic derangement that can be secondary to many diseases and can contribute to many forms of disease, most notably cardiovascular disease. It is closely related to “hyperlipidemia” (elevated levels of lipids) and “hyperlipoproteinemia” (elevated levels of lipoproteins). Conditions with elevated concentrations of oxidized LDL particles, especially “small dense LDL” (sdLDL) particles, are associated with atheroma formation in the walls of arteries, a condition known as atherosclerosis, which is the principal cause of coronary heart disease and other forms of cardiovascular disease. In contrast, HDL particles (especially large HDL) have been identified as a mechanism by which cholesterol and inflammatory mediators can be removed from atheroma. Increased concentrations of HDL correlate with lower rates of atheroma progressions and even regression.

Elevated levels of the lipoprotein fractions, LDL, IDL and VLDL are regarded as atherogenic (prone to cause atherosclerosis). Levels of these fractions correlate with the extent and progress of atherosclerosis. Conversely, the cholesterol can be within normal limits, yet be made up primarily of small LDL and small HDL particles, under which conditions atheroma growth rates would still be high. In contrast, however, if LDL particle number is low (mostly large particles) and a large percentage of the HDL particles are large, then atheroma growth rates are usually low, even negative, for any given cholesterol concentration.

Further described herein is a significant reduction in plasma cholesterol levels after treatment with the CREB antisense oligonucleotide. These studies indicate that inhibition of CREB expression can provide therapeutic benefit in subjects having metabolic disorders like obesity and Type 2 Diabetes, with the added benefit of preventing or reducing associated dyslipidemia that can also lead to the risk of cardiovascular disorders characterized by hypercholesterolemia and hypertriglyceridemia. Thus, antisense inhibitors of CREB could be candidate therapeutic agents for the treatment of conditions characterized by hypercholesterolemia, and hypertriglyceridemia, or conditions of dyslipidemia associated with NAFLD, Type 2 diabetes, obesity and other metabolic disorders.

Elevated blood glucose levels, elevated triglyceride levels or elevated cholesterol levels are considered a risk factor in the development and progression of atherosclerosis. Atherosclerosis is a disease affecting arterial blood vessels. It is a chronic inflammatory response in the walls of arteries, in large part due to the accumulation of macrophage white blood cells and promoted by low density (especially small particle) lipoproteins (plasma proteins that carry cholesterol and triglycerides) without adequate removal of fats and cholesterol from the macrophages by functional high density lipoproteins (HDL), (see apoA-1 Milano). It is commonly referred to as a “hardening” or “furring” of the arteries. It is caused by the formation of multiple plaques within the arteries. Atherosclerosis can lead to coronary heart disease, stroke, peripheral vascular disease, or other cardiovascular-related disorders.

Further described herein, are studies that show CREB-specific modulators, like antisense compounds, reducing blood glucose levels, elevated triglyceride levels and elevated cholesterol levels. Thus CREB-specific antisense oligonucleotides could be candidate therapeutic agents for the treatment of conditions characterized by the progression of atherosclerosis.

CREB-specific inhibitors are shown herein to reduce lipid levels. Accordingly, for therapeutics, a subject, preferably an animal, even more preferably a human, suspected of having a cardiovascular disorder associated with elevated lipid levels can be treated by modulating the expression of CREB. A subject is treated by administering a CREB-specific modulator, preferably a CREB-specific inhibitor, for example an antisense compounds targeting CREB.

A further embodiment is a method of treating cardiovascular disorders wherein, the CREB-specific modulator is a CREB-specific inhibitor, for example a CREB-specific antisense oligonucleotide.

The administration of CREB-specific modulators herein, include, but are not limited to proteins, peptides, polypeptides, antibodies, antisense compounds including oligonucleotides and antisense oligonucleotides, ssRNA, dsRNA molecules, ribozymes, triple helix molecules, siRNA and other RNAi compounds, and small molecule modulators. The antisense compounds included herein, can operate by an RNaseH or RNAi mechanism or by other known mechanism such as splicing.

Further described herein, are CREB-specific inhibitors, for example antisense compounds targeting CREB that reduce CREB mRNA and protein.

Also described herein, are CREB-specific inhibitors, for example antisense compounds targeting CREB that reduce liver CREB mRNA.

Also described herein, are CREB-specific inhibitors, for example antisense compounds targeting CREB that reduce white adipose tissue (WAT) CREB mRNA.

Included herein, are examples of antisense compounds targeting CREB and methods of their use prophylactically, for example, to prevent or delay the progression or development of cardiovascular disorders such as elevated cholesterol and/or triglyceride levels.

The administration of CREB-specific modulators herein, include, but are not limited to proteins, peptides, polypeptides, antibodies, antisense compounds including oligonucleotides and antisense oligonucleotides, ssRNA, dsRNA molecules, ribozymes, triple helix molecules, siRNA and other RNAi compounds, and small molecule modulators. The antisense compounds included herein, can operate by an RNaseH or RNAi mechanism or by other known mechanism such as splicing.

Various CREB-specific modulators targeting CREB, such as antisense compounds, can be utilized in pharmaceutical compositions by adding an effective amount of a compound to a suitable pharmaceutically acceptable diluent or carrier. Use of the compounds and methods of the invention may also be useful prophylactically to prevent such diseases or disorders, e.g., to prevent or delay undue weight gain, diabetes, other metabolic disorders, or cardiovascular disorders.

In one embodiment, a therapeutically effective amount of a CREB-specific antisense compound is administered to a subject having atherosclerosis. In a further embodiment a therapeutically effective amount of antisense compound targeted to a CREB nucleic acid is administered to a subject susceptible to atherosclerosis. Atherosclerosis is assessed directly through routine imaging techniques such as, for example, ultrasound imaging techniques that reveal carotid intimomedial thickness. Accordingly, treatment and/or prevention of atherosclerosis further include monitoring atherosclerosis through routine imaging techniques. In one embodiment, administration of a CREB-specific antisense compound leads to a lessening of the severity of atherosclerosis, as indicated by, for example, a reduction of carotid intimomedial thickness in arteries.

In a non-limiting embodiment, measurements of cholesterol, lipoproteins and triglycerides are obtained using serum or plasma collected from a subject. Methods of obtaining serum or plasma samples are routine, as are methods of preparation of the serum samples for analysis of cholesterol, triglycerides, and other serum markers.

A physician may determine the need for therapeutic intervention for subjects in cases where more or less aggressive blood glucose or triglyceride-lowering therapy is needed. The practice of the methods herein, may be applied to any altered guidelines provided by the NCEP, or other entities that establish guidelines for physicians used in treating any of the diseases or conditions listed herein, for determining coronary heart disease risk and diagnosing metabolic syndrome.

Further described herein, are antisense compounds targeting CREB that reduce triglycerides levels in Type 2 diabetic animals. Thus, antisense compounds targeting CREB are useful in treating, preventing or delaying cardiovascular disease. These findings are in contrast to a previous study showing a dominant-negative CREB decreased expression of hairy and enhancer of 1 (HES-1), a transcriptional repressor of peroxisome proliferator-activated receptor gamma (PPARγ) resulting in increased expression of PPARγ and increased hepatic triglyceride content independent of SREBP (Herzig, S., Hedrick, S., Morantte, I., Koo, S. H., Galimi, F., and Montminy, M. 2003. Nature 426:190-193). In this study, there was no increase in the level of PPARγ expression possibly reflecting differences in the models used or the method of knockdown.

Further described herein, are antisense compounds targeting CREB that reduce total plasma cholesterol in Type 2 diabetic animals. In related embodiment, reduced plasma cholesterol through antisense inhibition of CREB, is mediated by the upregulation of the rate-limiting step of bile acid synthesis catalyzed by Cyp7A1 leading to increased efflux of hepatic cholesterol into bile salts. Moreover, previous studies have described that glucagon signaling inhibits the transcription of Cyp7A1 in cell culture-based systems of rat and human hepatocytes (Song, K. H., and Chiang, J. Y. 2006, Hepatology, 43:117-125; Hylemon, P. B., Gurley, E. C., Stravitz, R. T., Litz, J. S., Pandak, W. M., Chiang, J. Y., and Vlahcevic, Z. R. 1992, J Biol Chem 267:16866-16871). Embodiments, as described herein, support a link between glucagon action and bile acid synthesis in rat liver since CREB inhibition effectively limits glucagon transcriptional signaling therefore increasing the expression of Cyp7A1 and promoting bile salt efflux.

Further described herein, are antisense compounds targeting CREB that reduce hepatic lipid content in Type 2 diabetic animals. Also incorporated herein, the reduced hepatic lipid content, includes, but is not limited to, a reduction in lipids such as triglycerides, diacylglycerols, and long chain CoAs.

In another embodiment, antisense compounds targeting CREB increase rate of fatty acid oxidation in Type 2 diabetic animals.

In another embodiment, antisense compounds targeting CREB increase rate of hepatic insulin sensitivity in Type 2 diabetic animals. In another embodiment, a CREB-specific modulator that decreases the hepatic expression of CREB mRNA, improves hepatic insulin sensitivity associated with fatty liver and hepatic insulin resistance.

In another embodiment, antisense compounds targeting CREB reduce hepatic diacylglycerol (DAG) content in Type 2 diabetic animals.

### Certain Combination Indications

In certain embodiments, the invention provides methods of treating a subject comprising administering one or more CREB-specific modulators. In certain embodiments, the subject has metabolic and cardiovascular-related disorders or conditions including, but not limited to obesity, lipoma, lipomatosis, diabetes (including Type 1 diabetes, Type 2 diabetes and Type 2 diabetes with dyslipidemia), dyslipidemia (including hyperlipidemia, hypertriglyceridemia, and mixed dyslipidemia), non-alcoholic fatty liver disease (NAFLD) (including hepatic steatosis and steatohepatitis), hyperfattyacidemia, metabolic syndrome, hyperglycemia, insulin resistance, hypercholesterolemia (including polygenic hypercholesterolemia), coronary heart disease (early onset coronary heart disease), elevated ApoB, or elevated cholesterol (including elevated LDL-cholesterol, elevated VLDL-cholesterol, elevated IDL-cholesterol, and elevated non-HDL cholesterol).

In one embodiment are methods for decreasing blood glucose levels or triglyceride levels, or alternatively methods for treating obesity or metabolic syndrome, by administering to a subject suffering from elevated glucose or triglyceride levels a therapeutically effective amount of a CREB-specific modulator targeted to a CREB nucleic acid. In another embodiment, a method of decreasing blood glucose or triglyceride levels comprises selecting a subject in need of a decrease in blood glucose or triglyceride levels, and administering to the subject a therapeutically effective amount of a CREB-specific modulator targeted to a CREB nucleic acid. In a further embodiment, a method of reducing risk of development of obesity and metabolic syndrome includes selecting a subject having elevated blood glucose or triglyceride levels and one or more additional indicators risk of development of obesity or metabolic syndrome, and administering to the subject a therapeutically effective amount of a CREB-specific modulator targeted to a CREB nucleic acid, for example a antisense compound.

In one embodiment, administration of a therapeutically effective amount of a CREB-specific modulator targeted a CREB nucleic acid is accompanied by monitoring of glucose levels or triglyceride levels in the serum of a subject, to determine a subject's response to administration of the CREB-specific modulator. A subject's response to administration of the CREB-specific modulator is used by a physician to determine the amount and duration of therapeutic intervention.

In one embodiment are methods for decreasing blood glucose levels or lipid levels, including cholesterol and triglyceride levels, or alternatively methods for treating metabolic disorders, such as obesity, or metabolic syndrome, or cardiovascular disorders, such as hypertriglyceridemia or hypercholesterolemia, by administering to a subject suffering from elevated glucose or triglyceride levels a therapeutically effective amount of a CREB-specific antisense compound. In another embodiment, a method of decreasing blood glucose or triglyceride levels comprises selecting a subject in need of a decrease in blood glucose or triglyceride levels, and administering to the subject a therapeutically effective amount of a CREB-specific antisense compound. In a further embodiment, a method of reducing risk of development of metabolic disorders, such as obesity, or metabolic syndrome, or cardiovascular disorders, such as hypertriglyceridemia or hypercholesterolemia includes selecting a subject having elevated blood glucose or triglyceride levels and one or more additional indicators risk of development of obesity or metabolic syndrome, and administering to the subject a therapeutically effective amount of a CREB-specific antisense compound.

In one embodiment, administration of a therapeutically effective amount of an antisense compound targeted a CREB nucleic acid is accompanied by monitoring of glucose levels or triglyceride levels in the serum of a subject, to determine a subject's response to administration of the antisense compound. A subject's response to administration of the antisense compound is used by a physician to determine the amount and duration of therapeutic intervention.

In certain embodiments, as described herein a pharmaceutical composition comprising an antisense compound targeted to CREB is for use in therapy. In certain embodiments, the therapy is the reduction of blood glucose, triglyceride or liver triglyceride in a subject. In certain embodiments, the therapy is the treatment of hypercholesterolemia, mixed dyslipidemia, atherosclerosis, a risk of developing atherosclerosis, coronary heart disease, a history of coronary heart disease, early onset coronary heart disease, one or more risk factors for coronary heart disease, type 2 diabetes, type 2 diabetes with dyslipidemia, dyslipidemia, hypertriglyceridemia, hyperlipidemia, hyperfattyacidemia, hepatic steatosis, non-alcoholic steatohepatitis, or non-alcoholic fatty liver disease. In additional embodiments, the therapy is the reduction of CHD risk. In certain the therapy is prevention of atherosclerosis. In certain embodiments, the therapy is the prevention of coronary heart disease.

In certain embodiments, as described herein pharmaceutical composition comprising an antisense compound targeted to CREB is used for the preparation of a medicament for reduction of blood glucose, triglyceride or liver triglyceride. In certain embodiments, as described herein pharmaceutical composition comprising an antisense compound targeted to CREB is used for the preparation of a medicament for reducing coronary heart disease risk. In certain embodiments, as described herein an antisense compound targeted to CREB is used for the preparation of a medicament for the treatment of hypercholesterolemia, mixed dyslipidemia, atherosclerosis, a risk of developing atherosclerosis, coronary heart disease, a history of coronary heart disease, early onset coronary heart disease, one or more risk factors for coronary heart disease, type 2 diabetes, type 2 diabetes with dyslipidemia, dyslipidemia, hypertriglyceridemia, hyperlipidemia, hyperfattyacidemia, hepatic steatosis, non-alcoholic steatohepatitis, or non-alcoholic fatty liver disease.

Antisense compounds targeting CREB, described herein, reduce cholesterol levels in normal animals fed a high-fat diet; more particularly reduce LDL-cholesterol. Thus, antisense compounds targeting CREB are useful in treating, preventing or delaying cardiovascular disease.

In one embodiment are methods for decreasing blood glucose and triglyceride levels, or alternatively methods for treating metabolic-related disorders and cardiovascular-related disorders, such as metabolic syndrome and atherosclerosis, by administering to a subject suffering from elevated glucose levels or insulin resistance a therapeutically effective amount of a CREB-specific modulator. In another embodiment, a method of decreasing blood glucose and triglyceride levels comprises selecting a subject in need of a decrease in blood glucose and triglyceride levels, and administering to the subject a therapeutically effective amount of a CREB-specific modulator. In a further embodiment, a method of reducing risk of development of metabolic-related disorders and cardiovascular-related disorders, such as metabolic syndrome and atherosclerosis, includes selecting a subject having elevated blood glucose and triglyceride levels and one or more additional indicators risk of development of metabolic-related disorders and cardiovascular-related disorders, and administering to the subject a therapeutically effective amount of a CREB-specific modulator, for example a antisense compound.

In one embodiment are methods for decreasing blood glucose levels or triglyceride levels, or alternatively methods for treating metabolic disorders, such as obesity or metabolic syndrome, and/or cardiovascular disorders, such as hypertriglyceridemia or hypercholesterolemia, by administering to a subject suffering from elevated glucose or triglyceride levels a therapeutically effective amount of a CREB-specific antisense compound. In another embodiment, a method of decreasing blood glucose or triglyceride levels comprises selecting a subject in need of a decrease in blood glucose or triglyceride levels, and administering to the subject a therapeutically effective amount of a CREB-specific antisense compound. In a further embodiment, a method of reducing risk of development of metabolic disorders, such as obesity, or metabolic syndrome, or cardiovascular disorders, such as hypertriglyceridemia or hypercholesterolemia includes selecting a subject having elevated blood glucose or triglyceride levels and one or more additional indicators risk of development of obesity or metabolic syndrome, and administering to the subject a therapeutically effective amount of a CREB-specific antisense compound.

In one embodiment, an antisense compound targeting CREB modulates CREB expression, processing or activity, wherein, that reduction of CREB expression in lowers plasma triglyceride and cholesterol concentrations in a T2DM animal and protects against fat induced hepatic steatosis and hepatic insulin resistance. In a further embodiment, since T2DM is often associated with hyperlipidemia and hypercholesterolemia in addition to hepatic insulin resistance, CREB reduces or treats T2DM.

In one embodiment, administration of a therapeutically effective amount of an antisense compound targeted a CREB nucleic acid is accompanied by monitoring of glucose levels or triglyceride levels in the serum of a subject, to determine a subject's response to administration of the antisense compound. A subject's response to administration of the antisense compound is used by a physician to determine the amount and duration of therapeutic intervention.

### Assays

Assays for certain metabolic and cardiovascular disease markers are known and understood by those of skill in the art to be useful for assessing the therapeutic effect of a pharmaceutical agent. Such markers include, but are not limited to, glucose, lipids, particularly cholesterol and triglycerides, lipoproteins such as LDL and HDL, proteins such as glycosolated hemoglobin, and other relevant cellular products.

Preferably, the cells within said fluids, tissues or organs being analyzed contain a nucleic acid molecule encoding CREB protein and/or the CREB protein itself. Samples of organs or tissues may be obtained through routine clinical biopsy. Samples of bodily fluid such as blood or urine are routinely and easily tested. For example blood glucose levels can be determined by a physician or even by the patient using a commonly available test kit or glucometer (for example, the Ascensia ELITE™ kit, Ascensia (Bayer), Tarrytown N.Y., or Accucheck, Roche Diagnostics). Alternatively or in addition, glycated hemoglobin (HbA.sub.1c) may be measured. HbA.sub.1c is a stable minor hemoglobin variant formed in vivo via posttranslational modification by glucose, and it contains predominantly glycated NH.sub.2-terminal .beta.-chains. There is a strong correlation between levels of HbA.sub.1c and the average blood glucose levels over the previous 3 months. Thus HbA.sub.1c is often viewed as the “gold standard” for measuring sustained blood glucose control (Bunn, H. F. et al.; 1978, Science. 200, 21-7). HbA.sub.1c can be measured by ion-exchange HPLC or immunoassay; home blood collection and mailing kits for HbA.sub.1c measurement are now widely available. Serum fructosamine is another measure of stable glucose control and can be measured by a calorimetric method (Cobas Integra, Roche Diagnostics).

### CREB-Specific Modulators

The modulatory agents included herein, will suitably affect, modulate or inhibit CREB expression. Suitable agents for reducing or modulating gene expression, processing and activity include, but are not restricted to proteins, peptides, polypeptides, antibodies, antisense compounds including oligonucleotides and antisense oligonucleotides, ssRNA, dsRNA molecules, ribozymes, triple helix molecules, siRNA and other RNAi compounds, and small molecule modulators. The antisense compounds included herein, can operate by an RNaseH or RNAi mechanism or by other known mechanism such as splicing. In a further embodiment, the CREB-specific modulator is a CREB-specific inhibitor, for example an antisense compound targeted to CREB that function to inhibit the translation, for example, of CREB- or CREB-encoding mRNA.

For example, in one non-limiting embodiment, the methods comprise the step of administering to a subject a therapeutically effective amount of a CREB-specific inhibitor. CREB-specific inhibitors, as presented herein, effectively inhibit the activity and/or expression. In one embodiment, the activity or expression of CREB in a subject is inhibited by about 10% in a target cell. Preferably, the activity or expression of CREB in a subject is inhibited by about 30%. More preferably, the activity or expression of CREB in a subject is inhibited by 50% or more. Thus, the CREB-specific modulators, for example oligomeric antisense compounds, modulate expression of CREB mRNA by at least 10%, by at least 20%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100%.

Accordingly, another embodiment herein provides methods for modulating CREB expression, activity, and/or processing comprising contacting CREB with a CREB-specific modulator, which can also be CREB-specific inhibitor. Representative CREB-specific modulators include, but are not limited to proteins, peptides, polypeptides, antibodies, antisense compounds including oligonucleotides and antisense oligonucleotides, ssRNA, dsRNA molecules, ribozymes, triple helix molecules, siRNA and other RNAi compounds, and small molecule modulators. The antisense compounds included herein, can operate by an RNaseH or RNAi mechanism or by other known mechanism such as splicing.

### Ribozymes

Ribozyme molecules designed to catalytically cleave CREB mRNA transcripts can also be used to prevent translation of CREB mRNAs and expression of CREB proteins For example, hammerhead ribozymes that cleave mRNAs at locations dictated by flanking regions that form complementary base pairs with the target mRNA might be used so long as the target mRNA has the following common sequence: 5′-UG-3′. See, e.g., Haseloff and Gerlach (1988) Nature 334:585-591. As another example, hairpin and hepatitis delta virus ribozymes may also be used. See, e.g., Bartolome et al. (2004) Minerva Med. 95(1):11-24. To increase efficiency and minimize the intracellular accumulation of non-functional mRNA transcripts, a ribozyme should be engineered so that the cleavage recognition site is located near the 5′ end of the target CREB mRNA. Ribozymes within the invention can be delivered to a cell using any of the methods as described below.

Other methods can also be used to reduce CREB gene expression in a cell. For example, CREB gene expression can be reduced by inactivating or “knocking out” the CREB gene or its promoter using targeted homologous recombination. See, e.g., Kempin et al., Nature 389: 802 (1997); Smithies et al. (1985) Nature 317:230-234; Thomas and Capecchi (1987) Cell 51:503-512; and Thompson et al. (1989) Cell 5:313-321. For example, a mutant, non-functional CREB gene variant (or a completely unrelated DNA sequence) flanked by DNA homologous to the endogenous CREB gene (either the coding regions or regulatory regions of the CREB gene) can be used, with or without a selectable marker and/or a negative selectable marker, to transfect cells that express CREB protein in vivo.

### Triple-Helix Molecule

CREB gene expression might also be reduced by targeting deoxyribonucleotide sequences complementary to the regulatory region of the CREB gene (i.e., the CREB promoter and/or enhancers) to form triple helical structures that prevent transcription of the CREB gene in target cells. See generally, Helene, C. (1991) Anticancer Drug Des. 6(6): 569-84; Helene, C., et al. (1992) Ann. N.Y. Acad. Sci. 660:27-36; and Maher, L. J. (1992) Bioassays 14(12): 807-15. Nucleic acid molecules to be used in this technique are preferably single stranded and composed of deoxyribonucleotides. The base composition of these oligonucleotides should be selected to promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, e.g., containing a stretch of G residues. These molecules will form a triple helix with a DNA duplex that is rich in GC pairs, in which the majority of the purine residues are located on a single strand of the targeted duplex, resulting in CGC triplets across the three strands in the triplex. The potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

The antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramide chemical synthesis. RNA molecules may be generated by in vitro and in vivo transcription of DNA sequences encoding the antisense RNA molecule. Such DNA sequences may be incorporated into a wide variety of vectors which incorporate suitable RNA polymerase promoters. Alternatively, antisense cDNA constructs that synthesize antisense RNA constitutively or inducibly, depending on the promoter used, can be introduced stably into cell lines.

siRNA/RNAi/dsRNA

The invention comprises CREB-specific modulators, for example siRNA, RNAi and dsRNA, that modulate CREB expression, activity, or processing. The use of short-interfering RNA (siRNA) is a technique known in the art for inhibiting expression of a target gene by introducing exogenous RNA into a living cell (Elbashir et al. 2001. Nature. 411:494-498). siRNA suppress gene expression through a highly regulated enzyme-mediated process called RNA interference (RNAi). RNAi involves multiple RNA-protein interactions characterized by four major steps: assembly of siRNA with the RNA-induced silencing complex (RISC), activation of the RISC, target recognition and target cleavage. Therefore, identifying siRNA-specific features likely to contribute to efficient processing at each step is beneficial efficient RNAi. Reynolds et al. provide methods for identifying such features. A. Reynolds et al., “Rational siRNA design for RNA interference”, Nature Biotechnology 22(3), March 2004.

In that study, eight characteristics associated with siRNA functionality were identified: low G/C content, a bias towards low internal stability at the sense strand 3′-terminus, lack of inverted repeats, and sense strand base preferences (positions 3, 10, 13 and 19). Further analyses revealed that application of an algorithm incorporating all eight criteria significantly improves potent siRNA selection. siRNA sequences that contain internal repeats or palindromes may form internal fold-back structures. These hairpin-like structures may exist in equilibrium with the duplex form, reducing the effective concentration and silencing potential of the siRNA. The relative stability and propensity to form internal hairpins can be estimated by the predicted melting temperatures (T_{M}). Sequences with high T_{M} values would favor internal hairpin structures.

siRNA can be used either ex vivo or in vivo, making it useful in both research and therapeutic settings. Unlike in other antisense technologies, the RNA used in the siRNA technique has a region with double-stranded structure that is made identical to a portion of the target gene, thus making inhibition sequence-specific. Double-stranded RNA-mediated inhibition has advantages both in the stability of the material to be delivered and the concentration required for effective inhibition.

The extent to which there is loss of function of the target gene can be titrated using the dose of double stranded RNA delivered. A reduction or loss of gene expression in at least 99% of targeted cells has been described. See, e.g., U.S. Pat. No. 6,506,559. Lower doses of injected material and longer times after administration of siRNA may result in inhibition in a smaller fraction of cells. Quantitation of gene expression in a cell show similar amounts of inhibition at the level of accumulation of target mRNA or translation of target protein.

The RNA used in this technique can comprise one or more strands of polymerized ribonucleotides, and modification can be made to the sugar-phosphate backbone as disclosed above. The double-stranded structure is often formed using either a single self-complementary RNA strand (hairpin) or two complementary RNA strands. RNA containing a nucleotide sequences identical to a portion of the target gene is preferred for inhibition, although sequences with insertions, deletions, and single point mutations relative to the target sequence can also be used for inhibition. Sequence identity may be optimized using alignment algorithms known in the art and through calculating the percent difference between the nucleotide sequences. The duplex region of the RNA could also be described in functional terms as a nucleotide sequence that is capable of hybridizing with a portion of the target gene transcript.

There are multiple ways to deliver siRNA to the appropriate target, CREB. Standard transfection techniques may be used, in which siRNA duplexes are incubated with cells of interest and then processed using standard commercially available kits. Electroporation techniques of transfection may also be appropriate. Cells or organisms can be soaked in a solution of the siRNA, allowing the natural uptake processes of the cells or organism to introduce the siRNA into the system. Viral constructs packaged into a viral particle would both introduce the siRNA into the cell line or organism and also initiate transcription through the expression construct. Other methods known in the art for introducing nucleic acids to cells may also be used, including lipid-mediated carrier transport, chemical-mediated transport, such as calcium phosphate, and the like.

For therapeutic uses, tissue-targeted nanoparticles may serve as a delivery vehicle for siRNA. These nanoparticles carry the siRNA exposed on the surface, which is then available to bind to the target gene to be silenced. Schiffelers, et al., Nucleic Acids Research 2004 32(19):e149. These nanoparticles may be introduced into the cells or organisms using the above described techniques already known in the art. RGD peptides have been described to be effective at targeting the neovasculature that accompanies the growth of tumors. Designing the appropriate nanoparticles for a particular illness is a matter of determining the appropriate targets for the particular disease.

Other delivery vehicles for therapeutic uses in humans include pharmaceutical compositions, intracellular injection, and intravenous introduction into the vascular system. Inhibition of gene expression can be confirmed by using biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression may be assayed using a reporter or drug resistance gene whose protein product can be easily detected and quantified. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

These techniques are well known and easily practiced by those skilled in the art. In humans, reduction of symptoms of illness will confirm inhibition of the target gene's expression in vivo.

Non-limiting examples of CREB-specific modulating RNAi, siRNA and dsRNA agents are: RNAi molecules, such as 5′-GCAAATGACAGTTCAAGCCC-3′,5′-GTACAGCTGGCTAACAATGG-3′,5′-GAGAGAGGTCCGTCTAATG-3′, siRNA, such as 5′-UACAGCUGGCUAACAAUGGdTdT-3′,5′-GGAGUCUGUGGAUAGUGUAtt-3′ and 5′-UACACUAUCCACAGACUCCtg-3′, Smartpool siRNA targeted to rat CREB NM_{—}031017, sc-35111 from Santa Cruz, 5′-TGGTCATCTAGTCACCGGTG-3′,5′-ACAGCTGGCTAACAATGG-3′,5′-GGUGGAAAAUGGACUGGCUtt-3′,

### Polyclonal and Monoclonal Antibodies

The invention comprises CREB-specific modulators, for example, polyclonal and monoclonal antibodies that bind to CREB polypeptides of the invention and modulate CREB expression, activity, or processing. The term “antibody” as used herein, refers to immunoglobulin molecules and immunologically active portions of immunoglobulin molecules, i.e., molecules that contain a binding site that specifically binds to an epitope (antigen, antigenic determinant). An antibody molecule that specifically binds to a polypeptide of the invention is a molecule that binds to an epitope present in said polypeptide or a fragment thereof, but does not substantially bind other molecules in a sample, e.g., a biological sample, which naturally contains the polypeptide. Examples of immunologically active portions of immunoglobulin molecules include F(ab) and F(ab′).sub.2 fragments which can be generated by treating the antibody with an enzyme such as pepsin. Polyclonal and/or monoclonal antibodies that specifically bind one form of the gene product but not to the other form of the gene product are also provided. Antibodies are also provided, that bind a portion of either the variant or the reference gene product that contains the polymorphic site or sites. The term “monoclonal antibody” or “monoclonal antibody composition”, as used herein, refers to a population of antibody molecules that are directed against a specific epitope and are produced either by a single clone of B cells or a single hybridoma cell line. A monoclonal antibody composition thus typically displays a single binding affinity for a particular polypeptide of the invention with which it immunoreacts.

Polyclonal antibodies can be prepared as known by those skilled in the art by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique (Kohler G and Milstein C, 1975), the human B cell hybridoma technique (Kozbor D et al, 1982), the EBV-hybridoma technique (Cole S P et al, 1984), or trioma techniques (Hering S et al, 1988). To produce a hybridoma an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (Bierer B et al, 2002). Moreover, the ordinarily skilled worker will appreciate that there are many variations of such methods that also would be useful. Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide (Hayashi N et al, 1995; Hay B N et al, 1992; Huse W D et al, 1989; Griffiths A D et al, 1993). Kits for generating and screening phage display libraries are commercially available.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. An antibody specific for a polypeptide of the invention can facilitate the purification of a native polypeptide of the invention from biological materials, as well as the purification of recombinant form of a polypeptide of the invention from cultured cells (culture media or cells). Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein and/or metabolite levels in tissues such as blood as part of a risk assessment, diagnostic or prognostic test for cardiovascular, diabetic, metabolic disorder, and obesity or as part of a clinical testing procedure, e.g., to, for example, determine the efficacy of a given treatment regimen. Antibodies can be coupled to various enzymes, prosthetic groups, fluorescent materials, luminescent materials, bioluminescent materials, and radioactive materials to enhance detection. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include .sup.125I, 131I, 35S or 3H.

Highly purified antibodies (e.g. monoclonal humanized antibodies specific to a polypeptide encoded by the CREB gene of this invention) may be produced using GMP-compliant manufacturing processes well known in the art. These “pharmaceutical grade” antibodies can be used in novel therapies modulating activity and/or function of a polypeptide encoded by the CREB gene of this invention or modulating activity and/or function of a metabolic pathway related to the CREB gene of this invention.

### Small Molecule and Other CREB-Specific Modulators and Effects of Modulating CREB Expression

Examples of other CREB-specific modulating agents showing the effects of impaired CREB signaling or silencing of CREB expression include, but are not limited to forskolin, isoproterenol; oxymatrine; GYKI 52466, CFM-2; flavin7; genistein; dopamine receptor, D3; insulin; 1,2-naphthoquinone; 2,5-dimethyl-4-hydroxy-3(2H)-furanone; aldosterone; A20; morphine; HBZ protein of human T-cell leukemia virus; phenylarsine oxide; melatonin; propofol; C-reactive protein; scopolamine; D1 receptor antagonist, SL327, MEK inhibitor; grape seed extract; L-type calcium channel ligands; ERK kinase inhibitor U0126; COOH-terminal binding protein (CtBP) corepressors; SB202190 and PD169316; 2-methylarachidonyl-(2′-fluoroethyl)amide (F-Me-AEA), anandamide; H89, wortmannin and the Akt inhibitor SH-6; cyclosporin A, tacrolimus; KG-501 (2-naphthol-AS-E-phosphate); piperine; 17beta-estradiol (E2); curcumin (diferuloylmethane); ICG-001; the thyroid hormone triiodothyronine (T3); HOX proteins; the beta isoform of the type 2 regulatory subunit (RIIbeta) of protein kinase A; the Presenilin1 (PS1)-dependent epsilon-cleavage product N-Cad/CTF2; the p38 mitogen-activated protein kinase inhibitor (MAPK), SB203580; inhibitors of the interleukin-1beta converting enzyme subfamily (caspase-1, -4, -5 and -13); tetrodotoxin, PKA inhibitor peptide, PKI; Polyglutamine disease protein, ataxin-3; RNA aptamers; cyclosporine; protein phosphatase-1 (PP-1) and PP-2A; ZDC(C)PR antagonist of ZNC(C)PR, PTX (inhibitor of G(o)/G(I) protein coupled receptor), GF109203x (inhibitor of PKC), PD98059 (inhibitor of MAPK); progesterone; cannabinol; Tip60 (Tat interactive protein); red wine polyphenols (RW-PF); nonsteroidal anti-inflammatory drugs (NSAIDs), sodium salicylate (NaSal); SB 203580 and PD 98059; candidate plasticity gene 16 (cpg16); A-CREB, a dominant-negative (D-N) inhibitor protein of CREB; cyclosporin A, FK506; glucocorticoids; the adenovirus E1A oncoprotein; antidepressant drugs, clomipramine, imipramine, fluoxetine, doxepin, desipramine, amitriptyline, maprotiline, mianserin, and trazodone; simvastatin; KG-501; neprilysin; paeonol; phencyclidine; beta-Arrestin-1; ginseng total saponin (GTS); 1-alpha-phosphatidylcholine beta-arachidonoyl-gamma-palmitoyl (PAPC); hypoxia; GABA(B) receptor (GABA(B)R); ICER; extrasynaptic NMDA receptors; Ro-31-8220; beta-eudesmol; BEL or AACOF(3); methylation at the CRE; panaxynol; MEK inhibitor UO126; cannabis; dopamine and cAMP-regulated phosphoprotein (DARPP-32); amphetamine; chronic lithium treatment; salt-inducible kinase (SIK); and chronic exposure to hypoxia; or platelet-derived growth factor BB (PDGF-BB).

The studied effects of impaired or reduced CREB signaling or silencing of CREB expression include, but are not limited to, decreased proliferation of stem cells, cell-cycle abnormalities, delayed leukemic infiltration, down-regulation of the expression of several genes for synthesis of triglycerides such as SCD1, attenuated response to nicotine, anisomycin-induced COX-2 expression, colorectal angiogenesis, a decrease in bcl-2 and an increase in oxidant-induced apoptosis, reversal of GIP-mediated antiapoptotic effect via the cyclic AMP (cAMP)/protein kinase A (PKA) cascade, antinociceptive effects (inhibition of peripheral noxious stimulation), inhibition of glucose-induced upstream stimulatory factor 2 (USF2) expression, inhibition of high glucose-induced thrombospondin1 (TSP1) gene expression and TGF-beta activity, enhancement of diabetic renal complications, blockage of COX-2 induction, involvement in inflammation and carcinogenesis, enhancement of apoptotic cell death of cells known for their sensitivity to the tumor necrosis factor (TNF)-related apoptosis-inducing ligand (TRAIL)/Apo2L cytotoxic action, reduction in Bcl-2, inhibition of glycogen synthase kinase-3 (GSK-3, suppression of excitatory postsynaptic potential (EPSP), blockage of PDB/TG-dependent expression of COX-2 and mPGES-1 mRNA, modulation of long-term synaptic facilitation (LTF), inhibition of calcium/calmodulin-dependent protein kinase IV (CaMKIV), modulation of emotional behavior, inhibition of antimycin-A-induced triglyceride accumulation in preadipocytes, inhibition of NOR-1 promoter activity, reduction in LDL-induced mitogenesis, enhanced colX expression in control and in TGF-beta and BMP-2-treated cultures, reduced expression of the mitochondrial-matrix enzyme 5-aminolaevulinate synthase (ALAS) gene, reduced long-term memory in the dorsolateral striatum, decrease in both PDGF-induced SMC migration and OPN expression, inhibition of the pro-apoptotic effect of FXa, a coagulation Factor, blockage of TNF activation, loss of immunity and cell viability, complete inhibition of induction of the c-fos gene, growth inhibition in B cells, prevention of hepatic fibrosis, antiproliferative action; inhibition of kidney tumor growth, prevention of infection of cells with arenavirus, reduced cocaine-induced signaling, inhibition of gluconeogenesis gene expression, inhibition of melanogenesis, impaired vascular reactivity, promotion of endothelial dysfunction, blockage of artery, inhibition of IL-2 production, impact on heart dis, impact on sleep in elderly humans, cause of amnesia, Decrease in IL-10, alteration of the antiinflammatory/proinflammatory balance, accentuation of inflammation, impact in atherothrombosis, suppression of the survival of newborn cells in the dentate gyms, inhibition cocaine-induced activation, Inhibition of aromatase activity, potential use in breast cancer treatment, Blockage of nicotine-induced signaling, inhibition of acetylation of histone, impact on colorectal angiogenesis, prevention of radiocontrast nephropathy, immunosuppressive action, Reduction of proinflammatory cytokines, reduced activity of class II MHC promoter, inhibition of HAT activity, Selective induction of apoptosis in transformed cells but not in normal colon cells, reduction in vitro growth of colon carcinoma cells, efficacy in the Min mouse and nude mouse xenograft models of colon cancer, influence in relevant processes such as cell proliferation, transformation, or tumorigenesis, association with hematopoiesis and leukemias, suppression of c-Fos production in T cells following activation via the TCR, immunosuppressive and anti-inflammatory actions in human T-cells, interference with CD86 gene transcription in the presence of activated NF-kappaB, interference with synaptic activity of Aroclor 1254-induction, polyglutamine disease, inhibition of proliferation of acinar, inhibition of learning and memory, inhibition of hCGalpha gene transcription, inhibition of interleukin-2 (IL-2) and immunoglobulin kappa chain expression in B cells, Antiproliferative effect, complete inhibition of cAMP-mediated, but only partially inhibited Ca2+- and NGF-mediated, transcription, reduction of Alzheimer's amyloid-beta 42 (Abeta 42)-induced neuron loss and intraneuronal Abeta 42 deposits, acceleration of age-dependent axon pathology and premature death in Drosophila, inhibition of oocyte meiotic resumption, increase in P2X(3) receptor transcription, down-regulation of melanin production via decreasing MITF expression and consequent mRNA and protein levels of tyrosinase, broad attenuation of GLP-1 signaling, decreased IRS-2 expression, impaired insulin secretion, schizophrenia, inhibition of CD2AP promoter activity in renal tubular epithelial cells, inhibition of the development of physical and psychological dependence on morphine, suppression of pre-BCR-mediated cell expansion, increase in anxiety, sucrose preference, and sensitivity to drugs of abuse and decrease in depression-like behavior, arrest in the cell cycle at the G(2)-M phase, and subsequent induction of apoptosis with the suppression of Bcl-2 and Bcl-xL expression, inhibition of proliferation of human umbilical vein endothelial cells (HUVEC), blockage of phenylpropanolamine (PPA)-induced anorexia and modification of neuropeptide Y (NPY) and superoxide dismutase-2 (SOD-2) mRNA content toward normal, sustained global ischaemia of both non-preconditioned and preconditioned hearts, down-regulation of the lysophosphatidic acid (LPA)-stimulated c-fos promoter activation, induction of subtle alterations in the emotional circuit in female rats, ending in depressive-like behavior, alteration of sensitivity to rewarding stimuli, reduction of PAI-1 induction by angiotensin II (ANG II), enhancement of the transcription of osteopontin (OPN), initiaon of age-dependent axon degeneration and shortening of lifespan, odorant deprivation, hypothyroidism, usage in the treatment of bipolar disorder, modulation of hippocampus-dependent long-term memory, pulmonary hypertension and modulation of SMC layers of atherogenic systemic arteries.

### Antisense Compounds

The invention also provides a method for delivering one or more of the above-described nucleic acid molecules into cells that express CREB protein. A number of methods have been developed for delivering antisense DNA or RNA into cells. For example, antisense molecules can be introduced directly into a cell by electroporation, liposome-mediated transfection, CaCl-mediated transfection, or using a gene gun. Modified nucleic acid molecules designed to target the desired cells (e.g., antisense oligonucleotides linked to peptides or antibodies that specifically bind receptors or antigens expressed on the target cell surface) can be used. To achieve high intracellular concentrations of antisense oligonucleotides (as may be required to suppress translation on endogenous mRNAs), a preferred approach utilizes a recombinant DNA construct in which the antisense oligonucleotide is placed under the control of a strong promoter (e.g., the CMV promoter).

Oligomeric compounds include, but are not limited to, oligonucleotides, oligonucleotides, oligonucleotide analogs, oligonucleotide mimetics, antisense compounds, antisense oligonucleotides, and siRNA. An oligomeric compound may be “antisense” to a target nucleic acid, meaning that it is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a nucleic acid to which it is targeted. In certain such embodiments, an antisense oligonucleotide has a nucleobase sequence that, when written in the 5′ to 3′ direction, comprises the reverse complement of the target segment of a nucleic acid to which it is targeted.

In certain embodiments, a CREB-specific antisense compound is 8 to 80, 12 to 50, 12 to 30, 10 to 50, 10 to 30, 18 to 24, 19 to 22 or 15 to 30 subunits in length, as described herein. In other words, antisense compounds are from 8 to 80, 12 to 50, 12 to 30, 10 to 50, 10 to 30 or 15 to 30 linked subunits. In certain embodiments, an antisense compound targeted to a CREB nucleic acid or CREB-specific nucleic acid is 12 to 30 subunits in length. In other words, antisense compounds are from 12 to 30 linked subunits. In certain embodiments, the antisense compound is 8 to 80, 12 to 50, 15 to 30, 18 to 24, 19 to 22, or 20 linked subunits. In certain embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In certain embodiments, the antisense compound is an antisense oligonucleotide, and the linked subunits are nucleotides.

In certain embodiments, a CREB-specific antisense compound nucleic acid is 10 to 30 nucleotides in length. In certain such embodiments, an antisense oligonucleotide targeted to a CREB nucleic acid is 10, 11, 12, 13, 14, 15, 16, 17, 18, 19, 20, 21, 22, 23, 24, 25, 26, 27, 28, 29, or 30 nucleotides in length.

In certain embodiments, as described herein, an antisense oligonucleotide targeted to a CREB nucleic acid is 10 to 30 nucleotides in length. In certain such embodiments, as described herein, an antisense oligonucleotide targeted to a CREB nucleic acid is 10, 11, 12, 13, 14, 15, 16, 17, 18, 19, 20, 21, 22, 23, 24, 25, 26, 27, 28, 29, or 30 nucleotides in length.

In certain embodiments, a CREB-specific antisense compound nucleic acid is 12 to 30 nucleotides in length, as described herein. In certain such embodiments, an antisense oligonucleotide targeted to a CREB nucleic acid is 12, 13, 14, 15, 16, 17, 18, 19, 20, 21, 22, 23, 24, 25, 26, 27, 28, 29, or 30 nucleotides in length.

In certain embodiments, an antisense oligonucleotide targeted to a CREB nucleic acid is 12 to 30 nucleotides in length, as described herein. In certain such embodiments, an antisense oligonucleotide targeted to a CREB nucleic acid is 12, 13, 14, 15, 16, 17, 18, 19, 20, 21, 22, 23, 24, 25, 26, 27, 28, 29, or 30 nucleotides in length.

In certain embodiments, a CREB-specific antisense compound nucleic acid is 12 to 50 nucleotides in length, as described herein. In certain such embodiments, an antisense oligonucleotide targeted to a CREB nucleic acid is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In certain embodiments, an antisense oligonucleotide targeted to a CREB nucleic acid is 12 to 50 nucleotides in length, as described herein. In certain such embodiments, an antisense oligonucleotide targeted to a CREB nucleic acid is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In certain embodiment, a CREB-specific antisense compound is 15 to 30 subunits in length, as described herein. In other words, antisense compounds are from 15 to 30 linked subunits. In certain such embodiments, the antisense compounds are 15, 16, 17, 18, 19, 20, 21, 22, 23, 24, 25, 26, 27, 28, 29, or 30 subunits in length.

In certain embodiments, an antisense oligonucleotide targeted to a CREB nucleic acid is 15 to 30 nucleotides in length, as described herein. In certain such embodiments, an antisense oligonucleotide targeted to a CREB nucleic acid is 15, 16, 17, 18, 19, 20, 21, 22, 23, 24, 25, 26, 27, 28, 29, or 30 nucleotides in length.

In certain embodiments, a CREB-specific antisense compound is 18 to 24 subunits in length, as described herein. In other words, antisense compounds are from 18 to 24 linked subunits. In one embodiment, the antisense compounds are 18, 19, 20, 21, 22, 23, or 24 subunits in length.

In certain embodiments, an antisense oligonucleotide targeted to a CREB nucleic acid is 18 to 24 nucleotides in length, as described herein. In certain such embodiments, an antisense oligonucleotide targeted to a CREB nucleic acid is 18, 19, 20, 21, 22, 23, or 24 nucleotides in length.

In certain embodiments, a CREB-specific antisense compound is 19 to 22 subunits in length, as described herein. In other words, antisense compounds are from 19 to 22 linked subunits. This embodies antisense compounds of 19, 20, 21, or 22 subunits in length.

In certain embodiments, an antisense oligonucleotide targeted to a CREB nucleic acid is 19 to 22 nucleotides in length, as described herein. In certain such embodiments, an antisense oligonucleotide targeted to a CREB nucleic acid is 19, 20, 21, or 22 nucleotides in length.

In certain embodiments, a CREB-specific antisense compound is 20 subunits in length, as described herein. In certain such embodiments, antisense compounds are 20 linked subunits in length.

In certain embodiments, as described herein, an antisense oligonucleotide targeted to a CREB nucleic acid is 20 nucleotides in length. In certain such embodiments, as described herein, an antisense oligonucleotide targeted to a CREB nucleic acid is 20 linked nucleotides in length. In certain embodiments, as described herein, a shortened or truncated antisense compound targeted to a CREB nucleic acid has a single subunit deleted from the 5′ end (5′ truncation), or alternatively from the 3′ end (3′ truncation). A shortened or truncated antisense compound targeted to a CREB nucleic acid may have two subunits deleted from the 5′ end or, alternatively, may have two subunits deleted from the 3′ end of the antisense compound. Alternatively, the deleted nucleosides may be dispersed throughout the antisense compound; for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two are more additional subunits are present, the added subunits may be adjacent to each other; for example, in an antisense compound having two subunits added to the 5′ end (5′ addition), or alternatively to the 3′ end (3′ addition), of the antisense compound. Alternatively, the added subunits may be dispersed throughout the antisense compound; for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target-specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and 28 and 42 nucleobase antisense oligonucleotides comprised of the sequence of two or three of the tandem antisense oligonucleotides respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

PCT/US2007/068404 describes incorporation of chemically-modified high-affinity nucleotides into short antisense compounds about 8-16 nucleobases in length and that such compounds are useful in the reduction of target RNAs in animals with increased potency and improved therapeutic index.

In certain embodiments, as described herein, antisense compounds targeted to CREB nucleic acid are short antisense compounds. In certain embodiments, as described herein, such short antisense compounds are oligonucleotide compounds. In certain embodiments, as described herein such short antisense compounds are about 8 to 16, preferably 9 to 15, more preferably 9 to 14, more preferably 10 to 14 nucleotides in length and comprise a gap region flanked on each side by a wing, wherein, each wing independently consists of 1 to 3 nucleotides. Preferred motifs include but are not limited to wing-deoxy gap-wing motifs selected from 3-10-3, 2-10-3, 2-10-2, 1-10-1, 2-8-2, 1-8-1, 3-6-3 or 1-6-1.

Antisense compounds targeted to CREB nucleic acid are synthesized in vitro and do not include antisense compositions of biological origin, or genetic vector constructs designed to direct the in vivo synthesis of antisense molecules.

In certain embodiments, as described herein, an antisense compound is targeted to a region of a CREB nucleic acid that does not contain a single nucleotide polymorphism (SNPs). In certain embodiments, as described herein, an antisense compound is targeted to a region of a CREB nucleic acid that does contain a single nucleotide polymorph (SNPs). A single nucleotide polymorphism refers to polymorphisms that are the result of a single nucleotide alteration or the existence of two or more alternative sequences which can be, for example, different allelic forms of a gene. A polymorphism may comprise one or more base changes including, for example, an insertion, a repeat, or a deletion. In certain embodiments, as described herein, an antisense oligonucleotide targeted to a CREB nucleic acid overlaps with a SNP at the following positions: 428, 432, 449, 996, 1011, 1044, 1317, 1565, 1617, 1618, 1671, 1711, 1722, 1836, 1911. In certain embodiments, as described herein, the compounds provided herein, that target a region of CREB nucleic acid that contains one or more SNPs will contain the appropriate base substitution, insertion, repeat or deletion such that the compound is fully complementary to the altered CREB nucleic acid sequence.

### Antisense Compound Motifs

In certain embodiments, as described herein, antisense compounds targeted to CREB nucleic acids have chemically modified subunits arranged in patterns, or motifs, to confer to the antisense compounds properties, such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may serve as a substrate for the cellular endonuclease RNase H, which cleaves the RNA strand of a RNA:DNA duplex.

Antisense compounds having a gapmer motif are considered chimeric antisense compounds. In a gapmer, an internal position having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. The regions of a gapmer are differentiated by the types of sugar moieties comprising each distinct region. The types of sugar moieties that are used to differentiate the regions of a gapmer may in some embodiments, as described herein include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH2)n-O-2′ bridge, where n=1 or 2). In general, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region.

In some embodiments, as described herein, an antisense compound targeted to CREB nucleic acids has a gap-widened motif. In other embodiments, as described herein, an antisense oligonucleotide targeted to CREB nucleic acids has a gap-widened motif.

PCT/US2006/0063730 describes incorporation of gap-widened antisense oligonucleotides having various wing-gap-wing motifs. In certain embodiments, as described herein, a gap-widened motif includes, but is not limited to, 5-10-5, 2-13-5, 3-14-3, 3-14-4 gapmer motif.

In one embodiment, a gap-widened antisense oligonucleotide targeted to a CREB nucleic acid has a gap segment of fourteen 2′-deoxyribonucleotides positioned between wing segments of three chemically modified nucleosides. In one embodiment, the chemical modification comprises a 2′-sugar modification. In another embodiment, the chemical modification comprises a 2′-MOE sugar modification.

In one embodiment, antisense compounds targeted to CREB nucleic acid possess a 5-10-5 gapmer motif.

### Target Nucleic Acids, Target Regions and Nucleotide Sequences

Nucleotide sequences that encode CREB include, without limitation, the following: GENBANK®Accession No. NM_{—}134442.2, and incorporated herein as SEQ ID NO: 1; nucleotides 6598000 to 6666000 of GENBANK® Accession No. NW_{—}047816.2, and incorporated herein as SEQ ID NO: 2; GENBANK® Accession No. NM_{—}031017.1, and incorporated herein as SEQ ID NO: 3; GENBANK® Accession No. CV116908.1 and incorporated herein as SEQ ID NO: 4; GENBANK® Accession No. BE114301.1_COMP and incorporated herein as SEQ ID NO: 5; GENBANK® Accession No. NM_{—}134443.1 and incorporated herein as SEQ ID NO: 6; GENBANK® Accession No. NM_{—}133828.1 and incorporated herein as SEQ ID NO: 7; GENBANK® Accession No. NM_{—}009952.1 and incorporated herein as SEQ ID NO: 8; nucleotides 42407947 to 42484927 of GENBANK® Accession No. NT_{—}039170.1 and incorporated herein as SEQ ID NO: 9; GENBANK® Accession No. X92497.1 and incorporated herein as SEQ ID NO: 10; GENBANK®Accession No. U46027.1 and incorporated herein as SEQ ID NO: 11; and GENBANK® Accession No. AK042595.1 and incorporated herein as SEQ ID NO: 12.

It is noted that some portions of these nucleotide sequences share identical sequence. For example, portions of SEQ ID NO: 1 are identical to portions of SEQ ID NO: 2; portions of SEQ ID NO: 1 are identical to portions of SEQ ID NO: 3; portions of SEQ ID NO: 1 are identical to portions of SEQ ID NO: 4, portions of SEQ ID NO: 1 are identical to portions of SEQ ID NO: 5, portions of SEQ ID NO: 1 are identical to portions of SEQ ID NO: 6; portions of SEQ ID NO: 1 are identical to portions of SEQ ID NO: 7, portions of SEQ ID NO: 1 are identical to portions of SEQ ID NO: 8, portions of SEQ ID NO: 1 are identical to portions of SEQ ID NO: 9, portions of SEQ ID NO: 1 are identical to portions of SEQ ID NO: 10, portions of SEQ ID NO: 1 are identical to portions of SEQ ID NO: 11, portions of SEQ ID NO: 1 are identical to portions of SEQ ID NO: 12; portions of SEQ ID NO: 2 are identical to portions of SEQ ID NO: 3, portions of SEQ ID NO: 2 are identical to portions of SEQ ID NO: 4, portions of SEQ ID NO: 2 are identical to portions of SEQ ID NO: 5, portions of SEQ ID NO: 2 are identical to portions of SEQ ID NO: 6, portions of SEQ ID NO: 2 are identical to portions of SEQ ID NO: 7, portions of SEQ ID NO: 2 are identical to portions of SEQ ID NO: 8, portions of SEQ ID NO: 2 are identical to portions of SEQ ID NO: 9. portions of SEQ ID NO: 2 are identical to portions of SEQ ID NO: 10, portions of SEQ ID NO: 2 are identical to portions of SEQ ID NO: 11, portions of SEQ ID NO: 2 are identical to portions of SEQ ID NO: 12; portions of SEQ ID NO: 3 are identical to portions of SEQ ID NO: 4, portions of SEQ ID NO: 3 are identical to portions of SEQ ID NO: 5, portions of SEQ ID NO: 3 are identical to portions of SEQ ID NO: 6, portions of SEQ ID NO: 3 are identical to portions of SEQ ID NO: 7, portions of SEQ ID NO: 3 are identical to portions of SEQ ID NO: 8, portions of SEQ ID NO: 3 are identical to portions of SEQ ID NO: 9, portions of SEQ ID NO: 3 are identical to portions of SEQ ID NO: 10, portions of SEQ ID NO: 3 are identical to portions of SEQ ID NO: 11, portions of SEQ ID NO: 3 are identical to portions of SEQ ID NO: 12; portions of SEQ ID NO: 4 are identical to portions of SEQ ID NO: 5, portions of SEQ ID NO: 4 are identical to portions of SEQ ID NO: 6, portions of SEQ ID NO: 4 are identical to portions of SEQ ID NO: 7; portions of SEQ ID NO: 4 are identical to portions of SEQ ID NO: 8, portions of SEQ ID NO: 4 are identical to portions of SEQ ID NO: 9, portions of SEQ ID NO: 4 are identical to portions of SEQ ID NO: 10, portions of SEQ ID NO: 4 are identical to portions of SEQ ID NO: 11, portions of SEQ ID NO: 4 are identical to portions of SEQ ID NO: 12; portions of SEQ ID NO: 5 are identical to portions of SEQ ID NO: 6, portions of SEQ ID NO: 5 are identical to portions of SEQ ID NO: 7, portions of SEQ ID NO: 5 are identical to portions of SEQ ID NO: 8, portions of SEQ ID NO: 5 are identical to portions of SEQ ID NO: 9, portions of SEQ ID NO: 5 are identical to portions of SEQ ID NO: 10, portions of SEQ ID NO: 5 are identical to portions of SEQ ID NO: 11, portions of SEQ ID NO: 5 are identical to portions of SEQ ID NO: 12; portions of SEQ ID NO: 6 are identical to portions of SEQ ID NO: 7, portions of SEQ ID NO: 6 are identical to portions of SEQ ID NO: 8, portions of SEQ ID NO: 6 are identical to portions of SEQ ID NO: 9, portions of SEQ ID NO: 6 are identical to portions of SEQ ID NO: 10, portions of SEQ ID NO: 6 are identical to portions of SEQ ID NO: 11, portions of SEQ ID NO: 6 are identical to portions of SEQ ID NO: 12; portions of SEQ ID NO: 7 are identical to portions of SEQ ID NO: 8; portions of SEQ ID NO: 7 are identical to portions of SEQ ID NO: 9, portions of SEQ ID NO: 7 are identical to portions of SEQ ID NO: 10, portions of SEQ ID NO: 7 are identical to portions of SEQ ID NO: 11, portions of SEQ ID NO: 7 are identical to portions of SEQ ID NO: 12; portions of SEQ ID NO: 8 are identical to portions of SEQ ID NO: 9, portions of SEQ ID NO: 8 are identical to portions of SEQ ID NO: 10, portions of SEQ ID NO: 8 are identical to portions of SEQ ID NO: 11, portions of SEQ ID NO: 8 are identical to portions of SEQ ID NO: 12; portions of SEQ ID NO: 9 are identical to portions of SEQ ID NO: 10, portions of SEQ ID NO: 9 are identical to portions of SEQ ID NO: 11, portions of SEQ ID NO: 9 are identical to portions of SEQ ID NO: 12; portions of SEQ ID NO: 10 are identical to portions of SEQ ID NO: 11, portions of SEQ ID NO: 10 are identical to portions of SEQ ID NO: 12; and portions of SEQ ID NO: 11 are identical to portions of SEQ ID NO: 12.

Accordingly, antisense compounds targeted to SEQ ID NO: 1 may also target SEQ ID NO: 2 and/or SEQ ID NO: 3, and/or SEQ ID NO: 4, and/or SEQ ID NO: 5, and/or SEQ ID NO: 6; and/or SEQ ID NO: 7, and/or SEQ ID NO: 8, and/or SEQ ID NO: 9, and/or SEQ ID NO: 10, and/or SEQ ID NO: 11, and/or SEQ ID NO: 12. Antisense compounds targeted to SEQ ID NO: 2 may also target SEQ ID NO: 1 and/or SEQ ID NO: 3, and/or SEQ ID NO: 4, and/or SEQ ID NO: 5, and/or SEQ ID NO: 6; and/or SEQ ID NO: 7, and/or SEQ ID NO: 8, and/or SEQ ID NO: 9, and/or SEQ ID NO: 10, and/or SEQ ID NO: 11, and/or SEQ ID NO: 12 and antisense compounds targeted to SEQ ID NO: 3 may also target SEQ ID NO: 1 and/or SEQ ID NO: 2 and/or SEQ ID NO: 4, and/or SEQ ID NO: 5, and/or SEQ ID NO: 6; and/or SEQ ID NO: 7, and/or SEQ ID NO: 8, and/or SEQ ID NO: 9, and/or SEQ ID NO: 10, and/or SEQ ID NO: 11, and/or SEQ ID NO: 12; and antisense compounds targeted to SEQ ID NO: 4 may also target SEQ ID NO: 1 and/or SEQ ID NO: 2 and/or SEQ ID NO: 3, and/or SEQ ID NO: 5, and/or SEQ ID NO: 6; and/or SEQ ID NO: 7, and/or SEQ ID NO: 8, and/or SEQ ID NO: 9, and/or SEQ ID NO: 10, and/or SEQ ID NO: 11, and/or SEQ ID NO: 12; and antisense compounds targeted to SEQ ID NO: 5 may also target SEQ ID NO: 1 and/or SEQ ID NO: 2 and/or SEQ ID NO: 3, and/or SEQ ID NO: 4, and/or SEQ ID NO: 6, and/or SEQ ID NO: 7, and/or SEQ ID NO: 8, and/or SEQ ID NO: 9, and/or SEQ ID NO: 10, and/or SEQ ID NO: 11, and/or SEQ ID NO: 12; and antisense compounds targeted to SEQ ID NO: 6 may also target SEQ ID NO: 1 and/or SEQ ID NO: 2 and/or SEQ ID NO: 3, and/or SEQ ID NO: 4, and/or SEQ ID NO: 5 and/or SEQ ID NO: 7, and/or SEQ ID NO: 8, and/or SEQ ID NO: 9, and/or SEQ ID NO: 10, and/or SEQ ID NO: 11, and/or SEQ ID NO: 12; and antisense compounds targeted to SEQ ID NO: 7 may also target SEQ ID NO: 1 and/or SEQ ID NO: 2 and/or SEQ ID NO: 3, and/or SEQ ID NO: 4, and/or SEQ ID NO: 5 and/or SEQ ID NO: 6, and/or SEQ ID NO: 8, and/or SEQ ID NO: 9, and/or SEQ ID NO: 10, and/or SEQ ID NO: 11, and/or SEQ ID NO: 12; and antisense compounds targeted to SEQ ID NO: 8 may also target SEQ ID NO: 1 and/or SEQ ID NO: 2 and/or SEQ ID NO: 3, and/or SEQ ID NO: 4, and/or SEQ ID NO: 5 and/or SEQ ID NO: 6, and/or SEQ ID NO: 7, and/or SEQ ID NO: 9, and/or SEQ ID NO: 10, and/or SEQ ID NO: 11, and/or SEQ ID NO: 12; and antisense compounds targeted to SEQ ID NO: 9 may also target SEQ ID NO: 1 and/or SEQ ID NO: 2 and/or SEQ ID NO: 3, and/or SEQ ID NO: 4, and/or SEQ ID NO: 5 and/or SEQ ID NO: 7, and/or SEQ ID NO: 8, and/or SEQ ID NO: 6, and/or SEQ ID NO: 10, and/or SEQ ID NO: 11, and/or SEQ ID NO: 12; and antisense compounds targeted to SEQ ID NO: 10 may also target SEQ ID NO: 1 and/or SEQ ID NO: 2 and/or SEQ ID NO: 3, and/or SEQ ID NO: 4, and/or SEQ ID NO: 5 and/or SEQ ID NO: 7, and/or SEQ ID NO: 8, and/or SEQ ID NO: 9, and/or SEQ ID NO: 6, and/or SEQ ID NO: 11, and/or SEQ ID NO: 12; and antisense compounds targeted to SEQ ID NO: 11 may also target SEQ ID NO: 1 and/or SEQ ID NO: 2 and/or SEQ ID NO: 3, and/or SEQ ID NO: 4, and/or SEQ ID NO: 5 and/or SEQ ID NO: 7, and/or SEQ ID NO: 8, and/or SEQ ID NO: 9, and/or SEQ ID NO: 10, and/or SEQ ID NO: 6, and/or SEQ ID NO: 12; and antisense compounds targeted to SEQ ID NO: 12 may also target SEQ ID NO: 1 and/or SEQ ID NO: 2 and/or SEQ ID NO: 3, and/or SEQ ID NO: 4, and/or SEQ ID NO: 5 and/or SEQ ID NO: 7, and/or SEQ ID NO: 8, and/or SEQ ID NO: 9, and/or SEQ ID NO: 10, and/or SEQ ID NO: 11, and/or SEQ ID NO: 6. Examples of such antisense compounds are shown in Tables 1, 12 and 13.

In certain embodiments, as described herein, antisense compounds target a CREB nucleic acid having the sequence of GENBANK® Accession No. NM_{—}134442.2 and incorporated herein as SEQ ID NO: 1. In certain such embodiments, as described herein, an antisense oligonucleotide targets SEQ ID NO: 1. In certain such embodiments, as described herein, an antisense oligonucleotide that is targeted to SEQ ID NO: 1 is at least 90% complementary to SEQ ID NO: 1. In certain such embodiments, as described herein, an antisense oligonucleotide that is targeted to SEQ ID NO: 1 is at least 95% complementary to SEQ ID NO: 1. In certain such embodiments, as described herein, an antisense oligonucleotide that is targeted to SEQ ID NO: 1 is 100% complementary to SEQ ID NO: 1. In certain embodiments, as described herein, an antisense oligonucleotide targeted to SEQ ID NO: 1 comprises a nucleotide sequence selected from the nucleotide sequences set forth in Table 12.

### Hybridization

For example, hybridization may occur between an antisense compound disclosed herein, and a CREB nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In one embodiment, the antisense compounds provided herein, are specifically hybridizable with a CREB nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a CREB nucleic acid).

Non-complementary nucleobases between an antisense compound and a CREB nucleic acid may be tolerated provided that the antisense compound remains able to specifically hybridize to a target nucleic acid. Moreover, an antisense compound may hybridize over one or more segments of a CREB nucleic acid such that intervening or adjacent segments are not involved in the hybridization event (e.g., a loop structure, mismatch or hairpin structure).

In some embodiments, as described herein, the antisense compounds provided herein, are at least 70%, at least 75%, at least 80%, at least 85%, at least 90%, at least 95%, at least 96%, at least 97%, at least 98% or at least 99% complementary to a CREB nucleic acid. Percent complementarity of an antisense compound with a target nucleic acid can be determined using routine methods.

In other embodiments, as described herein, the antisense compounds provided herein, are fully complementary (i.e., 100% complementary) to a target nucleic acid. For example, an antisense compound may be fully complementary to a CREB nucleic acid. As used herein, “fully complementary” means each nucleobase of an antisense compound is capable of precise base pairing with the corresponding nucleobases of a target nucleic acid.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In one embodiment, antisense compounds up to 20 nucleobases in length comprise no more than 4, no more than 3, no more than 2 or no more than 1 non-complementary nucleobase(s) relative to a target nucleic acid, such as a CREB nucleic acid.

In another embodiment, antisense compounds up to 30 nucleobases in length comprise no more than 6, no more than 5, no more than 4, no more than 3, no more than 2 or no more than 1 non-complementary nucleobase(s) relative to a target nucleic acid, such as a CREB nucleic acid.

The antisense compounds provided herein, also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In one embodiment, the antisense compounds are complementary to at least an 8 nucleobase portion of a target segment. In another embodiment, the antisense compounds are complementary to at least a 12 nucleobase portion of a target segment. In yet another embodiment, the antisense compounds are complementary to at least a 15 nucleobase portion of a target segment. Also contemplated are antisense compounds that are complementary to at least 9, 10, 11, 12, 13, 14, 15, 16, 17, 18, 19, or 20 or more nucleobase portion of a target segment, or a range defined by any two or more target segments.

In certain embodiments, as described herein, an antisense compounds provided herein include those comprising a portion which consists of at least 8, 9, 10, 11, 12, 13, 14, 15, 16, 17, 18, 19, 20, or 21 contiguous nucleobases of the nucleobase sequence as set forth in SEQ ID NOs: 2, 3, 4, 5, 6, 7, 8, 9, 10, 11, 12, 13, 14, 15, 16, and 17. In certain embodiments, as described herein, the antisense compounds are complementary to an equal-length portion of SEQ ID NOS: 1-12. In certain embodiments, as described herein, the antisense compounds are at least 75%, 80%, 85%, 90%, 95%, or 100% complementary to SEQ ID NOS: 1-12.

For example, antisense oligonucleotides with the following ISIS Nos exhibited at least 50% inhibition of CREB mRNA levels: 102713, 102631, 102643, 102666, 102746, 102639, 102685, 102709, 102743, 102678, 102654, 102726, 102705, 102737, 102717, 102650, 102674, 102702, 102658, 102729, 102734, 102647, 102697, 102670, and 102689. The target segments to which these antisense oligonucleotides are targeted are active target segments. The target regions to which these antisense oligonucleotides are targeted are active target regions. Accordingly, in some embodiments, as described herein an antisense compound as provided herein may include at least 8, 9, 10, 11, 12, 13, 14, 15, 16, 17, 18, 19, 20, or 21 contiguous nucleobases of the nucleobase sequence as set forth in SEQ ID NOs: 92, 93, 95, 97, 98, 99, 100, 101, 102, 104, 105, 106, 107, 109, 110, 111, 112, 113, 114, 115, 116, 118, 119, 121, and 122.

Antisense oligonucleotides with the following ISIS Nos exhibited at least 60% inhibition of CREB mRNA levels: 102705, 102737, 102717, 102650, 102674, 102702, 102658, 102729, 102734, 102647, 102697, 102670, or 102689. The target segments to which these antisense oligonucleotides are targeted are active target segments. The target regions to which these antisense oligonucleotides are targeted are active target regions. Accordingly, in some embodiments, as described herein an antisense compound as provided herein may include at least 8, 9, 10, 11, 12, 13, 14, 15, 16, 17, 18, 19, 20, or 21 contiguous nucleobases of the nucleobase sequence as set forth in SEQ ID NOs: 113, 119, 116, 100, 106, 112, 102, 92, 93, 99, 111, 105, or 110.

Antisense oligonucleotides with the following ISIS Nos exhibited at least 65% inhibition of CREB mRNA levels: 102717, 102650, 102674, 102702, 102658, 102729, 102734, 102647, 102697, 102670, or 102689. The target segments to which these antisense oligonucleotides are targeted are active target segments. The target regions to which these antisense oligonucleotides are targeted are active target regions. Accordingly, in some embodiments, as described herein an antisense compound as provided herein may include at least 8, 9, 10, 11, 12, 13, 14, 15, 16, 17, 18, 19, 20, or 21 contiguous nucleobases of the nucleobase sequence as set forth in SEQ ID NOs: 116, 100, 106, 112, 102, 92, 93, 99, 111, 105, or 110

Antisense oligonucleotides with the following ISIS Nos exhibited at least 70% inhibition of CREB mRNA levels: 102647, 102697, 102670, or 102689. The target segments to which these antisense oligonucleotides are targeted are active target segments. The target regions to which these antisense oligonucleotides are targeted are active target regions. Accordingly, in some embodiments, as described herein an antisense compound as provided herein may include at least 8, 9, 10, 11, 12, 13, 14, 15, 16, 17, 18, 19, 20, or 21 contiguous nucleobases of the nucleobase sequence as set forth in SEQ ID NOs: 99, 111, 105, or 110.

ISIS Nos 102697, 102670, and 102689 each exhibited at least 75% inhibition of CREB mRNA levels. The target segments to which these antisense oligonucleotides are targeted are active target segments. The target regions to which these antisense oligonucleotides are targeted are active target regions. Accordingly, in some embodiments, as described herein an antisense compound as provided herein may include at least 8, 9, 10, 11, 12, 13, 14, 15, 16, 17, 18, 19, 20, or 21 contiguous nucleobases of the nucleobase sequence as set forth in SEQ ID NO: 111, 105, or 110.

### Identity

The antisense compounds provided herein, may also have a defined percent identity to a particular nucleotide sequence, SEQ ID NO, or compound represented by a specific Isis number. As used herein, an antisense compound is identical to the sequence disclosed herein, if it has the same nucleobase pairing ability. For example, an RNA which contains uracil in place of thymidine in a disclosed DNA sequence would be considered identical to the DNA sequence since both uracil and thymidine pair with adenine. Shortened and lengthened versions of the antisense compounds described herein, as well as compounds having non-identical bases relative to the antisense compounds provided herein, also are contemplated. The non-identical bases may be adjacent to each other or dispersed throughout the antisense compound. Percent identity of an antisense compound is calculated according to the number of bases that have identical base pairing relative to the sequence to which it is being compared.

In one embodiment, the antisense compounds are at least 70%, at least 75%, at least 80%, at least 85%, at least 90%, at least 95% or 100% identical to one or more of the antisense compounds disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties, such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus-containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In one embodiment, antisense compounds targeted to CREB nucleic acid comprise one or more modified internucleoside linkages. In some embodiments, as described herein, the modified internucleoside linkages are phosphorothioate linkages. In other embodiments, as described herein, each internucleoside linkage of an antisense compound is a phosphorothioate internucleoside linkage.

### Modified Sugar Moieties

Antisense compounds targeted to a CREB nucleic acid may contain one or more nucleotides having modified sugar moieties. Sugar modifications may impart nuclease stability, binding affinity or some other beneficial biological property to the antisense compounds. The furanosyl sugar ring of a nucleoside can be modified in a number of ways including, but not limited to, addition of a substituent group, particularly at the 2′ position; bridging of two non-geminal ring atoms to form a bicyclic nucleic acid (BNA); and substitution of an atom or group, such as —S—, —N(R)— or —C(R₁)(R₂) for the ring oxygen at the 4′-position. Modified sugars include, but are not limited to, substituted sugars, especially 2′-substituted sugars having a 2′-F, 2′-OCH₂ (2′-OMe) or a 2′-O(CH₂)₂—OCH₃ (2′-O-methoxyethyl or 2′-MOE) substituent group; and bicyclic modified sugars (BNAs), having a 4′-(CH₂)ₙ—O-2′ bridge, where n=1 or n=2, including α-L-Methyleneoxy (4′-CH2-O-2′) BNA, β-D-Methyleneoxy (4′-CH2-O-2′) BNA and Ethyleneoxy (4′-(CH2)₂—O-2′) BNA. Bicyclic modified sugars also include (6′S)-6′ methyl BNA, Aminooxy (4′-CH2-O—N(R)-2′) BNA, Oxyamino (4′-CH2-N(R)—O-2′) BNA wherein, R is, independently, H, a protecting group, or C1-C10 alkyl. The substituent at the 2′ position can also be selected from alkyl, amino, azido, thio, O-allyl, O—C1-C10 alkyl, OCF₃, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C1-C10 alkyl. In certain embodiments, as described herein, such BNA-modified nucleotides are high-affinity nucleotides and their incorporation into antisense compounds allows for increased potency and improved therapeutic index. Methods for the preparation of modified sugars are well known to those skilled in the art.

Examples of nucleosides having modified sugar moieties include without limitation nucleosides comprising 5′-vinyl, 5′-methyl (R or S), 4′-S, 2′-F, 2′-OCH₃ and 2′-O(CH₂)₂OCH₃ substituent groups. The substituent at the 2′ position can also be selected from allyl, amino, azido, thio, O-allyl, O—C1-C10 alkyl, OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

Examples of bicyclic nucleic acids (BNAs) include without limitation nucleosides comprising a bridge between the 4′ and the 2′ ribosyl ring atoms. In certain embodiments, as described herein, antisense compounds provided herein include one or more BNA nucleosides wherein the bridge comprises one of the formulas: 4′-(CH₂)—O-2′ (LNA); 4′-(CH₂)—S-2; 4′-(CH₂)—O-2′ (LNA); 4′-(CH₂)₂—O-2′ (ENA); 4′-C(CH₃)₂—O-2′ (see PCT/US2008/068922); 4′-CH(CH₃)—O-2′ and 4′-CH(CH₂OCH₃)—O-2′ (see U.S. Pat. No. 7,399,845, issued on Jul. 15, 2008); 4′-CH₂—N(OCH₃)-2′ (see PCT/US2008/064591); 4′-CH₂—O—N(CH₃)-2′ (see published U.S. Patent Application US2004-0171570, published Sep. 2, 2004); 4′-CH₂—N(R)—O-2′ (see U.S. Pat. No. 7,427,672, issued on Sep. 23, 2008); 4′-CH₂—C(CH₃)-2′ and 4′-CH₂—C(═CH₂)-2′ (see PCT/US2008/066154); and wherein R is, independently, H, C₁-C₁₂ alkyl, or a protecting group. Each of the foregoing BNAs include various stereochemical sugar configurations including for example α-L-ribofuranose and β-D-ribofuranose (see PCT international application PCT/DK98/00393, published on Mar. 25, 1999 as WO 99/14226).

In certain embodiments, as described herein, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring such as one having one of the formula:

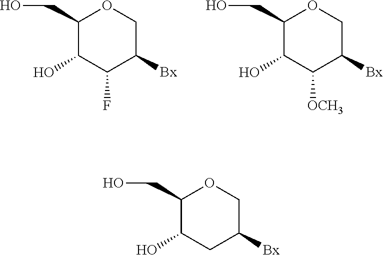

Many other bicyclo and tricyclo sugar surrogate ring systems are also know in the art that can be used to modify nucleosides for incorporation into antisense compounds (see for example review article: Leumann, Christian J.). Such ring systems can undergo various additional substitutions to enhance activity.

In nucleotides having modified sugar moieties, the nucleobase moieties (natural, modified or a combination thereof) are maintained for hybridization with an appropriate nucleic acid target.

In one embodiment, antisense compounds targeted to CREB nucleic acid comprise one or more nucleotides having modified sugar moieties. In a suitable embodiment, the modified sugar moiety is 2′-MOE. In other embodiments, as described herein, the 2′-MOE modified nucleotides are arranged in a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been described to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example, 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In one embodiment, antisense compounds targeted to a CREB nucleic acid comprise one or more modified nucleobases. In an additional embodiment, gap-widened antisense oligonucleotides targeted to CREB nucleic acid comprise one or more modified nucleobases. In some embodiments, as described herein, the modified nucleobase is 5-methylcytosine. In further embodiments, as described herein, each cytosine is a 5-methylcytosine.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties, such as nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of CREB nucleic acids can be tested in vitro in a variety of cell types. Cell types used for such analyses are available from commercial vendors (e.g. American Type Culture Collection, Manassas, Va.; Zen-Bio, Inc., Research Triangle Park, NC; Clonetics Corporation, Walkersville, Md.) and cells are cultured according to the vendor's instructions using commercially available reagents (e.g. Invitrogen Life Technologies, Carlsbad, Calif.). Illustrative cell types include, but are not limited to, HepG2 cells, HepB3 cells, and primary hepatocytes.

### In Vitro Testing of Antisense Oligonucleotides

Described herein, are methods for treatment of cells with antisense oligonucleotides, which can be modified appropriately for treatment with other antisense compounds.

In general, cells are treated with antisense oligonucleotides when the cells reach approximately 60-80% confluency in culture.

One reagent commonly used to introduce antisense oligonucleotides into cultured cells includes the cationic lipid transfection reagent LIPOFECTIN® (Invitrogen, Carlsbad, Calif.). Antisense oligonucleotides are mixed with LIPOFECTIN® in OPTI-MEM® 1 (Invitrogen, Carlsbad, Calif.) to achieve the desired final concentration of antisense oligonucleotide and a LIPOFECTIN® concentration that typically ranges 2 to 12 ug/mL per 100 nM antisense oligonucleotide.

Another reagent used to introduce antisense oligonucleotides into cultured cells includes LIPOFECTAMINE® (Invitrogen, Carlsbad, Calif.). Antisense oligonucleotides are mixed with LIPOFECTAMINE® in OPTI-MEM®1 reduced serum medium (Invitrogen, Carlsbad, Calif.) to achieve the desired concentration of antisense oligonucleotide and a LIPOFECTAMINE® concentration that typically ranges 2 to 12 ug/mL per 100 nM antisense oligonucleotide.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in replicates, the data are presented as the average of the replicate treatments.

The concentration of antisense oligonucleotides used varies from cell line to cell line. Methods to determine the optimal antisense oligonucleotide concentration for a particular cell line are well known in the art. Antisense oligonucleotides are typically used at concentrations ranging from 1 nM to 300 nM.

### RNA Isolation

RNA analysis can be performed on total cellular RNA or poly(A)+ mRNA. RNA is prepared using methods well known in the art, for example, using the TRIZOL® Reagent (Invitrogen, Carlsbad, Calif.), according to the manufacturer's recommended protocols.

### Analysis of Inhibition of Target Levels or Expression

Inhibition of the level or expression of CREB nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM® 7600, 7700, or 7900 Sequence Detection System (PE-Applied Biosystems, Foster City, Calif.), according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT, real-time-PCR reactions are carried out by methods well known to those skilled in the art.

Gene (or RNA) target quantities obtained by real time PCR are normalized using either the expression level of a gene whose expression is constant, such as GAPDH, or by quantifying total RNA using RIBOGREEN® (Invitrogen, Inc., Carlsbad, Calif.). GAPDH expression is quantified by real time PCR, by being run simultaneously with the target, multiplexing, or separately. Methods of RNA quantification by RIBOGREEN® are mentioned in Jones, L. J., et al, (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to CREB nucleic acid. Methods for designing real-time PCR probes and primers are well known in the art, and may include the use of software such as PRIMER EXPRESS® Software (Applied Biosystems, Foster City, Calif.).

### Analysis of Protein Levels

Antisense inhibition of CREB nucleic acids can be assessed by measuring CREB protein levels. Protein levels of CREB can be evaluated or quantitated in a variety of ways well known in the art, such as immunoprecipitation, Western blot analysis (immunoblotting), enzyme-linked immunosorbent assay (ELISA), quantitative protein assays, protein activity assays (e.g., caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Antibodies for the detection of human and rat CREB are commercially available.

### In Vivo Testing of Antisense Compounds

Antisense compounds, for example, antisense oligonucleotides, are tested in animals to assess their ability to inhibit expression of CREB and produce phenotypic changes, such as decreases in cholesterol. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, or subcutaneous. Calculation of antisense oligonucleotide dosage and dosing frequency is within the abilities of those skilled in the art, and depends upon factors such as route of administration and animal body weight. Following a period of treatment with antisense oligonucleotides, RNA is isolated from various tissues and changes in CREB nucleic acid expression are measured. Changes in CREB protein levels may also be measured.

### Combination Therapy

The invention also provides methods of combination therapy, wherein, one or more CREB-specific modulators of the invention and one or more other therapeutic/prophylactic agents are administered treat a condition and/or disease state as described herein. In various aspects, CREB-specific modulator(s) of the invention and the therapeutic/prophylactic agent (s) are co-administered as a mixture or administered subjectly. In one aspect, the route of administration is the same for the CREB-specific modulators (s) of the invention and the therapeutic/prophylactic agent(s), while in other aspects, the CREB-specific modulators (s) of the invention and the therapeutic/prophylactic agents (s) are administered by different routes. In one embodiment, the dosages of the CREB-specific modulator(s) of the invention and the therapeutic/prophylactic agent(s) are amounts that are therapeutically or prophylactically effective for each CREB-specific modulator when administered subjectly. Alternatively, the combined administration permits use of lower dosages than would be required to achieve a therapeutic or prophylactic effect if administered subjectly, and such methods are useful in decreasing one or more side effects of the reduced-dose CREB-specific modulator.

In certain embodiments, as described herein, one or more CREB-specific modulators are co-administered with one or more other CREB-specific modulators. In certain embodiments, as described herein, such one or more other CREB-specific modulators are designed to treat the same disease or condition as the one or more pharmaceutical compositions of the present invention. In certain embodiments, as described herein, such one or more other CREB-specific modulators are designed to treat a different disease or condition as the one or more pharmaceutical compositions of the present invention. In certain embodiments, as described herein, such one or more other CREB-specific modulators are designed to treat an undesired effect of one or more pharmaceutical compositions of the present invention. In certain embodiments, as described herein, one or more pharmaceutical compositions are co-administered with another CREB-specific modulator to treat an undesired effect of that other CREB-specific modulator. In certain embodiments, as described herein, one or more CREB-specific modulators of the present invention and one or more other CREB-specific modulators are administered at the same time. In certain embodiments, as described herein, one or more pharmaceutical compositions of the present invention and one or more other CREB-specific modulators are administered at different times. In certain embodiments, as described herein, one or more pharmaceutical compositions of the present invention and one or more other CREB-specific modulators are prepared together in a single formulation. In certain embodiments, as described herein, one or more pharmaceutical compositions of the present invention and one or more other CREB-specific modulators are prepared separately.

In certain embodiments, as described herein, CREB-specific modulators that may be co-administered with a pharmaceutical composition comprising a CREB-specific antisense compound include glucose-lowering agents and therapies. In some embodiments, as described herein, the glucose-lowering agent is a PPAR agonist (gamma, dual, or pan), a dipeptidyl peptidase (IV) inhibitor, a GLP-1 analog, insulin or an insulin analog, an insulin secretagogue, a SGLT2 inhibitor, a human amylin analog, a biguanide, an alpha-glucosidase inhibitor, a meglitinide, a thiazolidinedione, or a sulfonylurea.

In some embodiments, as described herein, the glucose-lowering therapeutic is a GLP-1 analog. In some embodiments, as described herein, the GLP-1 analog is exendin-4 or liraglutide.

In other embodiments, as described herein, the glucose-lowering therapeutic is a sulfonylurea. In some embodiments, as described herein, the sulfonylurea is acetohexamide, chlorpropamide, tolbutamide, tolazamide, glimepiride, a glipizide, a glyburide, or a gliclazide.

In some embodiments, as described herein, the glucose lowering drug is a biguanide. In some embodiments, as described herein, the biguanide is metformin, and in some embodiments, as described herein, blood glucose levels are decreased without increased lactic acidosis as compared to the lactic acidosis observed after treatment with metformin alone.

In some embodiments, as described herein, the glucose lowering drug is a meglitinide. In some embodiments, as described herein, the meglitinide is nateglinide or repaglinide.

In some embodiments, as described herein, the glucose-lowering drug is a thiazolidinedione. In some embodiments, as described herein, the thiazolidinedione is pioglitazone, rosiglitazone, or troglitazone.

In some embodiments, as described herein, blood glucose levels are decreased without greater weight gain than observed with rosiglitazone treatment alone.

In some embodiments, as described herein, the glucose-lowering drug is an alpha-glucosidase inhibitor. In some embodiments, as described herein, the alpha-glucosidase inhibitor is acarbose or miglitol.

In a certain embodiment, a co-administered glucose-lowering agent is an antisense oligonucleotide targeted to CREB.

In a certain embodiment, glucose-lowering therapy is therapeutic lifestyle change.

In certain such embodiments, as described herein, the glucose-lowering agent is administered prior to administration of a pharmaceutical composition of the present invention. In certain such embodiments, as described herein, the glucose-lowering agent is administered following administration of a pharmaceutical composition of the present invention. In certain such embodiments, as described herein the glucose-lowering agent is administered at the same time as a pharmaceutical composition of the present invention. In certain such embodiments, as described herein the dose of a co-administered glucose-lowering agent is the same as the dose that would be administered if the glucose-lowering agent was administered alone. In certain such embodiments, as described herein the dose of a co-administered glucose-lowering agent is lower than the dose that would be administered if the glucose-lowering agent was administered alone. In certain such embodiments, as described herein the dose of a co-administered glucose-lowering agent is greater than the dose that would be administered if the glucose-lowering agent was administered alone.

In certain embodiments, as described herein, CREB-specific modulators that may be co-administered with a pharmaceutical composition comprising a CREB-specific antisense compound include anti-obesity agents. Such anti-obesity agents therapeutics may be administered as described above for glucose lowering agents.

Further provided is a method of administering a CREB-specific antisense compound via injection and further including administering a topical steroid at the injection site.

In certain embodiments, as described herein, CREB-specific modulators that may be co-administered with a pharmaceutical composition of the present invention include lipid-lowering agents. In certain such embodiments, as described herein, CREB-specific modulators that may be co-administered with a pharmaceutical composition of the present invention include, but are not limited to atorvastatin, simvastatin, rosuvastatin, and ezetimibe. In certain such embodiments, as described herein, the lipid-lowering agent is administered prior to administration of a pharmaceutical composition of the present invention. In certain such embodiments, as described herein, the lipid-lowering agent is administered following administration of a pharmaceutical composition of the present invention. In certain such embodiments, as described herein the lipid-lowering agent is administered at the same time as a pharmaceutical composition of the present invention. In certain such embodiments, as described herein the dose of a co-administered lipid-lowering agent is the same as the dose that would be administered if the lipid-lowering agent was administered alone. In certain such embodiments, as described herein the dose of a co-administered lipid-lowering agent is lower than the dose that would be administered if the lipid-lowering agent was administered alone. In certain such embodiments, as described herein the dose of a co-administered lipid-lowering agent is greater than the dose that would be administered if the lipid-lowering agent was administered alone.

In certain embodiments, as described herein, a co-administered lipid-lowering agent is a HMG-CoA reductase inhibitor. In certain such embodiments, as described herein the HMG-CoA reductase inhibitor is a statin. In certain such embodiments, as described herein the statin is selected from atorvastatin, simvastatin, pravastatin, fluvastatin, and rosuvastatin.

In certain embodiments, as described herein, a co-administered lipid-lowering agent is a cholesterol absorption inhibitor. In certain such embodiments, as described herein, cholesterol absorption inhibitor is ezetimibe.

In certain embodiments, as described herein, a co-administered lipid-lowering agent is a co-formulated HMG-CoA reductase inhibitor and cholesterol absorption inhibitor. In certain such embodiments, as described herein the co-formulated lipid-lowering agent is ezetimibe/simvastatin.

In certain embodiments, as described herein, a co-administered lipid-lowering agent is a microsomal triglyceride transfer protein inhibitor (MTP inhibitor).

In certain embodiments, as described herein, a co-administered lipid-lowering agent is an oligonucleotide targeted to ApoB.

In certain embodiments, as described herein, a co-administered CREB-specific modulator is a bile acid sequestrant. In certain such embodiments, as described herein, the bile acid sequestrant is selected from cholestyramine, colestipol, and colesevelam.

In certain embodiments, as described herein, a co-administered CREB-specific modulator is a nicotinic acid. In certain such embodiments, as described herein, the nicotinic acid is selected from immediate release nicotinic acid, extended release nicotinic acid, and sustained release nicotinic acid.

In certain embodiments, as described herein, a co-administered CREB-specific modulator is a fibric acid. In certain such embodiments, as described herein, a fibric acid is selected from gemfibrozil, fenofibrate, clofibrate, bezafibrate, and ciprofibrate.

Further examples of CREB-specific modulators that may be co-administered with a pharmaceutical composition of the present invention include, but are not limited to, corticosteroids, including but not limited to prednisone; immunoglobulins, including, but not limited to intravenous immunoglobulin (IVIg); analgesics (e.g., acetaminophen); anti-inflammatory agents, including, but not limited to non-steroidal anti-inflammatory drugs (e.g., ibuprofen, COX-1 inhibitors, and COX-2, inhibitors); salicylates; antibiotics; antivirals; antifungal agents; antidiabetic agents (e.g., biguanides, glucosidase inhibitors, insulins, sulfonylureas, and thiazolidenediones); adrenergic modifiers; diuretics; hormones (e.g., anabolic steroids, androgen, estrogen, calcitonin, progestin, somatostan, and thyroid hormones); immunomodulators; muscle relaxants; antihistamines; osteoporosis agents (e.g., biphosphonates, calcitonin, and estrogens); prostaglandins, antineoplastic agents; psychotherapeutic agents; sedatives; poison oak or poison sumac products; antibodies; and vaccines.

In certain embodiments, as described herein, the pharmaceutical compositions of the present invention may be administered in conjunction with a lipid-lowering therapy. In certain such embodiments, as described herein, a lipid-lowering therapy is therapeutic lifestyle change. In certain such embodiments, as described herein, a lipid-lowering therapy is LDL apheresis.

### Glucose-Lowering Drugs/Agents/Therapeutics, Anti-Obesity Drugs/Agents/Therapeutics, Lipid-Lowering Drugs/Agents/Therapeutics

Compounds of the invention may be used in combination therapies, wherein, an additive effect is achieved by administering one or more compounds of the invention and one or more other suitable therapeutic/prophylactic compounds to treat a condition. Suitable therapeutic/prophylactic compound(s) include, but are not limited to, glucose-lowering agents (also referred to herein, as glucose-lowering drugs or glucose-lowering therapeutics), anti-obesity agents (also referred to herein, as anti-obesity drugs or anti-obesity therapeutics), and lipid lowering agents (also referred to herein, as lipid-lowering drugs or lipid-lowering therapeutics). Glucose lowering agents include, but are not limited to, PPAR agonists, dipeptidyl peptidase (IV) inhibitors, GLP-1 analogs, insulin or insulin analogs, insulin secretagogues, SGLT2 inhibitors, human amylin analogs, biguanides, or alpha-glucosidase inhibitors. Glucose lowering agents include, but are not limited to hormones, hormone mimetics, or incretin mimetics (e.g., insulin, including inhaled insulin, GLP-1 or GLP-1 analogs such as liraglutide, or exenatide), DPP(IV) inhibitors, a sulfonylurea (e.g., acetohexamide, chlorpropamide, tolbutamide, tolazamide, glimepiride, a glipizide, glyburide or a gliclazide), a biguanide (metformin), a meglitinide (e.g., nateglinide or repaglinide), a thiazolidinedione or other PPAR-gamma agonists (e.g., pioglitazone or rosiglitazone) an alpha-glucosidase inhibitor (e.g., acarbose or miglitol), antisense compounds targeted to CREB, or and antisense compound not targeted to CREB. Also included are dual PPAR-agonists (e.g., muraglitazar, being developed by Bristol-Myers Squibb, or tesaglitazar, being developed by Astra-Zeneca). Also included are other diabetes treatments in development (e.g. LAF237, being developed by Novartis; MK-0431, being developed by Merck; or rimonabant, being developed by Sanofi-Aventis). Also included are GLP-1 mimetics in development, including, but not limited to, those being developed by Roche, ConjuChem, Sanofi-Aventis, Teijin Pharma Limited, Ipsen Pharmaceuticals, and Servier Research Institute. Also included are SGLT2 inhibitors in development, including, but not limited to, those being developed by Glaxo Smith Kline or AVE2268 in development at Sanofi-Aventis. Also included are DPP(IV) inhibitors in development, including, but not limited to, those being developed by Novartis (e.g. vildagliptin), Merck, GSK, or BMS. Also included are glucokinase inhibitors in development. Anti-obesity agents include, but are not limited to, appetite suppressants (e.g. phentermine or Meridia™), fat absorption inhibitors such as orlistat (e.g. Xenical™), and modified forms of ciliary neurotrophic factor which inhibit hunger signals that stimulate appetite. Anti-obesity agents include peripheral or CNS-based agents. Lipid lowering agents include, but are not limited to, bile salt sequestering resins (e.g., cholestyramine, colestipol, and colesevelam hydrochloride), HMGCoA-reductase inhibitors (e.g., lovastatin, pravastatin, atorvastatin, simvastatin, and fluvastatin), nicotinic acid, fibric acid derivatives (e.g., clofibrate, gemfibrozil, fenofibrate, bezafibrate, and ciprofibrate), probucol, neomycin, dextrothyroxine, plant-stanol esters, cholesterol absorption inhibitors (e.g., ezetimibe), CETP inhibitors (e.g. torcetrapib, and JTT-705) MTP inhibitors (e.g., implitapide), inhibitors of bile acid transporters (apical sodium-dependent bile acid transporters), regulators of hepatic CYP7a, ACAT inhibitors (e.g. Avasimibe), estrogen replacement therapeutics (e.g., tamoxigen), synthetic HDL (e.g. ETC-216), anti-inflammatories (e.g., glucocorticoids), antisense compounds targeted to CREB, or an antisense compound not targeted to CREB. One or more of these drugs may be combined with one or more of the CREB-specific modulator inhibitors to achieve an additive therapeutic effect.

Diabetes agents, including insulin, other hormones and hormone analogs and mimetics, and other glucose lowering agents, including orally administered glucose lowering drugs, may also be combined with antisense inhibitors of CREB. The term “glucose-lowering agent” includes, but is not limited to, the sulfonylureas, biguanides, meglitinides, peroxisome proliferator-activated receptor-gamma (PPAR-gamma) agonists (e.g., thiazolidinediones) and alpha-glucosidase inhibitors.

Sulfonylureas work by stimulating beta-cell insulin secretion in the pancreas, and may also improve insulin sensitivity in peripheral tissues. Early sulfonylureas such as acetohexamide (Dymelor™), chlorpropamide (Diabinese™, Glucamide™), tolbutamide (Orinase™, Mobenol™), and tolazamide (Tolamide™, Tolinase™) have been generally replaced with newer sulfonureas with better side-effect profiles (specifically lower cardiovascular risk), such as glimepiride (Amaryl™), glipizide (Glucotrol™), glipizide extended release (Glucotrol XL™), glyburide (Micronase™, Euglucon™, Diabeta™), gliclazide (Diamicron™, and micronized glyburide (Glynase™) (Luna & Feinglos; AACE et al., 2002). Side effects of sulfonylureas include hypoglycemia and weight gain.

Biguanides such as Metformin (Glucophage™) work by decreasing hepatic glucose output and enhancing insulin sensitivity in hepatic and peripheral tissues. Metformin is contraindicated in patients with congestive heart failure or severe liver disease.

Meglitinides work by stimulating the beta cells in the pancreas to produce insulin Nateglinide (Starlix™) and repaglinide (Prandin™) are examples of this class.

Peroxisome proliferator-activated receptor-gamma (PPAR-gamma) agonists such as the thiazolidinediones enhance insulin sensitivity in muscle and adipose tissue and, to a lesser extent, inhibit hepatic glucose production. Thiazolidinediones include pioglitazone (Actos™) and rosiglitazone (Avandia™; GlaxoSmithKline). The first thiazolidinedione approved for use in the United States, troglitazone (Rezulin™), was withdrawn from the market because of severe liver toxicity. Thiazolidinediones also affect the lipid profiles of patients with type 2 diabetes. Studies have described that rosiglitazone is associated with increases in total, LDL, and HDL cholesterol levels, and either no changes or increases in triglyceride levels. Pioglitazone has been associated with mean decreases in triglyceride levels, mean increases in HDL cholesterol levels, and no consistent mean changes in LDL and cholesterol levels. Other potential side effects associated with thiazolidinediones include weight gain, slow onset of action, and potential liver toxicity (Luna & Feinglos, 2001).

New PPAR-gamma agonists are being developed; these include isaglitazone (netoglitazone) and the dual-acting PPAR agonists which have affinities for both PPAR-gamma and PPAR-alpha. Examples of dual-acting PPAR agonists are BMS-298585 and tesaglitazar. Agonists of other PPARs (e.g., alpha, delta) or pan-PPAR agonists may also be useful.

Alpha-glucosidase inhibitors inhibit an enzyme found in the lining of the small intestine that is responsible for the breakdown of complex carbohydrates before they are absorbed. Such inhibitors include acarbose (Precose™) and miglitol (Glyset™).

Oral glucose-lowering drugs are often used in combination to treat Type 2 diabetes. While many combinations of the above are possible, several are already marketed as a combined formulation (for example, Avandamet™ (Rosiglitazone+Metformin); Glucovance™ (glyburide/metformin); and Metaglip™ (glipizide/metformin). These and other combined formulations for treatment of diabetes or obesity may be administered in combination with one or more of the CREB-specific modulator inhibitors.

Other classes of glucose-lowering, diabetes drugs are being developed. As alternatives to regular insulin, which is administered by injection, insulin analogs such as insulin lispro (Humalog™) and insulin glargine (Lantus™) may be used. Both are given by injection as is regular insulin, but result in fewer hypoglycemic events than regular insulin. In addition the onset and duration of action with these is different from regular insulin. A follow-up analog to insulin glargine, insulin glulisine, is being developed by Aventis. Novo Nordisk is developing insulin detemir, a long-acting analog.

Alternative formulations/delivery methods for regular insulin are also being developed. Both liquid and dry powder inhaled insulin formulations are currently in late-stage development or have been recently approved—examples include recently approved Exubera™ (Nektar/Pfizer/Aventis), which is a powder, and AERx™ (Aradigm/Novo Nordisk), which is an aerosolized liquid. While inhaled insulin is expected to be viewed as more convenient and less invasive than injected insulin, the cost is expected to be much greater for inhaled insulin.

Several companies are developing oral formulations of insulin. Oralin™ (Generex Biotechnology) is the farthest along in development but there are others.

Other hormones and hormone mimetics being developed include pramlintide acetate (Symlin™), and GLP-1. GLP-1 receptor agonists and GLP-1 analogs are being evaluated for clinical use as antidiabetic agents. GLP-1 itself has a short half-life due to N-terminal degradation of the peptide by Dipeptidyl Peptidase (DPP-IV)-mediated cleavage at the position 2 alanine. This limits the clinical usefulness of native GLP-1 or synthetic versions thereof. Longer acting analogs have been developed, including Exendin-4 (Exenatide™, Exenatide LAR™), a DP IV-resistant GLP-1 analog and Liraglutide™, an acylated albumin-bound human GLP-1 analog.

DPP-IV inhibitors are also being explored as drugs and one (LAF-237, Novartis) is currently in advanced clinical trials. Glucagon inhibitors may also be useful for diabetes.

Other peptides such as pituitary adenylate cyclase-activating polypeptide (PACAP) and Peptide YY (PYY) (and its subpeptide PYY[3-36]) are also under study for diabetes and/or obesity (Yamamoto et al., 2003, Diabetes 52, 1155-1162; Pittner et al., Int. J. Obes. Relat. Metab. Disord. 2004, 28, 963-71).

Any of the aforementioned glucose-lowering drugs are useful in combination with one or more of the CREB-specific modulator inhibitors, such as a antisense inhibitor of CREB as described herein. One or more of these drugs may be combined in a single composition with one or more of the CREB-specific modulator inhibitors, or used in therapies for combined administration, i.e., sequential or concurrent administration thereof.

Antisense inhibition of CREB is described herein, below to reduce weight gain of animals on high-fat diets and may be useful in treatment of obesity. The use of weight loss agents has also been considered useful in diabetes management in general and for delaying or preventing the development or progression of frank Type 2 diabetes in patients with impaired glucose tolerance (Heymsfield S B, 2000, Archives of Internal Medicine, 160, 1321-1326). Thus, anti-obesity drugs are useful in combination with antisense inhibitors of CREB expression in pharmaceutical compositions or in combined therapeutic regimens. Examples of anti-obesity drugs (also called “diet drugs”) include, without limitation, appetite suppressants such as phentermine and Meridia™, fat absorption inhibitors or lipase inhibitors such as orlistat (Xenical™), and Axokine™, a modified form of ciliary neurotrophic factor, which inhibits hunger signals that stimulate appetite, CB-1 selective agonists such as Rimonabant, 5HT2c agonists, amylin analogues such as pramlintide, sibutramine, GLP-1 agonists such as Byetta. Other drugs or classes of drugs under evaluation for obesity are CBI inverse agonists, PYY, MCH4 and MTP inhibitors.

In certain embodiments, additional therapies or therapeutic agents may also include, for example, but are not limited to, insulin and insulin analogues; insulin secretagogues including sulphonylureas (for example glibenclamide, glipizide) and prandial glucose regulators (for example repaglinide, nateglinide); insulin sensitising agents including PPARg agonists (for example pioglitazone and rosiglitazone); agents that suppress hepatic glucose output (for example metformin); agents designed to reduce the absorption of glucose from the intestine (for example acarbose); agents designed to treat the complications of prolonged hyperglycemia; anti-obesity agents (for example sibutramine, orlistat, aP2 inhibitors (such as those disclosed in U.S. Ser. No. 09/519,079 filed Mar. 6, 2000); melanocortin receptor (MC4R) agonist, a melanin-concentrating hormone receptor (MCHR) antagonist, a growth hormone secretagogue receptor (GHSR) antagonist, an orexin receptor antagonist, a CCK (cholecystokinin) agonist, a GLP-1 agonists, NPY1 or NPY5 antagonist, a corticotropin releasing factor (CRF) antagonist, a histamine receptor-3 (H3) modulator, a PPAR.gamma. modulator, a PPAR.delta. modulator, a beta 3 adrenergic agonist, a lipase inhibitor, a serotonin (and dopamine) reuptake inhibitor, a erotonin receptor agonist (e.g. BVT-933), an aP2 inhibitor, a thyroid receptor agonist and/or an anorectic agent, anti-dyslipidaemia agents such as, HMG-CoA reductase inhibitors (statins, e.g. pravastatin); PPAR.alpha. agonists (fibrates, e.g. gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations); Antihypertensive agents such as, .beta. blockers (e.g. atenolol, inderal); ACE inhibitors (e.g. lisinopril); calcium antagonists (e.g., nifedipine); angiotensin receptor antagonists (e.g. candesartan), a antagonists and diuretic agents (e.g. furosemide, benzthiazide); Homeostasis modulators such as, antithrombotics, activators of fibrinolysis and antiplatelet agents; thrombin antagonists; factor Xa inhibitors; factor VIIa inhibitors); antiplatelet agents (e.g. aspirin, clopidogrel); anticoagulants (heparin and Low molecular weight analogues, hirudin) and warfarin; and Anti-inflammatory agents, such as non-steroidal anti-inflamatory drugs (e.g. aspirin) and steroidal anti-inflammatory agents (e.g. cortisone).

In certain embodiments, combination therapies with additional therapies, as provided herein, may be combined into a single composition or kept as separate compositions.

In certain embodiments, CREB-specific modulators or inhibitors may be administered at the same time or at different times with combined additional therapies or therapeutic agents. Examples of combined additional therapies or therapeutic agents that can be administered with a CREB-specific modulator or inhibitor include, without limitation, Avandamet (GlaxoSmithKline) a combination of Rosiglitazone and Metformin, Glucovance (Bristol-Myers Squibb) a combination of Metformin and Glyburide, Metaglip (Bristol-Myers Squibb) a combination of Metformin and Glipizide, Duetact (Takeda) a combination of Pioglitazone and Glimepirid, Janumet (Merck) a combination of Sitagliptin and Metformin HCl, or ACTOplus met (Takeda) a combination of Metformin and pioglitazone.

Any of the aforementioned is useful in combination with one or more of the CREB-specific modulator or inhibitor, such as any antisense inhibitor of CREB according to this invention. Combined compounds (two or more) may be used together or sequentially.

### Cholesterol-Lowering Drugs/Agents/Therapeutics and Triglyceride-Lowering Drugs/Agents/Therapeutics

The invention also provides methods of combination therapy, wherein, one or more compounds of the invention and one or more other therapeutic/prophylactic compounds are administered treat a condition and/or disease state as described herein. In various aspects, the compound(s) of the invention and the therapeutic/prophylactic compound(s) are co-administered as a mixture or administered subjectly. In one aspect, the route of administration is the same for the compound(s) of the invention and the therapeutic/prophylactic compound(s), while in other aspects, the compound(s) of the invention and the therapeutic/prophylactic compound(s) are administered by different routes. In one embodiment, the dosages of the compound(s) of the invention and the therapeutic/prophylactic compound(s) are amounts that are therapeutically or prophylactically effective for each compound when administered subjectly. Alternatively, the combined administration permits use of lower dosages than would be required to achieve a therapeutic or prophylactic effect if administered subjectly, and such methods are useful in decreasing one or more side effects of the reduced-dose compound.

In one aspect, a compound of the present invention and one or more other therapeutic/prophylactic compound(s) effective at treating a condition are administered wherein, both compounds act through the same or different mechanisms. Therapeutic/prophylactic compound(s) include, but are not limited to, bile salt sequestering resins (e.g., cholestyramine, colestipol, and colesevelam hydrochloride), cholesterol biosynthesis inhibitors, especially HMG CoA reductase inhibitors (such as atorvastatin, pravastatin, simvastatin, lovastatin, fluvastatin, cerivastatin, rosuvastatin, and pitivastatin (itavastatin/risivastatin)), nicotinic acid, fibric acid derivatives (e.g., clofibrate, gemfibrozil, fenofibrate, bezafibrate, and ciprofibrate), probucol, neomycin, dextrothyroxine, plant-stanol esters, cholesterol absorption inhibitors (e.g., ezetimibe and pamaqueside), implitapide, squalene synthetase inhibitors, bile acid sequestrants such as cholestyramine, inhibitors of bile acid transporters (apical sodium-dependent bile acid transporters), regulators of hepatic CYP7a, estrogen replacement therapeutics (e.g., tamoxigen), and anti-inflammatories (e.g., glucocorticoids).

Antisense inhibition of CREB is described herein, below to reduce plasma lipids of animals on high-fat diets and may be useful in treatment of cardiovascular disease.

Any of the aforementioned is useful in combination with one or more of the CREB-specific modulator inhibitors, such as any antisense inhibitor of CREB according to this invention. Combined compounds (two or more) may be used together or sequentially.

### Pharmaceutical Compositions and Formulations

Another embodiment includes compositions and formulations for CREB-specific modulators such as, for example, antisense compounds. The CREB-specific modulators, as described herein, may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). Oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C.sub.1-10 alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999 which is incorporated herein, by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancer surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally in granular form including sprayed dried particles, or complexed to form micro or nanoparticles. Oligonucleotide complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for oligonucleotides and their preparation are described in detail in U.S. application Ser. No. 08/886,829 (filed Jul. 1, 1997), Ser. No. 09/108,673 (filed Jul. 1, 1998), Ser. No. 09/256,515 (filed Feb. 23, 1999), Ser. No. 09/082,624 (filed May 21, 1998) and Ser. No. 09/315,298 (filed May 20, 1999) each of which is incorporated herein, by reference in their entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

CREB-specific modulators include compositions of, but are not limited to, solutions, emulsions, and liposome-containing formulations. These compositions may be generated from a variety of components that include, but are not limited to, preformed liquids, self-emulsifying solids and self-emulsifying semisolids.

The CREB-specific modulators of as described herein, include formulations, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions, as described herein, may be formulated into any of many possible dosage forms such as, but not limited to, tablets, capsules, gel capsules, liquid syrups, soft gels, suppositories, and enemas. The compositions, as described herein, may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

CREB-specific modulators may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the agents of the present invention.

Antisense oligonucleotides may be admixed with pharmaceutically acceptable active and/or inert substances for the preparation of pharmaceutical compositions or formulations. Compositions and methods for the formulation of CREB-specific modulators are dependent upon a number of criteria, including, but not limited to, route of administration, extent of disease, or dose to be administered.

Antisense compound targeted to a CREB nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment employed in the methods described herein, is a pharmaceutical composition comprising a CREB-specific antisense compound a CREB-specific antisense compound and a pharmaceutically acceptable diluent. In one embodiment, the pharmaceutically acceptable diluent is PBS. In other embodiments, as described herein, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

In certain embodiments, as described herein, pharmaceutical compositions comprise one or more oligonucleotides and one or more excipients. In certain such embodiments, as described herein, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, as described herein, a CREB-specific modulator is prepared using known techniques, including, but not limited to, mixing, dissolving, granulating, dragee-making, levigating, emulsifying, encapsulating, entrapping or tabletting processes.

In certain embodiments, as described herein, a CREB-specific modulator is a liquid (e.g., a suspension, elixir and/or solution). In certain of such embodiments, as described herein, a liquid CREB-specific modulator is prepared using ingredients known in the art, including, but not limited to, water, glycols, oils, alcohols, flavoring agents, preservatives, and coloring agents.

In certain embodiments, as described herein, a CREB-specific modulator is a solid (e.g., a powder, tablet, and/or capsule). In certain of such embodiments, as described herein, a solid CREB-specific modulator comprising one or more oligonucleotides is prepared using ingredients known in the art, including, but not limited to, starches, sugars, diluents, granulating agents, lubricants, binders, and disintegrating agents.

In certain embodiments, as described herein, a CREB-specific modulator is formulated as a depot preparation. Certain such depot preparations are typically longer acting than non-depot preparations. In certain embodiments, as described herein, such preparations are administered by implantation (e.g., subcutaneously or intravenously) or by intramuscular injection. In certain embodiments, as described herein, depot preparations are prepared using suitable polymeric or hydrophobic materials (e.g., an emulsion in an acceptable oil) or ion exchange resins, or as sparingly soluble derivatives, for example, as a sparingly soluble salt.

In certain embodiments, as described herein, a CREB-specific modulator comprises a delivery system. Examples of delivery systems include, but are not limited to, liposomes and emulsions. Certain delivery systems are useful for preparing certain CREB-specific modulators, including those comprising hydrophobic compounds. In certain embodiments, as described herein, certain organic solvents such as dimethylsulfoxide are used.

In certain embodiments, as described herein, a CREB-specific modulator comprises one or more tissue-specific delivery molecules designed to deliver the one or more CREB-specific modulators to specific tissues or cell types. For example, in certain embodiments, as described herein, CREB-specific modulators include liposomes coated with a tissue-specific antibody.

In certain embodiments, as described herein, a CREB-specific modulator comprises a co-solvent system. Certain of such co-solvent systems comprise, for example, benzyl alcohol, a nonpolar surfactant, a water-miscible organic polymer, and an aqueous phase. In certain embodiments, as described herein, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™, and 65% w/v polyethylene glycol 300. The proportions of such co-solvent systems may be varied considerably without significantly altering their solubility and toxicity characteristics. Furthermore, the identity of co-solvent components may be varied; for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, as described herein, a CREB-specific modulator comprises a sustained-release system. A non-limiting example of such a sustained-release system is a semi-permeable matrix of solid hydrophobic polymers. In certain embodiments, as described herein, sustained-release systems may, depending on their chemical nature, release CREB-specific modulators over a period of hours, days, weeks or months.

In certain embodiments, as described herein, a CREB-specific modulator is prepared for oral administration. In certain of such embodiments, as described herein, a CREB-specific modulator is formulated by combining one or more oligonucleotides with one or more pharmaceutically acceptable carriers. Certain of such carriers enable CREB-specific modulators to be formulated as tablets, pills, dragees, capsules, liquids, gels, syrups, slurries, suspensions and the like, for oral ingestion by a subject. In certain embodiments, as described herein, pharmaceutical compositions for oral use are obtained by mixing oligonucleotide and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, as described herein, such a mixture is optionally ground and auxiliaries are optionally added. In certain embodiments, as described herein, pharmaceutical compositions are formed to obtain tablets or dragee cores. In certain embodiments, as described herein, disintegrating agents (e.g., cross-linked polyvinyl pyrrolidone, agar, or alginic acid or a salt thereof, such as sodium alginate) are added.

In certain embodiments, as described herein, dragee cores are provided with coatings. In certain such embodiments, as described herein, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, as described herein, CREB-specific modulators for oral administration are push-fit capsules made of gelatin. Certain of such push-fit capsules comprise one or more CREB-specific modulators in admixture with one or more fillers, such as lactose, binders, such as starches, and/or lubricants, such as talc or magnesium stearate and, optionally, stabilizers. In certain embodiments, as described herein, CREB-specific modulators for oral administration are soft, sealed capsules made of gelatin and a plasticizer, such as glycerol or sorbitol. In certain soft capsules, one or more CREB-specific modulators are be dissolved or suspended in suitable liquids, such as fatty oils, liquid paraffin, or liquid polyethylene glycols. In addition, stabilizers may be added.

In certain embodiments, as described herein, CREB-specific modulators are prepared for buccal administration. Certain of such CREB-specific modulators are tablets or lozenges formulated in conventional manner.

In certain embodiments, as described herein, a CREB-specific modulator is prepared for administration by injection (e.g., intravenous, subcutaneous, intramuscular, etc.). In certain of such embodiments, as described herein, a CREB-specific modulator comprises a carrier and is formulated in aqueous solution, such as water, or physiologically compatible buffers, such as Hanks's solution, Ringer's solution, or physiological saline buffer. In certain embodiments, as described herein, other ingredients are included (e.g., ingredients that aid in solubility or serve as preservatives). In certain embodiments, as described herein, injectable suspensions are prepared using appropriate liquid carriers, suspending agents and the like. Certain CREB-specific modulators for injection are presented in unit dosage form, for example, in ampules or in multi-dose containers. Certain CREB-specific modulators for injection are suspensions, solutions or emulsions in oily or aqueous vehicles, and may contain formulatory agents such as suspending, stabilizing and/or dispersing agents. Certain solvents suitable for use in CREB-specific modulators for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the CREB-specific modulators to allow for the preparation of highly concentrated solutions.

In certain embodiments, as described herein, a CREB-specific modulator is prepared for transmucosal administration. In certain of such embodiments, as described herein, penetrants appropriate to the barrier to be permeated are used in the formulation. Such penetrants are generally known in the art.

In certain embodiments, as described herein, a CREB-specific modulator is prepared for administration by inhalation. Certain of such CREB-specific modulators for inhalation are prepared in the form of an aerosol spray in a pressurized pack or a nebulizer. Certain of such CREB-specific modulators comprise a propellant, for example, dichlorodifluoromethane, trichlorofluoromethane, dichlorotetrafluoroethane, carbon dioxide or other suitable gas. In certain embodiments, as described herein, using a pressurized aerosol, the dosage unit may be determined with a valve that delivers a metered amount. In certain embodiments, as described herein, capsules and cartridges for use in an inhaler or insufflator may be formulated. Certain of such formulations comprise a powder mixture of a CREB-specific modulator of the invention and a suitable powder base, such as lactose or starch.

In certain embodiments, as described herein, a CREB-specific modulator is prepared for rectal administration, such as a suppositories or retention enema. Certain of such CREB-specific modulators comprise known ingredients, such as cocoa butter and/or other glycerides.

In certain embodiments, as described herein, a CREB-specific modulator is prepared for topical administration. Certain of such pharmaceutical compositions comprise bland moisturizing bases, such as ointments or creams. Exemplary suitable ointment bases include, but are not limited to, petrolatum, petrolatum plus volatile silicones, lanolin and water in oil emulsions such as Eucerin™, available from Beiersdorf (Cincinnati, Ohio). Exemplary suitable cream bases include, but are not limited to, Nivea™. Cream, available from Beiersdorf (Cincinnati, Ohio), cold cream (USP), Purpose Cream™, available from Johnson & Johnson (New Brunswick, N.J.), hydrophilic ointment (USP) and Lubriderm™, available from Pfizer (Morris Plains, N.J.).

In certain embodiments, as described herein, a CREB-specific modulator comprises an oligonucleotide in a therapeutically effective amount. In certain embodiments, as described herein, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, as described herein, one or more oligonucleotides is formulated as a prodrug. In certain embodiments, as described herein, upon in vivo administration, a prodrug is chemically converted to the biologically, pharmaceutically or therapeutically more active form of the oligonucleotide. In certain embodiments, as described herein, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, as described herein, prodrugs are less water soluble than the corresponding active form. In certain instances, such prodrugs possess superior transmittal across cell membranes, where water solubility is detrimental to mobility. In certain embodiments, as described herein, a prodrug is an ester. In certain such embodiments, as described herein, the ester is metabolically hydrolyzed to carboxylic acid upon administration. In certain instances, the carboxylic acid-containing compound is the corresponding active form. In certain embodiments, as described herein, a prodrug comprises a short peptide (polyaminoacid) bound to an acid group. In certain of such embodiments, as described herein, the peptide is cleaved upon administration to form the corresponding active form.

In certain embodiments, as described herein, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

In certain embodiments, as described herein, a pharmaceutical composition comprising one or more CREB-specific modulators is useful for treating conditions or disorders in a mammalian, and particularly, in a human, subject. Suitable administration routes include, but are not limited to, oral, rectal, transmucosal, intestinal, enteral, topical, suppository, through inhalation, intrathecal, intraventricular, intraperitoneal, intranasal, intraocular and parenteral (e.g., intravenous, intramuscular, intramedullary, and subcutaneous). In certain embodiments, as described herein, pharmaceutical intrathecals are administered to achieve local rather than systemic exposures. For example, pharmaceutical compositions may be injected directly in the area of desired effect (e.g., in the renal or cardiac area).

In certain embodiments, as described herein, a pharmaceutical composition is administered in the form of a dosage unit (e.g., tablet, capsule, bolus, etc.). In certain embodiments, as described herein, such pharmaceutical compositions comprise an oligonucleotide in a dose selected from 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 270 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, and 800 mg. In certain such embodiments, as described herein, a pharmaceutical composition comprises a dose of oligonucleotide selected from 25 mg, 50 mg, 75 mg, 100 mg, 150 mg, 200 mg, 250 mg, 300 mg, 350 mg, 400 mg, 500 mg, 600 mg, 700 mg, and 800 mg. In certain embodiments, as described herein, a pharmaceutical composition comprises a dose of oligonucleotide selected from 50 mg, 100 mg, 150 mg, 200 mg, 250 mg, 300 mg, and 400 mg. In certain embodiments, as described herein, the dose is administered at intervals ranging from more than once per day, once per day, once per week, twice per week, three times per week, four times per week, five times per week, 6 times per week, once per month to once per three months, for as long as needed to sustain the desired effect.

In a further aspect, a CREB-specific modulator is a sterile-lyophilized oligonucleotide that is reconstituted with a suitable diluent, for example, sterile water for injection. The reconstituted product is administered as a subcutaneous injection or as an intravenous infusion after dilution into saline. The lyophilized drug product consists of the oligonucleotide which has been prepared in water for injection, adjusted to pH 7.0-9.0 with acid or base during preparation, and then lyophilized. The lyophilized oligonucleotide may be 25-800 mg of the oligonucleotide. It is understood that this encompasses 25, 50, 75, 100, 125, 150, 175, 200, 225, 250, 275, 300, 325, 350, 375, 425, 450, 475, 500, 525, 550, 575, 600, 625, 650, 675, 700, 725, 750, 775, and 800 mg of lyophilized oligonucleotide. The lyophilized drug product may be packaged in a 2 mL Type I, clear glass vial (ammonium sulfate-treated), stoppered with a bromobutyl rubber closure and sealed with an aluminum FLIP-OFF® overseal.

The compositions may additionally contain other adjunct components conventionally found in pharmaceutical compositions, at their art-established usage levels. Thus, for example, the compositions may contain additional, compatible, pharmaceutically-active materials, such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, for example, lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation

### Salts, Prodrugs and Bioequivalents

The CREB-specific modulators of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive or less active form that is converted to an active form (i.e., drug) within the body or cells thereof by the action of endogenous enzymes or other chemicals and/or conditions. In particular, prodrug versions of the oligonucleotides of the invention are prepared as SATE ((S-acetyl-2-thioethyl)phosphate) derivatives according to the methods described in International Patent Application Publication No. WO 93/24510, published Dec. 9, 1993; and International Patent Application Publication No. WO 94/26764, and U.S. Pat. No. 5,770,713.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. For oligonucleotides, examples of pharmaceutically acceptable salts and their uses are further described in U.S. Pat. No. 6,287,860, which is incorporated herein, in its entirety.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic and inorganic acid salts of the amines. Acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic-acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfoc acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides or antisense compounds, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. Sodium salts of antisense oligonucleotides are useful and are well accepted for therapeutic administration to humans. In another embodiment, sodium salts of dsRNA compounds are also provided.

### Excipients

In contrast to CREB-specific modulators that are carrier compounds, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administrations which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Also provided herein, where appropriate, methods as provided herein can be performed both in vitro and/or in vivo.

While the present invention has been described with specificity in accordance with certain embodiments, as described herein, the following examples serve only to illustrate the invention and are not intended to limit the same.

### EXAMPLES

### Non-Limiting Disclosure and Incorporation by Reference

While certain compounds, compositions and methods described herein have been described with specificity in accordance with certain embodiments, as described herein, the following examples serve only to illustrate the compounds described herein and are not intended to limit the same. Each of the references recited in the present application is incorporated herein by reference in its entirety.

The in vivo studies provided herein below are carried out in well characterized models of disease that are recognized by those of skill in the art as being predictive of therapeutic results in other animals, including humans.

### Example 1

### Antisense Inhibition of CREB in Rat A10 Cells

Antisense oligonucleotides targeted to a CREB nucleic acid were tested for their effects on CREB mRNA in vitro. Cultured A10 cells were transfected using lipofectin reagent with 90 nM antisense oligonucleotide for 4 hours. After a recovery period of approximately 24 hours, RNA was isolated from the cells and CREB mRNA levels were measured by quantitative real-time PCR. CREB mRNA levels were adjusted according to total RNA content, as measured by RIBOGREEN®. Results are presented as percent inhibition of CREB, relative to untreated control cells.

The chimeric antisense oligonucleotides in Table 1 were designed as 5-10-5 MOE gapmers. The gapmers are 20 nucleotides in length, wherein the central gap segment is comprised of 10 2′-deoxynucleotides and is flanked on both sides (in the 5′ and 3′ directions) by wings comprising 5 nucleotides each. Each nucleotide in the 5′ wing segment and each nucleotide in the 3′ wing segment has a 2′-MOE modification. The internucleoside linkages throughout each gapmer are phosphorothioate (P-S) linkages. All cytidine residues throughout each gapmer are 5-methylcytidines. “Rat target start site” indicates the 5′-most nucleotide to which the gapmer is targeted in the rat sequence. “Rat target stop site” indicates the 3′-most nucleotide to which the gapmer is targeted in the rat sequence. Each gapmer listed in Table 1 is targeted to rat target sequence SEQ ID NO: 3 (GENBANK Accession No. NM_{—}031017.1) or SEQ ID NO: 2 (GENBANK Accession No. NW_{—}047816.2_truncated from nucleotides_{—}6598000 to 6666000), or SEQ ID NO: 6 (GENBANK Accession No. NM_{—}134443.1), or SEQ ID NO: 5 (GENBANK Accession No. BE114301.1_COMP), or SEQ ID NO: 4 (GENBANK Accession No. CV116908.1).

The rat oligonucleotides also show cross reactivity, (i.e. ≦3 base mismatch) with the human CREB mRNA (GENBANK Accession No. NM_{—}134442.2), incorporated herein as SEQ ID NO: 1. “Human Target Start Site” indicates the 5′-most nucleotide in the human mRNA to which the antisense oligonucleotide is targeted. “Human Target Stop Site” indicates the 3′-most nucleotide in the human mRNA to which the antisense oligonucleotide is targeted. ‘Mismatches’ indicates the number of nucleobases by which the rat oligonucleotide is mismatched with the human gene sequence. ‘n/a’ indicates that there may be more than 3 mismatches between the rat oligonucleotide and the human gene sequence.

TABLE 1
Inhibition of rat CREB mRNA levels by chimeric antisense
oligonucleotides having 5-10-5 MOE wings and deoxy gap

| | Rat | Rat | | % | Human | Human | | | |
|---|---|---|---|---|---|---|---|---|---|
| | Target | Target | | inhibi- | Target | Target | | Rat | SEQ |
| ISIS | Start | Stop | Sequence | tion | Start | Stop | | Target | ID |
| No. | Site | Site | (5′ to 3′) | in rat | Site | Site | Mismatches | Sequence | NO. |
| 342367 | 136 | 155 | ACCTGG | 94 | 278 | 297 | 0 | NM_031017.1 | 13 |
| | | | GCTAAT | | | | | | |
| | | | GTGGCA | | | | | | |
| | | | AT | | | | | | |
| 342369 | 208 | 227 | GTCTGCC | 28 | 350 | 369 | 0 | NM_031017.1 | 14 |
| | | | CATTGG | | | | | | |
| | | | GCAGCT | | | | | | |
| | | | G | | | | | | |
| 342370 | 274 | 293 | GTTTGGA | 58 | 416 | 435 | 0 | NM_031017.1 | 15 |
| | | | CTTGTGG | | | | | | |
| | | | AGACTG | | | | | | |
| 342372 | 339 | 358 | CTGCAAT | 62 | 481 | 500 | 1 | NM_031017.1 | 16 |
| | | | AGTTGA | | | | | | |
| | | | AATCTG | | | | | | |
| | | | A | | | | | | |
| 342373 | 344 | 363 | ACTTTCT | 66 | 486 | 505 | 0 | NM_031017.1 | 17 |
| | | | GCAATA | | | | | | |
| | | | GTTGAA | | | | | | |
| | | | A | | | | | | |
| 342379 | 452 | 471 | ATCAGA | 59 | 594 | 613 | 0 | NM_031017.1 | 18 |
| | | | AGATAA | | | | | | |
| | | | GTCATTC | | | | | | |
| | | | A | | | | | | |
| 342380 | 457 | 476 | GGTGCA | 43 | 599 | 618 | 0 | NM_031017.1 | 19 |
| | | | TCAGAA | | | | | | |
| | | | GATAAG | | | | | | |
| | | | TC | | | | | | |
| 342381 | 526 | 545 | GTTACA | 44 | 668 | 687 | 0 | NM_031017.1 | 20 |
| | | | GTGGTG | | | | | | |
| | | | ATGGCA | | | | | | |
| | | | GG | | | | | | |
| 342382 | 559 | 578 | CCACTGC | 40 | 701 | 720 | 0 | NM_031017.1 | 21 |
| | | | TAGTTTG | | | | | | |
| | | | GTAAAT | | | | | | |
| 342390 | 658 | 677 | GCTGCAT | 43 | 800 | 819 | 0 | NM_031017.1 | 22 |
| | | | TGGTCAT | | | | | | |
| | | | GGTTAA | | | | | | |
| 342394 | 746 | 765 | AACAAC | 34 | 888 | 907 | 0 | NM_031017.1 | 23 |
| | | | AACTTG | | | | | | |
| | | | GTTGCTG | | | | | | |
| | | | G | | | | | | |
| 342395 | 751 | 770 | GCTTGA | 53 | 893 | 912 | 0 | NM_031017.1 | 24 |
| | | | ACAACA | | | | | | |
| | | | ACTTGGT | | | | | | |
| | | | T | | | | | | |
| 342396 | 756 | 775 | AGGCAG | 49 | 898 | 917 | 0 | NM_031017.1 | 25 |
| | | | CTTGAAC | | | | | | |
| | | | AACAAC | | | | | | |
| | | | T | | | | | | |
| 342405 | 1024 | 1043 | AGGTCCT | 46 | 1166 | 1185 | 0 | NM_031017.1 | 26 |
| | | | TAAGTG | | | | | | |
| | | | CTTTTAG | | | | | | |
| 342407 | 1057 | 1076 | TAAATCC | 55 | 1199 | 1218 | 0 | NM_031017.1 | 27 |
| | | | CAAATT | | | | | | |
| | | | AATCTG | | | | | | |
| | | | A | | | | | | |
| 342414 | 66553 | 66572 | AAGAAG | 23 | 1863 | 1882 | 0 | NW_047816. | 28 |
| | | | CAACAA | | | | | 2_TRUNC_ | |
| | | | CTGCCCT | | | | | 6598000_66 | |
| | | | A | | | | | 66000 | |
| 342416 | 303 | 322 | TTTTTAA | 23 | 445 | 464 | 0 | NM_031017.1 | 29 |
| | | | GTCCTTA | | | | | | |
| | | | CAGGAA | | | | | | |
| 342419 | 41507 | 41526 | ATGAATT | 18 | n/a | n/a | n/a | NW_047816. | 30 |
| | | | TTATTGT | | | | | 2_TRUNC_ | |
| | | | TACAAG | | | | | 6598000_66 | |
| | | | | | | | | 66000 | |
| 385913 | 351 | 370 | AGATTTT | 52 | 574 | 593 | 2 | NM_1134443.1 | 31 |
| | | | CTTGTAG | | | | | | |
| | | | GAAGGC | | | | | | |
| 385914 | 1003 | 1022 | CCATTTT | 82 | n/a | n/a | n/a | NM_134443.1 | 32 |
| | | | CACCAC | | | | | | |
| | | | AATAGG | | | | | | |
| | | | T | | | | | | |
| 385915 | 32 | 51 | GTCCATG | 82 | 174 | 193 | 3 | NM_031017.1 | 33 |
| | | | GTCATCT | | | | | | |
| | | | AGTCAC | | | | | | |
| 385916 | 142 | 161 | ATGGAT | 90 | 284 | 303 | 1 | NM_031017.1 | 34 |
| | | | ACCTGG | | | | | | |
| | | | GCTAAT | | | | | | |
| | | | GT | | | | | | |
| 385917 | 149 | 168 | TGCTGGC | 94 | 291 | 310 | 1 | NM_031017.1 | 35 |
| | | | ATGGAT | | | | | | |
| | | | ACCTGG | | | | | | |
| | | | G | | | | | | |
| 385918 | 157 | 176 | GCATGA | 87 | 299 | 318 | 1 | NM_031017.1 | 36 |
| | | | GCTGCTG | | | | | | |
| | | | GCATGG | | | | | | |
| | | | A | | | | | | |
| 385919 | 181 | 200 | GTTACA | 59 | 323 | 342 | 1 | NM_031017.1 | 37 |
| | | | GTGGGA | | | | | | |
| | | | GCAGAT | | | | | | |
| | | | GA | | | | | | |
| 385920 | 190 | 209 | TGCACTA | 56 | n/a | n/a | n/a | NM_031017.1 | 38 |
| | | | AGGTTA | | | | | | |
| | | | CAGTGG | | | | | | |
| | | | G | | | | | | |
| 385921 | 200 | 219 | ATTGGG | 63 | 342 | 361 | 3 | NM_031017.1 | 39 |
| | | | CAGCTG | | | | | | |
| | | | CACTAA | | | | | | |
| | | | GG | | | | | | |
| 385922 | 256 | 275 | TGAATA | 80 | 398 | 417 | 0 | NM_031017.1 | 40 |
| | | | ACTGAT | | | | | | |
| | | | GGCTGG | | | | | | |
| | | | GC | | | | | | |
| 385923 | 269 | 288 | GACTTGT | 41 | 411 | 430 | 0 | NM_031017.1 | 41 |
| | | | GGAGAC | | | | | | |
| | | | TGAATA | | | | | | |
| | | | A | | | | | | |
| 385924 | 296 | 315 | GTCCTTA | 63 | 438 | 457 | 0 | NM_031017.1 | 42 |
| | | | CAGGAA | | | | | | |
| | | | GACTGA | | | | | | |
| | | | A | | | | | | |
| 385925 | 308 | 327 | AAGTCTT | 55 | 450 | 469 | 0 | NM_031017.1 | 43 |
| | | | TTTAAGT | | | | | | |
| | | | CCTTAC | | | | | | |
| 385926 | 314 | 333 | GGAGAA | 30 | 456 | 475 | 0 | NM_031017.1 | 44 |
| | | | AAGTCTT | | | | | | |
| | | | TTTAAGT | | | | | | |
| 385927 | 382 | 401 | GAATCA | 70 | 524 | 543 | 0 | NM_031017.1 | 45 |
| | | | GTTACAC | | | | | | |
| | | | TATCCAC | | | | | | |
| 385928 | 388 | 407 | TTTTGGG | 61 | 530 | 549 | 0 | NM_031017.1 | 46 |
| | | | AATCAG | | | | | | |
| | | | TTACACT | | | | | | |
| 385929 | 441 | 460 | AGTCATT | 72 | 583 | 602 | 0 | NM_031017.1 | 47 |
| | | | CAAAAT | | | | | | |
| | | | TTTCCTG | | | | | | |
| 385930 | 446 | 465 | AGATAA | 24 | 588 | 607 | 0 | NM_031017.1 | 48 |
| | | | GTCATTC | | | | | | |
| | | | AAAATTT | | | | | | |
| 385931 | 463 | 482 | ACCCCTG | 74 | 605 | 624 | 1 | NM_031017.1 | 49 |
| | | | GTGCATC | | | | | | |
| | | | AGAAGA | | | | | | |
| 385932 | 473 | 492 | AATCCTT | 81 | 615 | 634 | 1 | NM_031017.1 | 50 |
| | | | GGCACC | | | | | | |
| | | | CCTGGTG | | | | | | |
| 385933 | 581 | 600 | CTGGGT | 52 | 723 | 742 | 0 | NM_031017.1 | 51 |
| | | | AATGGC | | | | | | |
| | | | AATATA | | | | | | |
| | | | CT | | | | | | |
| 385934 | 643 | 662 | GTTAATG | 50 | 785 | 804 | 1 | NM_031017.1 | 52 |
| | | | TCTGCAG | | | | | | |
| | | | GCCCTG | | | | | | |
| 385935 | 651 | 670 | TGGTCAT | 29 | 793 | 812 | 1 | NM_031017.1 | 53 |
| | | | GGTTAAT | | | | | | |
| | | | GTCTGC | | | | | | |
| 385936 | 663 | 682 | TGGCAG | 47 | 805 | 824 | 1 | NM_031017.1 | 54 |
| | | | CTGCATT | | | | | | |
| | | | GGTCAT | | | | | | |
| | | | G | | | | | | |
| 385937 | 670 | 689 | GGCTGA | 40 | 812 | 831 | 1 | NM_031017.1 | 55 |
| | | | GTGGCA | | | | | | |
| | | | GCTGCAT | | | | | | |
| | | | T | | | | | | |
| 385938 | 735 | 754 | GGTTGCT | 32 | 877 | 896 | 2 | NM_031017.1 | 56 |
| | | | GGGCAC | | | | | | |
| | | | TAGAAT | | | | | | |
| | | | C | | | | | | |
| 385939 | 781 | 800 | ATCTGGT | 18 | 923 | 942 | 1 | NM_0131017.1 | 57 |
| | | | ATGTTTG | | | | | | |
| | | | TACATC | | | | | | |
| 385940 | 984 | 1003 | TTTGGTT | 64 | 1126 | 1145 | 1 | NM_031017.1 | 58 |
| | | | TTCAAGC | | | | | | |
| | | | ACTGCC | | | | | | |
| 385941 | 1029 | 1048 | AGTAAA | 60 | 1171 | 1190 | 0 | NM_031017.1 | 59 |
| | | | GGTCCTT | | | | | | |
| | | | AAGTGC | | | | | | |
| | | | T | | | | | | |
| 385942 | 1036 | 1055 | TTGTGGC | 42 | 1178 | 1197 | 0 | NM_031017.1 | 60 |
| | | | AGTAAA | | | | | | |
| | | | GGTCCTT | | | | | | |
| 385943 | 1052 | 1071 | CCCAAA | 76 | 1194 | 1213 | 1 | NM_031017.1 | 61 |
| | | | TTAATCT | | | | | | |
| | | | GACTTGT | | | | | | |
| 385944 | 1067 | 1086 | GGTGAA | 51 | 1209 | 1228 | 0 | NM_031017.1 | 62 |
| | | | AATTTAA | | | | | | |
| | | | ATCCCA | | | | | | |
| | | | A | | | | | | |
| 385945 | 66183 | 66202 | CAAGAT | 44 | 1511 | 1530 | 3 | NW_047816. | 63 |
| | | | TTCATTT | | | | | 2_TRUNC_ | |
| | | | TCCTCAT | | | | | 6598000_66 | |
| | | | | | | | | 66000 | |
| 385946 | 66192 | 66211 | TAAGAA | 63 | n/a | n/a | n/a | NW_047816. | 64 |
| | | | AGCCAA | | | | | 2_TRUNC_ | |
| | | | GATTTCA | | | | | 6598000_66 | |
| | | | T | | | | | 66000 | |
| 385947 | 66248 | 66267 | GCACAA | 47 | 1536 | 1555 | 0 | NW_047816. | 65 |
| | | | ACCTTGA | | | | | 2_TRUNC_ | |
| | | | AATCATT | | | | | 6598000_66 | |
| | | | | | | | | 66000 | |
| 385948 | 66256 | 66275 | GGAGCT | 62 | 1544 | 1563 | 0 | NW_047816. | 66 |
| | | | CAGCAC | | | | | 2_TRUNC_ | |
| | | | AAACCTT | | | | | 6598000_66 | |
| | | | G | | | | | 66000 | |
| 385949 | 66308 | 66327 | CCACAC | 39 | 1599 | 1618 | 0 | NW_047816. | 67 |
| | | | ATTACTT | | | | | 2_TRUNC_ | |
| | | | CAGCTC | | | | | 6598000_66 | |
| | | | A | | | | | 66000 | |
| 385950 | 66354 | 66373 | CAATCA | 67 | 1645 | 1664 | 0 | NW_047816. | 68 |
| | | | ACACTTC | | | | | 2_TRUNC_ | |
| | | | TTCATTG | | | | | 6598000_66 | |
| | | | | | | | | 66000 | |
| 385951 | 66363 | 66382 | TCAATTT | 59 | 1654 | 1673 | 0 | NW_047816. | 69 |
| | | | GGCAAT | | | | | 2_TRUNC_ | |
| | | | CAACAC | | | | | 6598000_66 | |
| | | | T | | | | | 66000 | |
| 385952 | 66390 | 66409 | CATAATC | 44 | n/a | n/a | n/a | NW_047816. | 70 |
| | | | CACAAT | | | | | 2_TRUNC_ | |
| | | | GAAGTG | | | | | 6598000_66 | |
| | | | T | | | | | 66000 | |
| 385953 | 66400 | 66419 | AATAGTT | 56 | n/a | n/a | n/a | NW_047816. | 71 |
| | | | TTACATA | | | | | 2_TRUNC_ | |
| | | | ATCCAC | | | | | 6598000_66 | |
| | | | | | | | | 66000 | |
| 385954 | 959 | 978 | GTTCTCT | 56 | 1101 | 1120 | 1 | NM_031017.1 | 72 |
| | | | AAACAT | | | | | | |
| | | | TTCACAT | | | | | | |
| 385955 | 95 | 114 | CAGTTA | 63 | 1172 | 1191 | 1 | BE114301.1_ | 73 |
| | | | AGGTCCT | | | | | COMP | |
| | | | TAAGTG | | | | | | |
| | | | C | | | | | | |
| 385956 | 65906 | 65925 | CAGTCC | 38 | 1231 | 1250 | 2 | NW_047816. | 74 |
| | | | ATTTTCC | | | | | 2_TRUNC_ | |
| | | | ACCACA | | | | | 6598000_66 | |
| | | | A | | | | | 66000 | |
| 385957 | 65989 | 66008 | GTTGCTT | 43 | 1317 | 1336 | 1 | NW_047816. | 75 |
| | | | CCAGGC | | | | | 2_TRUNC_ | |
| | | | AGTTTTG | | | | | 6598000_66 | |
| | | | | | | | | 66000 | |
| 385958 | 66041 | 66060 | GTTGTGG | 40 | n/a | n/a | n/a | NW_047816. | 76 |
| | | | ACATTCA | | | | | 2_TRUNC_ | |
| | | | CAGTTT | | | | | 6598000_66 | |
| | | | | | | | | 66000 | |
| 385959 | 66134 | 66153 | GATTACT | 57 | 1462 | 1481 | 3 | NW_047816. | 77 |
| | | | TCTTGAG | | | | | 2_TRUNC_ | |
| | | | GGTGGT | | | | | 6598000_66 | |
| | | | | | | | | 66000 | |
| 385960 | 66144 | 66163 | ATAAGC | 50 | 1472 | 1491 | 3 | NW_047816. | 78 |
| | | | AAATGA | | | | | 2_TRUNC_ | |
| | | | TTACTTC | | | | | 6598000_66 | |
| | | | T | | | | | 66000 | |
| 385961 | 29 | 48 | ACAGGC | 62 | 7 | 26 | 3 | CV116908.1 | 79 |
| | | | AGCAGC | | | | | | |
| | | | AGCATC | | | | | | |
| | | | CC | | | | | | |
| 385962 | 459 | 478 | ACCTAG | 0 | n/a | n/a | n/a | CV116908.1 | 80 |
| | | | AACAAT | | | | | | |
| | | | GACTGA | | | | | | |
| | | | AC | | | | | | |
| 385963 | 749 | 768 | AGCATTT | 29 | n/a | n/a | n/a | CV116908.1 | 81 |
| | | | GCCATGT | | | | | | |
| | | | ATTGTA | | | | | | |
| 385964 | 19801 | 19820 | TATTTTA | 52 | n/a | n/a | n/a | NW_047816. | 82 |
| | | | TACCTGG | | | | | 2_TRUNC_ | |
| | | | GCTAAT | | | | | 6598000_66 | |
| | | | | | | | | 66000 | |
| 385965 | 26810 | 26829 | GCATGG | 13 | n/a | n/a | n/a | NW_047816. | 83 |
| | | | ATACCTA | | | | | 2_TRUNC_ | |
| | | | CAGAAA | | | | | 6598000_66 | |
| | | | A | | | | | 66000 | |
| 385966 | 29667 | 29686 | TTATCCT | 73 | n/a | n/a | n/a | NW_047816. | 84 |
| | | | CACCTG | | | | | 2_TRUNC_ | |
| | | | ACACATT | | | | | 6598000_66 | |
| | | | | | | | | 66000 | |
| 385967 | 40018 | 40037 | TTCCCAG | 58 | n/a | n/a | n/a | NW_047816. | 85 |
| | | | CTCTTCA | | | | | 2_TRUNC_ | |
| | | | TAATGG | | | | | 6598000_66 | |
| | | | | | | | | 66000 | |
| 385968 | 40214 | 40233 | CTCAGAT | 6 | n/a | n/a | n/a | NW_047816. | 86 |
| | | | AAATCC | | | | | 2_TRUNC_ | |
| | | | AAGGAT | | | | | 6598000_66 | |
| | | | C | | | | | 66000 | |
| 385969 | 41527 | 41546 | GTAATG | 31 | n/a | n/a | n/a | NW_047816. | 87 |
| | | | GCAACT | | | | | 2_TRUNC_ | |
| | | | AAAACC | | | | | 6598000_66 | |
| | | | CA | | | | | 66000 | |
| 385970 | 41710 | 41729 | ATTCTAT | 58 | n/a | n/a | n/a | NW_047816. | 88 |
| | | | TACCTTG | | | | | 2_TRUNC_ | |
| | | | AACAAC | | | | | 6598000_66 | |
| | | | | | | | | 66000 | |
| 385971 | 46488 | 46507 | CAATTTT | 37 | n/a | n/a | n/a | NW_047816. | 89 |
| | | | GGGTAG | | | | | 2_TRUNC_ | |
| | | | TCACTTT | | | | | 6598000_66 | |
| | | | | | | | | 66000 | |
| 385972 | 46802 | 46821 | AAAACT | 37 | n/a | n/a | n/a | NW_047816. | 90 |
| | | | TGGGAG | | | | | 2_TRUNC_ | |
| | | | GTAGAA | | | | | 6598000_66 | |
| | | | CT | | | | | 66000 | |

### Example 2

### Antisense Inhibition of CREB in Rat Primary Hepatocytes

Antisense oligonucleotides targeted to CREB (CREB antisense oligonucleotides: ISIS 385915, ISIS 385943, and ISIS 385967) were tested for antisense inhibition of CREB mRNA expression in rat primary hepatocytes. Rat hepatocytes were isolated by standard procedures and plated in a 96-well plate. Antisense oligonucleotides at a concentration of 150 nM and lipofectin (Invitrogen Corp.) were added to the hepatocytes for 4 hours in serum-free William's E media (Invitrogen Corp.). Antisense oligonucleotide and lipofectin were mixed in a ratio of 3 μg of lipofectin for every 1 ml of 100 nM antisense oligonucleotide concentration. After 4 hours, the hepatocyte media was changed to normal maintenance media (William's E media with 10% FBS and 10 nM insulin). After an incubation period of approximately 24 hours, RNA was isolated from the cells and CREB mRNA levels were measured by quantitative real-time PCR, as described herein, and the results are presented in Table 2.

Antisense modulation of CREB expression can be assayed in a variety of ways known in the art. For example, CREB mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation are taught in, for example, ‘RNA isolation and Characterization Protocols’ (Rapley and Manning, editors, Human Press Inc). Northern blot analysis is routine in the art and is taught in, for example, “molecular Biology of the Cell (Alberts, et al. Garland Science, Taylor & Francis Group, NY, pp 538-539). Real-time quantitative PCR can be conveniently accomplished using the commercially available ABI PRISM™ 7700 Sequence Detection System, available from PE-Applied Biosystems, Foster City, Calif. and used according to the manufacturer's instructions.

TABLE 2
Antisense inhibition of CREB in rat primary hepatocytes

| | SEQ | Target Nucleic acid | 5′ | 3′ | | | |
|---|---|---|---|---|---|---|---|
| Isis | ID | (Genbank Accession | Target | Target | Oligo | | % |
| No | NO | No.) | site | site | Sequence | Motif | Inhibition |
| 385915 | 33 | NW_047816.2_TRUNC_ | 19689 | 19708 | GTCCATGGTCATCTAG | 5-10-5 | 76 |
| | | 6598000_6666000 | | | TCAC | | |
| | | NM_031017.1 | 32 | 51 | | | |
| | | CV116908.1 | 196 | 215 | | | |
| 385943 | 61 | BE114301.1_COMP | 117 | 136 | CCCAAATTAATCTGAC | 5-10-5 | 65 |
| | | NM_031017.1 | 1052 | 1071 | TTGT | | |
| | | NM_134443.1 | 971 | 990 | | | |
| | | NW_047816.2_TRUNC_ | 65869 | 65888 | | | |
| | | 6598000_6666000 | | | | | |
| 385967 | 85 | NW_047816.2_ | 40018 | 40037 | TTCCCAGCTCTTCATA | 5-10-5 | 61 |
| | | TRUNC_6598000_ | | | ATGG | | |
| | | 6666000 | | | | | |

The CREB mRNA levels were normalized to total RNA content, as measured by RIBOGREEN®. Results are presented as percent inhibition of CREB, relative to untreated control cells. The antisense oligonucleotides inhibited CREB expression, by at least 60%.

The motif column indicates the wing-gap-wing motif of each antisense oligonucleotide. Antisense oligonucleotides were designed as 5-10-5 gapmers, where the gap segment comprises 2′-deoxynucleotides and each wing segment comprises 2′-MOE nucleotides. As illustrated in Table 2, a single nucleobase sequence may be represented by a 5-10-5 motif. “5′ target site” indicates the 5′-most nucleotide to which the antisense oligonucleotide is targeted on the indicated GENBANK Accession No.

It is expected that the relative inhibition levels of antisense oligonucleotides in vitro will be consistent across cell types which express an mRNA with which the antisense oligonucleotides are specifically hybridizable. This is also expected in vivo for cells to which the antisense oligonucleotides distribute.

These results confirm the reduction in CREB expression as a result of inhibition of CREB following treatment with ISIS 385915, ISIS 385943, and ISIS 385967.

### Example 3

### Antisense Inhibition of CREB in Rat Primary Hepatocytes: Effect on Fatty Acid Oxidation, Triglyceride Synthesis and Sterol Synthesis

To test the effect of inhibiting CREB expression on metabolism of lipids and fatty acids, an antisense oligonucleotide to CREB (CREB antisense oligonucleotide ISIS 385915) was tested to determine its effect on fatty acid oxidation, triglyceride synthesis and sterol synthesis. Primary rat hepatocytes were isolated, as described (Savage et al. 2006. J. Clin. Invest 116:817-824), and plated on collagen-coated-25-cm² flasks, for fatty oxidation measurement, and 60-mm plates, for de novo fatty acid and sterol synthesis measurements. The cells were transfected and incubated under normal conditions for 20-24 hours, and then fatty acid oxidation (oxidation of [¹⁴C]oleate to CO₂ and acid-soluble products), fatty acid synthesis (incorporation of [¹⁴C]acetic acid into fatty acids), and sterol synthesis (incorporation of [¹⁴C]acetic acid into sterols) were measured, as described by Yu et al (Yu et al. 2005. Hepatology 42: 362-371).

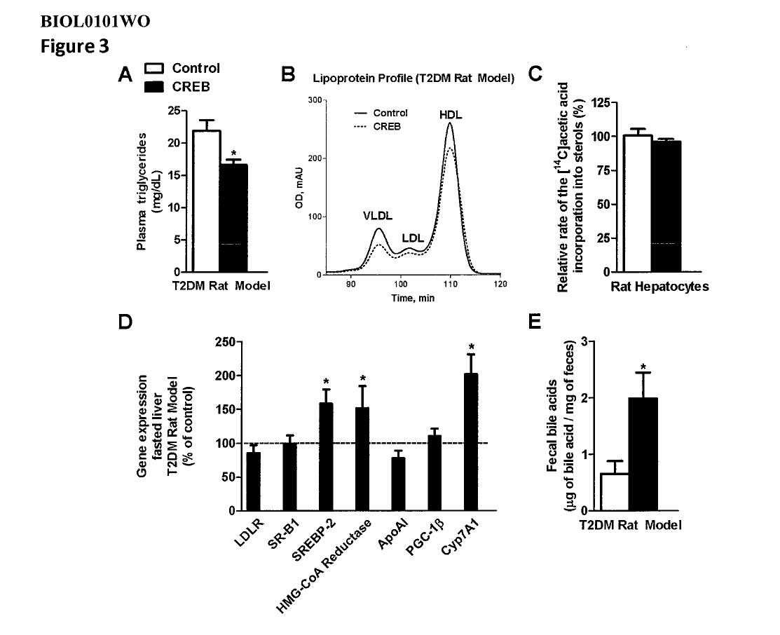

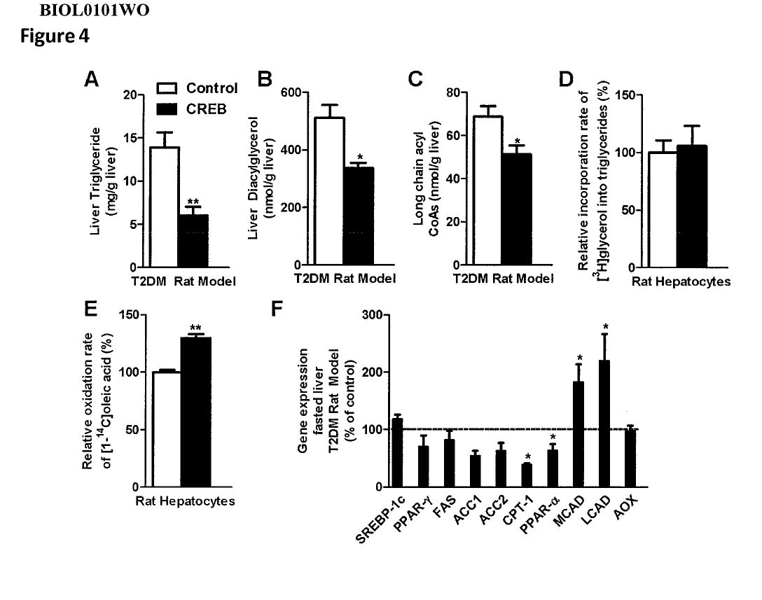

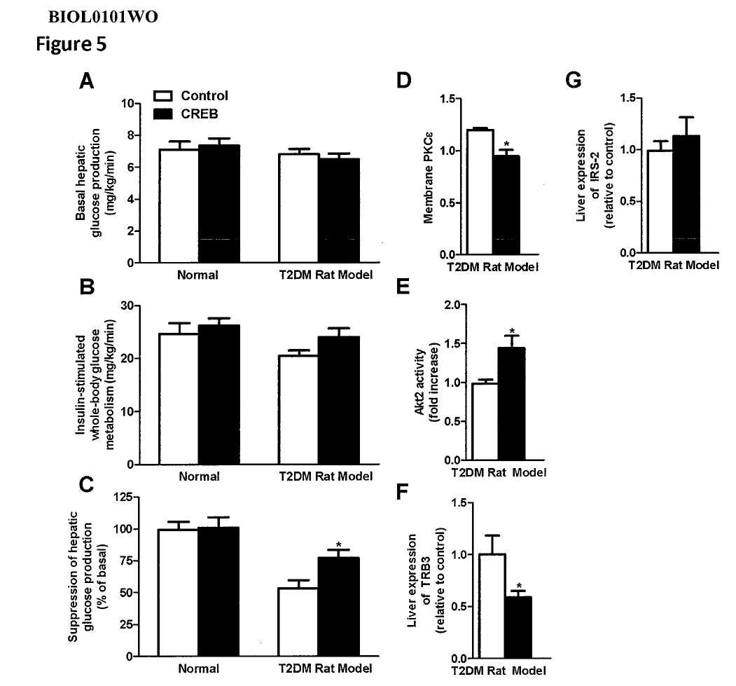

These results indicate that the CREB antisense oligonucleotide had no effect on the rate of sterol synthesis (100.0±7.2 in controls vs. 102±2.2 in CREB antisense oligonucleotide), as shown in FIG. 3C. However, the CREB antisense oligonucleotide decreased the rate of fatty acid synthesis by 30% (100.0±12.1 in controls vs. 69.9±2.8 in CREB antisense oligonucleotide) in primary rat hepatocytes. CREB antisense oligonucleotide-treated cells showed a 35% increase in fatty acid oxidation compared to the control cells, from 100.0±3.3 in controls to 134.7±5.7 in CREB antisense oligonucleotide treated cells, as shown in FIG. 4E.

These studies reveal that antisense inhibition of CREB can modulate metabolism of lipids, particularly fatty acids. Rat hepatocytes have been utilized in previous studies and the data was found to be predicative of in vivo results (Naritomi et al., Drug Metab Dispos. 2003 May; 31(5):580-8). Treatment of primary rat hepatocytes with an antisense inhibitor of CREB reduced fatty acid synthesis and increased fatty acid oxidation. Thus, antisense inhibitors of CREB could be candidate therapeutic agents for the treatment of conditions characterized by abnormal lipid metabolism, such as fatty liver diseases, dyslipidemia, or conditions that increase these disease risks, such as insulin resistance and obesity.

### Example 4

### Antisense Inhibition of CREB in Lean Rats

### Treatment

To evaluate the effects of CREB antisense inhibition on CREB mRNA levels in vivo, ISIS 385915 and ISIS 385922 were tested in male-Sprague-Dawley (MSD) rats fed on a normal diet (Labdiets #8604, Purina, St. Louis, Mo.). Treatment groups (4-6 mice each) were as follows: a group treated with ISIS 385915, a group treated with ISIS 385922, and a control group treated with saline. Oligonucleotide or saline was administered intraperitoneally twice weekly, for a period of 3 weeks; oligonucleotide doses were 50 mg/kg. After the treatment period, whole liver was collected for RNA analysis and blood was collected for plasma transaminase analysis.

### RNA Analysis

RNA was extracted from liver tissue for real-time PCR analysis of CREB. As shown in Table 3, both antisense oligonucleotides achieved significant reduction of CREB over the saline control. Results are presented as percent inhibition of CREB, relative to the control.

TABLE 3
Percent inhibition of CREB mRNA in MSD rats compared to the control

| | % inhibition |
|---|---|
| Saline | 0 |
| ISIS 385915 | 88 |
| ISIS 385922 | 67 |

### Measurement of Plasma Transaminase Levels

Elevated levels of plasma transaminases are often used clinically as potential indicators of liver damage. To evaluate the impact of ISIS 385915 and ISIS 385922 on hepatic function of mice described above, plasma concentrations of transaminases were measured using an automated clinical chemistry analyzer (Olympus AU400e, Melville, N.Y.). Measurements of alanine transaminase (ALT) and aspartate transaminase (AST) were taken after antisense oligonucleotide treatment, and are shown in Table 4.

TABLE 4
Effect of antisense inhibition on plasma transaminases (IU/L)

| | ALT- | ALT- | AST- | AST- |
|---|---|---|---|---|
| | Week 0 | Week 3 | Week 0 | Week 3 |
| Saline | 78 | 78 | 102 | 76 |
| ISIS 385915 | 86 | 89 | 89 | 55 |
| ISIS 385922 | 77 | 72 | 97 | 65 |

Together, these gene expression and transaminase concentration studies reveal that both oligonucleotides can specifically inhibit CREB gene expression without any hepatic toxicity.

Treatment with antisense inhibitors of CREB reduced CREB mRNA levels in liver tissue. Thus, antisense inhibitors of CREB are candidate therapeutic agents for the treatment of disorders characterized by increased CREB expression or activity in adipose and liver tissues (such as obesity, hepatic steatosis, NAFLD, NASH, dyslipidemia, insulin resistance and type 2 diabetes).

### Example 5

### Antisense Inhibition of CREB in High-Fat Fed Animal Model

### Treatment

To evaluate the effect of CREB antisense inhibition on CREB mRNA levels in vivo, ISIS 385915 was evaluated in normal male-Sprague-Dawley (MSD) rats fed a high fat diet. Treatment groups (4-6 mice each) were as follows: a group treated with ISIS 385915, a control group treated with saline, and a control group treated with an oligonucleotide not complementary to any known gene sequence (ISIS 141923, CCTTCCCTGAAGGTTCCTCC (SEQ ID NO: 94). Oligonucleotide or saline was administered intraperitoneally twice weekly, for a period of 4 weeks; oligonucleotide doses were 75 mg/kg/wk. After the treatment period, whole liver and white adipose tissue was collected for RNA and protein analyses.

### RNA Analysis

Liver and white adipose tissue RNA was isolated for real-time PCR analysis of CREB. Specifically, total RNA was isolated from fasted and fed rat liver tissue and fasted rat white adipose tissue (WAT). RNA was extracted using a commercially available kit with DNase digestion (QIAGEN RNeasy Kit; QIAGEN). RNA was reverse-transcribed into cDNA using Strata Script Reverse Transcriptase (Stratagene). The abundance of transcripts was assessed by real-time PCR on an Opticon 2 (Bio-Rad) with a SYBR Green detection system (Stratagene).

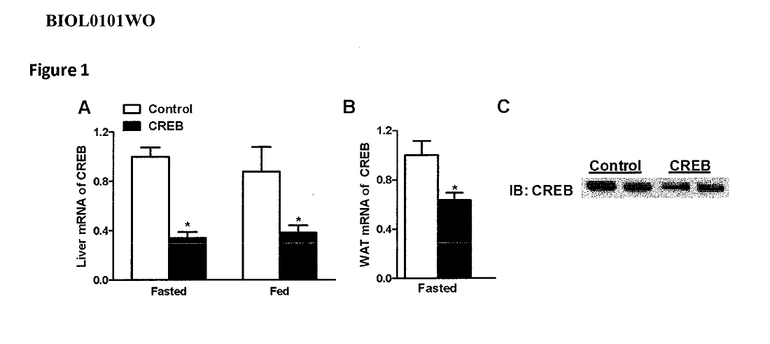

Liver mRNA of CREB expression was significantly reduced in both the fasted and fed rats in the CREB antisense oligonucleotide treated rats compared to the control rats. Treatment with ISIS 385915 resulted in 77% reduction in liver CREB mRNA levels in the fasted state and 64% in the fed state (FIG. 1A). WAT mRNA expression of CREB was also reduced in the CREB antisense oligonucleotide-treated, fasted rats compared to the control fasted rats. In WAT, CREB mRNA was reduced 44% in the fasted state (FIG. 1B).

### Protein Analysis

For western blot analysis, 20 mg of powdered tissue from the liver homogenate was dissolved in 200 μl of homogenization buffer and Laemmli sample buffer. After centrifugation for 15 minutes at 10,000 g, 40 μg of crude protein was then separated on a 4-12% gradient polyacrylamide gel in MOPS buffer system (Invitrogen). Subsequently, membranes were transferred to nitrocellulose membranes, and membranes were incubated in blocking buffer (5% milk) for 1 hour and immunoblotted with anti-CREB antibody (Cell signaling) overnight.

Immunoblots of CREB protein levels in isolated liver homogenates revealed a corresponding reduction of CREB protein levels compared to those with control antisense oligonucleotide treatment (FIG. 1C).

Together, the gene and protein expression studies reveal that antisense oligonucleotides can specifically inhibit CREB expression in fed and fasted tissues. Treatment of fasted and fed rats with an antisense inhibitor of CREB reduced both CREB mRNA and protein levels in both adipose and liver tissues. This indicates antisense inhibitors of CREB are candidate therapeutic agents for the treatment of disorders characterized by increased CREB expression or activity in adipose (such as adipogenesis and obesity) and liver tissues (such as hepatic steatosis, dyslipidemia, NAFLD and NASH) in both the fasted and fed states.

### Example 6

### Effects of Antisense Inhibition of CREB in a Rat Model of Type 2 Diabetes Mellitus (T2DM) on Metabolic Parameters

The high-fat diet-fed, Streptozotocin (STZ)-treated rat model provides a novel animal model for T2DM that simulates the human syndrome and is suitable for the testing of antidiabetic compounds for therapeutic use. This model of type 2 diabetes is a non-obese, outbred rat strain that replicates the natural history and metabolic characteristics of the human syndrome and is suitable for pharmaceutical research of therapeutic compounds (Reed et al, Metabolism 2000 49(11): 1390-4). Male Sprague-Dawley rats are fed normal chow (60% carbohydrate, 10% fat, 30% protein calories), or high-fat diet (26% carbohydrate, 59% fat, 15% protein calories, in which the major constitute is safflower oil) for 2 weeks and then injected with nicotinamide (175 mg/kg) by intraperitoneal injection followed by streptozotocin (STZ, 65 mg/kg) after 15 mins. Before Streptozotocin injections, high-fat diet fed rats had similar glucose concentrations to chow-fed rats, but significantly higher insulin, free fatty acid (FFA), and triglyceride (TG) concentrations. Streptozotocin injections increased glucose, insulin, FFA, and TG concentrations in high-fat diet-fed rats (Fat-fed/STZ rats) compared with chow-fed, STZ-injected rats. Fat-fed/STZ rats are not insulin deficient compared with normal chow-fed rats, but have hyperglycemia and a somewhat higher insulin response to an oral glucose challenge. In addition, insulin-stimulated adipocyte glucose clearance is reduced in fat-fed/STZ rats compared with both chow-fed and Chow-fed/STZ rats. Also, fat-fed/STZ rats are sensitive to the glucose lowering effects of metformin and troglitazone. This data shows that Fat-fed/STZ rats provide a novel animal model for type 2 diabetes that simulates the human syndrome, and is suitable for the testing of antidiabetic compounds.

To evaluate the effects of CREB antisense inhibition on metabolic parameters in both normal chow-fed rats and high fat-fed (T2DM) rats, the animals were treated with control antisense oligonucleotide (ISIS 141923) or CREB antisense oligonucleotide for 4 weeks and the following parameters were measured: body weight, WAT weight, plasma leptin, plasma adiponectin, ALT, total plasma cholesterol, HDL plasma cholesterol and plasma β-hydroxybutyrate. The results are shown in Table 5.

TABLE 5
Metabolic Parameters

| | Normal Diet | | T2D Rat Model | |
|---|---|---|---|---|
| | Control | CREB | Control | CREB |
| | ASO | ASO | ASO | ASO |
| Body Weight (g) | 349 | 342 | 356 | 340 |
| WAT Weight (g) | 2.4 | 2.2 | 4.1 | 2.8 |
| Plasma Leptin (ng/mL) | 1.3 | 1.1 | 2.3 | 1.0 |
| Plasma Adiponectin (ug/mL) | n.d. | n.d. | 4.4 | 3.7 |
| ALT (U/L) | n.d. | n.d. | 49 | 50 |
| Total Plasma Cholesterol | 56.0 | 41.8 | 46.0 | 23.3 |
| (mg/dL) | | | | |
| HDL Plasma Cholesterol | 21.6 | 16.9 | 21.7 | 9.7 |
| (mg/dL) | | | | |
| Plasma β-hydroxybutyrate | 1.23 | 0.94 | 1.61 | 1.41 |
| (mmol/L) | | | | |

n.d. = not determined

Elevated levels of plasma transaminases are often used clinically as potential indicators of liver damage. To evaluate the impact of ISIS 385915 on hepatic function of rats described, plasma concentrations of transaminases were measured using an automated clinical chemistry analyzer (Hitachi Olympus AU400e, Melville, N.Y.). Measurements of alanine transaminase (ALT) were taken at 0 weeks and after antisense oligonucleotide treatment. There was no increase in plasma ALT concentrations in rats dosed with CREB antisense oligonucleotide as compared with those treated with control antisense oligonucleotide.

In the normal chow-fed rats, there was a 25% decrease in total plasma cholesterol with CREB antisense oligonucleotide treatment. In the T2DM model, there was a 32% decrease in white adipose tissue weight, and a corresponding 56% decrease in fasting plasma leptin concentrations. It has been shown in previous studies that fasting serum leptin and insulin concentrations are highly correlated, and insulin sensitive subjects have lower leptin levels than insulin resistant subjects matched for fat mass. There was also a 49% decrease in total plasma cholesterol, and 12% decrease in plasma β-hydroxybutyrate, the latter indicating an improvement in diabetes by reduction of ketosis.

As shown in Table 5, plasma cholesterol and white adipose tissue weight were reduced after treatment with the CREB antisense oligonucleotide in the both T2DM models and the normal animal models, indicating that inhibition of CREB expression could have therapeutic benefit in subjects having cardiovascular disorders, like hypercholesterolemia, as well as related metabolic disorders, like obesity. Thus, antisense inhibitors of CREB could be candidate therapeutic agents for the treatment of conditions characterized by elevated cholesterol, or conditions of other related cardiovascular and metabolic disorders, such as obesity. Significantly, this data presents the ability to lower cholesterol and fat in vivo by specifically modulating CREB.

### Example 7

### Effect of Antisense Inhibition of CREB in a Rat Model of Type 2 Diabetes Mellitus (T2DM) on Plasma Insulin and Glucose Levels

Plasma glucose in rats treated as described in Example 6 was determined with a glucose oxidase method performed by a Beckman Glucose Analyzer II (Beckman Coulter). Plasma insulin and glucagon levels were determined using the LINCOplex system. The results are shown in FIG. 2, illustrating changes in fasting plasma glucose levels. In normal rats fed a standard rodent chow, CREB antisense oligonucleotide treatment had no effect on fasting plasma glucose concentration (FIG. 2A). However, treatment with the CREB antisense oligonucleotide in normal rats fed a standard rodent chow led to a 65% reduction in fasting plasma insulin concentrations (FIG. 2B). Thus, these animals were able to maintain normal glucose control despite a significant reduction in circulating insulin levels, indicating an improvement in insulin sensitivity.

CREB antisense oligonucleotide treatment in the T2DM rat model decreased fasting plasma glucose concentrations (FIG. 2A) and improved insulin sensitivity as exhibited by a 66% reduction in fasting plasma insulin concentrations (FIG. 2B).

Fasting plasma glucagon concentrations were similar in both antisense oligonucleotide treated groups and rat models (FIG. 2C), indicating that the lowering of glucose levels was a direct effect of antisense oligonucleotide treatment and not due to reduction in glucagon levels.

### Gluconeogenic Gene Expression Levels

The gluconeogenic enzymes, cytosolic phosphoenolpyruvate carboxykinase (PEPCK), mitochondrial PEPCK and the transcriptional co-activator peroxisomal proliferator activated receptor gamma coactivator-1-alpha (PGC-1α) mRNA levels were decreased by 43%, 55% and 54% respectively in the liver of the CREB antisense oligonucleotide groups (FIG. 2D). There was no observed difference in glucose-6-phosphatase (G6Pase) and hepatocyte nuclear factor 4α (HNF-4α).

The decreased expression of CREB mRNA led to decreased expression of the key gluconeogenic enzyme PEPCK which may partly explain the mechanism of the improved hepatic insulin sensitivity observed in the CREB antisense oligonucleotide treated rats.

In addition to reducing plasma glucose levels and improving insulin sensitivity, treatment with the CREB antisense oligonucleotide in the T2DM models showed a reduction in gluconeogenesis, through a reduction in mRNA expression of gluconeogenic genes. Gluconeogenesis is a major factor contributing to hyperglycemia in subjects with Type 2 diabetes. These results further indicate that inhibition of CREB expression shows therapeutic benefit in metabolic disorders, such as Type 2 diabetes, characterized by glucose and/or insulin dysregulation.

### Example 8

### Effect of Antisense Inhibition of CREB in the Rat T2DM Model—Levels on Plasma Triglycerides and Cholesterol

CREB antisense oligonucleotide, ISIS 385915 was tested for its ability to affect lipid metabolism in the rats with T2DM that received antisense oligonucleotide treatment, as described in Example 6. Triglycerides were extracted with the method of Bligh and Dyer (Bligh E G and Dyer W J 1959. Can J Biochem Physiol 37:911-917) and measured with the use of a commercially available triglyceride kit (DCL Triglyceride Reagent; Diagnostic Chemicals Ltd.). For cholesterol analysis, samples were pooled together and injected onto an Amersham Akta FPLC (Amersham Pharmacia Biotech) and eluted at a constant flow rate of 0.5 mL/min FPLC buffer (0.15M NaCl, 0.01M Na₂HPO₄, 0.1 mM EDTA, pH 7.5).

CREB antisense oligonucleotide treatment decreased plasma triglycerides by 24% as compared to control antisense oligonucleotide treatment (FIG. 3A). Total plasma cholesterol was also decreased in rats treated with CREB antisense oligonucleotide (Table 10). FPLC analysis demonstrated that the decrease in cholesterol was attributed to significant decreases in VLDL, LDL, and HDL cholesterol (FIG. 3B).

### Genes for Cholesterol Metabolism

CREB antisense oligonucleotide ISIS 385915 was tested for its ability to affect genes regulating cholesterol metabolism in the Streptozotocin (STZ)-treated rat models. To investigate the mechanism for the reduction in plasma cholesterol concentrations in CREB antisense oligonucleotide treated rats, rate of sterol synthesis was measured in primary rat hepatocytes. Results showed that there was no change with CREB antisense oligonucleotide treatment on the incorporation rate of [¹⁴C] acetic acid into cholesterol (FIG. 3C). However, RT-PCR analysis of genes from the hepatocytes of T2DM rats treated as described in Example 6 showed that genes relating to cholesterol metabolism, sterol regulatory element binding protein 2 (SREBP-2) and hydroxyl-methyl-glutaryl co-enzyme A (HMG-CoA) were both increased 50-60% after CREB antisense oligonucleotide treatment (FIG. 3D). The expression of the low density lipoprotein receptor (LDLR) and scavenger receptor class B type 1 (SR-B1) were unaltered with CREB antisense oligonucleotide treatment, suggesting that alterations in cholesterol uptake were not the cause of reduced plasma cholesterol concentrations (FIG. 4D).

### Bile Synthesis Rate

CREB antisense oligonucleotide ISIS 385915 was tested for its ability to affect the rate of bile synthesis. Feces collected during an overnight fast from rats described in Example 6 were homogenized at 20 mg feces in 500 μL of methanol and heated to reflux for one hour. Subsequently, the feces were rotated overnight, and the following morning a 100 μL aliquot was evaporated to dryness. The amount of bile acids present was measured using a commercially available kit (450-A Bile Acids; Trinity).

Fecal bile content was approximately three fold higher compared to the control antisense oligonucleotide treated rat (FIG. 3E). This suggests that increased cholesterol exported into fecal bile is one of the causes underlying the reduced plasma cholesterol concentration in the CREB antisense oligonucleotide treated rats.

The rate of bile acid synthesis was measured by quantifying the expression of cholesterol 7alpha-hydroxylase (Cyp7A1) by RT-PCR analysis. CREB antisense oligonucleotide treatment resulted in an approximately two fold increase in Cyp7A1 expression over the control group (FIG. 3D), which indicates an increase in the rate of bile synthesis.

These studies show a significant reduction in plasma cholesterol and triglyceride levels after treatment with the CREB antisense oligonucleotide. These studies indicate that inhibition of CREB expression can provide therapeutic benefit in subjects having metabolic disorders, like obesity and Type 2 diabetes, with the added benefit of preventing or reducing associated dyslipidemia that can also lead to increased risk for, cardiovascular disorders characterized by hypercholesterolemia and hypertriglyceridemia. Thus, antisense inhibitors of CREB are candidate therapeutic agents for the treatment of conditions characterized by hypercholesterolemia, and hypertriglyceridemia, or conditions of dyslipidemia associated with NAFLD, Type 2 diabetes, obesity and other metabolic disorders.

### Example 9

### Effects of Antisense Inhibition of CREB in the Rat T2DM Model—Hepatic Lipid Content

CREB antisense oligonucleotide, ISIS 385915 was tested for its ability to affect hepatic lipid content in rats. Primary rat hepatocytes treated with either antisense oligonucleotides to CREB or control antisense oligonucleotide described in Example 6, were incubated in a medium either containing [³H] glycerol or [¹⁴C] oleic acid to determine the rate of lipid synthesis and oxidation. The purification of diacylglycerols (DAGs) and long-chain fatty acyl-CoAs from the liver was performed according to the method of Bligh and Dyer (Bligh E G and Dyer W J 1959. Can Biochem Physiol 37:911-917). After purification, fatty acyl-CoA fractions were dissolved in methanol/H₂O (1:1, v/v) and subjected to liquid chromatography/mass spectrometry/mass spectrometry (LC/MS/MS) analysis. A turbo ion spray source was interfaced with an API 3000 tandem mass spectrometer (Applied Biosystems) in conjunction with 2 PerkinElmer 200 Series micro pumps and a 200 Series autosampler (PerkinElmer). Total DAG content was expressed as the sum of subject species.

As seen in FIGS. 4A, 4B, and 4C, intrahepatic lipids (triglycerides, diacylglycerols and long chain CoAs) were lowered with CREB antisense oligonucleotide treatment compared to the control antisense oligonucleotide treated T2DM rats. Liver triglycerides decreased from 14 mg/g liver to 6 mg/g liver, and liver diacylglycerol decreased from 500 nmol/g to 300 nmol/g; and long chain acyl CoAs decreased from 65 nmol/g liver to 50 nmol/g. CREB antisense oligonucleotide had no effect on the rate of triglyceride synthesis (FIG. 4D) but showed a 30% increased rate of fatty acid oxidation (FIG. 4E).

### Genes for Mitochondrial β-Oxidation

In accordance with the result above, ISIS 385915 was tested for its ability to regulate the genes involved in mitochondrial β-oxidation, as described in Example 6. The genes were assessed by RT-PCR in vivo.

The mRNA levels of long-chain acyl-CoA dehydrogenase (LCAD) and medium-chain acyl-CoA dehydrogenase (MCAD) were both increased 119% and 82% respectively in the CREB antisense oligonucleotide-treated group (FIG. 4F). These results support the increased fat oxidation seen in primary rat hepatocytes.

As shown in these studies, hepatic triglycerides, diacylglycerols and long chain CoAs were lowered when treated with the CREB antisense inhibitor, indicating that inhibition of CREB expression can have therapeutic benefit in subjects having disorders or conditions characterized by elevated liver triglycerides, diacylglycerols and long chain CoAs including metabolic disorders such as NAFLD and NASH. These studies also support the use of CREB inhibitors for more general metabolic disorders including Type 2 diabetes and obesity because NAFLD associated insulin resistance is one of the factors contributing to hyperglycemia in Type 2 diabetes.

### Example 10

### Effects of Antisense Inhibition of CREB in the Rat T2DM Model—Hepatic Insulin Sensitivity and Insulin Signaling

### Insulin Sensitivity

Effect of decreased CREB expression by ISIS 385915 on hepatic and peripheral insulin sensitivity was assessed using hyperinsulinemic-euglycemic clamps. Seven to nine days prior to the clamp, catheters were inserted into the right internal jugular vein extending to the right atrium and the left carotid artery extending into the aortic arch. Subsequently, the rats were fasted for 24 hours, and then infused with 99% [6, 6-²H] glucose (1.1 mg/kg prime, 0.1 mg/kg) infusion to assess basal glucose turnover. Ensuing the basal period, the hyperinsulinemic-euglycemic clamp was conducted for 140 minutes with a primed/continuous infusion of insulin (400 mU/kg prime over 5 minutes, 4 mU/kg per minute infusion subsequently) and a variable infusion of 20% dextrose spiked with approximately 2.5% [6,6-²H]glucose to maintain euglycemia.

Basal hepatic glucose production and insulin-stimulated peripheral glucose uptake rates were similar in both normal and T2DM rat models and in both control and CREB antisense oligonucleotide treated groups (FIGS. 5A and 5B). In contrast, there was a significant increase in hepatic insulin sensitivity in the CREB antisense oligonucleotide treated T2DM rat model over the control antisense oligonucleotide, as measured by a 45% inhibition of hepatic glucose production during the clamp (FIG. 5C).

### Genes Involved in Insulin Signaling

CREB antisense oligonucleotide treatment also reduced hepatic diacylglycerol (DAG) content, which is associated with a decrease in PKCε membrane translocation in the liver (FIG. 5D). These changes were associated with improved insulin signaling, as measured by increased insulin-stimulated Akt2 activity (FIG. 5E). There was a 41% reduction in the mRNA expression of tribbles homolog 3 (TRB3) (FIG. 5F), a putative Akt2 inhibitor, which may also contribute to the improved insulin activation of Akt2 in the livers of the CREB antisense oligonucleotide treated rats. In contrast to previous studies that used dominant negative polypeptide A-CREB or Ad CREB RNAi to knock down the expression of CREB in primary rat hepatocytes and found that it resulted in reduced expression of insulin receptor substrate 2(IRS-2) (Canettieri, G. et al. 2005. Cell Metab 2:331-338.), the studies herein, showed no difference in the mRNA expression of insulin receptor substrate 2 (IRS-2) expression between the CREB antisense oligonucleotide and control antisense oligonucleotide treated rats (FIG. 5G).

The study by Canettieri found that inhibition of CREB activity decreased hepatic insulin sensitivity by decreasing expression of IRS-2. In contrast to this finding, antisense oligonucleotide reduction of CREB expression resulted in no significant differences in IRS-2 mRNA expression in liver and significant improvement in hepatic insulin responsiveness. Thus, in contrast to the previous studies of Canettieri et al., supra, these results unexpectedly showed increased hepatic insulin responsiveness and therefore decreased insulin resistance upon inhibition of CREB expression. Without being bound to a particular theory, it is believed that at least three specific mechanisms lead to improved hepatic insulin sensitivity achieved by antisense reduction of CREB. First, CREB antisense oligonucleotide lowered intrahepatic DAG concentrations, with associated decreases in PKCε activation. Previous studies have demonstrated that PKCε binds to the insulin receptor leading to reduced insulin receptor-β kinase activity. Moreover, decreasing expression of PKCε, using a similar antisense oligonucleotide approach protected rats from fat-induced hepatic insulin resistance (Samuel, V. T. et. al., 2007, J Clin Invest 117:739-745). Second, decreased expression of CREB resulted in decreased expression of PGC-1α and PPARα and subsequently TRB3 expression. TRB3 inhibits Akt2 activity leading to reduced insulin signaling and previous studies have shown that knock down of hepatic TRB3 improved hepatic insulin responsiveness (Koo, S. H., et. al., 2004, Nat Med 10:530-534.). Lastly, decreased expression of CREB led to decreased expression of the key gluconeogenic enzyme PEPCK. It is likely that these mechanisms also contribute to the improved hepatic insulin sensitivity observed in the CREB antisense oligonucleotide treated rats during the hyperinsulinemic-euglycemic clamp.

Together, these results indicate that antisense inhibitors of CREB are candidate therapeutic agents for the treatment of conditions characterized by elevated glucose levels, increased insulin resistance (particularly hepatic insulin resistance), cholesterol levels, lipids levels, including triglycerides levels, and other related cardiovascular and metabolic disorders.

CREB-specific inhibitors, such as antisense oligonucleotides to CREB, are candidate therapeutic agents for the treatment of both metabolic and cardiovascular disorders, such as Type 2 diabetes, obesity and hypercholesterolemia, and any combination thereof.

The in vivo studies provided herein are carried out in well characterized models of disease that are recognized by those of skill in the art as being predictive of therapeutic results in other animals, including humans.

### Example 11

### Effect of Antisense Inhibition of CREB in Zucker Diabetic Fatty (ZDF) Rat Model

### Treatment

The leptin receptor deficient Zucker diabetic fatty (ZDF) rat is another useful model for the investigation of type 2 diabetes. Diabetes develops spontaneously in these male rats at ages 8-10 weeks, and is associated with hyperphagia, polyuria, polydipsia, and impaired weight gain, symptoms which parallel the clinical symptoms of diabetes (Phillips M S, et al., 1996, Nat Genet. 13, 18-19).

ZDF/GmiCrl-fa/fa (ZDF) male rats were purchased from Charles River Laboratories (Wilmington, Mass., USA). Treatment groups (4-6 mice each) were as follows: a group treated with ISIS 385915, a group treated with control oligonucleotide ISIS 141923, and a control group treated with saline. Oligonucleotide or saline was administered intraperitoneally twice weekly, for a period of 7 weeks; oligonucleotide doses were 25 mg/kg. After the treatment period, whole liver was collected for RNA analysis.

### RNA Analysis

Liver and white adipose tissue RNA was isolated for real-time PCR analysis of CREB. The results are presented in Table 6. Liver mRNA of CREB expression was significantly reduced in the CREB antisense oligonucleotide treated rats compared to the control rats. Treatment with ISIS 385915 resulted in 39% reduction in liver CREB mRNA levels and 18% in WAT.

TABLE 6
Percent inhibition of CREB mRNA in the WAT and Liver

| | liver | WAT |
|---|---|---|
| Saline | 0 | 0 |
| ISIS 385915 | 39 | 18 |

### Effect on Plasma Glucose and Insulin Levels

Plasma glucose levels in rats treated as described above, was determined using an automated clinical chemistry analyzer (Olympus AU400e, Melville, N.Y.). Plasma insulin levels were determined using an ELISA kit from ALPCO Diagnostics. The results, as shown in Tables 8 and 9, illustrate changes in fed and fasted plasma glucose and insulin levels at weeks 0, 2, 3, 5, 6 and 6.5.

TABLE 7
Effect of antisense oligonucleotides
on plasma glucose levels (mg/dL)

| | | | | | Week | Week |
|---|---|---|---|---|---|---|
| | Week | Week | Week | Week | 6 4 hr | 6.5 over- |
| | 0 fed | 2 fed | 3 fed | 5 fed | fast | night fast |
| Saline | 250 | 494 | 451 | 462 | 501 | 353 |
| ISIS 141923 | 259 | 471 | 471 | 371 | 443 | 321 |
| ISIS 385915 | 252 | 370 | 262 | 259 | 308 | 175 |

TABLE 8
Effects of antisense oligonucleotides on plasma insulin levels (ng/mL)

| | Week 5 | Week 6.5 |
|---|---|---|
| | fed | fasted |
| Saline | 5 | 3 |
| ISIS 141923 | 7 | 3 |
| ISIS 385915 | 15 | 8 |

In ZDF rats, treatment with CREB antisense oligonucleotide ISIS 385915 led to a 44% reduction in fed plasma glucose levels after 5 weeks, and 50% reduction in fasted glucose levels after 6.5 weeks. Insulin levels for the same time period did not significantly change. This finding further confirms the insulin sensitizing and glucose lowering effects after treatment with a CREB antisense oligonucleotide.

### Effect on Glucose Tolerance

Glucose tolerance was measured via the oral glucose tolerance test (OGTT) at week 4. The rats were fasted overnight and then an oral administration of glucose at 0.75 mg/kg was given. Blood glucose levels were measured before the glucose challenge and at different time points after challenge up to 120 min.

As presented in Table 9, in CREB antisense oligonucleotides treated rats, initial glucose levels were lower and the increase in glucose levels during the OGTT assay was less than in the saline control. Therefore, antisense oligonucleotide treated mice had better glucose tolerance as compared to the saline controls.

TABLE 9
Effect of antisense oligonucleotides
on glucose levels (mg/dL) during OGTT

| | 0 min | 15 min | 30 min | 60 min | 90 min | 120 min |
|---|---|---|---|---|---|---|
| Saline | 185 | 334 | 407 | 419 | 448 | 371 |
| ISIS 141923 | 158 | 300 | 361 | 363 | 362 | 327 |
| ISIS 385915 | 130 | 214 | 246 | 288 | 287 | 257 |

### Effect on Triglyceride and Lipid Levels

ISIS 385915 was tested for its ability to affect lipid metabolism in ZDF rats that received antisense oligonucleotide treatment, as described. Blood was obtained and analyzed for cholesterol and plasma lipids. Measurements were taken at 0 weeks, 2 weeks, 3 weeks, 4 weeks, 5 weeks, 6 weeks and 6.5 weeks and the results are presented in Tables 10 and 11.

The data indicates that administration of ISIS 385915, relative to saline treated control or antisense control (ISIS 141923), effectively reduced cholesterol levels (Table 10). At 6.5 weeks, cholesterol levels were reduced by over 23% (from 184 mg/dL in saline group to 141 mg/dL) in CREB Antisense oligonucleotide treated groups. Inhibition of CREB, as shown herein, is an effective means for decreasing plasma cholesterol in this model. These results confirm that inhibitors of CREB can be useful for the treatment of hypercholesterolemia associated with dysregulated metabolic states such as insulin resistance and obesity.

TABLE 10
Effect of antisense oligonucleotides
on plasma cholesterol levels (mg/dL)

| | | | | | Week | Week |
|---|---|---|---|---|---|---|
| | Week | Week | Week | Week | 6 4 hr | 6.5 o/n |
| | 0 fed | 2 fed | 3 fed | 5 fed | fasted | fasted |
| Saline | 124 | 174 | 188 | 165 | 164 | 184 |
| ISIS 141923 | 113 | 162 | 188 | 174 | 164 | 190 |
| ISIS 385915 | 120 | 138 | 140 | 119 | 115 | 141 |

TABLE 11
Effect of antisense oligonucleotides
on plasma triglyceride levels (mg/dL)

| | | | | | Week | Week |
|---|---|---|---|---|---|---|
| | Week | Week | Week | Week | 6 4 hr | 6.5 o/n |
| | 0 fed | 2 fed | 3 fed | 5 fed | fasted | fasted |
| Saline | 472 | 1070 | 879 | 641 | 429 | 515 |
| ISIS 141923 | 376 | 589 | 577 | 284 | 344 | 439 |
| ISIS 385915 | 430 | 655 | 711 | 510 | 375 | 435 |

### Example 12

### Antisense Inhibition of Human CREB: A549 Cells

Antisense oligonucleotides targeted to a CREB nucleic acid were tested for their effects on CREB mRNA in vitro. Cultured A549 cells at a density of 6000 cells per well in a 96-well plate were treated with 150 nM of antisense oligonucleotide. The A549 cell line is a well characterized human cell line known to express CREB and is therefore useful for assessing the effectiveness of antisense oligonucleotides for inhibiting CREB. After a treatment period of approximately 4 hours and a recovery period of approximately 24 hours, RNA was isolated from the cells and CREB mRNA levels were measured by quantitative real-time PCR, as described herein. CREB mRNA levels were adjusted according to total RNA content as measured by RIBOGREEN®. Results are presented as percent inhibition of CREB, relative to untreated control cells. Antisense oligonucleotides targeted to SEQ ID NO: 1 (GENBANK Accession No. NM_{—}134442.2) are listed in Table 12.

The chimeric antisense oligonucleotides in Table 12 were designed as 5-10-5 MOE gapmers. The gapmers are 20 nucleotides in length, wherein the central gap segment is comprised of 10 2′-deoxynucleotides and is flanked on both sides (in the 5′ and 3′ directions) by wings comprising 5 nucleotides each. Each nucleotide in the 5′ wing segment and each nucleotide in the 3′ wing segment has a 2′-MOE modification. The internucleoside linkages throughout each gapmer are phosphorothioate (P-S) linkages. All cytidine residues throughout each gapmer are 5-methylcytidines. “Target Start site” indicates the 5′-most nucleotide which the antisense oligonucleotide is targeted on the indicated GENBANK Accession No. NM_{—}134442.2 (SEQ ID NO: 1). “Target Stop site” indicates the 3′-most nucleotide which the antisense oligonucleotide is targeted on the indicated GENBANK Accession No. NM_{—}134442.2 (SEQ ID NO: 1).

TABLE 12
Antisense inhibition of CREB in human cells (Cell type A549)

| | Target Start | Target Stop | | % | SEQ |
|---|---|---|---|---|---|
| ISIS No. | site | Site | Sequence (5′ to 3′) | inhibition | ID NO |
| 102631 | 77 | 96 | ACACACCGCGTCAAACTACA | 54 | 95 |
| 102634 | 137 | 156 | CCGTCACTGCTTTCGTTCAC | 31 | 96 |
| 102639 | 163 | 182 | TTTAGTTACCGGTGGTACAA | 56 | 97 |
| 102643 | 165 | 184 | CATTTAGTTACCGGTGGTAC | 55 | 98 |
| 102647 | 167 | 186 | GTCATTTAGTTACCGGTGGT | 71 | 99 |
| 102650 | 169 | 188 | TGGTCATTTAGTTACCGGTG | 66 | 100 |
| 102654 | 171 | 190 | CATGGTCATTTAGTTACCGG | 59 | 101 |
| 102658 | 173 | 192 | TCCATGGTCATTTAGTTACC | 67 | 102 |
| 102662 | 175 | 194 | ATTCCATGGTCATTTAGTTA | 41 | 103 |
| 102666 | 177 | 196 | AGATTCCATGGTCATTTAGT | 55 | 104 |
| 102670 | 179 | 198 | CCAGATTCCATGGTCATTTA | 79 | 105 |
| 102674 | 181 | 200 | CTCCAGATTCCATGGTCATT | 66 | 106 |
| 102678 | 183 | 202 | GGCTCCAGATTCCATGGTCA | 57 | 107 |
| 102682 | 207 | 226 | TGCATCTCCACTCTGCTGGT | 41 | 108 |
| 102685 | 247 | 266 | CTTGAACTGTCATTTGTTGG | 56 | 109 |
| 102689 | 324 | 343 | AGTTACGGTGGGAGCAGATG | 82 | 110 |
| 102693 | 348 | 367 | CTGCCCATTGGGCAGCTGTA | 24 | 91 |
| 102697 | 392 | 411 | ACTGATGGCTGGGCCGCCTG | 76 | 111 |
| 102702 | 655 | 674 | TGGCAGGTGCTGAAGTCTCC | 66 | 112 |
| 102705 | 705 | 724 | CTGTCCACTGCTAGTTTGGT | 61 | 113 |
| 102709 | 773 | 792 | AGGCCCTGTACCCCATCGGT | 56 | 114 |
| 102713 | 795 | 814 | ATTGGTCATGGTTAATGTTT | 53 | 115 |
| 102717 | 838 | 857 | GTGCATACTGTAGAATGGTA | 65 | 116 |
| 102721 | 994 | 1013 | GCTGTGTAGGAAGTGCTGGG | 35 | 117 |
| 102726 | 1023 | 1042 | CTCTCTCTTTCGTGCTGCTT | 59 | 118 |
| 102729 | 1179 | 1198 | TTTGTGGCAGTAAAGGTCCT | 69 | 92 |
| 102734 | 1196 | 1215 | ATCCCAAATTAATCTGATTT | 69 | 93 |
| 102737 | 1243 | 1262 | TTGTGGCCAAGCCAGTCCAT | 62 | 119 |
| 102740 | 1322 | 1341 | CTGTAGTTGCTTTCAGGCAG | 41 | 120 |
| 102743 | 1407 | 1426 | GGCGTTGAAAATTTCTTGAG | 56 | 121 |
| 102746 | 1506 | 1525 | TTTTCTTTTCCTCATTTCTC | 55 | 122 |
| 102749 | 1612 | 1631 | TTATGCATGCGGCCCACACA | 42 | 123 |
| 102752 | 1740 | 1759 | TCCTTCAATACCATGCTAAA | 20 | 124 |
| 102754 | 1837 | 1856 | AGCTGTATTAGTACAGAATG | 39 | 125 |
| 102756 | 1915 | 1934 | GGTTACTTCTTTTAATGTAT | 27 | 126 |
| 102758 | 1979 | 1998 | GCTTTGTACTTTTATTTACT | 41 | 127 |
| 102760 | 2159 | 2178 | GTGGTATGTAAGTGCAATGG | 47 | 128 |
| 102762 | 2278 | 2297 | TTCTCTGTTAAATTGTTAAT | 9 | 129 |
| 102765 | 2389 | 2408 | TGCAGTACAGCAGTCATTCA | 45 | 130 |
| 102767 | 2512 | 2531 | CAGGAATTAAAATTATAAAA | 8 | 131 |

Antisense oligonucleotides with the following ISIS Nos exhibited at least 50% inhibition of CREB mRNA levels: 102713, 102631, 102643, 102666, 102746, 102639, 102685, 102709, 102743, 102678, 102654, 102726, 102705, 102737, 102717, 102650, 102674, 102702, 102658, 102729, 102734, 102647, 102697, 102670, and 102689. The target segments to which these antisense oligonucleotides are targeted are active target segments. The target regions to which these antisense oligonucleotides are targeted are active target regions.

Antisense oligonucleotides with the following ISIS Nos exhibited at least 60% inhibition of CREB mRNA levels: 102705, 102737, 102717, 102650, 102674, 102702, 102658, 102729, 102734, 102647, 102697, 102670, and 102689. The target segments to which these antisense oligonucleotides are targeted are active target segments. The target regions to which these antisense oligonucleotides are targeted are active target regions.

Antisense oligonucleotides with the following ISIS Nos exhibited at least 65% inhibition of CREB mRNA levels: 102717, 102650, 102674, 102702, 102658, 102729, 102734, 102647, 102697, 102670, and 102689. The target segments to which these antisense oligonucleotides are targeted are active target segments. The target regions to which these antisense oligonucleotides are targeted are active target regions.

Antisense oligonucleotides with the following ISIS Nos exhibited at least 70% inhibition of CREB mRNA levels: 102647, 102697, 102670, and 102689. The target segments to which these antisense oligonucleotides are targeted are active target segments. The target regions to which these antisense oligonucleotides are targeted are active target regions.

ISIS Nos 102697, 102670, and 102689 each exhibited at least 75% inhibition of CREB mRNA levels. The target segments to which these antisense oligonucleotides are targeted are active target segments. The target regions to which these antisense oligonucleotides are targeted are active target regions.

As provided herein, the antisense oligonucleotide sequences in Table 12 are designed to the human CREB mRNA sequence in regions that are homologous to the rat sequence. It is expected that the relative inhibition levels of antisense oligonucleotides in vitro will be consistent across cell types which express an mRNA with which the antisense oligonucleotides are specifically hybridizable. This is also expected in vivo regarding cells to which the antisense oligonucleotides distribute.

The in vivo studies provided herein are carried out in well characterized models of disease that are recognized by those of skill in the art as being predictive of therapeutic results in other animals, including humans.

### Example 13

### Antisense Inhibition of Murine CREB: b.END Cells

Antisense oligonucleotides targeted to a CREB nucleic acid were tested for their effects on CREB mRNA in vitro. Cultured b.END cells at a density of 4,000 cells per well were transfected using lipofectin reagent with 75 nM antisense oligonucleotide for 4 hours. After a recovery period of approximately 24 hours, RNA was isolated from the cells and CREB mRNA levels were measured by quantitative real-time PCR. CREB mRNA levels were adjusted according to total RNA content, as measured by RIBOGREEN®. Results are presented as percent inhibition of CREB, relative to untreated control cells.

The chimeric antisense oligonucleotides in Table 13 were designed as 5-10-5 MOE gapmers. The gapmers are 20 nucleotides in length, wherein the central gap segment is comprised of 10 2′-deoxynucleotides and is flanked on both sides (in the 5′ and 3′ directions) by wings comprising 5 nucleotides each. Each nucleotide in the 5′ wing segment and each nucleotide in the 3′ wing segment has a 2′-MOE modification. The internucleoside linkages throughout each gapmer are phosphorothioate (P-S) linkages. All cytidine residues throughout each gapmer are 5-methylcytidines. “Mouse target start site” indicates the 5′-most nucleotide to which the gapmer is targeted. “Mouse target stop site” indicates the 3′-most nucleotide to which the gapmer is targeted. Each gapmer listed in Table 13 is targeted to SEQ ID NO: 7 (GENBANK Accession No. NM_{—}133828.1), or SEQ ID NO: 8 (GENBANK Accession No. NM_{—}009952.1), or SEQ ID NO: 9 (GENBANK Accession No. NT_{—}039170.1_truncated from nucleotides_{—}42407947 to 42484927), or SEQ ID NO: 10 (GENBANK Accession No. X92497.1), SEQ ID NO: D (GENBANK Accession No. U46027.1) or SEQ ID NO: 11 (GENBANK Accession No. AK042595.1).

The mouse oligonucleotides also show cross reactivity, (i.e. ≦3 base mismatch) with the human CREB mRNA (GENBANK Accession No. NM_{—}134442.2), incorporated herein as SEQ ID NO: 1. “Human Target Start Site” indicates the 5′-most nucleotide in the human mRNA to which the antisense oligonucleotide is targeted. “Human Target Stop Site” indicates the 3′-most nucleotide in the human mRNA to which the antisense oligonucleotide is targeted. ‘Mismatches’ indicates the number of nucleobases by which the mouse oligonucleotide is mismatched with the human gene sequence. “n/a” indicates that the mouse oligonucleotide may have greater than 3 base mismatch with the human gene sequence.

TABLE 13
Inhibition of murine CREB mRNA levels by chimeric antisense
oligonucleotides having 5-10-5 MOE wings and deoxy gap

| | Mouse | Mouse | | | Human | Human | | | |
|---|---|---|---|---|---|---|---|---|---|
| | Target | Target | | | Target | Target | | Mouse | SEQ |
| ISIS | Start | Stop | Sequence | % | Start | Stop | Mis- | Target | ID |
| No. | Site | Site | (5′ to 3′) | inhibition | Site | Site | matches | Sequence | NO |
| 342352 | 45 | 64 | GTCAAA | 84 | 68 | 87 | 0 | NM_133828.1 | 132 |
| | | | CTACAC | | | | | | |
| | | | CTCCGC | | | | | | |
| | | | CG | | | | | | |
| 342353 | 55 | 74 | AACACA | 42 | 78 | 97 | 0 | NM_133828.1 | 133 |
| | | | CCGCGT | | | | | | |
| | | | CAAACT | | | | | | |
| | | | AC | | | | | | |
| 342354 | 71 | 90 | ATTCTC | 69 | 94 | 113 | 0 | NM_133828.1 | 134 |
| | | | TCCCCC | | | | | | |
| | | | ACGTAA | | | | | | |
| | | | CA | | | | | | |
| 342355 | 81 | 100 | CTGGAG | 51 | 104 | 123 | 0 | NM_133828.1 | 135 |
| | | | TTTTAT | | | | | | |
| | | | TCTCTC | | | | | | |
| | | | CC | | | | | | |
| 342356 | 116 | 135 | CTCCGT | 65 | 139 | 158 | 0 | NM_133828.1 | 136 |
| | | | CACTGC | | | | | | |
| | | | TTTCGT | | | | | | |
| | | | TC | | | | | | |
| 342357 | 121 | 140 | AGCTCC | 58 | 144 | 163 | 0 | NM_133828.1 | 137 |
| | | | TCCGTC | | | | | | |
| | | | ACTGCT | | | | | | |
| | | | TT | | | | | | |
| 342358 | 133 | 152 | ACCGGT | 78 | 156 | 175 | 0 | NM_133828.1 | 138 |
| | | | GGTACA | | | | | | |
| | | | AGCTCC | | | | | | |
| | | | TC | | | | | | |
| 342359 | 141 | 160 | ATTTAG | 39 | 164 | 183 | 0 | NM_133828.1 | 139 |
| | | | TTACCG | | | | | | |
| | | | GTGGTA | | | | | | |
| | | | CA | | | | | | |
| 342360 | 183 | 202 | GCATCT | 62 | 206 | 225 | 0 | NM_133828.1 | 140 |
| | | | CCACTC | | | | | | |
| | | | TGCTGG | | | | | | |
| | | | TT | | | | | | |
| 342361 | 225 | 244 | GCTTGA | 32 | 248 | 267 | 0 | NM_133828.1 | 141 |
| | | | ACTGTC | | | | | | |
| | | | ATTTGT | | | | | | |
| | | | TG | | | | | | |
| 342362 | 230 | 249 | GCTGGG | 13 | 253 | 272 | 0 | NM_133828.1 | 142 |
| | | | CTTGAA | | | | | | |
| | | | CTGTCA | | | | | | |
| | | | TT | | | | | | |
| 342363 | 235 | 254 | CTGTGG | 61 | 258 | 277 | 0 | NM_133828.1 | 143 |
| | | | CTGGGC | | | | | | |
| | | | TTGAAC | | | | | | |
| | | | TG | | | | | | |
| 342364 | 240 | 259 | GCAATC | 80 | 263 | 282 | 0 | NM_133828.1 | 144 |
| | | | TGTGGC | | | | | | |
| | | | TGGGCT | | | | | | |
| | | | TG | | | | | | |
| 342365 | 245 | 264 | ATGTGG | 62 | 268 | 287 | 0 | NM_133828.1 | 145 |
| | | | CAATCT | | | | | | |
| | | | GTGGCT | | | | | | |
| | | | GG | | | | | | |
| 342366 | 250 | 269 | GGCTAA | 68 | 273 | 292 | 0 | NM_133828.1 | 146 |
| | | | TGTGGC | | | | | | |
| | | | AATCTG | | | | | | |
| | | | TG | | | | | | |
| 342367 | 255 | 274 | ACCTGG | 66 | 278 | 297 | 0 | NM_133828.1 | 13 |
| | | | GCTAAT | | | | | | |
| | | | GTGGCA | | | | | | |
| | | | AT | | | | | | |
| 342368 | 289 | 308 | AGCAG | 73 | 312 | 331 | 0 | NM_133828.1 | 147 |
| | | | ATGATG | | | | | | |
| | | | TTGCAT | | | | | | |
| | | | GAG | | | | | | |
| 342369 | 327 | 346 | GTCTGC | 20 | 350 | 369 | 0 | NM_133828.1 | 14 |
| | | | CCATTG | | | | | | |
| | | | GGCAGC | | | | | | |
| | | | TG | | | | | | |
| 342370 | 393 | 412 | GTTTGG | 61 | 416 | 435 | 0 | NM_133828.1 | 15 |
| | | | ACTTGT | | | | | | |
| | | | GGAGA | | | | | | |
| | | | CTG | | | | | | |
| 342371 | 398 | 417 | GAACTG | 56 | 421 | 440 | 0 | NM_133828.1 | 148 |
| | | | TTTGGA | | | | | | |
| | | | CTTGTG | | | | | | |
| | | | GA | | | | | | |
| 342372 | 416 | 435 | CTGCAA | 54 | 481 | 500 | 1 | NM_133828.1 | 16 |
| | | | TAGTTG | | | | | | |
| | | | AAATCT | | | | | | |
| | | | GA | | | | | | |
| 342373 | 421 | 440 | ACTTTC | 53 | 486 | 505 | 0 | NM_133828.1 | 17 |
| | | | TGCAAT | | | | | | |
| | | | AGTTGA | | | | | | |
| | | | AA | | | | | | |
| 342374 | 426 | 445 | TCTTCA | 48 | 491 | 510 | 0 | NM_133828.1 | 149 |
| | | | CTTTCT | | | | | | |
| | | | GCAATA | | | | | | |
| | | | GT | | | | | | |
| 342375 | 431 | 450 | GTGAAT | 74 | 496 | 515 | 0 | NM_133828.1 | 150 |
| | | | CTTCAC | | | | | | |
| | | | TTTCTG | | | | | | |
| | | | CA | | | | | | |
| 342376 | 436 | 455 | CTCCTG | 54 | 501 | 520 | 0 | NM_133828.1 | 151 |
| | | | TGAATC | | | | | | |
| | | | TTCACT | | | | | | |
| | | | TT | | | | | | |
| 342377 | 498 | 517 | TAGGAA | 48 | 563 | 582 | 0 | NM_133828.1 | 152 |
| | | | GGCCTC | | | | | | |
| | | | CTTGAA | | | | | | |
| | | | AG | | | | | | |
| 342378 | 503 | 522 | TCCTGT | 69 | 568 | 587 | 0 | NM_133828.1 | 153 |
| | | | AGGAA | | | | | | |
| | | | GGCCTC | | | | | | |
| | | | CTT | | | | | | |
| 342379 | 529 | 548 | ATCAGA | 30 | 594 | 613 | 0 | NM_133828.1 | 18 |
| | | | AGATAA | | | | | | |
| | | | GTCATT | | | | | | |
| | | | CA | | | | | | |
| 342380 | 534 | 553 | GGTGCA | 53 | 599 | 618 | 0 | NM_133828.1 | 19 |
| | | | TCAGAA | | | | | | |
| | | | GATAAG | | | | | | |
| | | | TC | | | | | | |
| 342381 | 603 | 622 | GTTACA | 61 | 668 | 687 | 0 | NM_133828.1 | 20 |
| | | | GTGGTG | | | | | | |
| | | | ATGGCA | | | | | | |
| | | | GG | | | | | | |
| 342382 | 636 | 655 | CCACTG | 56 | 701 | 720 | 0 | NM_133828.1 | 21 |
| | | | CTAGTT | | | | | | |
| | | | TGGTAA | | | | | | |
| | | | AT | | | | | | |
| 342383 | 663 | 682 | CCTCCC | 53 | 728 | 747 | 0 | NM_133828.1 | 153 |
| | | | TGGGTA | | | | | | |
| | | | ATGGCA | | | | | | |
| | | | AT | | | | | | |
| 342384 | 668 | 687 | TTGCTC | 76 | 733 | 752 | 0 | NM_133828.1 | 154 |
| | | | CTCCCT | | | | | | |
| | | | GGGTAA | | | | | | |
| | | | TG | | | | | | |
| 342385 | 673 | 692 | CTGTAT | 71 | 738 | 757 | 0 | NM_133828.1 | 155 |
| | | | TGCTCC | | | | | | |
| | | | TCCCTG | | | | | | |
| | | | GG | | | | | | |
| 342386 | 678 | 697 | GCCAGC | 58 | 743 | 762 | 0 | NM_133828.1 | 156 |
| | | | TGTATT | | | | | | |
| | | | GCTCCT | | | | | | |
| | | | CC | | | | | | |
| 342387 | 683 | 702 | TGTTAG | 62 | 748 | 767 | 0 | NM_133828.1 | 157 |
| | | | CCAGCT | | | | | | |
| | | | GTATTG | | | | | | |
| | | | CT | | | | | | |
| 342388 | 688 | 707 | ACCATT | 67 | 753 | 772 | 0 | NM_133828.1 | 158 |
| | | | GTTAGC | | | | | | |
| | | | CAGCTG | | | | | | |
| | | | TA | | | | | | |
| 342389 | 711 | 730 | TGCAGG | 66 | 776 | 795 | 0 | NM_133828.1 | 159 |
| | | | CCCTGT | | | | | | |
| | | | ACCCCA | | | | | | |
| | | | TC | | | | | | |
| 342390 | 735 | 754 | GCTGCA | 66 | 800 | 819 | 0 | NM_133828.1 | 22 |
| | | | TTGGTC | | | | | | |
| | | | ATGGTT | | | | | | |
| | | | AA | | | | | | |
| 342391 | 756 | 775 | GTAGTA | 55 | 821 | 840 | 0 | NM_133828.1 | 160 |
| | | | CCCGGC | | | | | | |
| | | | TGAGTG | | | | | | |
| | | | GC | | | | | | |
| 342392 | 761 | 780 | GAATGG | 61 | 826 | 845 | 0 | NM_133828.1 | 161 |
| | | | TAGTAC | | | | | | |
| | | | CCGGCT | | | | | | |
| | | | GA | | | | | | |
| 342393 | 817 | 836 | AACTTG | 64 | 882 | 901 | 0 | NM_133828.1 | 162 |
| | | | GTTGCT | | | | | | |
| | | | GGGCAC | | | | | | |
| | | | TA | | | | | | |
| 342394 | 823 | 842 | AACAAC | 45 | 888 | 907 | 0 | NM_133828.1 | 23 |
| | | | AACTTG | | | | | | |
| | | | GTTGCT | | | | | | |
| | | | GG | | | | | | |
| 342395 | 828 | 847 | GCTTGA | 8 | 893 | 912 | 0 | NM_133828.1 | 24 |
| | | | ACAACA | | | | | | |
| | | | ACTTGG | | | | | | |
| | | | TT | | | | | | |
| 342396 | 833 | 852 | AGGCA | 43 | 898 | 917 | 0 | NM_133828.1 | 25 |
| | | | GCTTGA | | | | | | |
| | | | ACAACA | | | | | | |
| | | | ACT | | | | | | |
| 342397 | 931 | 950 | AGGCTG | 38 | 996 | 1015 | 0 | NM_133828.1 | 163 |
| | | | TGTAGG | | | | | | |
| | | | AAGTGC | | | | | | |
| | | | TG | | | | | | |
| 342398 | 936 | 955 | TCAGCA | 38 | 1001 | 1020 | 0 | NM_133828.1 | 164 |
| | | | GGCTGT | | | | | | |
| | | | GTAGGA | | | | | | |
| | | | AG | | | | | | |
| 342399 | 941 | 960 | CTTCTT | 44 | 1006 | 1025 | 0 | NM_133828.1 | 165 |
| | | | CAGCAG | | | | | | |
| | | | GCTGTG | | | | | | |
| | | | TA | | | | | | |
| 342400 | 946 | 965 | TGCTGC | 38 | 1011 | 1030 | 0 | NM_133828.1 | 166 |
| | | | TTCTTC | | | | | | |
| | | | AGCAG | | | | | | |
| | | | GCT | | | | | | |
| 342401 | 969 | 988 | ATTAGA | 38 | 1034 | 1053 | 0 | NM_133828.1 | 167 |
| | | | CGGACC | | | | | | |
| | | | TCTCTC | | | | | | |
| | | | TT | | | | | | |
| 342402 | 979 | 998 | CCTGTT | 35 | 1044 | 1063 | 0 | NM_133828.1 | 168 |
| | | | CTTCAT | | | | | | |
| | | | TAGACG | | | | | | |
| | | | GA | | | | | | |
| 342403 | 1053 | 1072 | TCAAGC | 44 | 1118 | 1137 | 0 | NM_133828.1 | 169 |
| | | | ACTGCC | | | | | | |
| | | | ACTCTG | | | | | | |
| | | | TT | | | | | | |
| 342404 | 1091 | 1110 | GTGCTT | 52 | 1156 | 1175 | 0 | NM_133828.1 | 170 |
| | | | TTAGCT | | | | | | |
| | | | CCTCAA | | | | | | |
| | | | TC | | | | | | |
| 342405 | 1101 | 1120 | AGGTCC | 74 | 1166 | 1185 | 0 | NM_133828.1 | 26 |
| | | | TTAAGT | | | | | | |
| | | | GCTTTT | | | | | | |
| | | | AG | | | | | | |
| 342406 | 1128 | 1147 | CCAAAT | 47 | 1193 | 1212 | 0 | NM_133828.1 | 171 |
| | | | TAATCT | | | | | | |
| | | | GATTTG | | | | | | |
| | | | TG | | | | | | |
| 342407 | 1134 | 1153 | TAAATC | 23 | 1199 | 1218 | 0 | NM_133828.1 | 27 |
| | | | CCAAAT | | | | | | |
| | | | TAATCT | | | | | | |
| | | | GA | | | | | | |
| 342408 | 1242 | 1261 | CAGGCA | 59 | 1309 | 1328 | 0 | NM_133828.1 | 172 |
| | | | GTTTTG | | | | | | |
| | | | CGCATA | | | | | | |
| | | | GA | | | | | | |
| 342409 | 1263 | 1282 | ATGAAA | 64 | 1330 | 1349 | 0 | NM_133828.1 | 173 |
| | | | TTCTGT | | | | | | |
| | | | AGTTGC | | | | | | |
| | | | TT | | | | | | |
| 342410 | 1290 | 1309 | CACAGT | 9 | 1357 | 1376 | 0 | NM_133828.1 | 174 |
| | | | TTAATG | | | | | | |
| | | | CAAAA | | | | | | |
| | | | GCA | | | | | | |
| 342411 | 1300 | 1319 | TTGGAA | 59 | 1367 | 1386 | 0 | NM_133828.1 | 175 |
| | | | CATTCA | | | | | | |
| | | | CAGTTT | | | | | | |
| | | | AA | | | | | | |
| 342412 | 1356 | 1375 | TCTCTT | 50 | 1423 | 1442 | 0 | NM_133828.1 | 176 |
| | | | CATGAT | | | | | | |
| | | | TCCTGG | | | | | | |
| | | | CG | | | | | | |
| 342413 | 1605 | 1624 | ACACTT | 60 | 1639 | 1658 | 0 | NM_133828.1 | 177 |
| | | | CTTCAT | | | | | | |
| | | | TGCACC | | | | | | |
| | | | TT | | | | | | |
| 342414 | 1805 | 1824 | AAGAA | 28 | 1863 | 1882 | 0 | NM_133828.1 | 28 |
| | | | GCAACA | | | | | | |
| | | | ACTGCC | | | | | | |
| | | | CTA | | | | | | |
| 342415 | 353 | 372 | TACAGG | 28 | 433 | 452 | 0 | NM_009952.1 | 178 |
| | | | AAGACT | | | | | | |
| | | | GAACTG | | | | | | |
| | | | TT | | | | | | |
| 342416 | 365 | 384 | TTTTTA | 11 | 445 | 464 | 0 | NM_009952.1 | 29 |
| | | | AGTCCT | | | | | | |
| | | | TACAGG | | | | | | |
| | | | AA | | | | | | |
| 342417 | 395 | 414 | TAGTTG | 27 | 475 | 494 | 1 | NM_009952.1 | 179 |
| | | | AAATCT | | | | | | |
| | | | GAGTTC | | | | | | |
| | | | CG | | | | | | |
| 342418 | 37898 | 37917 | TATTAC | 31 | n/a | n/a | n/a | NT_039170.1_ | 180 |
| | | | TCACTG | | | | | TRUNC_ | |
| | | | TACTGC | | | | | 42407947_42 | |
| | | | CC | | | | | 484927 | |
| 342419 | 41674 | 41693 | ATGAAT | 0 | n/a | n/a | n/a | NT_039170.1_ | 180 |
| | | | TTTATT | | | | | TRUNC_ | |
| | | | GTTACA | | | | | 42407947_42 | |
| | | | AG | | | | | 484927 | |
| 342420 | 42334 | 42353 | AGCTCT | 62 | n/a | n/a | n/a | NT_039170.1_ | 181 |
| | | | ATATTC | | | | | TRUNC_ | |
| | | | CTTTTA | | | | | 42407947_42 | |
| | | | AA | | | | | 484927 | |
| 342421 | 185 | 204 | GCATGG | 44 | n/a | n/a | n/a | X92497.1 | 182 |
| | | | ATACCG | | | | | | |
| | | | GTGGTA | | | | | | |
| | | | CA | | | | | | |
| 342422 | 91 | 110 | TTTAGT | 47 | n/a | n/a | n/a | X92497.1 | 183 |
| | | | TACCAA | | | | | | |
| | | | CACTCC | | | | | | |
| | | | GC | | | | | | |
| 342423 | 469 | 488 | CCTGAG | 32 | n/a | n/a | n/a | U46027.1 | 184 |
| | | | GCAGTG | | | | | | |
| | | | TACTGC | | | | | | |
| | | | CC | | | | | | |
| 342424 | 76434 | 76453 | AACTGT | 9 | n/a | n/a | n/a | NT_039170.1_ | 185 |
| | | | CCAGAC | | | | | TRUNC_ | |
| | | | AGAAG | | | | | 42407947_42 | |
| | | | GAT | | | | | 484927 | |
| 342425 | 877 | 896 | CTCTAT | 22 | n/a | n/a | n/a | AK042595.1 | 186 |
| | | | ATTCCT | | | | | | |
| | | | TGAACA | | | | | | |
| | | | AC | | | | | | |
| 342426 | 42697 | 42716 | ACAAA | 50 | n/a | n/a | n/a | NT_039170.1_ | 187 |
| | | | AGATGT | | | | | TRUNC_ | |
| | | | TCTACT | | | | | 42407947_42 | |
| | | | TGG | | | | | 484927 | |

### Example 14

### Dose-Dependent Antisense Inhibition of Murine CREB mRNA in MHT Cells

Antisense oligonucleotides from Example 13 (see Table 13), exhibiting in vitro inhibition of murine CREB, were tested at various doses in MHT cells. The MHT cell line was created by immortalizing mouse hepatocytes with SV40 large T antigen (Yamamoto et al., 2003. Hepatology. 37: 528-533). Cells were plated at a density of 10,000 cells per well and transfected using lipofectin reagent with 0.465 nM, 0.9375 nM, 1.875 nM, 3.75 nM, 7.5 nM, 15 nM, 30 nM and 60 nM concentrations of antisense oligonucleotide, as specified in Table 14. After a treatment period of approximately 16 hours, RNA was isolated from the cells and CREB mRNA levels were measured by quantitative real-time PCR. CREB mRNA levels were adjusted according to total RNA content, as measured by RIBOGREEN®. Results are presented as percent inhibition of CREB, relative to untreated control cells. As illustrated in Table 14, CREB mRNA levels were reduced in a dose-dependent manner in antisense oligonucleotide treated cells.

TABLE 14
Dose-dependent antisense inhibition of murine CREB in MHT
cells via transfection of oligonucleotides with lipofectin

| ISIS | 60 | 30 | 15 | 7.5 | 3.75 | 1.875 | 0.9375 | 0.465 |
|---|---|---|---|---|---|---|---|---|
| No. | nM | nM | nM | nM | nM | nM | nM | nM |
| 342352 | 91 | 89 | 83 | 75 | 67 | 46 | 39 | 31 |
| 342364 | 84 | 77 | 67 | 57 | 39 | 23 | 31 | 25 |

### Example 15

### Dose-Dependent Antisense Inhibition of Murine CREB mRNA in Primary Mouse Hepatocytes

Antisense oligonucleotides from Example 13 (see Table 13), exhibiting in vitro inhibition of murine CREB, were tested at various doses in primary mouse hepatocytes. Cells were plated at a density of 10,000 cells per well and transfected using lipofectin reagent with 1.56 nM, 3.125 nM, 6.25 nM, 12.5 nM, 25 nM, 50 nM, 100 nM and 200 nM concentrations of antisense oligonucleotide, as specified in Table 15. After a treatment period of approximately 16 hours, RNA was isolated from the cells and CREB mRNA levels were measured by quantitative real-time PCR. CREB mRNA levels were adjusted according to total RNA content, as measured by RIBOGREEN®. Results are presented as percent inhibition of CREB, relative to untreated control cells. As illustrated in Table 15, CREB mRNA levels were reduced in a dose-dependent manner in antisense oligonucleotide treated cells.

TABLE 15
Dose-dependent antisense inhibition of murine CREB in primary mouse
hepatocytes via transfection of oligonucleotides with lipofectin

| ISIS | 200 | 100 | 50 | 25 | 12.5 | 6.25 | 3.125 | 1.56 |
|---|---|---|---|---|---|---|---|---|
| No. | nM | nM | nM | nM | nM | nM | nM | nM |
| 342352 | 87 | 77 | 66 | 50 | 49 | 21 | 19 | 17 |
| 342364 | 64 | 63 | 37 | 53 | 30 | 6 | 3 | 9 |

### Example 16

### Antisense Inhibition of CREB In Vivo

Antisense oligonucleotides targeted to murine CREB mRNA (GENBANK Accession No. NM_{—}133828.1, incorporated herein as SEQ ID NO: 7) showing statistically significant dose-dependent inhibition from the in vitro study were evaluated in vivo. C57BL/6 mice were treated with ISIS 342352 (GTCAAACTACACCTCCGCCG, target start site 45, incorporated herein as SEQ ID NO: 132).

### Treatment

C57BL/6 mice were injected with 50 mg/kg of ISIS 342352 twice a week for 3 weeks. A control group of mice was injected with phosphate buffered saline (PBS) twice a week for 3 weeks. Mice were sacrificed after the treatment period, the liver was harvested for RNA analysis and plasma was collected for transaminase analysis.

### RNA Analysis

RNA was extracted from liver tissue for real-time PCR analysis of CREB. As shown in Table 16, ISIS 342352 achieved significant reduction of murine CREB over the saline control. Results are presented as percent inhibition of CREB, relative to the saline control.

TABLE 16
Percent inhibition of murine CREB mRNA
in C57BL/6 mice compared to the saline control

| | % inhibition |
|---|---|
| Saline | 0 |
| ISIS 342352 | 67 |

### Measurement of Plasma Transaminase Levels

To evaluate the impact of ISIS 342352 on hepatic function of mice described above, plasma concentrations of transaminases were measured using an automated clinical chemistry analyzer (Hitachi Olympus AU400e, Melville, N.Y.). Measurements of alanine transaminase (ALT) and aspartate transaminase (AST) were taken after antisense oligonucleotide treatment, and presented in Table 17.

TABLE 17
Effect of antisense inhibition on plasma transaminases (U/L)

| | ALT | ALT | AST | AST |
|---|---|---|---|---|
| | week 0 | week 2 | week 0 | week 2 |
| Saline | 27 | 30 | 54 | 53 |
| ISIS 342352 | 25 | 37 | 44 | 50 |

Together, the studies reveal that ISIS 342352 can specifically inhibit CREB gene expression without significant hepatic toxicity.

Thus, antisense inhibitors of CREB are candidate therapeutic agents for the treatment of disorders characterized by increased CREB expression or activity in liver tissues (such as hepatic steatosis, NAFLD and NASH).

### Example 17

### Effects of Antisense Inhibition of CREB in the ob/ob Mouse Model of Obesity

### Treatment

Leptin is a hormone produced by fat that regulates appetite. Deficiency of this hormone in both humans and in non-human animals, leads to obesity. ob/ob mice have a mutation in the leptin gene which results in obesity and hyperglycemia. As such, these mice are a useful model for the investigation of obesity and diabetes and related conditions provided herein. These mice models are also useful for testing compounds, compositions and methods designed to treat, prevent or ameliorate such conditions.

The effects of antisense inhibition of CREB were investigated in the ob/ob mouse model of obesity. Male ob/ob (C57/BL/6J-Lepr ob) mice at 7 weeks of age were purchased from Jackson Laboratories (Bar Harbor, Me.). During a 1 week acclimation period and throughout the study, mice were fed a diet with a fat content of 10-15% (Labdiets #5015, Purina, St. Louis, Mo.). The mice were injected with 25 mg/kg of ISIS 342352 twice a week for 4 weeks. A control group of mice was injected with phosphate buffered saline (PBS) twice a week for 4 weeks. Mice were sacrificed after the treatment period, liver was harvested and plasma was collected.

Measurement of CREB mRNA Expression

ISIS 342352 inhibited CREB mRNA expression in the liver by 89% and in the white adipose tissue by 46% compared to saline control mice as shown in Table 18.

TABLE 18
Percent inhibition of CREB mRNA in the WAT and Liver

| | Liver | WAT |
|---|---|---|
| Saline | 0 | 0 |
| ISIS 342352 | 89 | 46 |

### Effect on Plasma Glucose Levels

Plasma glucose in mice treated, as described was determined using an automated clinical chemistry analyzer (Olympus AU400e, Melville, N.Y.). The results are shown in Table 19, illustrating changes in fed and fasted plasma glucose levels at weeks 0, 2, 3 and 4.

TABLE 19
Effect of antisense oligonucleotides on plasma glucose levels (mg/dL)

| | Week 0 | Week 2 | Week 3 | Week 4 |
|---|---|---|---|---|
| | fed | fed | fasted | fed |
| Saline | 397 | 491 | 243 | 481 |
| ISIS 342352 | 416 | 393 | 234 | 337 |

In ob/ob mice fed with normal rodent chow, treatment with CREB antisense oligonucleotides led to a 30% reduction in fed plasma glucose levels after 4 weeks. This data indicates that reduction of CREB expression caused a reduction in glucose levels, indicating an improvement in the diabetic state.

### Effect on Triglyceride Levels

ISIS 342352 was tested for its ability to affect lipid metabolism in ob/ob mice that received antisense oligonucleotide treatment, as described. Blood samples were collected from the mice at various time points and analyzed, and on week 4, the mice were sacrificed and blood and liver tissue were obtained and analyzed for triglyceride content. The data in Table 20 demonstrates 24% and 29% reduction of triglyceride levels in the blood and liver of ISIS 342352-treated mice at week 4 compared to the saline controls. The data presented in Table 23 confirms that CREB antisense inhibition results in decrease in triglyceride levels in different tissues of this mice model, indicating the effectiveness of CREB antisense oligonucleotides as lipid lowering agents, particularly in models of dyslipidemia.

TABLE 20
Effect of antisense oligonucleotides on plasma triglyceride levels (mg/dL)

| | Week 0 | Week 2 | Week 3 | Week 4 |
|---|---|---|---|---|
| Saline-plasma | 231 | 212 | 82 | 175 |
| Saline liver | n.d. | n.d. | n.d. | 176 |
| ISIS 342352-plasma | 230 | 171 | 86 | 133 |
| ISIS 342352-liver | n.d. | n.d. | n.d. | 125 |

This data indicates that CREB antisense oligonucleotides may be used for treatment of conditions like hyperlipidemia and hyperglycemia and other disorders related to elevated glucose levels or triglyceride level, for example, atherosclerosis, obesity, and diabetes.

### Example 18

### Antisense Inhibition of CREB in the Diet-Induced Model of Obesity (DIO)

The C57BL/6 mouse strain is reported to be susceptible to hyperlipidemia-induced atherosclerotic plaque formation and is accepted as a model for diet-induced obesity for human (C. Gallou-Kabani et al, Obesity (2007) 15, 1996-2005). To induce hyperlipidemia, these mice were fed a high-fat diet and used in the following studies to evaluate the effects of ISIS 342352 in a model of diet-induced obesity.

### Treatment

Male C57BL/6 mice at 7 weeks of age were placed on a high-fat diet containing 58% calories from fat (Research Diet D12492, Research Diets Inc., New Brunswick, N.J.) for 3 months. The mice were divided into four treatment groups. The first group received subcutaneous injections of ISIS 342352 at a dose of 25 mg/kg twice per week for 6 weeks. The second group received subcutaneous injections of ISIS 141923 at a dose of 25 mg/kg twice per week for 6 weeks. The third control group received subcutaneous injections of saline twice weekly for 6 weeks. Saline-injected lean mice also served as a control group.

Inhibition of CREB mRNA

At the end of the six week treatment period, the mice were sacrificed and CREB mRNA expression was measured in liver by real-time PCR.

The results shown in Table 21 are expressed as percent expression relative to high-fat saline-treated mice. The data shows that the antisense oligonucleotide inhibited CREB expression compared to both the controls.

TABLE 21
Percent mRNA expression
in ASO treated mice relative to high-fat saline control

| | % |
|---|---|
| | inhibition |
| High-fat saline | 0 |
| control | |
| ISIS 342352 | 63 |
| Lean saline | 0 |
| Control | |

### Effect on Food Intake Levels

The accumulated food intake of the animals was monitored over 6 weeks. The results are shown in Table 22 and indicate that neither the controls nor the CREB antisense oligonucleotide had a significant impact on amount of food consumed by the mice.

TABLE 22
Effect of antisense oligonucleotides on food intake in DIO mice (g)

| | Week 1 | Week 2 | Week 3 | Week 4 | Week 5 | Week 6 |
|---|---|---|---|---|---|---|
| saline | 55 | 108 | 164 | 217 | 276 | 333 |
| ISIS 141923 | 49 | 98 | 149 | 196 | 248 | 296 |
| ISIS 342352 | 49 | 99 | 147 | 193 | 243 | 289 |

### Effect on Total Body Weight

Treatment in mice with ISIS 342352 resulted in relatively no change in body weight over a period of 6 weeks compared to high-fat saline control animals. The weekly measurements of body weights of the various treatment groups are shown in Table 23. ‘n.d.’ implies the data was not recorded for that week.

TABLE 23
Effect of antisense oligonucleotides on total body weight (g)

| | Week 0 | Week 1 | Week 2 | Week 3 | Week 4 | Week 6 |
|---|---|---|---|---|---|---|
| High-fat | 38 | 38 | 39 | 39 | 39 | 40 |
| saline control | | | | | | |
| ISIS 141923 | 39 | 39 | 40 | 41 | 40 | 41 |
| ISIS 342352 | 39 | 40 | 41 | 40 | 40 | 40 |
| Lean saline | 29 | 29 | 29 | n.d | n.d | 29 |
| control | | | | | | |

### Effect on Serum Cholesterol Levels

Blood was obtained and analyzed for serum cholesterol. Measurements were taken at 0 week, 3 weeks, 5 weeks and 6 weeks.

The data indicate that administration of ISIS 342352, relative to the saline treated control, effectively reduces cholesterol levels from increasing (Table 24) At 6 weeks, total cholesterol levels were reduced by about 19% (from 285 mg/dL to 231 mg/dL) compared to the saline control. HDL cholesterol levels were not significantly reduced. This data shows that ASO inhibition of CREB is an effective means for decreasing cholesterol in a diet-induced obesity model. Therefore, inhibitors of CREB are useful for the treatment of hyperlipidemia associated with dysregulated metabolic states such as obesity. Since antisense oligonucleotide treatment did not have any effect on body weight and food intake, this implies that the reduction in cholesterol levels is not due to lack of food consumption but secondary to inhibition of CREB expression.

TABLE 24
Effect of antisense oligonucleotides on plasma cholesterol levels (mg/dL)

| | | | 5 weeks | |
|---|---|---|---|---|
| | 0 week | 3 week | 6-hr | 6 week |
| | fed | fed | fasting | fed |
| saline | 215 | 237 | 262 | 285 |
| ISIS 141923 | 222 | 249 | 273 | 266 |
| ISIS 342352 | 219 | 232 | 241 | 231 |
| Lean control | 98 | 98 | 103 | 109 |

### Effect on Insulin Levels

Plasma insulin levels were determined using an ELISA kit from ALPCO Diagnostics. The results are shown in Table 25, illustrating changes in fed and fasted plasma insulin levels at weeks 0, 3 and 5.

Treatment with ISIS 342352 led to a 41% reduction respectively in fasted insulin levels after 5 weeks. This data indicates that reduction of CREB caused an improvement in insulin sensitivity.

TABLE 25
Effect of antisense oligonucleotides on plasma insulin levels (ng/mL)

| | 0 | 3 | |
|---|---|---|---|
| | week | weeks | 5 weeks |
| | fed | fed | fasted |
| saline | 5.7 | 4.9 | 4.9 |
| ISIS 141923 | 5.8 | 5.7 | 3.9 |
| ISIS 342352 | 5.9 | 2.8 | 2.9 |
| Lean control | 1.6 | 1.0 | 1.3 |

### Effect on Insulin Sensitivity

Insulin sensitivity in mice was measured via the insulin tolerance test (ITT). The ISIS 342352-treated and saline-treated mice were fasted for 4 hours and insulin was injected at 0.7 U/kg. The sensitivity of the mice to insulin was measured via measurements of plasma glucose levels.

As presented in Table 26, significantly lower glucose levels were observed in ISIS 342352 treated mice at the beginning of the assay as well as during the entire period of ITT compared to the saline controls. This finding confirms that CREB inhibition by treatment with CREB antisense oligonucleotide demonstrated an improvement in insulin sensitivity.

TABLE 26
Effect of antisense oligonucleotides
on blood glucose levels (mg/dL) during ITT

| | 0 min | 20 min | 40 min | 70 min |
|---|---|---|---|---|
| saline | 210 | 97 | 63 | 44 |
| ISIS 141923 | 207 | 104 | 59 | 36 |
| ISIS 342352 | 163 | 73 | 51 | 33 |
| Lean control | 194 | 102 | 81 | 65 |

### Effect on Glucose Tolerance

Glucose tolerance in mice was measured via the intraperitoneal glucose tolerance test (IPGTT). The mice were fasted overnight and then an intraperitoneal injection of glucose at 0.75 g/kg was given. Blood glucose levels were measured before the glucose challenge and at different time points after challenge up to 120 min.

As presented in Table 27, significantly lower glucose levels were observed in ISIS 342352 treated mice during the beginning of the study as well as the entire period of GTT (for example, at 120 min, ISIS 342352-treated: 207 mg/dL vs. saline: 233 mg/dL). Thus, the mice treated with ISIS 342352 are able to tolerate exogenous glucose better than the control. This finding confirms the decrease in plasma glucose and increase in glucose tolerance after treatment with CREB antisense oligonucleotide.

TABLE 27
Effect of antisense oligonucleotides
on glucose levels (mg/dL) during IPGTT

| | 0 min | 30 min | 60 min | 90 min | 120 min |
|---|---|---|---|---|---|
| saline | 163 | 308 | 285 | 227 | 233 |
| ISIS 141923 | 198 | 325 | 301 | 263 | 235 |
| ISIS 342352 | 179 | 281 | 246 | 215 | 207 |
| Lean control | 165 | 243 | 203 | 189 | 182 |

Further confirming, as provided herein, the present invention provides CREB-specific modulators that modulate or inhibit CREB expression, activity, or processing. Such agents are candidate therapeutic agents for the treatment of both metabolic and cardiovascular disorders, such as Type 2 diabetes, obesity and hypercholesterolemia, or any combination thereof.

The in vivo studies provided herein are carried out in well characterized models of disease that are recognized by those of skill in the art as being predictive of therapeutic results in other animals, including humans.

### Other Embodiments

The detailed description set-forth above is provided to aid those skilled in the art in practicing the present invention. However, the invention described and claimed herein, is not to be limited in scope by the specific embodiments disclosed herein because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which does not depart from the spirit or scope of the present inventive discovery. Such modifications are also intended to fall within the scope of the appended claims.

### REFERENCES CITED

All publications, patents, patent applications and other references cited in this application are incorporated herein, by reference in their entirety for all purposes to the same extent as if each subject publication, patent, patent application or other reference was specifically and subjectively indicated to be incorporated by reference in its entirety for all purposes. Citation of a reference herein, shall not be construed as an admission that such is prior art to the present invention.

1. A method of treating, preventing, or ameliorating a metabolic or cardiovascular disease in an animal, comprising administering to the animal having a therapeutically effective amount of a CREB inhibitor, wherein the metabolic or cardiovascular disease is treated, prevented or ameliorated in the animal.

2. The method of claim 1, wherein the metabolic or cardiovascular disease is obesity, diabetes, atherosclerosis, dyslipidemia, coronary heart disease, non-alcoholic fatty liver disease (NAFLD), hyperfattyacidemia or metabolic syndrome, or a combination thereof.

3. (canceled)

4. The method of claim 3, wherein the disease the dyslipidemia is hyperlipidemia.

5. The method of claim 4, wherein the hyperlipidemia is hypercholesterolemia, hypertriglyceridemia, or both hypercholesterolemia and hypertriglyceridemia.

6. The method of claim 2, wherein the NAFLD is hepatic steatosis or steatohepatitis.

7. The method of claim 2, wherein the diabetes is type 2 diabetes or type 2 diabetes with dyslipidemia.

8. The method of claim 1, wherein the administering results in a reduction of triglyceride levels, cholesterol levels; insulin resistance; glucose levels, body weight, body fat, adipose tissue mass, or any combination thereof.

9. (canceled)

10. The method of claim 1, wherein the administering results in improved insulin sensitivity.

11. (canceled)

12. A method of decreasing triglyceride levels, cholesterol levels, glucose levels, insulin resistance, body weight, body fat content or any combination thereof in a human by administering a CREB inhibitor.

13. The method of claim 12, wherein the CREB inhibitor is any of the group consisting of a nucleic acid, a peptide, or an antibody inhibitor.

14. The method of claim 12, wherein the CREB inhibitor is a nucleic acid.

15. The method of claim 14, wherein the nucleic acid is a modified oligonucleotide.

16. The method of claim 15, wherein the modified oligonucleotide consists of 12 to 30 linked nucleosides.

17. The method of claim 16, wherein said modified oligonucleotide is a single-stranded oligonucleotide.

18. The method of claim 17, wherein the nucleobase sequence of the modified oligonucleotide is 100% complementary to human CREB.

19. The method of claim 17, wherein at least one internucleoside linkage is a modified internucleoside linkage.

20. The method of claim 19, wherein each internucleoside linkage is a phosphorothioate internucleoside linkage.

21. The method of claim 17, wherein at least one nucleoside contains a modified sugar.

22. The method of claim 21, wherein the modified sugar comprises a 2′-O-methoxyethyl sugar moiety.

23. The method of claim 21, wherein the modified sugar is a bicyclic nucleic acid sugar moiety.

24. The method of claim 17, wherein at least one nucleoside comprises a modified nucleobase.

25. The method of claim 23, wherein each of the at least one bicyclic nucleic acid sugar moiety comprises a 4′-CH(CH3)-O-2′ bridge.

26. The method of claim 21, comprising at least one tetrahydropyran modified nucleoside wherein a tetrahydropyran ring replaces the furanose ring.

27. The method of claim 26, wherein each of the at least one tetrahydropyran modified nucleoside has the structure: 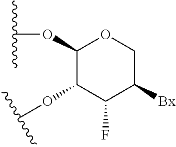
wherein Bx is an optionally protected heterocyclic base moiety.

28. The method of claim 17, wherein the modified oligonucleotide consists of 10 to 30 linked nucleosides having a nucleobase sequence comprising at least 10 contiguous nucleobases of a nucleobase sequence recited in SEQ ID NOs: 13 to 187.

29. The method of claim 1, wherein the administering comprises parenteral administration.

30. The method of claim 29, wherein the parenteral administration comprises subcutaneous or intravenous administration.

31. The method of claim 1, comprising co-administering a CREB inhibitor and at least one additional therapy.

32. The method of claim 31, wherein the CREB inhibitor and additional therapy, are administered concomitantly.

33. The method of claim 31, wherein the CREB inhibitor is administered and the additional therapy are administered in the same formulation.

34.-53. (canceled)

54. A method comprising identifying a animal having a metabolic or cardiovascular disease and administering to said animal a therapeutically effect amount of a composition comprising a modified oligonucleotide consisting of 12 to 30 linked nucleosides having a nucleobase sequence complementary to SEQ ID NO: 1, as measured over the entirety of said modified oligonucleotide.

55. The method of claim 24, wherein the modified nucleobase is a 5-methylcytosine.

56. The method of claims 17, wherein the modified oligonucleotide comprises:
a gap segment consisting of linked deoxynucleotides;
a 5′ wing segment consisting of linked nucleosides;
a 3′ wing segment consisting of linked nucleosides;
wherein the gap segment is positioned between eh 5′ wing segment and the 3′ wing segment and wherein each nucleoside of each wing segment comprise a modified sugar.

57. The method of claim 56, wherein the oligonucleotide comprises:
a. a gap segment consisting of ten linked deoxynucleosides;
b. a 5′ wing segment consisting of five linked nucleosides;
c. a 3′ wing segment consisting of five linked nucleosides;
wherein the gap segment is positioned between the 5′ wing segment and the 3′ wing segment, wherein each nucleoside of each wing segment comprises a 2′-O-methoxyethyl sugar, wherein each internucleoside linkage of said modified oligonucleotide is a phosphorothioate linkage, and wherein each cytosine in said modified oligonucleotide is a 5′-methylcytosine.

58. The method of claim 1, wherein the animal is a human.
